(19) United States
(12) Patent Application Publication
(10) Pub. No.: US 2009/0069288 A1
(43) Pub. Date: Mar. 12, 2009

(54) Novel therapeutic compounds

(76) Inventors: Eric C. Breinlinger, Charlton, MA (US); Kevin P. Cusack, Holden, MA (US); Adrian D. Hobson, Shrewsbury, MA (US); Bin Li, Ashland, MA (US); Thomas D. Gordon, Medway, MA (US); Robert H. Stoffel, Harvard, MA (US); Grier A. Wallace, Sterling, MA (US); Pintipa Grongsaard, Shrewsbury, MA (US); Lu Wang, Northborough, MA (US); Lu Wang, Worcester, MA (US)

Correspondence Address: ABBOTT BIORESEARCH, 100 RESEARCH DRIVE, WORCESTER, MA, 01605-4314, US

(21) Appl. No.: 12/218,364

(22) Filed: Jul. 15, 2008

Related U.S. Application Data
(60) Provisional application No. 60/959,631, filed on Jul. 16, 2007.

(51) Int. Cl. A61K 31/4196; C07D 249/10; C07D 403/12; C07D 261/14; A61K 31/42; A61K 31/415; C07D 231/14; A61P 35/00

(52) U.S. Cl. 514/210.18; 548/266.8; 514/383; 548/245; 514/380; 514/406; 548/374.1

(57) ABSTRACT

Disclosed herein are novel compounds of Formula (I),

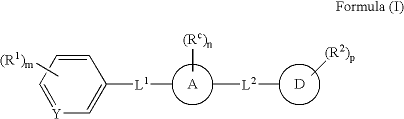

wherein the variables are defined as herein. The compounds of Formula (I) are useful as kinase inhibitors and as such would be useful in treating certain conditions and diseases, especially inflammatory conditions and diseases as well as proliferative disorders such as cancer.

### RELATED APPLICATIONS

This application claims priority to and the benefit of U.S. Provisional Application No. 60/959,631, filed Jul. 16, 2007.

### BACKGROUND OF THE INVENTION

Sphingosine-1-phosphate (SIP) is part of sphingomyelin biosynthetic pathway and is known to affect multiple biological processes. S1P is formed through phosphorylation of sphingosine by sphingosine kinases (SK1 and SK2) and it is degraded through cleavage by sphingosine lyase to form palmitaldehyde and phosphoethanolamine or through dephosphorylation by phospholipid phosphatases. It is present at high levels (˜500 nM) in serum and it is found in most tissues. It can be synthesized in a wide variety of cells in response to several stimuli, which include cytokines, growth factors and G protein-coupled receptor (GPCR) ligands. The GPCRs that bind S1P (currently know as the S1P receptors S1P1-5), couple through pertusis toxin sensitive (Gi) pathways as well as pertusis toxin insensitive pathways to stimulate a variety of processes. The individual receptors of the SIP family are both tissue and response specific and so are attractive as therapeutic targets.

S1P evokes many responses from cells and tissues. In particular, S1P has been shown to be an agonist at all five GPCRs, S1P1 (Edg-1), S1P2 (Edg-5), S1P3 (Edg-3), S1P4 (Edg-6) and S1P5 (Edg-8). The action of S1P at the S1P receptors has been linked to resistance to apoptosis, changes in cellular morphology, cell migration, growth, differentiation, cell division, angiogenesis and modulation of the immune system via alterations of lymphocyte trafficking. Therefore, S1P receptors are targets for therapy of, e.g., neoplastic diseases, autoimmune disorders and tissue rejection in transplantation. These receptors also share 50-55% amino acid identity with three other lysophospholipid receptors, LPA1, LPA2, and LPA3 of the structurally related lysophosphatidic acid (LPA).

GPCRs are excellent drug targets with numerous examples of marketed drugs across multiple disease areas. GPCRs are cell surface receptors that bind hormones on the extracellular surface of the cell and transduce a signal across the cellular membrane to the inside of the cell. The internal signal is amplified through interaction with G proteins that in turn interact with various second messenger pathways. This transduction pathway is manifested in downstream cellular responses that include cytoskeletal changes, cell motility, proliferation, apoptosis, secretion and regulation of protein expression to name a few. S1P receptors make good drug targets because individual receptors are expressed in different tissues and signal through different pathways making the individual receptors both tissue and response specific. Tissue specificity of the S1P receptors is desirable because development of an agonist or antagonist selective for one receptor localizes the cellular response to tissues containing that receptor, limiting unwanted side effects. Response specificity of the S1P receptors is also of importance because it allows for the development of agonists or antagonists that initiate or suppress certain cellular responses without affecting other responses. For example, the response specificity of the S1P receptors could allow for an S1P mimetic that initiates platelet aggregation without affecting cell morphology.

The physiologic implications of stimulating individual S1P receptors are largely unknown due in part to a lack of receptor type selective ligands. Isolation and characterization of S1P analogs that have potent agonist or antagonist activity for S1P receptors have been limited.

S1P1 for example is widely expressed and the knockout causes embryonic lethality due to large vessel rupture. Adoptive cell transfer experiments using lymphocytes from S1P1 knockout mice have shown that S1P1 deficient lymphocytes sequester to secondary lymph organs. Conversely, T cells overexpressing S1P1 partition preferentially into the blood compartment rather than secondary lymph organs. These experiments provide evidence that S1P1 is the main sphingosine receptor involved in lymphocyte homing and trafficking to secondary lymphoid compartments.

Currently, there is a need for novel, potent, and selective agents, which are agonists or antagonists of the individual receptors of the SIP receptor family in order to address unmet medical needs associated with agonism or antagonism of the individual receptors of the S1P receptor family.

### SUMMARY OF THE INVENTION

In one embodiment the invention provides a composition of Formula (I)

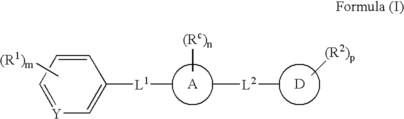

and pharmaceutically acceptable salts, isomers, prodrugs and biologically active metabolites thereof wherein
Y is N or CH;
A is selected from the group consisting of optionally substituted heteroaryl, optionally

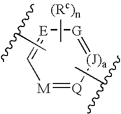

substituted heterocyclyl and
wherein
a is 0 or 1 and E, G, J, Q and M are each independently selected from the group consisting of CR^{a}, O, N and S provided that at least one of E, G, J, Q and M is CR^{a};
   no more than one of E, G, J, Q and M is O; and
   no more than one of E, G, J, Q and M is S;
L¹ and L² are each independently selected from the group consisting of a bond, —C(O)NH—, —NHC(O)—, —SO₂NH—, —NHSO₂—, —CH₂N(H)—, —N(H)CH₂—, —CH₂S— and —SCH₂—, provided that either L¹ or L² is a bond but L¹ and L² are not bonds at the same time;
D is selected from the group consisting of aryl, heteroaryl, heterocyclyl and (C₃-C₉)cycloalkyl;
R¹ and R² are each independently selected from the group consisting of halogen, CF₃, CN, OH, OCF₃, optionally substituted (C₁-C₆)alkyl, —C(O)—O—(C₁-C₆)alkyl, NR^{a}R^{b}, —(CH₂)ₓ-optionally substituted aryl, —(CH₂)ₓ-optionally substituted (C₃-C₆)cyclyl, —(CH₂)ₓ-optionally substituted heteroaryl, —(CH₂)ₓ-optionally substituted heterocyclyl, —NR^{a}-optionally substituted (C₃-C₆)cycloalkyl, —O-optionally substituted (C₁-C₆)alkyl, —O-optionally substituted (C₃-C₆)cycloalkyl, —O-heterocyclyl —O-aryl, —O-heteroaryl, —NR^{a}-optionally substituted heteraryl, —NR^{a}-optionally substituted aryl, SO₂NR^{a}R^{b} and CH₂NR^{a}NR provided that R¹ and R² are not both —(CH₂)ₓ-optionally substituted heterocyclyl or —(CH₂)ₓ-optionally substituted heteroaryl at the same time;
R^{a} and R^{b} are each independently selected from H and optionally substituted (C₁-C₆)alkyl;
R^{c} is independently selected from the group consisting of CF₃, CCl₃, optionally substituted (C₁-C₆) alkyl, —C(O)-optionally substituted (C₁-C₆)alkyl, —C(O)—O-optionally substituted (C₁-C₆)alkyl and oxo;
m is 0, 1 or 2;
n is 0, 1 or 2;
p is 0, 1 or 2; and
x is 0, 1 or 2;
provided that the compound is not

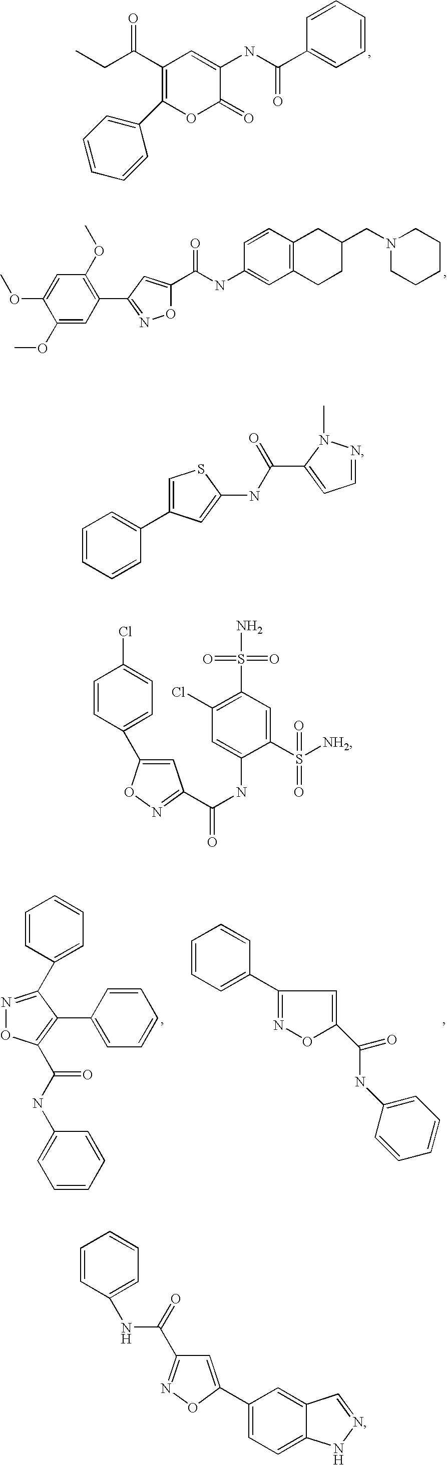

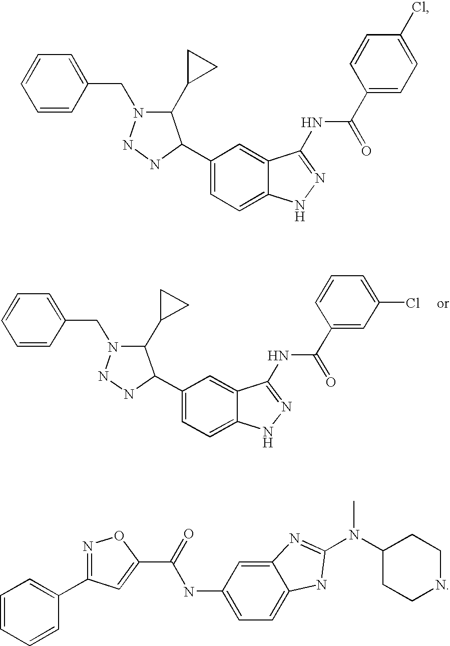

In another embodiment, the invention provides a compound according to Formula (I), wherein A is

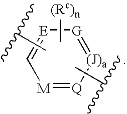

In another embodiment the invention provides a compound according to any of the foregoing embodiments wherein A is selected from the optionally substituted group consisting of furanyl, imidazolyl, isoxazolyl, oxadiazolyl, oxazolyl, pyranyl, pyrazolyl, pyrrolyl, thiazolyl, thienyl and 1H-[1,2,4]triazolyl.

In another embodiment the invention provides a compound according to any of the foregoing embodiments wherein D is selected from the group consisting of benzofuranyl, indanyl, indazolyl, indolyl, 2,3-dihydro-1H-indolyl, oxadiazolyl, phenyl, pyrazolyl, pyridinyl, tetrahydroisoquinolinyl, tetrahydroquinolinyl, thienyl and

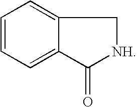

In another embodiment the invention provides a compound according to any of the foregoing embodiments wherein R¹ and R² are independently selected from the group consisting of Br, Cl, CF₃, CN, OH, OCF₃, CH₃, —CH(CH₃)₂, OCH₃, OCH(CH₃)₂, —C(O)OCH₂CH₃, optionally substituted (C₁-C₆)alkyl, —NR^{a}-optionally substituted (C₁-C₆)alkyl, N(CH₃)₂, —(CH₂)ₓ-optionally substituted aryl, —(CH₂)ₓ-optionally substituted (C₃-C₆)cycloalkyl, —(CH₂)ₓ-optionally substituted heteroaryl, morpholinyl, —(CH₂)— optionally substituted azetidinyl, —(CH₂)-optionally substituted pyrrolidinyl, —(CH₂)-optionally substituted piperidinyl, —NH-optionally substituted (C₃-C₆)alkyl, NH-optionally substituted (C₃-C₆)cycloalkyl, —O-optionally substituted (C₃-C₆)cycloalkyl and —O-tetrahydrofuranyl.

In another embodiment the invention provides a compound according to any of the foregoing embodiments wherein A is selected from the optionally substituted group consisting of isoxazolyl, oxadiazolyl, oxazolyl, pyranyl, pyrazolyl, thienyl and 1H-[1,2,4]triazolyl.

In another embodiment, the invention provides a compound according to any of the foregoing embodiments wherein Y is CH.

In another embodiment the invention provides a compound according to any of the foregoing embodiments wherein L¹ and L² are selected from the group consisting of a bond, —C(O)NH—, —NHC(O)—, SO₂NH— and —NHSO₂—.

In another embodiment the invention provides a compound according to any of the foregoing embodiments wherein D is selected from the group consisting of indanyl, indazolyl, phenyl, pyrazolyl, tetrahydroisoquinolinyl, tetrahydroquinolinyl and

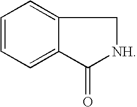

In another embodiment the invention provides a compound according to any of the foregoing embodiments wherein R¹ and R² are each independently selected from the group consisting of C₁, CF₃, CH₃, OCF₃, OCH₃, OCH(CH₃)₂, —C(O)OCH₂CH₃, N(CH₃)₂, morpholinyl, —(CH₂)-optionally substituted azetidinyl, —(CH₂)-optionally substituted pyrrolidinyl, —(CH₂)— optionally substituted piperidinyl, NH-optionally substituted (C₃-C₆)cycloalkyl and —O-tetrahydrofuranyl.

In another embodiment the invention provides a compound according to any of the foregoing embodiments wherein D is selected from the group consisting of indanyl, phenyl, pyrazolyl, tetrahydroisoquinolinyl, and tetrahydroquinolinyl.

In another embodiment the invention provides a compound according to any of the foregoing embodiments wherein A is selected from the optionally substituted group consisting of isoxazolyl, oxadiazolyl, oxazolyl, pyranyl, pyrazolyl, pyrrolyl, thiazolyl, thienyl and 1H-[1,2,4]triazolyl.

In another embodiment the invention provides a compound according to any of the foregoing embodiments wherein L¹ and L² are selected from the group consisting of a bond, —C(O)NH— and —NHC(O)—.

In another embodiment the invention provides a compound according to any of the foregoing embodiments wherein R¹ and R² are each independently selected from the group consisting of C₁, CF₃, CH₃, OCF₃, OCH₃, OCH(CH₃)₂, —C(O)OCH₂CH₃, N(CH₃)₂, —(CH₂)-optionally substituted azetidinyl, —(CH₂)-optionally substituted pyrrolidinyl, —(CH₂)-optionally substituted piperidinyl, and NH-optionally substituted (C₃-C₆)cycloalkyl.

In another embodiment the invention provides a compound according to any of the foregoing embodiments wherein A is selected from the optionally substituted group consisting of isoxazolyl, oxadiazolyl, pyranyl, pyrazolyl, thienyl and 1H-[1,2,4]triazolyl.

In another embodiment the invention provides a compound according to any of the foregoing embodiments wherein D is selected from the group consisting of indanyl, phenyl and pyrazolyl.

In another embodiment the invention provides a compound according to any of the foregoing embodiments wherein each R^{c} is independently selected from the group consisting of CF₃, CCl₃, t-butyl, —C(O)—OCH₂CH₃, —C(O)—OCH₂CH₂CH₃, and oxo.

In another embodiment the invention provides a compound according to any of the foregoing embodiments and pharmaceutically acceptable salts thereof wherein A is selected from the optionally substituted group consisting of isoxazolyl, pyrazolyl, thienyl and 1H-[1,2,4]-triazolyl.

In another embodiment the invention provides a compound according to any of the foregoing embodiments wherein each R^{c} is independently selected from the group consisting of CF₃, CCl₃, —C(O)—OCH₂CH₃, —C(O)—OCH₂CH₂CH₃ and oxo.

In another embodiment the invention provides a method of treating a condition in a patient comprising administering a therapeutically effective amount of a compound of the present invention or a physiologically acceptable salt thereof to said patient, wherein said condition is selected from the group consisting of rheumatoid arthritis, osteoarthritis, asthma, chronic obstructive pulmonary disease, sepsis, psoriasis, psoriatic arthritis, inflammatory bowel disease, Crohn's disease, lupus, multiple sclerosis, juvenile chronic arthritis, Lyme arthritis, reactive arthritis, septic arthritis, spondyloarthropathy, systemic lupus erythematosus, an ocular condition, a cancer, a solid tumor, fibrosarcoma, osteoma, melanoma, retinoblastoma, a rhabdomyosarcoma, glioblastoma, neuroblastoma, teratocarcinoma, an cancers such as lung, breast, stomach, bladder, colon, pancreas, ovarian, prostate and rectal cancer and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, alpha-1 antitrypsin deficiency, amyotrophic lateral sclerosis, anemia, angina pectoris, anterior horn cell degeneration, anti cd3 therapy, antiphospholipid syndrome, anti-receptor hypersensitivity reactions, hypersensitivity reactions, hyperkinetic movement disorders, hypersensitivity pneumonitis, hypertension, hypokinetic movement disorders, aortic and peripheral aneurysms, hypothalamic-pituitary-adrenal axis evaluation, aortic dissection, arterial hypertension, arteriosclerosis, arteriovenous fistula, ataxia, spinocerebellar degenerations, streptococcal myositis, structural lesions of the cerebellum, Subacute sclerosing panencephalitis, Syncope, syphilis of the cardiovascular system, systemic anaphylaxis, systemic inflammatory response syndrome, systemic onset juvenile rheumatoid arthritis, T-cell or FAB ALL, Telangiectasia, thromboangitis obliterans, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, atrial fibrillation (sustained or paroxysmal), atrial flutter, atrioventricular block, B cell lymphoma, bone graft rejection, bone marrow transplant (BMT) rejection, small bowel transplant rejection, spinal ataxia, bundle branch block, Burkitt's lymphoma, burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia, chronic alcoholism, chronic inflammatory pathologies, chronic lymphocytic leukemia, chronic salicylate intoxication, colorectal carcinoma, congestive heart failure, conjunctivitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetic ateriosclerotic disease, Diffuses Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, Epstein Barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, gram negative sepsis, gram positive sepsis, granulomas due to intracellular organisms, hairy cell leukemia, Hallerrorden-Spatz disease, hay fever, heart transplant rejection, hemachromatosis, hemodialysis, hemolytic uremic syndrome/thrombolytic thrombocytopenic purpura, hemorrhage, idiopathic pulmonary fibrosis, antibody mediated cytotoxicity, Asthenia, infantile spinal muscular atrophy, inflammation of the aorta, influenza A, ionizing radiation exposure, iridocyclitis/uveitis/optic neuritis, juvenile rheumatoid arthritis, juvenile spinal muscular atrophy, kidney transplant rejection, legionella, leishmaniasis, lipedema, liver transplant rejection, lymphederma, malaria, malignant Lymphoma, malignant histiocytosis, malignant melanoma, meningococcemia, metabolic/idiopathic, migraine headache, mitochondrial multi-system disorder, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, Kaposi's sarcoma, Hodgkin's disease, lymphoma, myeloma, leukaemia, malignant ascites, hematopoietic cancers, Crow-Fukase (POEMS) syndrome (polyneuropathy, organomegaly, endocrinopathy, monoclonal gammopathy, and skin changes syndrome), a diabetic condition such as insulin-dependent diabetes mellitus glaucoma, diabetic retinopathy or microangiopathy, sickle cell anaemia, chronic inflammation, synovitis, glomerulonephritis, graft rejection, Lyme disease, von Hippel Lindau disease, pemphigoid, Paget's disease, fibrosis, sarcoidosis, cirrhosis, thyroiditis, hyperviscosity syndrome, Osler-Weber-Rendu disease, chronic occlusive pulmonary disease, asthma or edema following burns, trauma, radiation, stroke, hypoxia, ischemia, ovarian hyperstimulation syndrome, post perfusion syndrome, post pump syndrome, post-MI cardiotomy syndrome, preeclampsia, menometrorrhagia, endometriosis, pulmonary hypertension, infantile hemangioma, or infection by Herpes simplex, Herpes Zoster, human immunodeficiency virus, parapoxvirus, protozoa or toxoplasmosis, Progressive supranucleo Palsy, primary pulmonary hypertension, radiation therapy, Raynaud's phenomenon and disease, Refsum's disease, regular narrow QRS tachycardia, renovascular hypertension, restrictive cardiomyopathy, sarcoma, senile chorea, Senile Dementia of Lewy body type, shock, skin allograft, skin changes syndrome, ocular or macular edema, ocular neovascular disease, scleritis, radial keratotomy, uveitis, vitritis, myopia, optic pits, chronic retinal detachment, post-laser treatment complications, conjunctivitis, Stargardt's disease, Eales disease, retinopathy, macular degeneration, restenosis, ischemia/reperfusion injury, ischemic stroke, vascular occlusion, carotid obstructive disease, ulcerative colitis, inflammatory bowel disease, diabetes, diabetes mellitus, insulin dependent diabetes mellitus, allergic diseases, dermatitis scleroderma, graft versus host disease, organ transplant rejection (including but not limited to bone marrow and solid organ rejection), acute or chronic immune disease associated with organ transplantation, sarcoidosis, disseminated intravascular coagulation, Kawasaki's disease, nephrotic syndrome, chronic fatigue syndrome, Wegener's granulomatosis, Henoch-Schoenlein purpurea, microscopic vasculitis of the kidneys, chronic active hepatitis, septic shock, toxic shock syndrome, sepsis syndrome, cachexia, infectious diseases, parasitic diseases, acquired immunodeficiency syndrome, acute transverse myelitis, Huntington's chorea, stroke, primary biliary cirrhosis, hemolytic anemia, malignancies, Addison's disease, idiopathic Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, peripheral vascular disorders, peritonitis, pernicious anemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis A, Hepatitis B, Hepatitis C, His bundle arrythmias, HIV infection/HIV neuropathy, common varied immunodeficiency (common variable hypogammaglobulinemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, pneumocystis carinii pneumonia, pneumonia, connective tissue disease associated interstitial lung disease, mixed connective tissue disease, associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalnia, pulmonary hypertension secondary to connective tissue disease, acute and chronic pain (different forms of pain), Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, toxicity, transplants, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci infection, mental disorders (e.g., depression and schizophrenia), Th2 Type and Th1 Type mediated diseases, and diseases involving inappropriate vascularization, e.g., diabetic retinopathy, retinopathy of prematurity, choroidal neovascularization due to age-related macular degeneration, and infantile hemangiomas in human beings. In addition, such compounds may be useful in the treatment of disorders such as ascites, effusions, and exudates, including, e.g., macular edema, cerebral edema, acute lung injury, adult respiratory distress syndrome, proliferative disorders such as restenosis, fibrotic disorders such as hepatic cirrhosis and atherosclerosis, mesangial cell proliferative disorders such as diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, preeclampsia, menometrorrhagia, cat scratch fever, rubeosis, neovascular glaucoma and retinopathies such as those associated with diabetic retinopathy, retinopathy of prematurity, age-related macular degeneration, acute idiopathic polyneuritis, acuter or chronic immune disease associated with organ transplantation, acute inflammatory demyelinating polyradiculoneuropathy, acute ischemia, adult Still's disease, allergy, anaphylaxis, anti-phospholipid antibody syndrome, aplastic anemia, atopic eczema, atopic dermatitis, autoimmune dermatitis, autoimmune diabetes, autoimmune disorder associated with streptococcus infection, autoimmune enteropathy, autoimmune hepatitis, autoimmune hearing loss, autoimmune lymphoproliferative syndrome, autoimmune myocarditis, autoimmune neutropenia, autoimmune premature ovarian failure, autoimmune thrombocytopenia, autoimmune uveitis, Behcet's disease, blepharitis, bronchiectasis, bullous pemphigoid, catastrophic antiphospholipid syndrome, celiac disease, cervical spondylosis, chronic ischemia, cicatricial pemphigoid, clinical isolated syndrome with risk for multiple sclerosis, childhood onset psychiatric disorder, dacrocystitis, dermatomyositis, disc herniation, disc prolapse, drug induced immune hemolytic anemia, endophthalmitis, episcleritis, erythema multiforme, erythema multiforme major, gestational pemphigoid, Guillain-Barre syndrome, heart failure, Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF/UIP, iritis, keratitis, keratojuntivitis sicca, Kussmaul disease or Kussmaul-Meier disease, Landry's paralysis, Langerhan's cell hisiocytosis, livedo reticularis, microscopic polyangiitis, morbus bechterev, motor neuron disorders, mucous membrane pemphigoid, primary progressive multiple sclerosis, secondary progressive multiple sclerosis, relapsing remitting multiple sclerosis, multiple organ failure, myelodysplastic syndrome, nerve root disorder, neuropathy, Non-A Non-B hepatitis, osteolysis, ovarian cancer, pauciarticular JRA, peripheral artery occlusive disease (PAOD), periphral vascular disease (PVD), peripheral artery disease, phlebitis, polychondritis, polymyalgia rheumatica, poliosis, polyarticular JRA, polyendocrine deficiency syndrome, polymyositis, post-pump syndrome, primary parkinsonism, prostatitis, psoratic arthropathy, pure red cell aplasia, primary adrenal insufficiency, Reiter's disease, recurrent neuromyelitis optica, rheumatic heart disease, SAPHO (synovitis, acne, pustulosis, hyperostosis, and osteitis), scleroderma, secondary amyloidosis, shock lung, sciatica, secondary adrenal insufficiency, septic arthritis, seronegative arthopathy, silicone associated connective tissue disease, Sneddon-Wilkson Dermatosis, spondilitis ankylosans, Stevens-Johnson Syndrome, systemic inflammatory response syndrome, temporal arteritis, toxoplasmic retinitis, toxic epidermal necrolysis, TRAPS (Tumor Necrosis factor receptor), type 1 allergic reaction, type II diabetes, urticaria, usual interstitial pneumonia, vernal conjunctivitis, viral retinitis, Vogt-Koyanagi-Harada syndrome (VKH syndrome) and wet macular degeneration.

The teachings of all references, including journal articles, patents and published patent applications, are incorporated herein by reference in their entirety.

### DETAILED DESCRIPTION OF THE INVENTION

In this invention, the following definitions are applicable:

A “therapeutically effective amount” is an amount of a compound of the present invention that inhibits, totally or partially, the progression of a disease condition or alleviates, at least partially, one or more symptoms of the condition. A therapeutically effective amount can also be an amount that is prophylactically effective in preventing a disease or symptoms associate with said disease. The amount that is therapeutically effective will depend upon a patient's size, gender, the condition to be treated, the severity of the condition, the result sought, as well as other variables well known to a skilled practitioner. For a given patient, a therapeutically effective amount can be determined by methods known to those of skill in the art.

“Pharmaceutically acceptable salts” refers to those salts which retain the biological effectiveness and properties of the free bases and which are obtained by reaction with inorganic acids, e.g., hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluene-sulfonic acid, citric acid, fumaric acid, maleic acid, succinic acid, benzoic acid, salicylic acid, lactic acid, tartaric acid (e.g. (+) or (−) tartaric acid or mixtures thereof), amino acids (e.g. (+) or (−) amino acids or mixtures thereof), and the like. These salts can be prepared by methods known to those skilled in the art.

Certain compounds of Formula (I) that have acidic substituents may exist as salts with pharmaceutically acceptable bases. The present invention includes such salts. Examples of such include sodium salts, potassium salts, lysine salts and arginine salts. These salts may be prepared by methods known to those skilled in the art.

Certain compounds of Formula (I) and their salts may exist in more than one crystal form and the scope of the present invention includes each crystal form and mixtures thereof.

Certain compounds of Formula (I) and their salts may also exist in the form of solvates, e.g., hydrates, and the scope of the present invention includes each solvate and mixtures thereof.

Certain compounds of Formula (I) may comprise one or more chiral centers, and exist in different optically active forms. When compounds of Formula (I) comprise one chiral center, the compounds exist in two enantiomeric forms and the present invention includes both enantiomers and mixtures of enantiomers, such as racemic mixtures. The enantiomers may be resolved by methods known to those skilled in the art, e.g., by formation of diastereoisomeric salts which may be separated, e.g., by crystallization; formation of diastereoisomeric derivatives or complexes which may be separated, e.g., by crystallization, gas-liquid or liquid chromatography; selective reaction of one enantiomer with an enantiomer-specific reagent, e.g., enzymatic esterification; or gas-liquid or liquid chromatography in a chiral environment, e.g. on a chiral support, e.g., silica with a bound chiral ligand or in the presence of a chiral solvent. It will be appreciated that where the desired enantiomer is converted into another chemical entity by one of the separation procedures described above, a further step may be used to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of Formula (I) comprises more than one chiral center, it may exist in diastereoisomeric forms. The diastereoisomeric compounds may be separated by methods known to those skilled in the art, e.g., chromatography or crystallization and the individual enantiomers may be separated as described above. The present invention includes each diastereoisomer of compounds of Formula (I) and mixtures thereof.

Certain compounds of Formula (I) may exist in different tautomeric forms or as different geometric isomers, and the present invention includes each tautomer and/or geometric isomer of compounds of Formula (I) and mixtures thereof.

Certain compounds of Formula (I) may exist in different stable conformational forms that may be separable. Torsional asymmetry due to restricted rotation about an asymmetric single bond, e.g., because of steric hindrance or ring strain, may permit separation of different conformers. The present invention includes each conformational isomer of compounds of Formula (I) and mixtures thereof.

Certain compounds of Formula (I) may exist in zwitterionic form and the present invention includes each zwitterionic form of compounds of Formula (I) and mixtures thereof.

As used herein the term “pro-drug” refers to an agent that is converted into the parent drug in vivo by a physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The pro-drug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial.

Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the pro-drug is enzymatically or chemically cleaved to deliver the ultimate drug in the blood or tissue.

Exemplary pro-drugs upon cleavage release a corresponding free acid, and such hydrolyzable ester-forming residues of the compounds of this invention include but are not limited to carboxylic acid substituents (e.g., —(CH₂)C(O)OH or a moiety that comprises a carboxylic acid) wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)-ethyl having from 5 to 8 carbon atoms, N-(alkoxycarbonyl)-aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of Formula (I) wherein the free hydrogen of the hydroxyl substituent (e.g., R¹ contains hydroxyl) is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonyl-amino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, P(O)(OH)₂, —P(O)(O(C₁-C₆)alkyl)₂ or glycosyl (the radical resulting from detachment of the hydroxyl of the hemiacetal of a carbohydrate).

The term “heterocyclic” or “heterocyclyl”, as used herein, include non-aromatic, ring systems, including, but not limited to, monocyclic, bicyclic and tricyclic rings, which can be completely saturated or which can comprise one or more units of unsaturation (for the avoidance of doubt, the degree of unsaturation does not result in an aromatic ring system) and have 3 to 12 atoms including at least one heteroatom, such as nitrogen, oxygen, or sulfur. For purposes of exemplification, which should not be construed as limiting the scope of this invention, the following are examples of heterocyclic rings: azepines, azetidinyl, morpholinyl, oxopiperidinyl, oxopyrrolidinesyl, piperazinyl, piperidinyl, pyrrolidinyl, quinicludinyl, thiomorpholinyl, tetrahydropyranyl and tetrahydrofuranyl.

The term “heteroaryl” as used herein, include aromatic ring systems, including, but not limited to, monocyclic, bicyclic and tricyclic rings, and have 3 to 12 atoms including at least one heteroatom, such as nitrogen, oxygen, or sulfur. For purposes of exemplification, which should not be construed as limiting the scope of this invention: azaindole, benzo(b)thienyl, benzimidazolyl, benzofuranyl, benzoxazolyl, benzothiazolyl, benzothiadiazolyl, benzoxadiazolyl, furans, imidazoles, imidazopyridine, indole, indolinyl, indazoles, isoindolinyl, isoxazoles, isothiazoles, oxadiazoles, oxazoles, purine, pyrans, pyrazines, pyrazoles, pyridines, pyrimidines, pyrroles, pyrrolo[2,3-d]pyrimidine, pyrazolo[3,4-d]pyrimidine), quinolines, quinazolines, triazoles, thiazoles, thiophenyl, tetrahydroindole, tetrazoles, thiadiazoles, thienyls, thiomorpholines, triaozles or tropanyl.

When the term “substituted heterocyclic” (or heterocyclyl) or “substituted heteroaryl” is used, what is meant is that the heterocyclic group is substituted with one or more substituents that can be made by one of ordinary skill in the art and results in a composition that is an agonist or antagonist of the sphingosine receptor family. For purposes of exemplification, which should not be construed as limiting the scope of this invention, typical substituents for a heterocycle of this invention are each independently selected from the optionally substituted group consisting of alkenyl, alkoxy, alkoxyalkoxy, alkoxyalkyl, alkoxycarbonyl, alkoxycarbonylheterocycloalkoxy, alkyl, alkylcarbonyl, alkylester, alkyl-O—C(O)—, alkyl-heterocyclyl, alkyl-cycloalkyl, alkyl-nitrile, alkynyl, amido groups, amino, aminoalkyl, aminocarbonyl, carbonitrile, carbonylalkoxy, carboxamido, CF₃, CN, —C(O)OH, —C(O)H, —C(O)—C(CH₃)₃, —OH, —C(O)O-alkyl, —C(O)O-cycloalkyl, —C(O)O-heterocyclyl, —C(O)-alkyl, —C(O)-cycloalkyl, —C(O)-heterocyclyl, cycloalkyl, dialkylaminoalkoxy, dialkylaminocarbonylalkoxy, dialkylaminocarbonyl, halogen, heterocyclyl, a heterocycloalkyl group, heterocyclyloxy, hydroxy, hydroxyalkyl, nitro, OCF₃, oxo, phenyl, —SO₂CH₃, —SO₂CR₃, tetrazolyl, thienylalkoxy, trifluoromethylcarbonylamino, trifluoromethylsulfonamido, heterocyclylalkoxy, heterocyclyl-S(O)ₚ, cycloalkyl-S(O)ₚ, alkyl-S—, heterocyclyl-S, heterocycloalkyl, cycloalkylalkyl, heterocycolthio, cycloalkylthio, -Z¹⁰⁵-C(O)N(R)₂, -Z¹⁰⁵-N(R)—C(O)-Z²⁰⁰, -Z¹⁰⁵-N(R)—S(O)₂-Z²⁰⁰, -Z¹⁰⁵-N(R)—C(O)—N(R)-Z²⁰⁰, —N(R)—C(O)R, —N(R)—C(O)OR, OR—C(O)-heterocyclyl-OR, R_{c} and —CH₂OR_{c};
wherein R₃ is C₁-C₄ alkyl, C₃-C₆ cycloalkyl or phenyl;
wherein p is 0, 1 or 2;
wherein R_{c} for each occurrence is independently hydrogen, optionally substituted alkyl, optionally substituted aryl, —(C₁-C₆)—NR^{d}R^{e}, -E-(CH₂)ₜ—NR_{d}Rₑ, -E-(CH₂)ₜ—O-alkyl, -E-(CH₂)ₜ—S-alkyl, or -E-(CH₂)ₜ—OH;
   wherein t is an integer from about 1 to about 6;
Z¹⁰⁵ for each occurrence is independently a covalent bond, alkyl, alkenyl or alkynyl; and
Z²⁰⁰ for each occurrence is independently selected from an optionally substituted group selected from the group consisting of alkyl, alkenyl, alkynyl, phenyl, alkyl-phenyl, alkenyl-phenyl or alkynyl-phenyl;
E is a direct bond, O, S, S(O), S(O)₂, or NR_{f}, wherein R_{f} is H or alkyl and R_{d} and Rₑ are independently H, alkyl, alkanoyl or SO₂-alkyl; or R_{d}, Rₑ and the nitrogen atom to which they are attached together to form a five- or six-membered heterocyclic ring.

An “heterocycloalkyl” group, as used herein, is a heterocyclic group that is linked to a compound by an aliphatic group having from one to about eight carbon atoms. For example, a typical heterocycloalkyl group is a morpholinomethyl group.

As used herein, “aliphatic” or “an aliphatic group” or notations such as “(C₀-C₈)” include straight chained or branched hydrocarbons which are completely saturated or which comprise one or more units of unsaturation, and, thus, includes alkyl, alkenyl, alkynyl and hydrocarbons comprising a mixture of single, double and triple bonds. When the group is a C₀ it means that the moiety is not present or in other words, it is a bond. As used herein, “alkyl” means C₁-C₈ and includes straight chained or branched hydrocarbons, which are completely saturated. Typical alkyls are methyl, ethyl, propyl, butyl, pentyl, hexyl and isomers thereof. As used herein, “alkenyl” and “alkynyl” means C₂-C₈ and includes straight chained or branched hydrocarbons that comprise one or more units of unsaturation, one or more double bonds for alkenyl and one or more triple bonds for alkynyl.

As used herein, aromatic groups (or aryl groups) include aromatic carbocyclic ring systems (e.g., phenyl and cyclopentyldienyl) and fused polycyclic aromatic ring systems (e.g., naphthyl, biphenylenyl and 1,2,3,4-tetrahydronaphthyl).

As used herein, cycloalkyl means C₃-C₁₂ monocyclic or multicyclic (e.g., bicyclic, tricyclic, etc.) hydrocarbons that is completely saturated or has one or more unsaturated bonds but does not amount to an aromatic group. Typical examples of a cycloalkyl group are cyclopropyl, cyclobutyl, cyclopentyl, cyclopentenyl, cyclohexyl and cyclohexenyl.

As used herein, amido group means —NHC(═O)—.

As used herein, acyloxy group means —OC(O)R.

As used herein, sulfanyl group means —S—. In particular, the term “alkylsulfanylalkyl” refers to -alkyl-5-alkyl.

As used herein, many moieties or substituents are termed as being either “substituted” or “optionally substituted”. When a moiety is modified by one of these terms, unless otherwise noted, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: alkenyl groups, alkoxy group (which itself can be substituted, such as —O—C₁-C₆-alkyl-OR, —O—C₁-C₆-alkyl-N(R)₂, and OCF₃), alkoxyalkoxy, alkoxycarbonyl, alkoxycarbonylpiperidinyl-alkoxy, alkyl groups (which itself can also be substituted, such as —C₁-C₆-alkyl-OR, —C₁-C₆-alkyl-N(R)₂, and —CF₃), alkylamino, alkylcarbonyl, alkylester, alkylnitrile, alkylsulfonyl, amino, aminoalkoxy, CF₃, COH, COOH, CN, cycloalkyl, dialkylamino, dialkylaminoalkoxy, dialkylaminocarbonyl, dialkylaminocarbonylalkoxy, dialkylaminosulfonyl, esters (—C(O)—OR, where R is groups such as alkyl, heterocycloalkyl (which can be substituted), heterocyclyl, etc., which can be substituted), halogen or halo group (F, Cl, Br, I), hydroxy, morpholinoalkoxy, morpholinoalkyl, nitro, oxo, OCF₃, optionally substituted phenyl, S(O)₂CH₃, S(O)₂CF₃, and sulfonyl, N-alkylamino or N,N-dialkylamino (in which the alkyl groups can also be substituted).

### Methods of Use

The present invention provides compositions described by general Formula (I) that are effective as antagonists or agonists of the G protein-coupled S1P receptor family. These compounds reduce the number of circulating and infiltrating T- and B-lymphocytes affording a beneficial immunosuppressive effect.

The present invention also provides compounds that exhibit activity within the S1P receptor family.

In a related aspect, the invention provides a method for modulating receptors of the S1P family in a subject (e.g., human) suffering from a disorder in which modulation of S1P activity is beneficial, comprising administering to the subject a compound of Formula (I) such that modulation of S1P activity in the subject is effected.

In another related aspect, the invention provides a method of modulating sphingosine 1-phosphate receptor 1 activity comprising contacting a cell with one or more compounds of Formula (I).

A compound of Formula (I) or a salt thereof or pharmaceutical compositions comprising a therapeutically effective amount thereof is useful in the treatment of a disorder selected from the group consisting of CNS system disorders, arthritis, rheumatoid arthritis, osteoarthritis, juvenile chronic arthritis, Lyme arthritis, psoriatic arthritis, reactive arthritis, and septic arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection (including but not limited to bone marrow and solid organ rejection), acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immuno-deficiency Related Diseases, Hepatitis B, Hepatitis C, common varied immuno-deficiency (common variable hypogammaglobulinaemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, hashimoto's thyroiditis, hay fever, heart transplant rejection, hemachromatosis, hemodialysis, hemolytic uremic syndrome/thrombolytic thrombocytopenic purpura, hemorrhage, hepatitis (A), His bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignamt Lymphoma, malignant histiocytosis, malignant melanoma, meningitis, meningococcemia, metabolic/idiopathic, migraine headache, mitochondrial multi.system disorder, mixed connective tissue disease, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carnii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, and diseases involving inappropriate vascularization, e.g., diabetic retinopathy, retinopathy of prematurity, choroidal neovascularization due to age-related macular degeneration, and infantile hemangiomas in human beings. In addition, such compounds may be useful in the treatment of disorders such as, edema, ascites, effusions, and exudates, including, e.g., macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as glomerulonephritis, diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, Crow-Fukase syndrome (POEMS), preeclampsia, menometrorrhagia, cat scratch fever, rubeosis, neovascular glaucoma and retinopathies such as those associated with diabetic retinopathy, retinopathy of prematurity, age-related macular degeneration or a central nervous system disorder. In addition, these compounds can be used as active agents against solid tumors, malignant ascites, von Hippel Lindau disease, hematopoietic cancers and hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

### Combination Therapy

Compounds of Formula (I) of the invention can be used alone or in combination with one or more therapeutic agents to treat disease. It should be understood that the compounds of the present invention may be used alone or in combination with additional agents, e.g., a therapeutic agent, said additional agents being selected by the skilled artisan for its intended purpose. For example, the additional agents may be one or more therapeutic agents art-recognized as being useful to treat a disease or condition being treated by a compound of the present invention. The additional agents also can be agents that imparts beneficial attributes to the therapeutic composition e.g., agents that affects the viscosity of the composition.

It should further be understood that the combinations which are to be included within this invention are those combinations useful for their intended purpose. The agents set forth below are for illustrative purposes and not intended to be limiting. The combinations, which are part of this invention, may be compounds of the present invention and one or more additional agents selected from the lists below.

Typical combinations are non-steroidal anti-inflammatory drug(s) also referred to as NSAIDS that include ibuprofen. Other combinations are corticosteroids including prednisolone; the well known side-effects of steroid use can be reduced or even eliminated by tapering the steroid dose required when treating patients in combination with the S1P receptor agonists or antagonists of this invention. Non-limiting examples of therapeutic agents for rheumatoid arthritis with which a compound of Formula (I) of the invention can be combined include the following: cytokine suppressive anti-inflammatory drug(s) (CSAIDs); antibodies to or antagonists of other human cytokines or growth factors, e.g., TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, and PDGF. S/T kinase inhibitors of the invention can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Typical combinations of therapeutic agents may interfere at different points in the autoimmune and subsequent inflammatory cascade; examples include TNF antagonists such as chimeric, humanized or human TNF antibodies, D2E7 (HUMIRA™), (PCT Publication No. WO 97/29131), CA2 (REMICADE™), CDP 571, and soluble p55 or p75 TNF receptors, derivatives, thereof, (p75TNFR1gG (Enbrel™) or p55TNFR1gG (Lenercept™), and also TNFα converting enzyme (TACE) inhibitors; similarly IL-1 inhibitors (Interleukin-1-converting enzyme inhibitors, IL-1RA etc.) may be effective for the same reason. Other combinations include Interleukin 11. Yet other combinations include other key players of the autoimmune response that may act parallel to, dependent on, or in concert with IL-18 function; e.g., are IL-12 antagonists including IL-12 antibodies or soluble IL-12 receptors, or IL-12 binding proteins. It has been demonstrated that IL-12 and IL-18 have overlapping but distinct functions and a combination of antagonists to both may be most effective. Yet another combination are non-depleting anti-CD4 inhibitors. Yet other combinations include antagonists of the co-stimulatory pathway CD80 (B7.1) or CD86 (B7.2) including antibodies, soluble receptors or antagonistic ligands.

A compound of Formula (I) of the invention may also be combined with other agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), β-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, e.g., ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNF or IL-1 (e.g. IRAK, NIK, IKK, p38 or MAP kinase inhibitors), IL-1β converting enzyme inhibitors, T-cell signalling inhibitors such as kinase inhibitors, metalloproteinase inhibitors, sulfasalazine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™ and p55TNFRIgG (Lenercept™)), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, and Mesopram. Combinations include methotrexate or leflunomide and in moderate or severe rheumatoid arthritis cases, cyclosporine and anti-TNF antibodies as noted above.

Non-limiting examples of therapeutic agents for inflammatory bowel disease with which a compound of Formula (I) of the invention may be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-11 monoclonal antibodies; anti-IL-6 monoclonal antibodies; growth factors; elastase inhibitors; pyridinyl-imidazole compounds; antibodies to or antagonists of other human cytokines or growth factors, e.g., TNF, LT, IL-1, IL-2, IL-6, IL-7, IL-8, IL-12, IL-15, IL-16, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, e.g., ibuprofen; corticosteroids such as prednisolone; phosphodiesterase inhibitors; adenosine agonists; antithrombotic agents; complement inhibitors; adrenergic agents; agents which interfere with signalling by proinflammatory cytokines such as TNFα or IL-1 (e.g. IRAK, NIK, IKK, or MAP kinase inhibitors); IL-1β converting enzyme inhibitors; TNFα converting enzyme inhibitors; T-cell signalling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sL-1RI, sL-1RII, sIL-6R) and antiinflammatory cytokines (e.g. IL-4, IL-10, IL-11, IL-13 and TGFβ). Typical examples of therapeutic agents for Crohn's disease with which a compound of formula (I), (Ia), (Ib), or (Ic) can be combined include the following: TNF antagonists, e.g., anti-TNF antibodies, D2E7 (PCT Publication No. WO 97/29131; HUMIRA™), CA2 (REMICADE™), CDP 571, TNFR-Ig constructs, (p75TNFRIgG (ENBREL™) and p55TNFRIgG (Lenercept™)) inhibitors and PDE4 inhibitors.

A compound of Formula (I) can be combined with corticosteroids, e.g., budenoside and dexamethasone; sulfasalazine, 5-aminosalicylic acid; olsalazine; and agents which interfere with synthesis or action of proinflammatory cytokines such as IL-1, e.g., IL-1β converting enzyme inhibitors and IL-1ra; T cell signaling inhibitors, e.g., tyrosine kinase inhibitors 6-mercaptopurines; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-γ.

Non-limiting examples of therapeutic agents for multiple sclerosis with which a compound of Formula (I) can be combined include the following: corticosteroids; prednisolone; methylprednisolone; azathioprine; cyclophosphamide; cyclosporine; methotrexate; 4-aminopyridine; tizanidine; interferon-β1a (Avonex®; Biogen); interferon-β1b (Betaseron®; Chiron/Berlex); interferon α-n3) (Interferon Sciences/Fujimoto), interferon-α (Alfa Wassermann/J&J), interferon β1A-IF (Serono/Inhale Therapeutics), Peginterferon α2b (Enzon/Schering-Plough), Copolymer 1 (Cop-1; Copaxone®; Teva Pharmaceutical Industries, Inc.); hyperbaric oxygen; intravenous immunoglobulin; clabribine; antibodies to or antagonists of other human cytokines or growth factors and their receptors, e.g., TNF, LT, IL-1, IL-2, IL-6, IL-7, IL-8, IL-12, IL-23, IL-15, IL-16, EMAP-II, GM-CSF, FGF, and PDGF. A compound of formula (I), (Ia), (Ib), or (Ic) can be combined with antibodies to cell surface molecules such as CD2, CD3, CD4, CD8, CD19, CD20, CD25, CD28, CD30, CD40, CD45, CD69, CD80, CD86, CD90 or their ligands. A compound of Formula (I), (Ia), (Ib), or (Ic) may also be combined with agents such as methotrexate, cyclosporine, FK506, rapamycin, mycophenolate mofetil, leflunomide, NSAIDs, e.g., ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

Suitable examples of therapeutic agents for multiple sclerosis in which a compound of Formula (I) can be combined include interferon-β, e.g., IFNβ1a and IFNβ1b; copaxone, corticosteroids, caspase inhibitors, e.g., inhibitors of caspase-1, IL-1 inhibitors, TNF inhibitors, and antibodies to CD40 ligand and CD80.

A compound of Formula (I) may also be combined with agents, such as alemtuzumab, dronabinol, daclizumab, mitoxantrone, xaliproden hydrochloride, fampridine, glatiramer acetate, natalizumab, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-β2, tiplimotide, VLA-4 antagonists (e.g., TR-14035, VLA4 Ultrahaler, Antegran-ELAN/Biogen), interferon-γ antagonists and IL-4 agonists.

Non-limiting examples of therapeutic agents for angina with which a compound of Formula (I) of the invention may be combined include the following: aspirin, nitroglycerin, isosorbide mononitrate, metoprolol succinate, atenolol, metoprolol tartrate, amLodipine besylate, diltiazem hydrochloride, isosorbide dinitrate, clopidogrel bisulfate, nifedipine, atorvastatin calcium, potassium chloride, furosemide, simvastatin, verapamil HCl, digoxin, propranolol hydrochloride, carvedilol, lisinopril, spironolactone, hydrochlorothiazide, enalapril maleate, nadolol, ramipril, enoxaparin sodium, heparin sodium, valsartan, sotalol hydrochloride, fenofibrate, ezetimibe, bumetanide, losartan potassium, lisinopril/hydrochlorothiazide, felodipine, captopril and bisoprolol fumarate.

Non-limiting examples of therapeutic agents for ankylosing spondylitis with which a compound of Formula (I) can be combined include the following: ibuprofen, diclofenac, misoprostol, naproxen, meloxicam, indomethacin, diclofenac, celecoxib, rofecoxib, sulfasalazine, methotrexate, azathioprine, minocyclin, prednisone, etanercept, and infliximab.

Non-limiting examples of therapeutic agents for asthma with which a compound of Formula (I) can be combined include the following: albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin HCl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine HCl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone and metaproterenol sulfate.

Non-limiting examples of therapeutic agents for COPD with which a compound of Formula (I) can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol HCl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, cilomilast and roflumilast.

Non-limiting examples of therapeutic agents for HCV with which a compound of Formula (I) can be combined include the following: Interferon-alpha-2a, Interferon-alpha-2b, Interferon-alpha con1, Interferon-alpha-n1, pegylated interferon-alpha-2a, pegylated interferon-alpha-2b, ribavirin, peginterferon alfa-2b+ribavirin, ursodeoxycholic acid, glycyrrhizic acid, thymalfasin, Maxamine, VX497 and any compounds that are used to treat HCV through intervention with the following targets: HCV polymerase, HCV protease, HCV helicase, and HCV IRES (internal ribosome entry site).

Non-limiting examples of therapeutic agents for Idiopathic Pulmonary Fibrosis with which a compound of Formula (I) can be combined include the following: prednisone, azathioprine, albuterol, colchicine, albuterol sulfate, digoxin, Interferon-γ, methylprednisolone sod succ, lorazepam, furosemide, lisinopril, nitroglycerin, spironolactone, cyclophosphamide, ipratropium bromide, actinomycin D, alteplase, fluticasone propionate, levofloxacin, metaproterenol sulfate, morphine sulfate, oxycodone HCl, potassium chloride, triamcinolone acetonide, tacrolimus anhydrous, calcium, Interferon-α, methotrexate, mycophenolate mofetil and Interferon-γ-1β.

Non-limiting examples of therapeutic agents for myocardial infarction with which a compound of Formula (I)) can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril HCl/mag carb, bumetanide, alteplase, enalaprilat, amiodarone hydrochloride, tirofiban HCl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine HCl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, and cariporide.

Non-limiting examples of therapeutic agents for psoriasis with which a compound of Formula (I) can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, and sulfasalazine.

Non-limiting examples of therapeutic agents for psoriatic arthritis with which a compound of Formula (I) can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulndac, betamethasone diprop augmented, inflixirnab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept and efalizumab.

Non-limiting examples of therapeutic agents for restenosis with which a compound of Formula (I) can be combined include the following: sirolimus, paclitaxel, everolimus, tacrolimus, ABT-578, and acetaminophen.

Non-limiting examples of therapeutic agents for sciatica with which a compound of Formula (I) can be combined include the following: hydrocodone bitartrate/apap, rofecoxib, cyclobenzaprine HCl, methylprednisolone, naproxen, ibuprofen, oxycodone HCl/acetaminophen, celecoxib, valdecoxib, methylprednisolone acetate, prednisone, codeine phosphate/apap, tramadol HCl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone HCl, tizanidine HCl, diclofenac sodium/misoprostol, propoxyphene napsylate/apap, asa/oxycod/oxycodone ter, ibuprofen/hydrocodone bit, tramadol HCl, etodolac, propoxyphene HCl, amitriptyline HCl, carisoprodol/codeine phos/asa, morphine sulfate, multivitamins, naproxen sodium, orphenadrine citrate, and temazepam.

Suitable examples of therapeutic agents for SLE (Lupus) with which a compound of Formula (I) can be combined include the following: NSAIDS, e.g., diclofenac, naproxen, ibuprofen, piroxicam, indomethacin; COX2 inhibitors, e.g., celecoxib, rofecoxib, valdecoxib; anti-malarials, e.g., hydroxychloroquine; steroids, e.g., prednisone, prednisolone, budenoside, dexamethasone; cytotoxics, e.g., azathioprine, cyclophosphamide, mycophenolate mofetil, methotrexate; inhibitors of PDE4 or purine synthesis inhibitor, e.g., Cellcept®.

A compound of Formula (I) may also be combined with agents such as sulfasalazine, 5-aminosalicylic acid, olsalazine, Imuran® and agents which interfere with synthesis, production or action of proinflammatory cytokines such as IL-1, e.g., caspase inhibitors like IL-1β converting enzyme inhibitors and IL-1ra.

A compound of Formula (I) may also be used with T cell signaling inhibitors, e.g., tyrosine kinase inhibitors; or molecules that target T cell activation molecules, e.g., CTLA-4-IgG or anti-B7 family antibodies, anti-PD-1 family antibodies.

A compound of Formula (I) can be combined with IL-11 or anti-cytokine antibodies, e.g., fonotolizumab (anti-IFNg antibody), or anti-receptor receptor antibodies, e.g., anti-IL-6 receptor antibody and antibodies to B-cell surface molecules.

A compound of Formula (I) may also be used with LJP 394 (abetimus), agents that deplete or inactivate B-cells, e.g., Rituximab (anti-CD20 antibody), lymphostat-B (anti-BlyS antibody), TNF antagonists, e.g., anti-TNF antibodies, D2E7 (PCT Publication No. WO 97/29131; HUMIRA™), CA2 (REMICADE™), CDP 571, TNFR-Ig constructs, (p75TNFRIgG (ENBREL™) and p55TNFRIgG (LENERCEPT™)).

In the compositions of the present invention the active compound may, if desired, be associated with other compatible pharmacologically active ingredients. For example, the compounds of this invention can be administered in combination with one or more other therapeutic agents that are known to treat a disease or condition described herein. For example, with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate.

Additional pharmaceutical agents include, but are not limited to, anti-edemic steroids, NSAIDS, ras inhibitors, anti-TNF agents, anti-IL1 agents, antihistamines, PAF-antagonists, COX-1 inhibitors, COX-2 inhibitors, NO synthase inhibitors, Akt/PTB inhibitors, IGF-1R inhibitors, PKC inhibitors, PI3 kinase inhibitors, calcineurin inhibitors and immunosuppressants.

The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deletrious effects of a hyperproliferative disorder, angiogenesis, vascular hyperpermeability or edema than the administration of either substance alone. In the treatment of malignant disorders combinations with antiproliferative or cytotoxic chemotherapies or radiation are included in the scope of the present invention.

One or more compounds of the invention can be administered to a subject, such as a human patient, individually or in pharmaceutical compositions where they are mixed with biologically suitable carriers or excipient(s) at doses to treat or ameliorate a disease or condition as described herein. Mixtures of these compounds can also be administered to a subject as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

### Pharmaceutical Compositions and Modes of Administration

Suitable routes of administration may, e.g., include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer a compound in a local rather than in a systemic manner, e.g., via injection of the compound directly into an edematous site, often in a depot or sustained release formulation.

Furthermore, one may administer a compound in a targeted drug delivery system, e.g., in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries that facilitate processing of the active compounds into preparations that can be used pharmaceutically. Proper formulation is dependent upon the route of administration chosen.

For injection, agents of the invention may be formulated in aqueous solutions, typically in physiologically compatible buffers such as Hanks' solution, Ringer's solution, or physiological saline buffer. For transmucosal administration, penetrants appropriate to the barrier to be permeated can be used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated.

Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, e.g., maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally comprising gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can comprise the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (e.g., subcutaneously or intramuscularly or by intramuscular injection). Thus, e.g., the compounds may be formulated with suitable polymeric or hydrophobic materials (e.g., as an emulsion in an acceptable oil) or ion exchange resins, or as sparingly soluble derivatives, e.g., as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 400, made up to volume in absolute ethanol. The VPD co-solvent system (VPD:5W) comprises VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: e.g., other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Pharmaceutical compositions suitable for use in the present invention include compositions wherein the active ingredients are present in an effective amount to achieve its intended purpose. More specifically, a therapeutically effective amount means an amount effective to prevent development of or to alleviate existing symptoms of a subject being treated. Determination of the effective amounts is well within the capability of those skilled in the art.

### Dosage

For any compound used in a method of the present invention, the therapeutically effective dose can be estimated initially from cellular assays. For example, a dose can be formulated in cellular and animal models to achieve a circulating concentration range that includes the EC₅₀ as determined in cellular assays (i.e., the concentration of the test compound which achieves a half-maximal inhibition of a given receptor activity). In some cases it is appropriate to determine the EC₅₀ in the presence of 3 to 5% serum albumin since such a determination approximates the binding effects of plasma protein on the compound. Such information can be used to more accurately determine useful doses in humans. Further, advantageous compounds for systemic administration effectively modulate receptors of the S1P family in intact cells at levels that are safely achievable in plasma.

A therapeutically effective dose refers to that amount of the compound that results in amelioration of symptoms in a subject. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds that exhibit high therapeutic indices are suitable. The data obtained from these cell culture assays and animal studies can be used in formulating a range of dosage for use in humans. The dosage of such compounds lies typically within a range of circulating concentrations that include the ED₅₀ with little or no toxicity. The dosage may vary within this range depending upon the dosage form employed and the route of administration utilized. The exact formulation, route of administration and dosage can be chosen by a practitioner in view of a patient's condition. (See e.g., Fingl et al., 1975, in “The Pharmacological Basis of Therapeutics”, Ch. 1 p 1). In the treatment of crises, the administration of an acute bolus or an infusion approaching the MTD may be advantageous to obtain a rapid response.

Dosage amount and interval may be adjusted individually to provide plasma levels of the active moiety which are sufficient to modulate receptors of the S1P family, or minimal effective concentration (MEC). The MEC will vary for each compound but can be estimated from in vitro data; e.g. the concentration necessary to achieve 50-90% inhibition of binding of the natural ligand using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen that maintains plasma levels above the MEC for 10-90% of the time, or between 30-90%, or between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device that may contain one or more unit dosage forms containing the active ingredient. The pack may, e.g., comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

### Exemplary Formulations

In some formulations it may be beneficial to use compounds of the present invention in the form of particles of very small size, e.g., as obtained by fluid energy milling.

The use of compounds of the present invention in the manufacture of pharmaceutical compositions is illustrated by the following description. In this description the term “active compound” denotes any compound of the invention but particularly any compound that is the final product of one of the following examples.

a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

b) Tablets

Tablets may be prepared, e.g., from the following ingredients:

| | Parts by weight |
|---|---|
| Active compound | 10 |
| Lactose | 190 |
| Maize starch | 22 |
| Polyvinylpyrrolidone | 10 |
| Magnesium stearate | 3 |

The active compound, lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

c) Enteric Coated Tablets

Tablets may be prepared by the method described in (b) above. The tablets may be enteric coated in a conventional manner using a solution of 20% cellulose acetate phthalate and 3% diethyl phthalate in ethanol:dichloromethane (1:1).

d) Suppositories

In the preparation of suppositories, e.g., 100 parts by weight of active compound can be incorporated in 1300 parts by weight of triglyceride suppository base and the mixture formed into suppositories each containing a therapeutically effective amount of active ingredient.

The present invention also comprises the use of a compound of Formula (I) as a medicament.

A further aspect of the present invention provides the use of a compound of Formula (I) or a salt thereof in the manufacture of a medicament for treating vascular hyperpermeability, angiogenesis-dependent disorders, proliferative diseases and/or disorders of the immune system in mammals, e.g., humans.

The present invention also provides a method of treating vascular hyperpermeability, inappropriate neovascularization, proliferative diseases and/or disorders of the immune system which comprises the administration of a therapeutically effective amount of a compound of Formula (I) to a mammal, e.g., humans, in need thereof.

### S1P Receptor GTPγS Assays

The [³⁵S]GTPγS binding assay can be run using both scintillation proximity assay (SPA) and filtration methods. Both formats are in 96 well plates and utilize membranes from a stable CHO human cell lines overexpressing S1P₁, S1P₂, S1P₃, S1P₄ or S1P₅. Compound stocks were made up to 10 mM using DMSO and serial dilutions were carried out using 100% DMSO. Compounds were transferred to 96 well plates to yield a final DMSO concentration of 0.5% for all assays. Frozen membranes were thawed and diluted in assay buffer containing 20 mM HEPES pH 7.4, 0.1% fatty acid-free BSA, 100 mM NaCl, 5 mM MgCl₂ and 10 μM GDP. For the SPA assay membranes are premixed with WGA-SPA beads to yield a final concentration per well of 5 μg membrane and 500 μg of bead. For the filtration assay, membranes are added directly to the incubation plate at 5 μg per well. The assay begins with the addition of 10 μL of compound, followed by 40 μL of the membrane or membrane/bead mixture to each well of the assay plate. Next, 50 μL of 0.4 nM [³⁵S]GTPS is added to each well and incubated for 30 minutes. For the SPA assay the plates are spun and then read on the Topcount. For the filtration assay the plate is harvested onto GF-C filtration plates using a Packard 96 well harvester.

### Inhibition of [³³P]S1P Binding to S1P Receptors

Radioligand binding was carried out using membranes from transiently transfected HEK or CHO cells overexpressing S1P1, S1P2, S1P3, S1P4 or S1P5. All compounds are dissolved in DMSO and serial dilutions were carried out in DMSO prior to addition to assay buffer. Final assay DMSO concentrations are 1% (v/v). [³³P]S1P is purchased from PerkinElmer and used at 50 pM in all assays. Frozen membranes are thawed and resuspended in assay buffer containing 50 mM HEPES pH 7.4, 100 mM NaCl, 10 mM MgCl₂ and 0.1% fatty acid free BSA. Membrane is added to give 5-10 μg of membrane per well. Non-specific binding is determined in the presence of cold 1 μM SIP. Incubations are carried out at room temperature for 45-60 minutes except for S1P4 that is run at 4° C. for 90 minutes. Reactions were terminated by rapid vacuum filtration using GF/C filtration plates and a Packard 96 well harvester. Plates are dried before adding Microscint to each well, sealed and counted on a Topcount.

### GENERAL PROCEDURES AND EXAMPLES

The general synthetic schemes that were utilized to construct the majority of compounds disclosed in this application are described below in Schemes 1-22. These schemes are provided for illustrative purposes only and are not to be construed as limiting the scope of the invention. The following examples are for illustrative purposes and are not to be construed as limiting the scope of the present invention.

### Abbreviations

| | |
|---|---|
| BEMP | 2-tert-Butylimino-2-diethylamino-1,3-dimethyl-perhydro- |
| | 1,3,2-diazaphosphorine |
| DCE | 1,2-Dichloroethane |
| DCM | Dichloromethane |
| DIBAL-H | Diisobutylaluminum hydride |
| DIAD | Diisopropyl azodicarboxylate |
| DIEA | N,N-Diisopropylethylamine |
| DMF | N,N-Dimethylformamide |
| DMAP | 4-Dimethylaminopyridine |
| DMSO | Dimethyl sulfoxide |
| EtOAc | Ethyl acetate |
| HATU | O-(7-Azabenzotriazol-1-yl)-N,N,N′,N′-tetramethyluronium |
| | hexafluorophosaphate |
| HBTU | O-Benzotriazol-1-yl-N,N,N′,N′-tetramethyluronium |
| | hexafluorophosaphate |
| HCl | Hydrochloric acid |
| HPLC | High performance liquid chromatography |
| LCMS | Liquid chromatography mass spec |
| MeOH | Methanol |
| NaOH | Sodium hydroxide |
| p-TsOH | para-Toluene sulfonic acid |
| RP | Reverse phase |
| Rₜ | Retention time |
| RT | Room temperature |
| TBAF | Tetrabutyl ammonium fluoride |
| TBDMS-Cl | tert-Butyldimethylchlorosilane |
| TFFH | Tetramethyl fluoroformamidium hexafluoro phosphate |
| THF | Tetrahydrofuran |
| TLC | Thin Layer Chromatography |
| W | Watts |

Unless otherwise noted, all starting materials were purchased from Aldrich, Acros or Alfa Aesar.

### Analytical Methods

Analytical data is defined either within the general procedures or in the tables found within the examples. Unless otherwise stated, all ¹H or ¹³C NMR data were collected on a Varian Mercury Plus 400 MHz or a Bruker DRX 400 MHz instrument; chemical shifts are quoted in parts per million (ppm). High performance liquid chromatography (HPLC) analytical data are either detailed within individual experiments or referenced in Table 1.

TABLE 1
List of HPLC methods

| | HPLC Conditions |
|---|---|
| | Unless indicated otherwise mobile phase A was 10 mM ammonium acetate, |
| Method | mobile phase B was HPLC grade acetonitrile. |
| a | 5-95% B over 3.7 min with a hold at 95% B for 1 min (1.3 mL/min flow |
| | rate). 4.6 × 50 mm Zorbax XDB C18 column (5 μm particles). Detection |
| | methods are diode array (DAD) and evaporative light scattering (ELSD) |
| | detection as well as pos/neg electrospray ionization. |
| b | 5-60% B over 1.5 min then 60-95% B to 2.5 min with a hold at 95% B for |
| | 1.2 min (1.3 mL/min flow rate). 4.6 × 30 mm Vydac Genesis C8 column (4 μm |
| | particles). Detection methods are diode array (DAD) and evaporative |
| | light scattering (ELSD) detection as well as pos/neg electrospray ionization. |
| c | 30-95% B over 2.0 min with a hold at 95% B for 1.7 min (1.0 mL/min flow |
| | rate). 4.6 × 30 mm Vydac Genesis C8 column (4 μm particles). Detection |
| | methods are diode array (DAD) and evaporative light scattering (ELSD) |
| | detection as well as pos/neg electrospray ionization. |
| d | The column used for the chromatography was a 4.6 × 30 mm Vydac Genesis |
| | C8 column (4 μm particles). The gradient was 30-95% B in 2 min then hold |
| | at 95% B to 5.7 min (1.3 mL/min flow rate). Mobile phase A was 10 mM |
| | ammonium acetate, mobile phase B was HPLC grade acetonitrile. Detection |
| | methods are diode array (DAD) and evaporative light scattering (ELSD) |
| | detection as well as pos/neg electrospray ionization. |
| e | The gradient was 5-95% B in 3.7 min with a hold at 95% B for 1 min (1.3 mL/min |
| | flow rate). 4.6 × 50 mm Zorbax XDB C18 column (5 μm particles). |
| | Mobile phase A was 10 mM ammonium acetate, mobile phase B was HPLC |
| | grade acetonitrile. Detection methods are diode array (DAD) and |
| | evaporative light scattering (ELSD) detection as well as pos/neg atmospheric |
| | pressure chemical ionization (APCI). |
| f | The gradient was 5-95% B in 3.7 min with a hold at 95% B for 1 min (1.3 mL/min |
| | flow rate). 4.6 × 50 mm Zorbax XDB C18 column (5 μm particles). |
| | Mobile phase A was water with 0.1% formic acid, mobile phase B was |
| | HPLC grade acetonitrile. Detection methods are diode array (DAD) and |
| | evaporative light scattering (ELSD) detection as well as pos/neg electrospray |
| | ionization. |
| g | The column used for the chromatography is a 4.6 × 50 mm MAC-MOD Halo |
| | C18 column (2.7 μm particles). The gradient was 5-60% B in 1.5 min then |
| | 60-95% B to 2.5 min with a hold at 95% B for 1.2 min (1.3 mL/min flow |
| | rate). Mobile phase A was 10 mM ammonium acetate, mobile phase B was |
| | HPLC grade acetonitrile. Detection methods are diode array (DAD) and |
| | evaporative light scattering (ELSD) detection as well as pos/neg electrospray |
| | ionization. |
| h | The column used for the chromatography is a 4.6 × 50 mm MAC-MOD Halo |
| | C8 column (2.7 μm particles). The gradient was 5-60% B in 0.75 min then |
| | 60-95% B to 1.15 min with a hold at 95% B for 0.75 min (1.3 mL/min flow |
| | rate). Mobile phase A was 10 mM ammonium acetate, mobile phase B was |
| | HPLC grade acetonitrile. Detection methods are diode array (DAD) and |
| | evaporative light scattering (ELSD) detection as well as pos/neg electrospray |
| | ionization. |
| i | The column used for the chromatography is a 4.6 × 50 mm MAC-MOD Halo |
| | C8 column (2.7 μm particles). The gradient was 5-60% B in 1.5 min then |
| | 60-95% B to 2.5 min with a hold at 95% B for 1.2 min (1.3 mL/min flow |
| | rate). Mobile phase A was 10 mM ammonium acetate, mobile phase B was |
| | HPLC grade acetonitrile. Detection methods are diode array (DAD) and |
| | evaporative light scattering (ELSD) detection as well as pos/neg electrospray |
| | ionization. |
| j | The column used for the chromatography is a 4.6 × 50 mm MAC-MOD Halo |
| | C8 column (2.7 μm particles). The gradient was 30-60% B in 1.50 min then |
| | 60-95% B to 2.5 min with a hold at 95% B for 1.2 min (1.3 mL/min flow |
| | rate). Mobile phase A was 10 mM ammonium acetate, mobile phase B was |
| | HPLC grade acetonitrile. Detection methods are diode array (DAD) and |
| | evaporative light scattering (ELSD) detection as well as pos/neg electrospray |
| | ionization. |
| k | The column used for the chromatography is a 4.6 × 50 mm MAC-MOD Halo |
| | C8 column (2.7 μm particles). The gradient was 30-60% B in 0.75 min then |
| | 60-95% B to 1.15 min with a hold at 95% B for 0.75 min (1.3 mL/min flow |
| | rate). Mobile phase A was 10 mM ammonium acetate, mobile phase B was |
| | HPLC grade acetonitrile. Detection methods are diode array (DAD) and |
| | evaporative light scattering (ELSD) detection as well as pos/neg electrospray |
| | ionization. |
| l | The column used for the chromatography was a 4.6 × 30 mm Vydac Genesis |
| | C8 column (4 μm particles). The gradient was 5-35% B in 4 min then 35-95% |
| | B to 6 min with a hold at 95% B for 1.7 min (1.3 mL/min flow rate). |
| | Mobile phase A was water with 0.1% formic acid, mobile phase B was |
| | HPLC grade acetonitrile. Detection methods are diode array (DAD) and |
| | evaporative light scattering (ELSD) detection as well as pos/neg electrospray |
| | ionization. |
| m | The column used for the chromatography was a 4.6 × 50 mm MAC-MOD |
| | Halo C8 column (4 μm particles). The gradient was 5-60% B in 1.5 min |
| | then 60-95% B to 2.5 min with a hold at 95% B for 1.2 min (1.3 mL/min |
| | flow rate). Mobile phase A was 10 mM ammonium acetate, mobile phase B |
| | was HPLC grade acetonitrile. Detection methods are diode array (DAD) and |
| | evaporative light scattering (ELSD) detection as well as pos/neg atmospheric |
| | pressure chemical ionization (APCI). |
| n | Samples were purified by preparative HPLC on a Phenomenex Luna C8(2) 5 |
| | um 100 Å AXIA column (30 mm × 75 mm). A gradient of acetonitrile (A) |
| | and 0.1% trifluoroacetic acid in water (B) was used, at a flow rate of |
| | 50 mL/min (0-0.5 min 10% A, 0.5-6.0 min linear gradient 10-100% A, 6.0-7.0 min |
| | 100% A, 7.0-8.0 min linear gradient 100-10% A). Samples were |
| | injected in 1.5 mL DMSO:MeOH (1:1). An Agilent 1100 Series Purification |
| | system was used, consisting of the following modules: Agilent 1100 Series |
| | LC/MSD SL mass spectrometer with API-electrospray source; two Agilent |
| | 1100 Series preparative pumps; Agilent 1100 Series isocratic pump; Agilent |
| | 1100 Series diode array detector with preparative (0.3 mm) flow cell; |
| | Agilent active-splitter, IFC-PAL fraction collector/autosampler. The make- |
| | up pump for the mass spectrometer used 3:1 methanol:water with 0.1% |
| | formic acid at a flow rate of 1 mL/min. Fraction collection was automatically |
| | triggered when the extracted ion chromatogram (EIC) for the target mass |
| | exceeded the threshold specified in the method. The system was controlled |
| | using Agilent Chemstation (Rev B.10.03), Agilent A2Prep, and Leap |
| | FractPal software, with custom Chemstation macros for data export. |

### General Synthetic Schemes

The general synthetic schemes that were utilized to construct the majority of compounds disclosed in herein are described below in schemes 1-22.

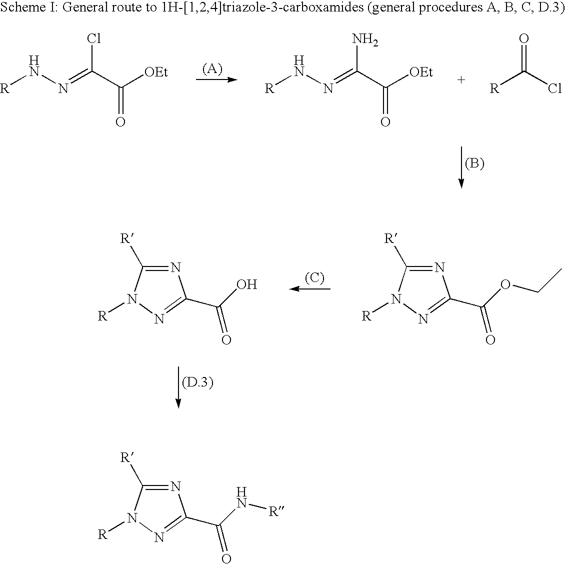

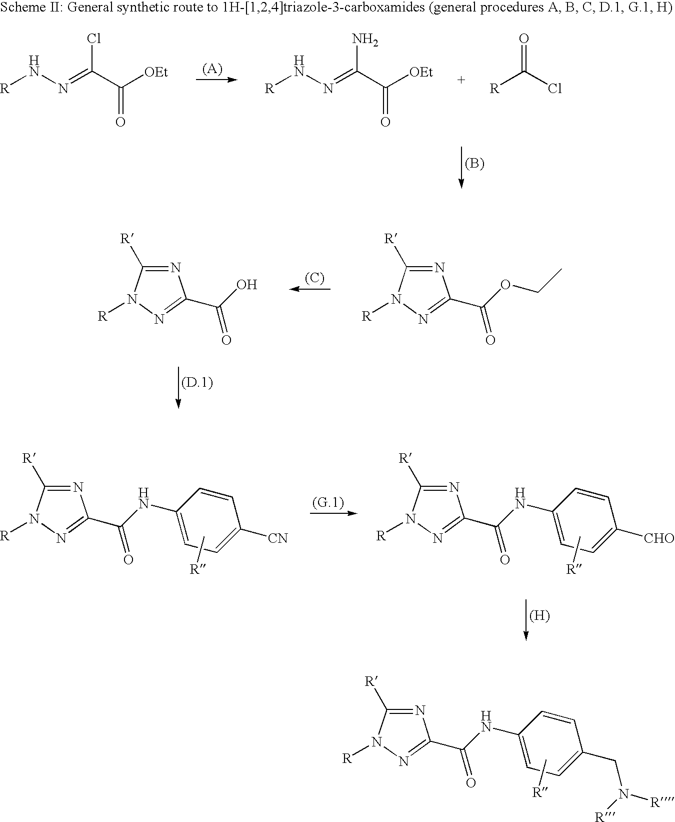

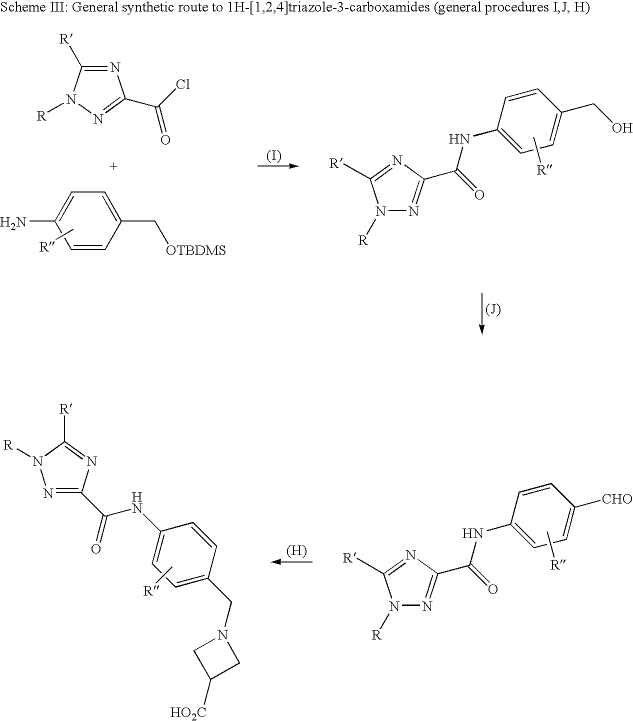

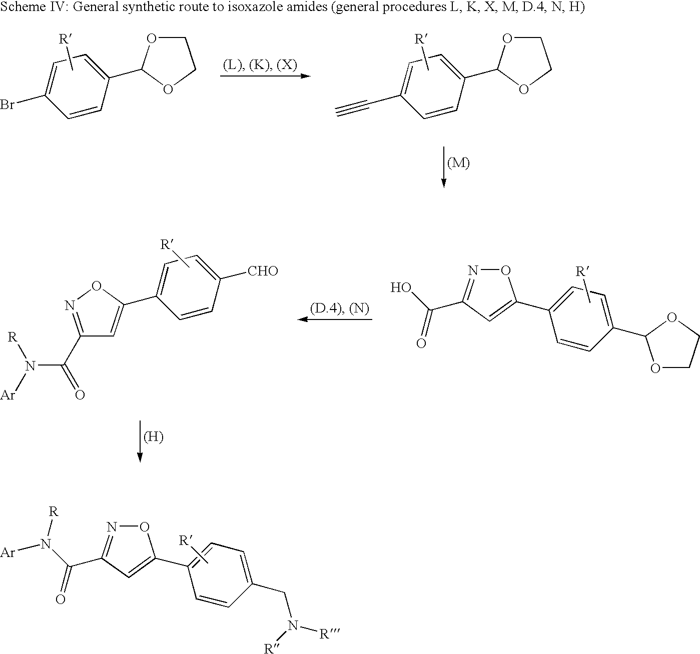

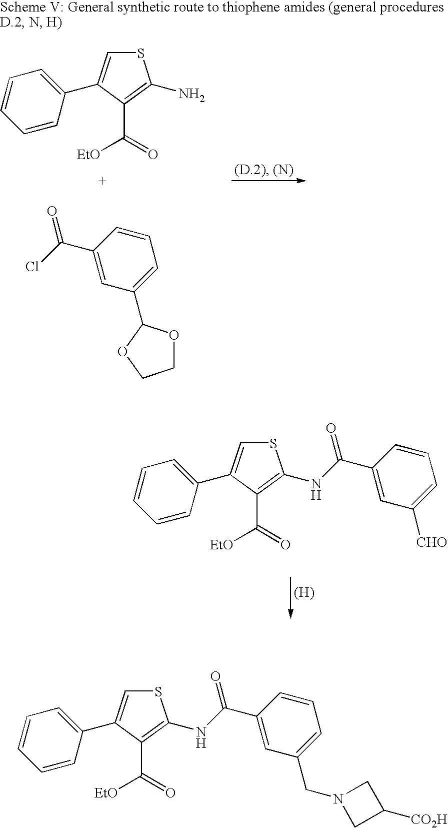

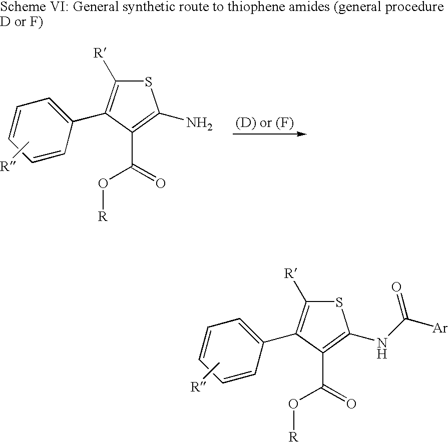

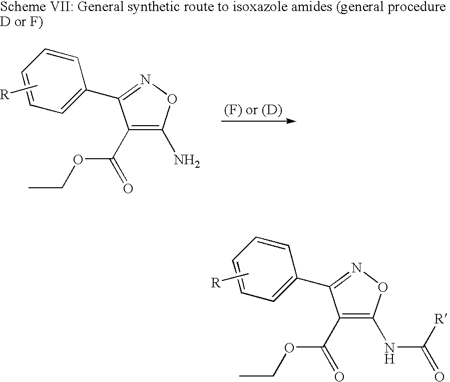

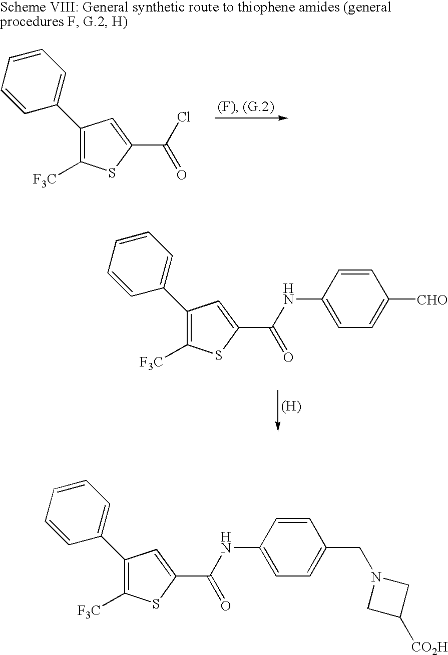

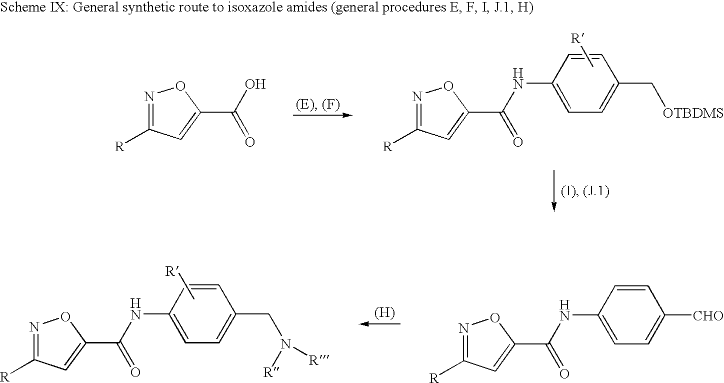

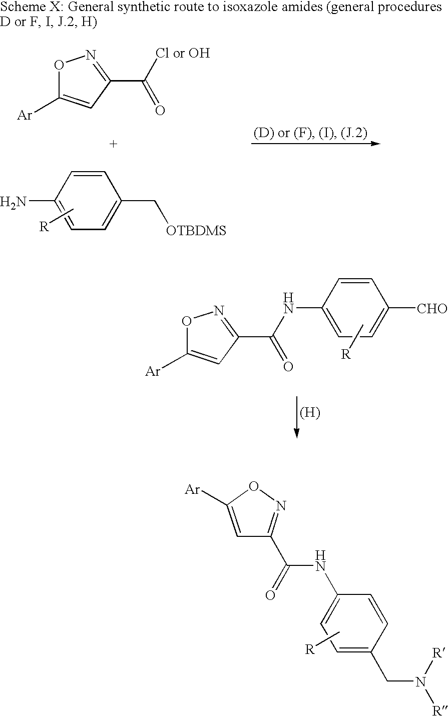

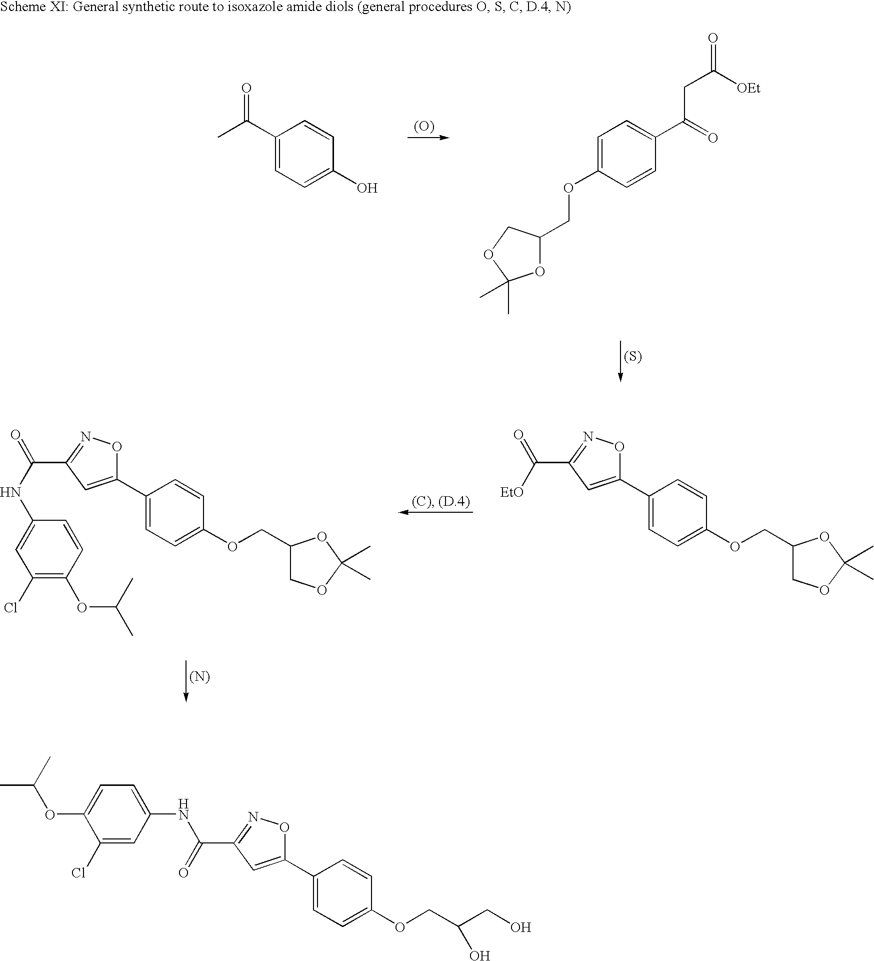

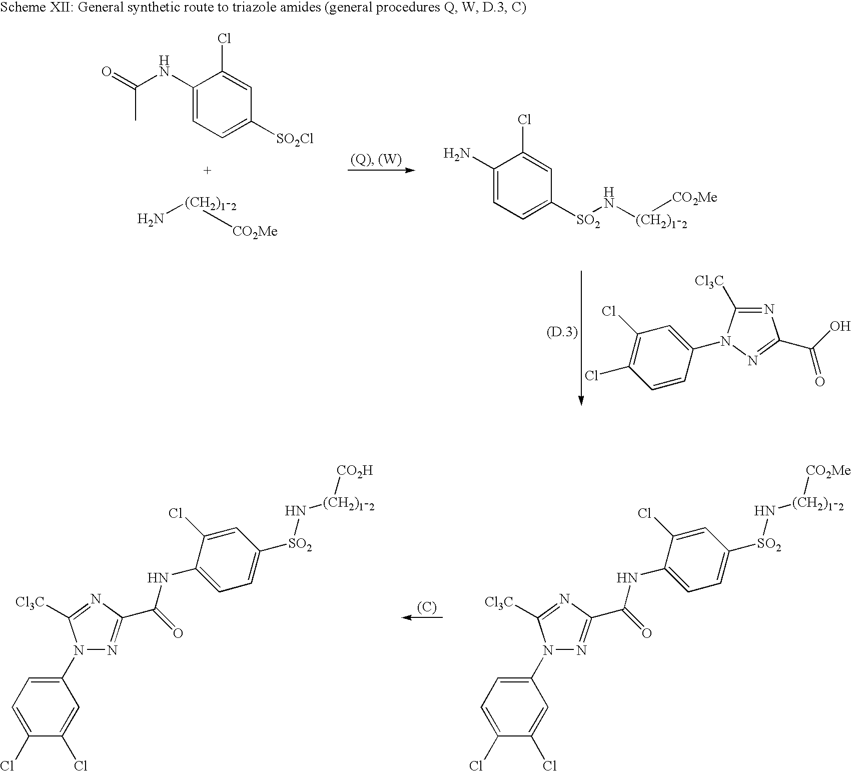

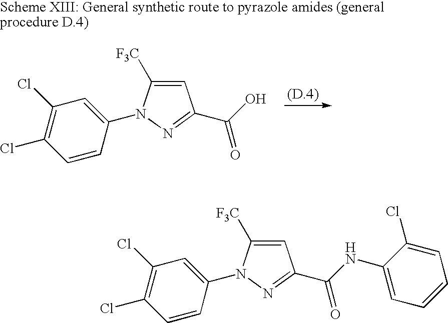

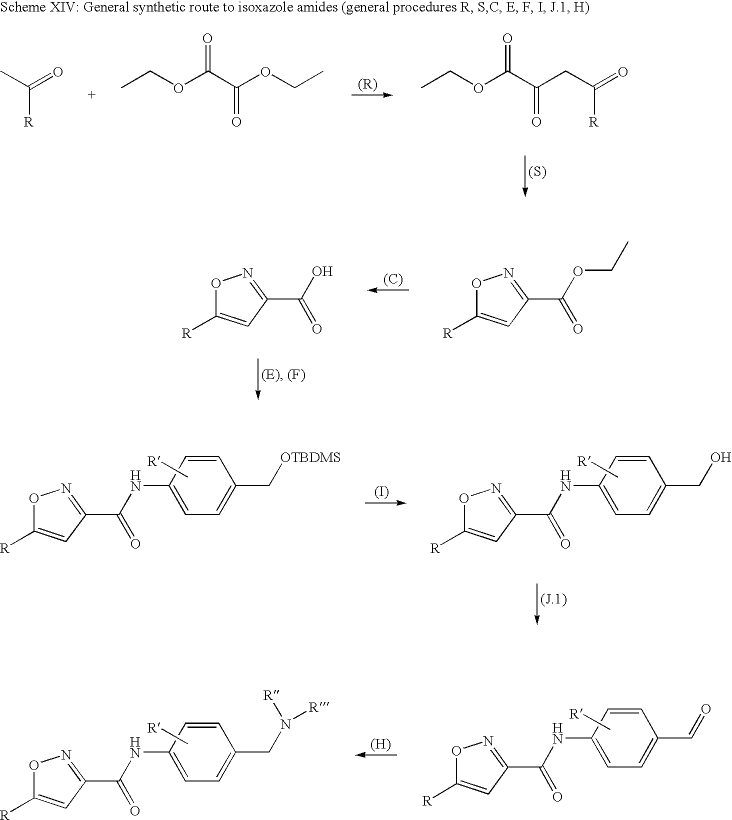

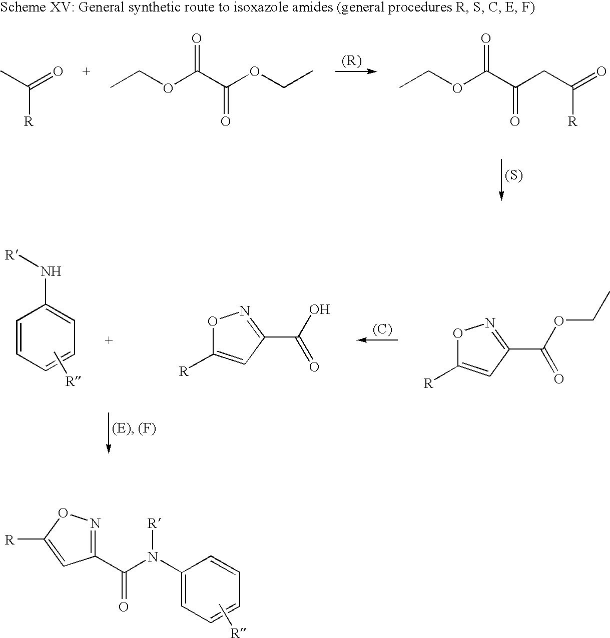

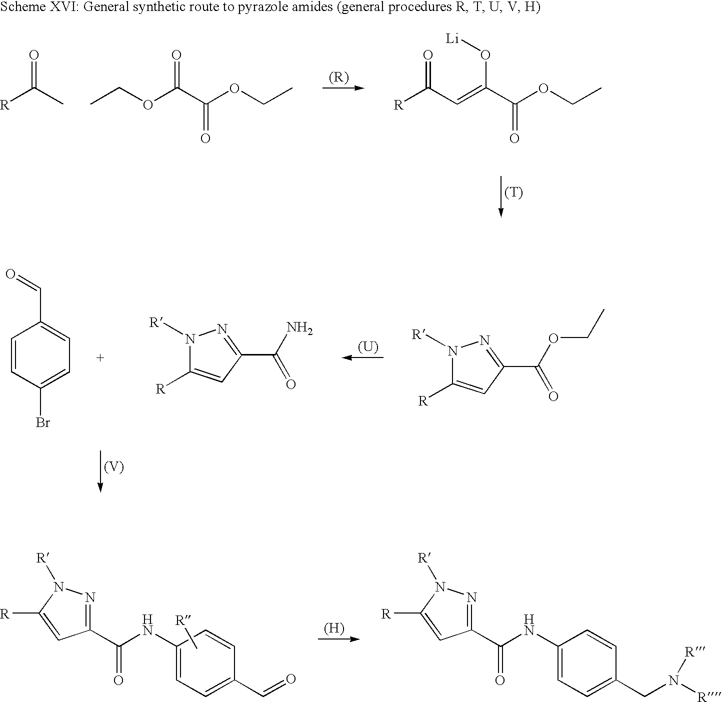

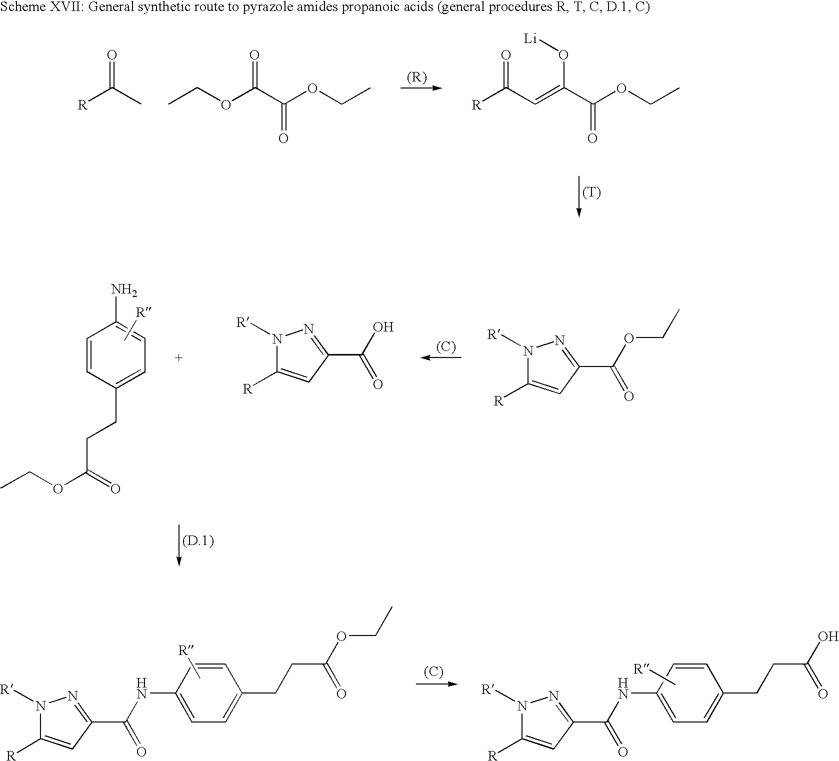

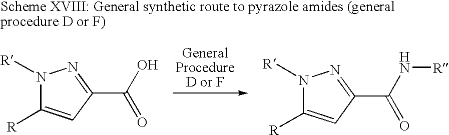

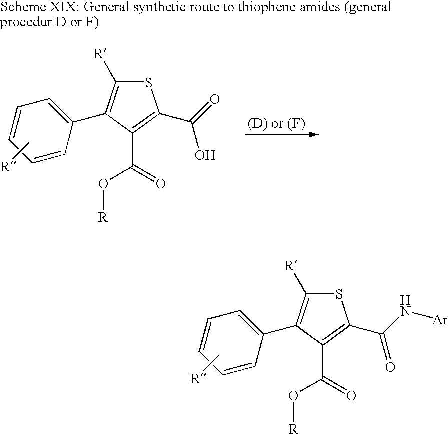

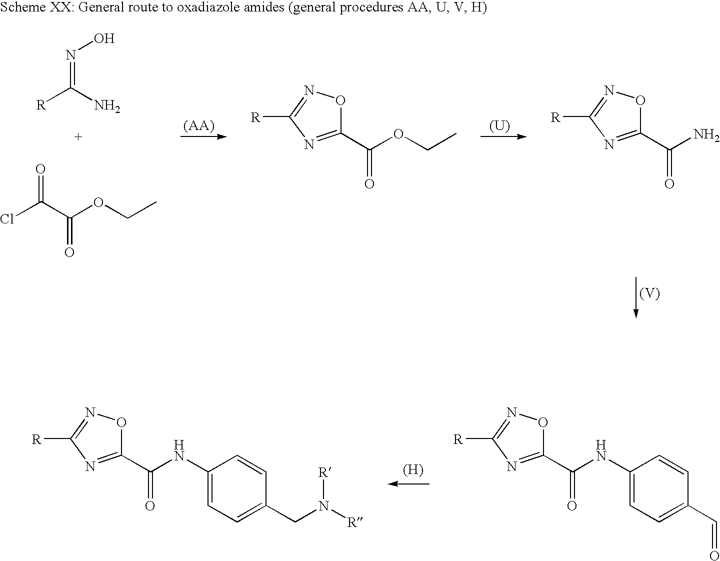

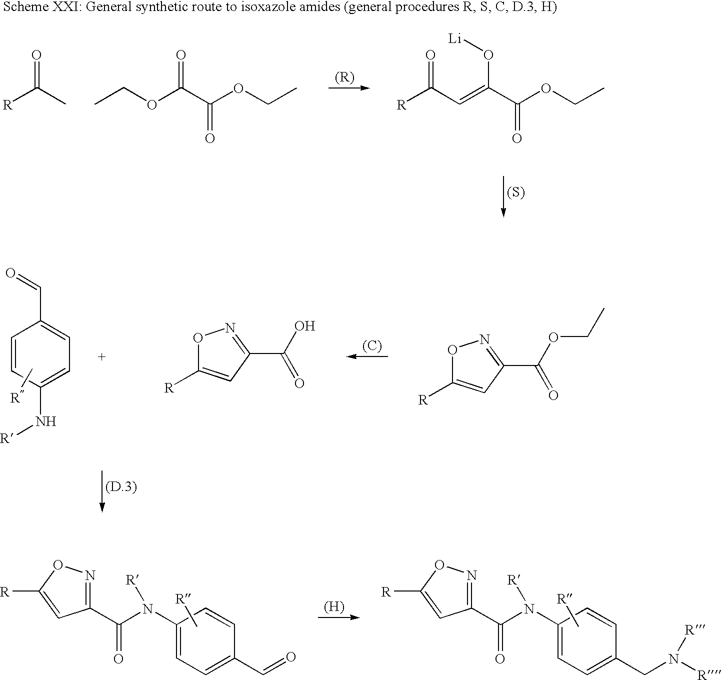

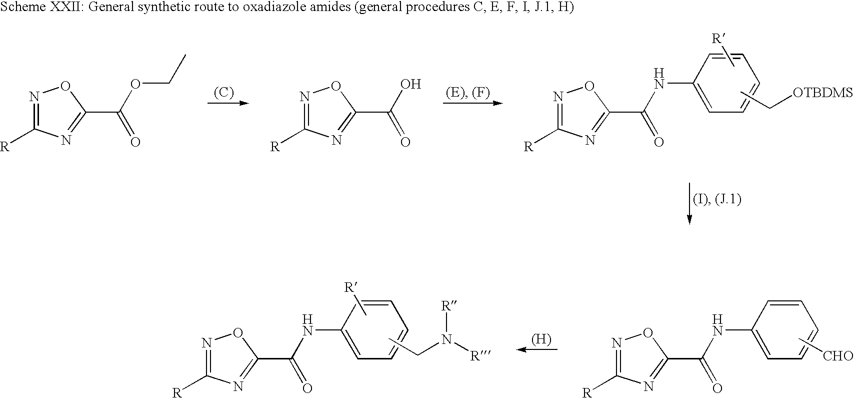

### List of General Procedures

General procedure A: Aminolysis of an aryl or heteroaryl 2-chlorohydrazono acetate
General procedure B: Cyclization of an amino hydrazono acetate to a triazole
General procedure C: Hydrolysis of an ester
General procedure D.1: Amide bond formation via TFFH coupling
General procedure D.2: Amide bond formation via BEMP/HBTU
General procedure D.3: Amide bond formation via POCl₃/pyridine
General procedure D.4: Amide bond formation via HATU
General procedure E: Formation of an acid chloride from carboxylic acid
General procedure F: Reaction of an acid chloride with an amine
General procedure G.1: Reduction of a nitrile to an aldehyde with DIBAL
General procedure G.2: Reduction of a nitrile to aldehyde with sodium dihydrogen phosphate monohydrate and Raney nickel
General procedure H: Reductive amination of an aldehyde
General procedure I: Deprotection of a silyl protected alcohol
General procedure J.1: Oxidation of an alcohol to an aldehyde with barium manganate
General procedure J.2: Oxidation of an alcohol to an aldehyde with manganese oxide
General procedure K: Sonagashira coupling of an alkyne with an aryl bromide
General procedure L: Protection of an aldehyde as the acetal
General procedure M: Formation of an isoxazole from an alkyne
General procedure N: Deprotection of an acetal to an aldehyde
General procedure O: Mitsunobu reaction of an alcohol
General procedure P: Reaction of a B-ketoester to form a 3-Phenyl-isoxazole-5-carboxylic acid ethyl ester
General procedure Q: Reaction of a sulfonyl chloride with an amine
General procedure R: Reaction of a ketone with diethyl oxalate
General procedure S: Cyclization to a 5-arylisoxazole-3-carboxylate
General procedure T: Cyclization to a pyrazole
General procedure U: Aminolysis of an ester to an amide
General procedure V: Buchwald coupling of an amide to an aryl bromide
General procedure W: Deprotection of an acetamide to an amine
General procedure X: Deprotection of a silyl protected alkyne
General procedure Y: Michael addition of a amine to an α,β-unsaturated ester
General procedure Z: Deprotection of a Boc-protected amine
General procedure AA: Cyclization of a hydroxyamidine to an oxadiazole
General procedure BB: Reduction of a nitro compound to an amine

### EXAMPLE OF USE OF GENERAL PROCEDURES

The general procedure letter codes constitute a synthetic route to the final product. A worked example of how the route is determined is given below using “Ex. #” as a non-limiting illustration. The synthesis of Ex. #A1, Table A was completed using general procedures A, B, C, D.3 which translates into the scheme shown below.

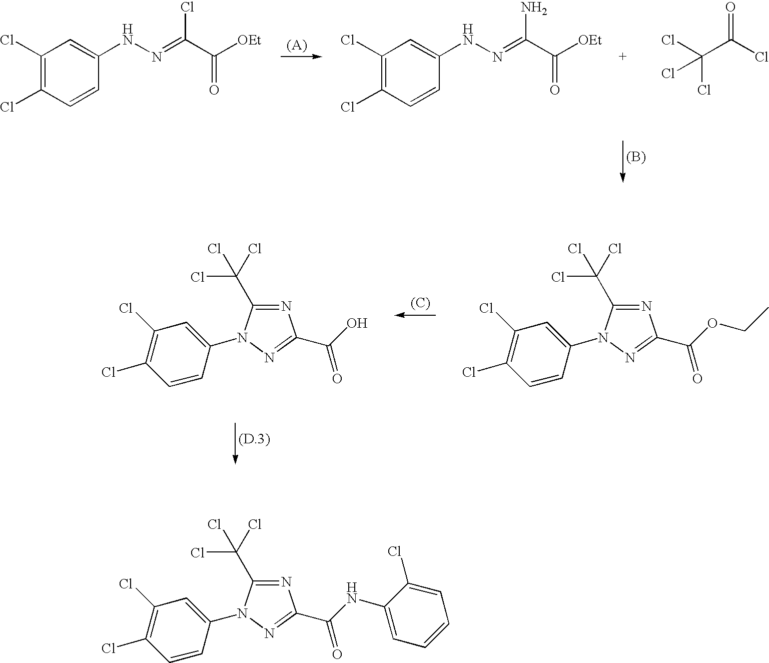

Unless otherwise noted, all starting materials were purchased from Aldrich, Acros or Alfa Aesar.

### General Procedures

The following describe the synthetic methods illustrated by the foregoing General Procedures schemes and are followed by an example of a compound that was synthesized by the General Procedure. The specific conditions and reagents noted in the following are not to be construed as limiting the scope of the instant invention and are provided for illustrative purposes only.

General Procedure A: Aminolysis of an Aryl or Heteroaryl 2-chlorohydrazono acetate

Ammonia gas is bubbled through a stirred solution of the desired 2-chlorohydrazono acetate in an organic solvent (typically dioxane). On completion of the reaction as monitored by TLC the resulting salts are removed by filtration and the filtrate concentrated to provide the desired product.

### Exemplification of General Procedure A

### Preparation of (Z)-ethyl 2-amino-2-(2-phenylhydrazono)acetate

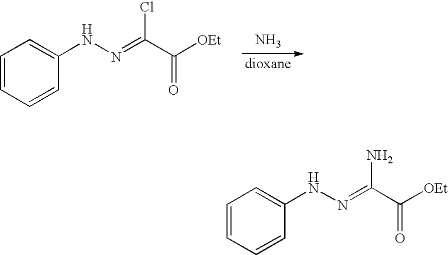

Ammonia was bubbled through a stirring solution of (Z)-ethyl 2-chloro-2-(2-phenylhydrazono)acetate (Oakwood) (20 g, 88 mmol) in dioxane (120 mL). The reaction was monitored by TLC. After the reaction was complete, the resulting yellow suspension was filtered to remove the salts and the filtrate concentrated to afford (Z)-ethyl 2-amino-2-(2-phenylhydrazono)acetate (18 g, 84 mmol, 95% yield) as a yellow solid. LCMS (Table 1, Method a) Rₜ=3.05 min, m/z 208.09 (M+H)⁺; ¹H NMR (400 MHz, DMSO-d6) δ ppm 8.64 (s, 1H), 7.16-7.20 (m, 2H), 7.01 (ddd, 2H, J=1.45, 2.45, 2.98 Hz), 6.71 (tt, 2H, J=1.15, 1.15, 7.34, 7.34 Hz), 5.86 (2, 2H), 4.22 (q, 2H, J=7.10 Hz), 1.28 (t, 3H, J=7.10 Hz).

### General Procedure B: Cyclization of an Amino Hydrazono Acetate to a Triazole

To a suspension of the 2-amino hydrazono acetate (1 eq.) in an organic solvent (e.g., toluene) is added 1-5 eq. (e.g., 2.3 eq.) of the appropriate acid chloride dropwise over about 15 minutes. The mixture is then heated at about reflux for about 1040 hours (e.g., 28 hours). After cooling to ambient temperature the reaction mixture is transferred to a separatory funnel and diluted with an organic solvent (e.g., ethyl acetate). The organic layer is washed with saturated sodium bicarbonate and then water. The organic solvent is then removed under vacuum to provide the desired triazole ester.

### Exemplification of General Procedure B

### Preparation of ethyl 1-phenyl-5-(trichloromethyl)-1H-1,2,4-triazole-3-carboxylate

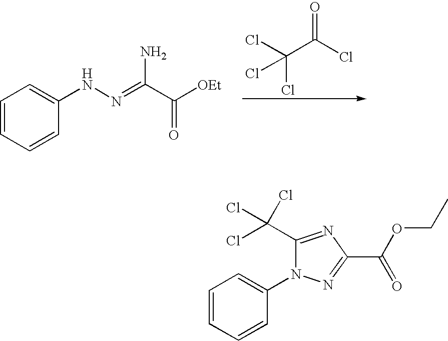

To a suspension of (Z)-ethyl 2-amino-2-(2-phenylhydrazono)acetate (18 g, 87 mmol) in toluene (174 mL) was added trichloroacetyl chloride (22 mL, 197 mmol) dropwise over 15 min. The reaction mixture was heated at reflux for 28 h. The reaction was cooled to ambient temperature. The reaction mixture was transferred to separatory funnel and ethyl acetate (50 mL) was added. The organic layer was washed with saturated sodium bicarbonate (3×100 mL) and water (3×100 mL). Concentration afforded ethyl 1-phenyl-5-(trichloromethyl)-1H-1,2,4-triazole-3-carboxylate (29 g, 87 mmol, 100% yield) as an orange solid. LCMS (Table 1, Method a) Rₜ=3.78 min, m/z: 335.94 (M+H)⁺; ¹H NMR (400 MHz, DMSO-d6) δ ppm 7.60-7.72 (m, 5H), 4.40 (q, 2H, J=7.20 Hz), 1.34 (t, 3H, J=7.20 Hz).

### General Procedure C: Hydrolysis of an Ester

The ester (1 eq.) is stirred in mixture of organic solvent (e.g., THF or dioxane) and water at about ambient temperature. One to ten eq. (e.g., 3 eq.) of a hydroxide base (e.g., lithium hydroxide or sodium hydroxide) is then added in single portion. The reaction is then stirred at about room temperature for about 10-40 hours (e.g., 24 hours). The reaction is acidified with an acid (e.g., acetic acid) to a pH of about 5. The solvents are then removed under vacuum and the crude material purified by flash column chromatography or semi-prep LCMS to provide the desired carboxylic acid.

### Exemplification of General Procedure C

### Hydrolysis of ethyl 1-phenyl-5-(trichloromethyl)-1H-1,2,4-triazole-3-carboxylate to 1-phenyl-5-(trichloromethyl)-1H-1,2,4-triazole-3-carboxylic acid

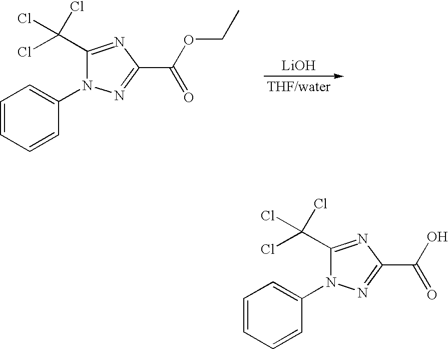

Ethyl 1-phenyl-5-(trichloromethyl)-1H-1,2,4-triazole-3-carboxylate (1.2 g, 3.59 mmol) in THF (8.69 mL) and water (2.174 mL) at ambient temperature was added lithium hydroxide monohydrate (0.451 g, 10.76 mmol) as a single portion. The reaction mixture was stirred at ambient temperature for 24 hours. The reaction mixture was neutralized by the careful addition of acetic acid to ca. pH 5. The solvent was removed in vacuo and the crude material purified by flash column chromatography in 20% methanol/dichloromethane to afford 1-phenyl-5-(trichloromethyl)-1H-1,2,4-triazole-3-carboxylic acid (1.02 g, 3.33 mmol, 93% yield) as a white solid. LCMS (Table 1, Method a) Rₜ 1.80 min, m/z (M−H)⁻ 307.25.

### General Procedure D.1: Amide Bond Formation Via TFFH Coupling

To a solution of 1-(3,4-dichlorophenyl)-5-(trichloromethyl)-1H-1,2,4-triazole-3-carboxylic acid in an organic solvent (e.g., DCM) is added 1 to 10 equivalents of TFFH (e.g., 1 equivalents), followed by 1 to 10 equivalents of an aryl amine (e.g., 1.0 equivalents). The reaction mixture is stirred at about 0° C. to 100° C. (e.g., 20° C.) for about 12 to 48 hours (e.g., 24 hours). The reaction mixture is concentrated in vacuo and the product can be purified by chromatography.

### Exemplification of General Procedure D.1

### Preparation of N-(2-chloro-4-cyanophenyl)-1-(3,4-dichlorophenyl)-5-(trichloromethyl)-1H-1,2,4-triazole-3-carboxamide

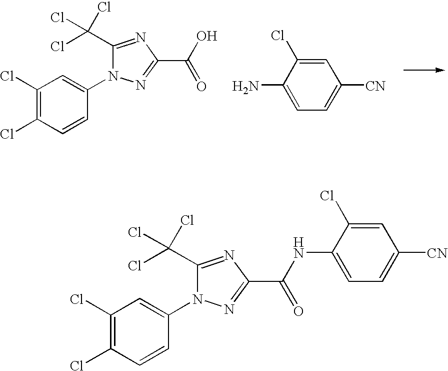

1-(3,4-Dichlorophenyl)-5-(trichloromethyl)-1H-1,2,4-triazole-3-carboxylic acid (1.6 g, 4.26 mmol) in DCM (20 mL) was added TFFH (1.13 g, 4.26 mmol). The reaction mixture was stirred at ambient temperature for 5 minutes before addition of 4-amino-3-chlorobenzonitrile (0.650 g, 4.26 mmol). The reaction mixture was stirred for 24 hours at ambient temperature. The solvent was removed in vacuo and the crude material purified by flash column chromatography in 50% ethyl acetate/heptane to afford N-(2-chloro-4-cyanophenyl)-1-(3,4-dichlorophenyl)-5-(trichloromethyl)-1H-1,2,4-triazole-3-carboxamide (406 mg, 0.796 mmol, 18% yield) as a white solid. LCMS (Method a) Rₜ 3.22 min; m/z: (M+H)⁺ 510.168.

### General Procedure D.2: Amide Bond Formation Via BEMP/HBTU

A carboxylic acid (1.3 eq.), an amine (1. eq.), BEMP (3.9 eq.), and HBTU (1.3 eq.) are combined in an organic solvent (e.g., acetonitrile). The mixture is heated to about 140° C. in a microwave (e.g., Biotage) for about 30 minutes. The solvent is removed under reduced pressure and the residue purified by flash column chromatography to provide the desired product.

### Exemplification of General Procedure D.2

### Preparation of ethyl 2-(3-(1,3-dioxolan-2-yl)-benzamido)-4-phenylthiophene-3-carboxylate

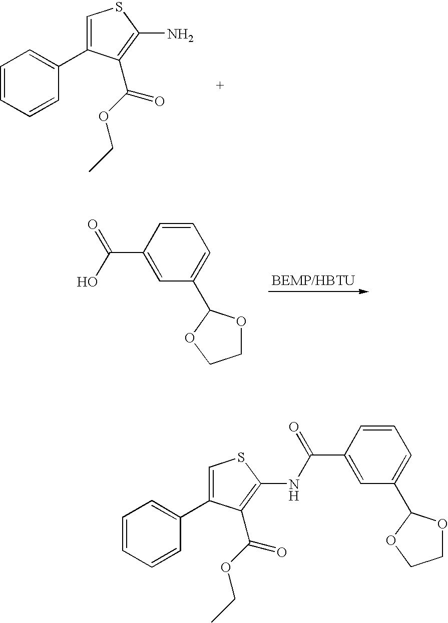

A 5 mL microwave vial equipped with a stirring bar was charged with 3-(1,3-dioxolan-2-yl)benzoic acid (306 mg, 1.577 mmol), ethyl 2-amino-4-phenylthiophene-3-carboxylate (300 mg, 1.213 mmol), BEMP (1.369 mL, 4.73 mmol), HBTU (598 mg, 1.577 mmol), and acetonitrile (3.5 mL). The vessel was capped and the reaction heated to 140° C. for 30 min under microwave irradiation (Biotage Optimizer, 300 W). Solvent was removed under reduced pressure, and the crude material purified by flash column chromatography (Analogix System; 80 g column; 100% heptane ramping to 50% ethyl acetate in heptane over 30 min) to give ethyl 2-(3-(1,3-dioxolan-2-yl)benzamido)-4-phenylthiophene-3-carboxylate (138 mg, 26.6%). LCMS (Table 1, Method c) Rₜ=3.03 min, m/z=422.38 (M−H)⁻; ¹H NMR (400 MHz, DMSO-d6) δ ppm 11.98 (s, 1H), 8.03 (s, 1H), 8.01-7.95 (m, 1H), 7.76 (d, J=7.69 Hz, 1H), 7.68 (t, J=7.66 Hz, 1H), 7.42-7.28 (m, 5H), 7.01 (s, 1H), 5.88 (s, 1H), 4.16-4.10 (m, 2H), 4.10-3.98 (m, 4H), 0.96 (t, J=7.11 Hz, 3H).

### General Procedure D.3: Amide Bond Formation Via POCl3/Pyridine

A solution of the carboxylic acid (1 eq.) and the amine (1 eq.) in an organic solvent (e.g., pyridine) is cooled to about −15° C. To the reaction is added cautiously 1-10 eq. (e.g., 2 eq.) of phosphorous oxychloride with rapid stirring. The reaction is stirred at about −15° C. for about 0-30 min (e.g., 10 min.) until the reaction is complete as monitored by TLC. The reaction mixture is quenched by the addition of water and extracted with an organic solvent (e.g., ethyl acetate). The combined extracts are washed with an aqueous base (e.g., saturated sodium bicarbonate) and then washed with brine. The organic solution is then dried over an anhydrous salt (e.g., sodium sulfate), filtered, and concentrated under vacuum. The residue is purified by flash column chromatography to provide the desired product.

### Exemplification of General Procedure D.3

### Example #1

### Preparation of N-(2-chlorophenyl)-1-phenyl-5-(trichloromethyl)-1H-1,2,4-triazole-3-carboxamide

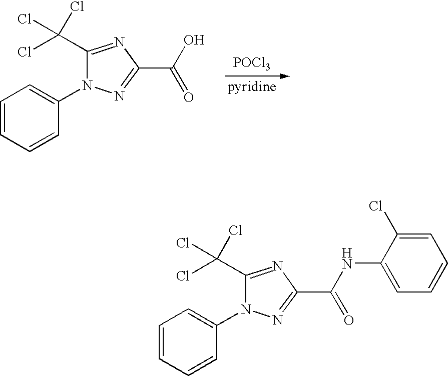

1-Phenyl-5-(trichloromethyl)-1H-1,2,4-triazole-3-carboxylic acid (100 mg, 0.199 mmol) and 2-chloroaniline (25.4 mg, 0.199 mmol) in dry pyridine (596 μL) was cooled to −15° C. To the reaction mixture was added phosphorus oxychloride (37.1 μL, 0.398 mmol) slowly over 2 minutes with vigorous stirring. The reaction mixture was stirred at −15° C. for 10 minutes and monitored by TLC. TLC in 50% ethyl acetate/heptane after 10 minutes indicated there was no starting material present in the reaction mixture. The reaction mixture was quenched by addition of ice water (15 mL) and extracted with ethyl acetate (3×10 mL). The combined organics were washed with saturated NaHCO₃ (15 mL) and brine (3×10 mL). The combined organics were dried over Na₂SO₄ and concentrated in vacuo. The crude material was purified by flash column chromatography in 50% ethyl acetate/heptane to afford N-(2-chlorophenyl)-1-phenyl-5-(trichloromethyl)-1H-1,2,4-triazole-3-carboxamide (65 mg, 0.150 mmol, 75% yield) as a white solid. RP-HPLC (Method a) Rₜ 3.06 min, m/z: (M+H)⁺ 415.24. ¹H NMR (400 MHz, MeOH-d4) δ ppm 8.24 (dd, 1H), 7.66 (m, 5H), 7.52 (dd, 1H), 7.39 (m, 1H), 7.24 (dd, 1H).

### General Procedure D.4: Amide Bond Formation Via HATU

Carboxylic acid (1 eq.) is suspended in an organic solvent (e.g., DMF). To this is added an organic base (e.g., diisopropylethylamine, 3 eq.). The resulting mixture is stirred for about 5 minutes. HATU (1.5 eq.) is added as a solution in the organic solvent of choice. The amine (1 eq.) is added and the reaction is stirred at ambient temperature for 2-24 hours (e.g., 16 hours). The solvents are removed in vacuo and the residue purified by flash column chromatography to give the desired product.

### Exemplification of General Procedure D.4

### Preparation of 5-(4-(1,3-dioxolan-2-yl)phenyl)-N-(3-chloro-4-isopropoxyphenyl)isoxazole-3-carboxamide

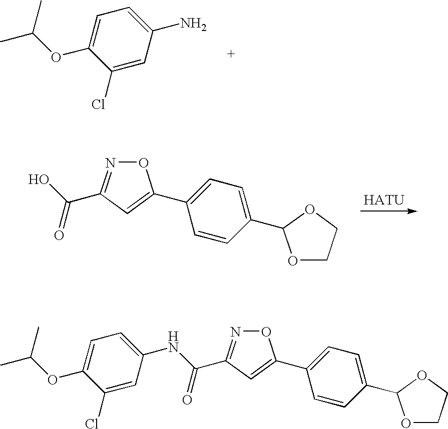

5-(4-(1,3-Dioxolan-2-yl)phenyl)isoxazole-3-carboxylic acid (0.243 g, 0.930 mmol) was suspended in DMF (2.0 mL). To this was added DIEA (0.486 mL, 2.79 mmol). The resulting mixture was stirred for about 5 min., then HATU (0.531 g, 1.395 mmol) was added as solution in DMF (1.5 mL), followed by 3-chloro-4-isopropoxyaniline (0.173 g, 0.930 mmol). The reaction was stirred at ambient temperature overnight. Solvent was removed in vacuo and the residue was purified directly via Analogix flash chromatography system using RediSep® RS 40 g column, with a gradient of 0-50% EtOAc/Heptane over 35 min. at 30 mL/min. to give 5-(4-(1,3-dioxolan-2-yl)phenyl)-N-(3-chloro-4-isopropoxyphenyl)isoxazole-3-carboxamide (180 mg, 0.38 mmol) as white solid. ¹H NMR (400 MHz, CD₂Cl₂) δ ppm 8.51 (s, 1H), 7.87 (d, J=8.1 Hz, 2H), 7.82 (d, J=2.7 Hz, 1H), 7.63 (dd, J=3.6, 5.0 Hz, 2H), 7.48 (dd, J=2.7, 8.9 Hz, 1H), 7.09 (t, J=0.9 Hz, 1H), 7.00 (d, J=8.8 Hz, 1H), 5.85 (s, 1H), 4.57 (td, J=6.1, 12.1 Hz, 1H), 4.09 (m, 4H), 1.38 (dd, J=0.6, 6.1 Hz, 6H).

### General Procedure D.5

### Amide Bond Formation Via HOBT/EDC

A carboxylic acid (1-2 equiv, e.g., 1 eq.), an amine (1-2 eq., e.g., 1 eq.), HOBT (1-3 eq., e.g., 1.5 eq.), and EDC (1-3 eq., e.g., 1.1 equiv.) are combined in an organic solvent (e.g., DMF). The mixture is stirred at about 0-70° C. (e.g., about 23° C.) for about 2-24 h (e.g., about 6 h). The solvent is removed under reduced pressure and the residue purified by crystallization or trituration from an appropriate solvent or solvents, or by chromatography to give the target compound.

### Exemplification of General Procedure D.5

### Example #2

### Preparation of tert-butyl 4-(5-(1H-indazol-5-yl)isoxazole-3-carboxamido)piperidine-1-carboxylate

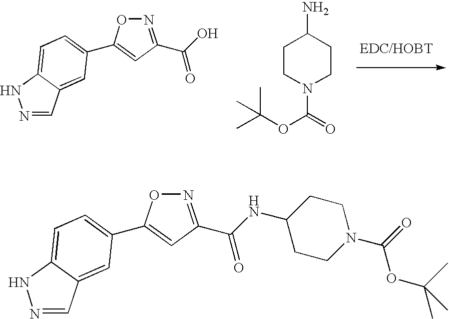

To a solution of 5-(1H-indazol-5-yl)isoxazole-3-carboxylic acid (0.150 g, 0.654 mmol), in DMF (8.0 mL) was added tert-butyl 4-aminopiperidine-1-carboxylate (0.131 g, 0.654 mmol), HOBT (0.110 g, 0.720 mmol), and EDC (0.138 g, 0.720 mmol). The resulting mixture was stirred under an atmosphere of nitrogen at ambient temperature for four hours. After four hours, reaction mixture was diluted with water (4 mL) and resulting precipitate was collected by filtration. Solid was washed with a mixture of 1:1 water:DMF (2×5 mL) and water (2×5 mL) and dried overnight in a vacuum oven to yield tert-butyl 4-(5-(1H-indazol-5-yl)isoxazole-3-carboxamido)piperidine-1-carboxylate (0.213 g, 78%). LCMS (Table 1, Method g): Rₜ=2.15 min.; MS m/z: 410.42 (M+H)⁺. ¹H NMR (400 MHz, d6-DMSO) ppm 13.35 (s, 1H), 8.71 (d, J=8.15 Hz, 1H), 8.37 (s, 1H), 8.21 (s, 1H), 7.86 (dd, J=8.71, 1.16 Hz, 1H), 7.67 (d, J=8.81 Hz, 1H), 7.28 (d, J=0.42 Hz, 1H), 4.04-3.86 (m, 3H), 2.92-2.72 (m, 2H), 1.75 (dd, J=12.44, 2.76 Hz, 2H), 1.52-1.42 (m, 2H), 1.40 (d, J=7.22 Hz, 9H).

General Procedure E: Formation of an Acid Chloride from Carboxylic Acid

To a solution of a carboxylic acid (1 eq.) in a chlorinated solvent (e.g., dichloromethane) is added 1-10 drops (e.g., 3 drops) of DMF. The reaction is cooled to about 0-5° C. in an ice bath. 1-10 eq. (e.g., 4 eq.) of a chlorinating agent (e.g., oxalyl chloride) is added dropwise over about 10 minutes. The reaction is stirred for 0-60 minutes (e.g., 10 minutes) and the cooling removed. The reaction is then stirred for about 0-120 minutes (e.g., 40 minutes) at which time the solvents are removed under vacuum to give the desired acid chloride.

### Exemplification of General Procedure E

### Preparation of 1-(3,4-dichlorophenyl)-5-(trichloromethyl)-1H-1,2,4-triazole-3-carbonyl chloride

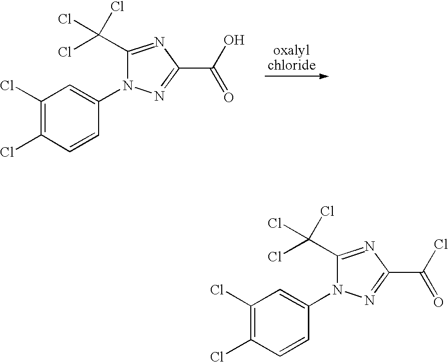

To a solution of 1-(3,4-dichlorophenyl)-5-(trichloromethyl)-1H-1,2,4-triazole-3-carboxylic acid (0.98 g, 2.61 mmol) in dichloromethane (305 mL) was added DMF (3 drops). The solution was cooled by ice-bath (0-5° C.). Oxalyl chloride (0.343 mL, 3.92 mmol) was added dropwise over 10 min. After stirring at 0° C. for 10 min, the ice-bath was removed and the reaction mixture was stirred at ambient temperature for 40 min. The solvents were removed under reduced pressure to afford 1-(3,4-dichlorophenyl)-5-(trichloromethyl)-1H-1,2,4-triazole-3-carbonyl chloride (1.02 g, 2.61 mmol, 99% yield) as a yellow oil. ¹H NMR (400 MHz, CDCl₃) δ ppm 7.66 (d, 1H), 7.61 (d, 1H), 7.50 (dd, 1H).

General Procedure F: Reaction of an Acid Chloride with an Amine

To a suspension of the acid chloride (1 eq.) in an organic solvent (e.g., toluene or DCM) is added the amine (1 eq.), followed by an organic amine base (e.g., triethylamine 1-10 eq, e.g., 1.5 eq.) dropwise. The resulting mixture is heated at 20-150° C. (e.g., 110° C.) for about 10 minutes to about 24 hours. Heating is stopped and the reaction mixture diluted with a suitable organic solvent (e.g., DCM). The organic layer is washed with saturated bicarbonate solution, then 0.6 M HCl solution. The organic layer is washed with brine, dried over an anhydrous salt (e.g., magnesium sulfate), filtered, and concentrated. The product can be isolated by trituration followed by filtration, by flash chromatography, or semi-prep LCMS.

### Exemplification of General Procedure F

### Preparation of N-(4-cyanophenyl)-4-phenyl-5-(trifluoromethyl)thiophene-2-carboxamide

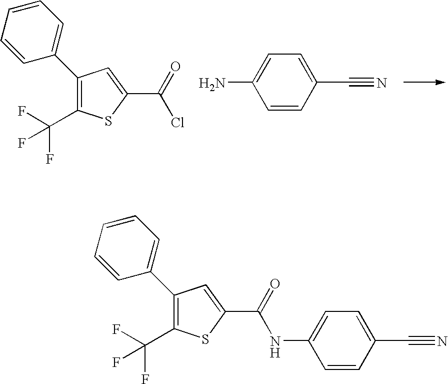

To a suspension of 4-phenyl-5-(trifluoromethyl)thiophene-2-carbonyl chloride (0.750 g, 2.58 mmol) in dry toluene (5.16 mL) was added 4-aminobenzonitrile (0.305 g, 2.58 mmol), followed by triethylamine (0.539 mL, 3.87 mmol) dropwise. The resulting mixture was heated to 110° C. overnight. Heating was stopped and the reaction mixture was diluted with DCM (150 mL). The organic layer was washed with saturated bicarbonate solution, then 0.6 M HCl solution. The organic layer was washed with brine (150 mL), dried MgSO₄ and concentrated to yield light brown solid. The solid was triturated with EtOAc/Heptane mixture to remove trace impurity. The mixture was filtered and dried to yield N-(4-cyanophenyl)-4-phenyl-5-(trifluoromethyl)thiophene-2-carboxamide (827 mg, 2.22 mmol) as an off-white solid. ¹H NMR (400 MHz, CDCl₃) δ ppm 7.83 (d, J=0.5 Hz, 1H), 7.76 (m, 2H), 7.68 (m, 2H), 7.58 (m, 1H), 7.46 (m, 5H).

General Procedure G.1: Reduction of a Nitrile to an Aldehyde with DIBAL-H

A solution of DIBAL-H (1 eq.) in a chlorinated solvent (e.g., dichloromethane) under an inert atmosphere (e.g., nitrogen) is cooled to about −78° C. A solution of the nitrile (1 eq.) is added dropwise over about 1-10 minutes (e.g., 5 minutes) and the reaction mixture stirred at about −78° C. for about 0.5-3 hours (e.g., 1 hour). The reaction is quenched with water and the reaction is warmed to ambient temperature. The aqueous layer is extracted with an organic solvent (e.g., dichloromethane). The combined extracts are washed with brine and dried over an anhydrous salt (e.g., magnesium sulfate), filtered and concentrated. The residue is purified by flash column chromatography to give the desired product.

### Exemplification of General Procedure G.1

### Preparation of N-(2-chloro-4-formylphenyl)-1-(3,4-dichlorophenyl)-5-(trichloromethyl)-1H-1,2,4-triazole-3-carboxamide

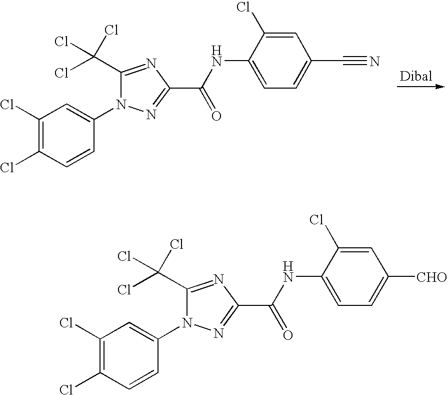

N-(2-Chloro-4-cyanophenyl)-1-(3,4-dichlorophenyl)-5-(trichloromethyl)-1H-1,2,4-triazole-3-carboxamide (0.403 g, 0.790 mmol) in DCM (4.32 mL) at −78° C. was added to DIBAL-H in DCM (0.790 mL, 0.790 mmol) dropwise over 5 minutes. The reaction mixture was stirred at −78° C. for 1 hour. TLC (30% ethyl acetate/heptane) indicated no starting material present. Water (10 mL) was added and the reaction mixture was allowed to warm slowly to ambient temperature over 1 hour. The aqueous layer was extracted with DCM (3×15 mL). The combined organics were washed with brine (15 mL), dried over MgSO₄ and concentrated in vacuo. The crude material was purified by flash column chromatography in 30% ethyl acetate/heptane to afford N-(2-chloro-4-formylphenyl)-1-(3,4-dichlorophenyl)-5-(trichloromethyl)-1H-1,2,4-triazole-3-carboxamide as a white solid (132 mg, 0.257 mmol, 33%). LCMS (Method a) Rₜ 2.94 min; m/z: (M+H)⁺ 507.96.

### General Procedure G.2

### Reduction of a Nitrile to Aldehyde with Sodium Dihydrogen Phosphate Monohydrate and Raney Nickel

The nitrile (1 eq.) is dissolved in a mixture of pyridine, acetic acid, and water and cooled to about 0° C. in an ice bath. Sodium dihydrogen phosphate monohydrate (1-20 eq, e.g., 17 eq.) is added as a solid. Raney nickel (about 0.42 g per 1.4 mmol nitrile) is added. The mixture was heated under nitrogen to about 60° C. for about 1-3 hours (e.g., 1.5 hours). The reaction is cooled to room temperature and filtered through Celite® and washed through with ethanol and ethyl acetate. The filtrate is concentrated under vacuum. The residue is partitioned between an organic solvent (e.g., ethyl acetate) and water. The organic layer is washed with 5% citric acid, saturated sodium bicarbonate, and then brine. The organic solution is dried over an anhydrous salt (e.g., magnesium sulfate), filtered, and concentrated. The residue is purified by flash column chromatography to provide the desired product.

### Exemplification of General Procedure G.2

### Preparation of N-(4-formylphenyl)-4-phenyl-5-(trifluoromethyl)thiophene-2-carboxamide

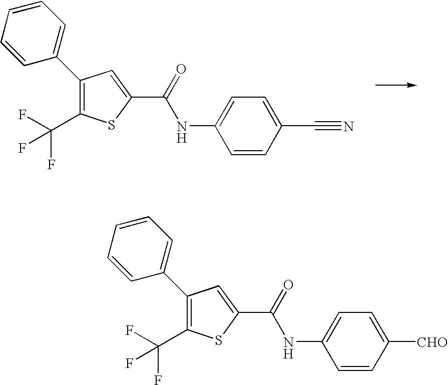

N-(4-cyanophenyl)-4-phenyl-5-(trifluoromethyl)thiophene-2-carboxamide (0.951 g, 2.55 mmol) was dissolved in a mixture of pyridine (33.6 mL), acetic acid (16.80 mL) and water (16.80 mL), then cooled to 0° C. in an ice bath. Sodium dihydrogen phosphate monohydrate (4.60 g, 43.4 mmol) was added as a solid, followed by the addition of raney nickel (0.42 g per 1.4 mmol nitrile) as a water slurry. The mixture was heated to 60° C. under an atmosphere of nitrogen for 1.5 hrs. The reaction mixture was cooled to ambient temperature and filtered through Celite®. The Celite® cake was washed with ethanol (20 mL) and EtOAc (20 mL). The filtrate was concentrated in vacuo to give crude product as a green liquid. The crude material was taken up in EtOAc (150 mL) and water (150 mL). The organic layer was washed with 5% aqueous citric acid (150 mL), saturated NaHCO₃ solution (150 mL), and brine (150 mL). The organic layer was dried (MgSO₄) and concentrated to yield crude solid. The residue was dissolved in EtOAc and purified via Analogix FC system using RediSep® RS 40 g column, with a gradient of 0-100% EtOAc/Heptane over 30 min. at 30 mL/min. to give N-(4-formylphenyl)-4-phenyl-5-(trifluoromethyl)thiophene-2-carboxamide (698 mg, 1.86 mmol) as an off-white solid. ¹H NMR (400 MHz, CDCl₃) δ ppm 9.97 (s, 1H), 7.93 (d, J=8.6 Hz, 2H), 7.82 (t, J=10.9 Hz, 3H), 7.58 (d, J=1.2 Hz, 1H), 7.44 (d, J=9.1 Hz, 5H).

### General Procedure H: Reductive Amination of an Aldehyde

The aldehyde (1 eq.) is suspended in a mixture of an alcoholic solvent (e.g., methanol) and chlorinated solvent (e.g., dichloroethane). The amine (1.05 eq.) and acetic acid (2-15 eq., e.g., 8.7 eq.) are added and the mixture stirred for about 30 minutes. A reducing agent (e.g., sodium cyanoborohydride, 0.5 eq) is added and the reaction stirred at ambient temperature for 4-24 hours (e.g., about 16 hours). The resulting solid is collected by vacuum filtration and washed with water and then cold methanol. The solid is dried to provide the desired product.

### Exemplification of General Procedure H

### Example #3

### Preparation of 1-(4-(4-phenyl-5-(trifluoromethyl)thiophene-2-carboxamido)benzyl)azetidine-3-carboxylic acid

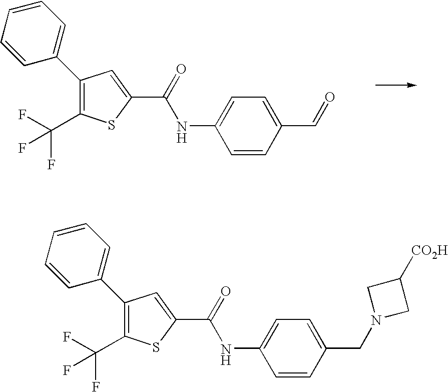

N-(4-Formylphenyl)-4-phenyl-5-(trifluoromethyl)thiophene-2-carboxamide (0.150 g, 0.400 mmol) was suspended in a mixture of MeOH (5.0 mL) and DCE (5.0 mL) to give clear solution. To this was added azetidine-3-carboxylic acid (0.042 g, 0.420 mmol) as solid, followed shortly by acetic acid (0.2 mL, 3.49 mmol). The resulting mixture was stirred at ambient temperature for 30 min. under the atmosphere of nitrogen, then sodium cyanoborohydride (0.013 g, 0.200 mmol) was added in one portion. The reaction was stirred at ambient temperature overnight. The resulting white suspension was filtered. The collected solid was washed with water and cold MeOH, and air-dried to yield 1-(4-(4-phenyl-5-(trifluoromethyl)thiophene-2-carboxamido)benzyl)azetidine-3-carboxylic acid (149 mg, 0.32 mmol). LCMS (Table 1, Method c) Rₜ=1.98 min, m/z 461.27 (M+H)⁺. ¹H NMR (400 MHz, DMSO-d6) δ ppm 11.96-12.45 (m, 1H), 10.45 (s, 1H), 8.19 (d, J=1.2 Hz, 1H), 7.66 (d, J=8.5 Hz, 2H), 7.52 (m, 5H), 7.27 (d, J=8.5 Hz, 2H), 3.51 (s, 2H), 3.35 (m, 2H), 3.19 (t, J=6.4 Hz, 3H).

### General Procedure I: Deprotection of a Silyl Protected Alcohol

The silyl protected alcohol (1 eq.) is dissolved in an organic solvent (e.g., THF). TBAF (1-5 eq., e.g., 2 eq.) is added and the reaction stirred for about 0.5-24 h (e.g., 1 hour). The reaction is concentrated under vacuum. The residue is partitioned between ethyl acetate and 5% HCl. The layers are separated and the organic layer washed with 10% HCl, saturated sodium bicarbonate, and then water. The organic solution is dried over an anhydrous salt (e.g., sodium sulfate), filtered, and concentrated to afford the desired product that is used without further purification.

### Exemplification of General Procedure I

### Preparation of 3-phenyl-isoxazole-5-carboxylic acid (4-hydroxymethyl-phenyl)-amide

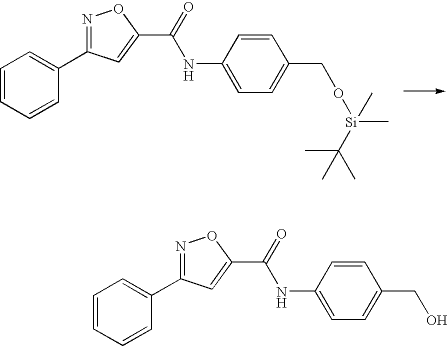

In a 100 mL round-bottomed flask was stirred N-(4-((tertbutyldimethylsilyloxy)methyl)phenyl)-3-phenylisoxazole-5-carboxamide (0.223 g, 0.546 mmol) in THF (20 mL) to give an orange solution. TBAF (1.092 mL, 1.092 mmol) was added to the solution and the reaction mixture turned into a dark orange-green solution. After stirring at room temperature for about 60 minutes LCMS and TLC indicated the reaction was complete. The reaction was concentrated and the residue was partitioned between EtOAc and HCl (5%). The organic layer was washed with HCl (10%), saturated NaHCO₃, and water. The organic solution was dried over sodium sulfate, filtrated and concentrated to afford 3-phenyl-isoxazole-5-carboxylic acid (4-hydroxymethyl-phenyl)-amide (188 mg) as a yellow solid that was used directly in the next step without purification.

General Procedure J.1: Oxidation of an Alcohol to an Aldehyde with Barium Manganate

The alcohol (1 eq.) is dissolve in a chlorinated solvent (e.g., 1,2-dichloroethane). Barium manganate (1-10 eq., e.g., 5 eq.) was added and the reaction heated to about 55° C. for 1-20 hours (e.g., 1 hour). The reaction is cooled to ambient temperature and filtered through Celite® and the filter cake is washed with and organic solvent (e.g., dichloromethane). The filtrate is concentrated under vacuum to provide the desired product that is used without further purification.

### Exemplification of General Procedure J.1

### Preparation of N-(4-formylphenyl)-3-phenylisoxazole-5-carboxamide

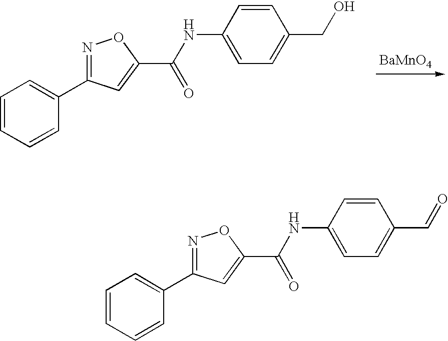

To a suspension of N-(4-(hydroxymethyl)phenyl)-3-phenylisoxazole-5-carboxamide (161 mg, 0.546 mmol) in DCE (15 mL) was added barium manganate (777 mg, 2.73 mmol). The black suspension was heated at 55° C. for 1 hr. The reaction was cooled to ambient temperature and filtered through Celite® and was washed by dichloromethane. The filtrate was concentrated to afford N-(4-formylphenyl)-3-phenylisoxazole-5-carboxamide (92 mg, 57.6%). LCMS (Table 1, Method a) Rₜ=3.62 min, MS m/z: 293.23 (M+H)⁺; ¹H NMR (400 MHz, DMSO-d₆) δ 11.17 (s, 1H), 9.94 (s, 1H), 8.06 (d, 2H, J=8.61 Hz), 7.93-8.08 (m, 4H), 7.56-7.62 (m, 3M), 7.54 (s, 1H).

General Procedure J.2: Oxidation of an Alcohol to an Aldehyde with Manganese Oxide

The alcohol (1 eq.) and manganese dioxide (1-10 eq., e.g., 5 eq.) are combined in a chlorinated solvent (e.g., 1,2-dichloroethane). The mixture was heated at about 60° C. for about 0.5-20 hours (e.g., 1 h). The mixture is filtered and the solvents are removed under vacuum. The resulting solid is collected by vacuum filtration and washed with a non-polar organic solvent (e.g., heptane) to provide the desired product.

### Exemplification of General Procedure J.2

### Preparation of N-(4-formylphenyl)-5-phenylisoxazole-3-carboxamide

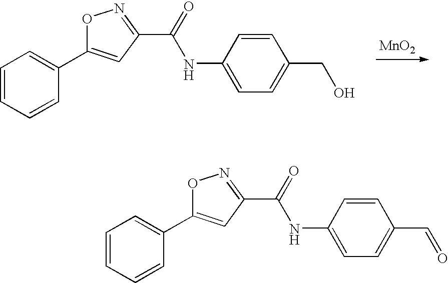

N-(4-(Hydroxymethyl)phenyl)-5-phenylisoxazole-3-carboxamide (0.2 g, 0.680 mmol) and manganese dioxide (0.295 g, 3.40 mmol) were combined in 1,2-dichloroethane (13.59 mL) in a sealed vial. The mixture was heated at 60° C. for about 1 h. The mixture was filtered through syringe filter and the solvents were removed under vacuum. The resulting solid was washed with ether. Heptane (15 mL) was added. The resulting solid was collected by vacuum filtration and washed with heptane to provide N-(4-formylphenyl)-5-phenylisoxazole-3-carboxamide (0.154 g, 0.527 mmol, 78% yield) as a yellow solid on drying briefly in a vacuum oven. LCMS (Table 1, Method a) Rₜ=3.63 min, MS m/z: 293.25 (M+H)⁺.

General Procedure K: Sonagashira Coupling of an Alkyne with an Aryl Bromide

The aryl bromide (1 eq.) and alkyne (2 eq.) are stirred in a mixture of organic solvent (e.g., dioxane) and organic base (e.g., triethylamine). The mixture is degassed with an inert gas (e.g., nitrogen). Palladium catalyst (e.g., bis(triphenylphosphine)palladium(II) dichloride, 0.1 eq.) and copper (I) iodide (0.2 eq) are added and the reaction heated to about 85° C. for about 4-24 hours (e.g., about 16 hours). The reaction is cooled to ambient temperature and the reaction partitioned between an organic solvent (e.g., diethyl ether) and brine. The layers are separated and the washed with brine, dried over an anhydrous salt (e.g., magnesium sulfate), filtered, and concentrated. The residue is purified by flash column chromatography to provide the desired product.

### Exemplification of General Procedure K

### Preparation of ((4-(1,3-dioxolan-2-yl)phenyl)ethynyl)trimethylsilane

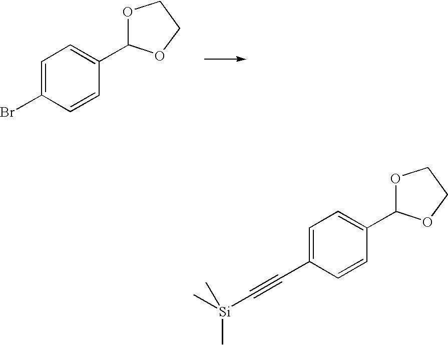

2-(4-Bromophenyl)-1,3-dioxolane (11.97 g, 52.3 mmol) and ethynyltrimethylsilane (10.26 g, 105 mmol) was suspended in mixture of dioxane (55 mL) and triethylamine (50 mL). The resulting mixture was degassed for about 5 min., then bis(triphenylphosphine)palladium(II) dichloride (0.367 g, 0.523 mmol) and copper(I) iodide (0.199 g, 1.045 mmol) were added. The reaction was heated to about 85° C. overnight. The reaction was cooled to ambient temperature and more ethynyltrimethylsilane (5.13 g, 52.3 mmol) was added. The reaction mixture was heated to about 85° C. for 3.5 hrs. Heating was stopped and the crude reaction mixture was partitioned into a mixture of saturated brine (200 mL) and diethyl ether (200 mL). The organic layer was separated, washed with brine (200 mL), dried (MgSO₄) and concentrated to yield about 14 g of crude dark brown oil. The crude residue was purified via Analogix FC system using RediSep® RS 330 g column, with a gradient of 0-20% EtOAc in heptane over 35 min. at 40 mL/min. to give ((4-(1,3-dioxolan-2-yl)phenyl)ethynyl)trimethylsilane (11.31 g, 45.9 mmol) as dark brown oil. ¹H NMR (400 MHz, CD₂Cl₂) δ ppm 7.48 (m, 2H), 7.42 (m, 2H), 5.77 (s, 1H), 4.10 (m, 2H), 4.02 (m, 2H), 0.30 (, 9H).

### General Procedure L: Protection of an Aldehyde as the Acetal

To a solution of aldehyde (1 eq.) and an acid (e.g., p-toluenesulfonic acid monohydrate, 0.1 eq.) in an organic solvent (e.g., toluene) is added ethylene glycol (2 eq.). The reaction is heated to reflux collecting water in Dean-Stark trap. Heating is continued for 2-20 hours (e.g., 6 hours) at which time additional ethylene glycol (8 eq.) is added and heating continued for an additional 2-20 hours (e.g., 4 hours) if the reaction is not yet complete. The reaction is cooled and partitioned between an organic solvent (e.g., ethyl acetate) and saturated sodium bicarbonate solution. The organic layer is washed with brine, dried over an anhydrous salt (e.g., magnesium sulfate), filtered, and concentrated to give the desired product.

### Exemplification of General Procedure L

### Preparation of 2-(4-bromophenyl)-1,3-dioxolane

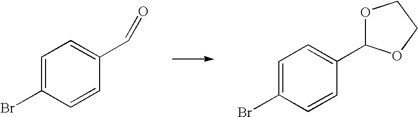

To a solution of 4-bromobenzaldehyde (10.0 g, 54.0 mmol) and p-toluenesulfonic acid monohydrate (1.028 g, 5.40 mmol) in toluene (270 mL) under an atmosphere of nitrogen was added ethylene glycol (6.03 mL, 108 mmol). The resulting mixture was heated to reflux in a Dean-Stark apparatus for about 6 hours. More ethylene glycol (24.11 mL, 432 mmol) was added and the reaction was heated to reflux for additional 4 hours. The reaction was cooled to ambient temperature, and then partitioned into saturated sodium bicarbonate solution and ethyl acetate. The organic phase was separated, washed successively with brine, dried (MgSO₄) and concentrated to yield 2-(4-bromophenyl)-1,3-dioxolane (12.97 g, 56.6 mmol) as light yellow liquid. ¹H NMR (400 MHz, CD₂Cl₂) δ ppm 7.55 (t sext., J=1.0, 3.4 Hz, 2H), 7.37 (m, 2H), 5.75 (s, 1H), 4.09 (m, 2H), 4.02 (m, 2H).

General Procedure M: Formation of an Isoxazole from an Alkyne

Alkyne (1 eq.) and (Z)-ethyl 2-chloro-2-(hydroxyimino)acetate (2.2 eq.) are combined in an organic solvent (e.g., toluene). An organic base (e.g., triethylamine, 2.5 eq.) was added and the reaction was heated to about 90° C. under an atmosphere of nitrogen for about 2-20 hours (e.g., about 16 hours). The reaction is cooled and then partitioned between 1 M HCl and diethylether. The layers are separated and the aqueous layer extracted with diethylether. The combined organics are washed with brine, dried over an anhydrous salt (e.g., magnesium sulfate), filtered, and concentrated. The residue is purified by flash column chromatography to give the desired product.

### Exemplification of General Procedure M

### Preparation of ethyl 5-(4-(1,3-dioxolan-2-yl)phenyl)isoxazole-3-carboxylate

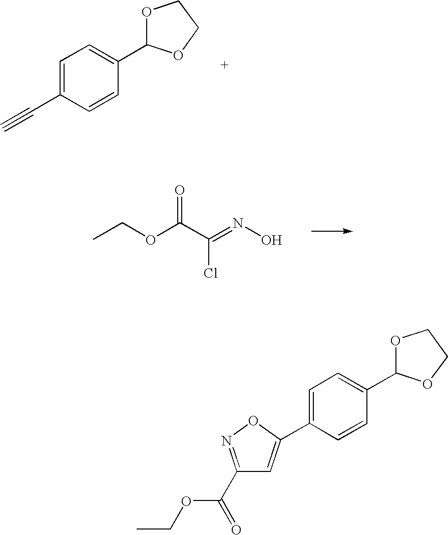

To a solution of 2-(4-ethynylphenyl)-1,3-dioxolane (7.18 g, 30.9 mmol) and (Z)-ethyl 2-chloro-2-(hydroxyimino)acetate (10.31 g, 68.0 mmol) in toluene (30.9 mL) was added triethylamine (10.77 mL, 77 mmol) slowly dropwise. Triethyl ammonium salt was observed to commence immediately out of solution. The reaction mixture was stirred at 90° C. under an atmosphere of nitrogen overnight. Heating was removed. The resulting mixture was partitioned in 1M HCl solution and diethyl ether. The organic layer was separated. The aqueous layer was back-extracted with diethyl ether. The combined organic phase were washed with brine (50 mL), dried (MgSO₄) and concentrated to give crude brown oil. The crude residue was purified via Analogix FC system using RediSep® RS 330 g column, with a gradient of 0-30% EtOAc/Heptane over 35 min. at 30 mL/min to give ethyl 5-(4-(1,3-dioxolan-2-yl)phenyl)-isoxazole-3-carboxylate (5.41 g, 18.7 mmol) as off-white solid. ¹H NMR (400 MHz, CD₂Cl₂) δ ppm 7.85 (m, 2H), 7.62 (d, J=8.1 Hz, 2H), 6.97 (d, J=0.9 Hz, 1H), 5.84 (s, 1H), 4.45 (dq, J=0.9, 7.1, 7.1 Hz, 2H), 4.08 (m, 4H), 1.44 (m, 3H).

General Procedure N: Deprotection of an acetal to an aldehyde

Acetal (1 eq.) is dissolved in an organic solvent (e.g., THF). To this is added HCl (2 eq.) as a 1 M solution in water. The reaction is stirred at ambient temperature for 1-10 hours (e.g., 4.5 hours). One M NaOH solution (2 eq. of NaOH) is added to neutralize the pH. The reaction mixture is taken up in brine and diethyl ether. The layers are separated. The organic layer is dried over an anhydrous salt (e.g., magnesium sulfate) and concentrated to near dryness. The resulting precipitate is filtered, rinsed with diethyl ether and dried to yield the desired product.

### Exemplification of General Procedure N

### Preparation of N-(3-chloro-4-isopropoxyphenyl)-5-(4-formylphenyl)isoxazole-3-carboxamide

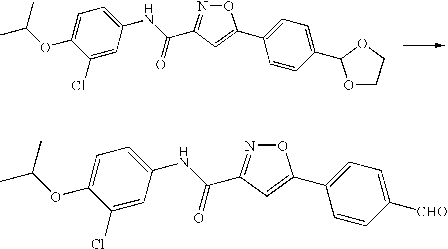

5-(4-(1,3-Dioxolan-2-yl)phenyl)-N-(3-chloro-4-isopropoxyphenyl)isoxazole-3-carboxamide (0.1803 g, 0.420 mmol) was dissolved in THF (8.41 mL). To this was added HCl (0.841 mL, 0.841 mmol) as 1 M solution in water. The reaction was stirred at ambient temperature for 4.5 hrs. Then 1 M NaOH solution (0.841 mL, 0.841 mmol) was added to neutralize the pH. The reaction mixture was taken up in brine and diethyl ether. The organic layer was dried (MgSO₄) and concentrated to near dryness. The resulting precipitate was filtered, rinsed with diethyl ether and dried to yield N-(3-chloro-4-isopropoxyphenyl)-5-(4-formylphenyl)isoxazole-3-carboxamide (135 mg, 0.31 mmol) as a white solid. ¹H NMR (400 MHz, CD₂Cl₂) δ ppm 10.09 (s, 1H), 8.51 (s, 1H), 8.04 (s, 4H), 7.82 (d, J=2.5 Hz, 1H), 7.49 (dd, J=2.5, 8.9 Hz, 1H), 7.22 (s, 1H), 7.01 (d, J=8.9 Hz, 1H), 4.57 (td, J=6.1, 12.1 Hz, 1H), 4.09 (m, 4H).

### General Procedure O: Mitsunobu Reaction of an Alcohol

Triphenylphosphine (1.05 eq.) is dissolved in an organic solvent (e.g., THF). The mixture is cooled to about 0° C. in an ice bath. Diisopropylazodicarboxylate (1.05 eq.) is added dropwise over about 0-30 minutes (e.g., 10 minutes) and stirred at about 0° C. for 10-60 minutes (e.g., 30 minutes). The phenol (1.05 eq) and alcohol (1 eq.) are added to the mixture over about 30 minutes. The reaction is stirred at about 0° C. for about 0.5-20 hours (e.g., 2 hours) and then stirred for an additional 0.5-20 hours (e.g., 16 hours) at ambient temperature. The reaction is concentrated to dryness and the residue triturated with an organic solvent (e.g., diethylether). The resulting solid is removed by vacuum filtration and the filtrate concentrated under vacuum. The resulting residue is purified by flash column chromatography to give the desired product.

### Exemplification of General Procedure O

### Preparation of 1-(4-((2,2-dimethyl-1,3-dioxolan-4-yl)methoxy)phenyl)ethanone

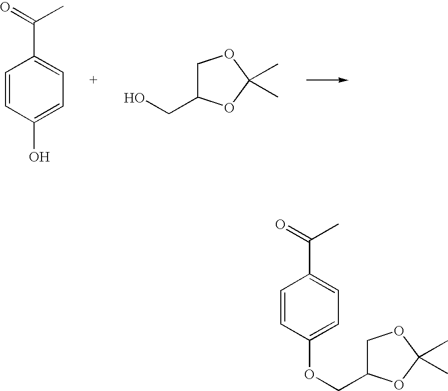

Into a 250 mL round-bottomed was added triphenylphosphine (3.13 g, 11.92 mmol) and THF (120 mL) to give a colorless solution. The solution was cooled to 0° C. Diisopropyl azodicarboxylate (2.317 mL, 11.92 mmol) was added dropwise over 10 minutes. The reaction mixture was stirred at 0° C. for 30 min. Then a colorless solution of 1-(4-hydroxyphenyl)ethanone (1.623 g, 11.92 mmol) and (2,2-dimethyl-1,3-dioxolan-4-yl)methanol (1.407 mL, 11.35 mmol) was added to the mixture over 30 minutes. The mixture was stirred for 2 hours at 0° C. and then overnight at ambient temperature. The reaction mixture was concentrated to dryness and the residue triturated with ether. The white solid was filtered off and the filtrate was concentrated to afford an yellow viscous oil which was purified via Analogix (0-40% EtOAc/Heptane over 40 minutes; RS-120 Si column) to give 1-(4-((2,2-dimethyl-1,3-dioxolan-4-yl)methoxy)phenyl)ethanone (2.352 g, 83% yield) as a white solid. LCMS (Table 1, Method b) Rₜ=1.97 min (no ionization); ¹H NMR (400 MHz, DMSO-d6) δ 7.93 (d, 2H), 7.06 (d, 2H), 4.40-4.46 (m, 1H), 4.05-4.15 (m, 3H), 3.75-3.78 (m, 1H), 2.52 (s, 3H), 1.36 (s, 3H), 1.31 (s, 3H).

General Procedure Q: Reaction of a Sulfonyl Chloride with an Amine

To a suspension of an amine hydrochloride salt (2 eq.) in a suitable organic solvent (e.g., dichloromethane) is added an organic base (e.g., triethylamine). Sulfonyl chloride (1 eq.) is added and the reaction is stirred at ambient temperature for 2-24 hours (e.g., 16 hours). The reaction mixture is washed with 10% HCl. The aqueous layer is extracted with an organic solvent (e.g., dichloromethane). The combined organic extracts are washed with water and brine. The organic solution is then dried over an anhydrous salt (e.g., magnesium sulfate), filtered, and concentrated to provide the product that was used without further purification.

### Exemplification of General Procedure Q

### Preparation of methyl 2-(4-acetamido-3-chlorophenylsulfonamido)acetate

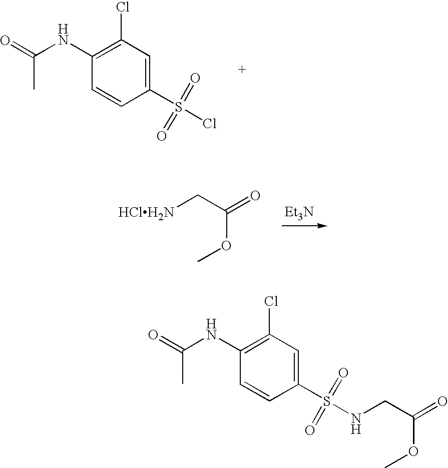

To a suspension of amino-acetic acid methyl ester hydrochloride (0.937 g, 7.46 mmol) in DCM (37.3 mL) was added triethylamine (2.183 mL, 15.66 mmol). 4-Acetamido-3-chlorobenzene-1-sulfonyl chloride (1 g, 3.73 mmol) was added in one portion and the reaction mixture was stirred at ambient temperature overnight. The reaction mixture was washed with HCl (10%, 25 mL). The aqueous layer was extracted with DCM (20 mL). The combined DCM layers were washed with water (20 mL) and brine (20 mL). The organic layer was dried over MgSO₄, filtered and concentrated to afford methyl 2-(4-acetamido-3-chlorophenylsulfonamido)acetate (0.93 g, 2.61 mmol, 70.0% yield) as a pale yellow solid. LCMS (Table 1, Method a) Rₜ=2.46 min, MS m/z: 321.06 (M+H)⁺; ¹H NMR (400 MHz, DMSO-d6) δ 9.72 (s, 1H), 8.30 (t, 1H, J=6.20 Hz), 8.05 (d, 1H, J=8.62 Hz), 7.84 (d, 1H, J=2.06 Hz), 7.70 (dd, 1H, J=8.47, 2.03 Hz), 3.76 (d, 2H, J=6.21 Hz), 3.53 (s, 3H), 2.16 (s, 3H).

General Procedure R: Reaction of a Ketone with Diethyl Oxalate

An alkalai metal (e.g., sodium, 3 eq.) is dissolved in an alcohol solvent (e.g., ethanol). Ketone (1 eq.) and diethyl oxalate (1.5 eq.) are added and the reaction heated to about reflux for about 0.5-10 hours (e.g., 2 hours). The reaction is cooled and diluted with an organic solvent (e.g., ethyl acetate) and then washed with 2 M HCl solution. The organic layer is then concentrated to dryness and purified by flash column chromatography to provide the desired product.

### Exemplification of General Procedure R

### Preparation of ethyl 4-(4-((2,2-dimethyl-1,3-dioxolan-4-yl)methoxy)phenyl)-2,4-dioxobutanoate

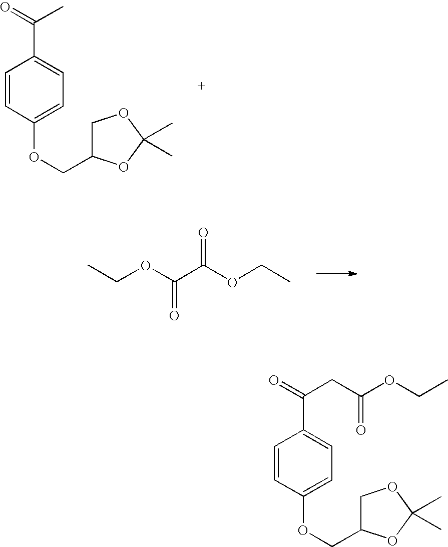

Sodium (0.648 g, 28.2 mmol) was added to ethanol (30 mL) and the mixture was stirred until the sodium completely dissolved. 1-(4-((2,2-dimethyl-1,3-dioxolan-4-yl)methoxy)phenyl)ethanone (2.352 g, 9.40 mmol) and diethyl oxalate (1.914 mL, 14.10 mmol) were added to the solution. The mixture was heated to reflux for 2 hours. After cooling to ambient temperature, the mixture was diluted with EtOAc and washed with 2M HCl solution. The organic layer was concentrated to dryness and purified via Analogix (5-55% EtOAc/heptane over 30 minutes) to give 4-(4-((2,2-dimethyl-1,3-dioxolan-4-yl)methoxy)phenyl)-2,4-dioxobutanoate as a light yellow solid (2.074 g, 63%). LCMS (Table 1, Method b) Rₜ=2.31 min, m/z 351.29 (M+H)⁺; ¹H NMR (400 MHz, DMSO-d6) δ 8.08 (d, 2H), 7.13 (d, 2H), 4.44 (m, 1H), 4.32 (q, 2H), 4.09-4.20 (m, 3H), 3.77 (m, 1H), 1.36 (s, 3H), 1.32 (s, 3H), 1.31 (t, 3H).

### Exemplification of General Procedure R

### Second Example

### Preparation of lithium (Z)-1-ethoxy-3-methyl-1,4-dioxo-4-phenylbut-2-en-2-olate

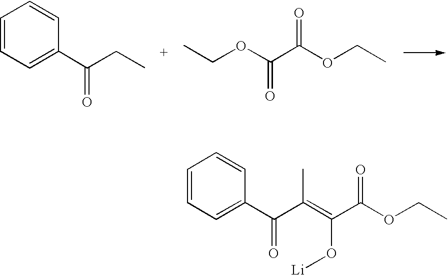

To a 3-neck flask fitted with an addition funnel and a mechanical stirrer was added diethyl ether (165 mL) and LHMDS (5.72 g, 34.2 mmol). The mixture is stirred and cooled to −78° C. under nitrogen and then propiophenone (5.51 g, 41.1 mmol) in diethyl ether (35 mL) was added dropwise at such a rate that the reaction temperature did not exceed −75° C. When the addition is complete, the reaction is stirred for an additional 30 minutes at −78° C., then diethyl oxalate (5.0 g, 34.2 mmol) in diethyl ether (25 mL) was added in one portion. The mixture was stirred briefly at −78° C. and was then stirred at room temperature for 18 h. Product was filtered off, washed with ether (2×40 mL) and dried in a vacuum oven to yield lithium (Z)-1-ethoxy-3-methyl-1,4-dioxo-4-phenylbut-2-en-2-olate (5.70 g, 69%) as a yellow solid. LCMS (Table 1, Method a) Rₜ=3.38 min, m/z 241.25 (M+H)⁺; ¹H NMR (400 MHz, DMSO-d6) δ 7.20-7.30 (m, 5H), 3.75 (q, 2H), 1.57 (s, 3H), 1.11 (t, 3H).

General Procedure S: Cyclization to a 5-arylisoxazole-3-carboxylate

A mixture of β-diketone (1 eq.) and hydroxylamine hydrochloride (1.10 eq, e.g., 3 eq.) in an alcoholic solvent (e.g., ethanol) is heated to reflux for 1-24 h (e.g., 3 h). The reaction is cooled to room temperature and the crude reaction mixture is poured into water. The aqueous mixture is extracted with a suitable organic solvent (e.g., EtOAc). The combined exctracts are dried over an anhydrous salt (e.g., sodium sulfate), filtered, and concentrated. The residue may be purified via flash column chromatography, recrystallization, or trituration to give the desired product.

### Exemplification of General Procedure S

### Preparation of ethyl 5-(4 isobutylphenyl)-isoxazole-3-carboxylate

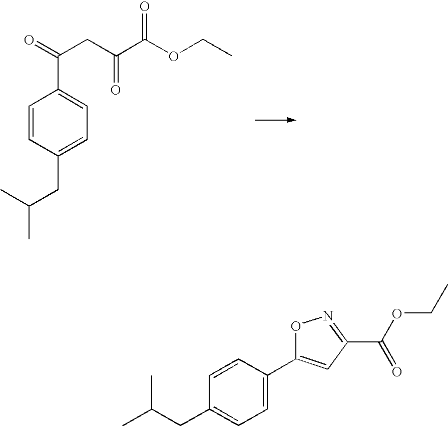

A mixture of ethyl 4-(4-isobutylphenyl)-2,4-dioxobutanoate (14.86 g, 53.8 mmol) and hydroxylamine hydrochloride (11.21 g, 161 mmol) in ethanol (120 mL) was heated to reflux for 3 hours. The reaction was cooled to room temperature and the crude reaction mixture was poured into water. The aqueous mixture was extracted with EtOAc (3×150 mL). The combined extracts were dried over sodium sulfate, filtered, and concentrated. The residue was purified via flash column chromatography (0-50% EtOAc/heptane over 45 min; Redi-Sep column, 330 g). The product fractions were concentrated to dryness to give ethyl 5-(4-isobutylphenyl)isoxazole-3-carboxylate (13.5 g, 49.4 mmol, 92% yield) as a white solid: LCMS (Table 1, Method b) Rt=2.67 min, m/z 274.19 (M+H)⁺.

### General Procedure T: Cyclization to a Pyrazole

The lithium salt of a β-keto-ester is dissolved in an alcoholic solvent such as methanol, ethanol or isopropanol, and optionally a co-solvent such as DMF or DMSO, containing an equivalent amount of a mono-substituted hydrazine salt, such as hydrochloride, hydrobromide, sulfate, or tosylate, and the mixture is stirred for 1-24 h at room temperature to 100° C., e.g., at about 50° C. for 4-24 h. The reaction is cooled to room temperature and concentrated, and the residue is taken up in an organic solvent such as methylene chloride, ethyl acetate or toluene and washed with aqueous solvents to remove salts. The organic layer is dried, filtered and concentrated. The crude product may be further purified by crystallization or chromatography on silica gel.

### Exemplification of General Procedure T

### Preparation of ethyl 1-isobutyl-5-phenyl-1H-pyrazole-3-carboxylate

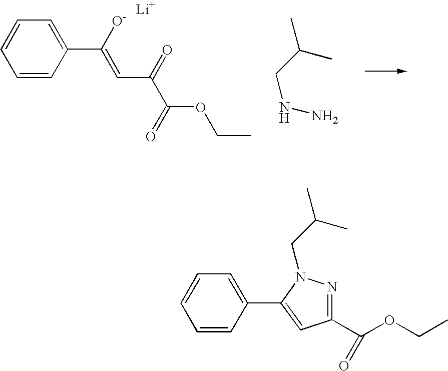

To a suspension of lithium (Z)-1-ethoxy-1,4-dioxo-4-phenylbut-2-en-2-olate (3.40 g, 15.03 mmol) in Ethanol (25 mL) and DMF (10.0 mL) was add isobutylhydrazine sulfate (2.80 g, 15.03 mmol) and the reaction was heated at 50° C. for 18 h. The reaction was cooled and concentrated. The residue was dissolved in ethyl acetate (100 μL) and washed with saturated sodium chloride solution, dried (Na₂SO₄), filtered and concentrated. The crude product was further purified on silica gel using a gradient from 5% to 25% ethyl acetate in heptane as eluant. Clean product fractions were combined and concentrated to yield ethyl 1-isobutyl-5-phenyl-1H-pyrazole-3-carboxylate (3.85 g, 94%) as an oil. LCMS (Table 1, Method b) Rt=2.44 min, m/z 273.23 (M+H)+; 1H NMR (400 MHz, DMSO-d6) δ 7.40-7.50 (m, 5H), 6.78 (s, 1H), 4.25 (q, 2H), 3.99 (d, 2H), 1.97 (s, 3H), 1.27 (t, 3H), 0.66 (d, 6H).

### General Procedure U: Aminolysis of an Ester to an Amide

A solution of ester in alcoholic ammonia is sealed in a steel vessel and heated at 60-120° C., e.g., 100° C., for 4-24 h, e.g., about 18 h. The reaction is cooled to room temperature and either filtered or concentrated. The crude product may be further purified by crystallization or by chromatography on silica gel.

### Exemplification of General Procedure U

### Preparation of 1-isobutyl-5-phenyl-1H-pyrazole-3-carboxamide

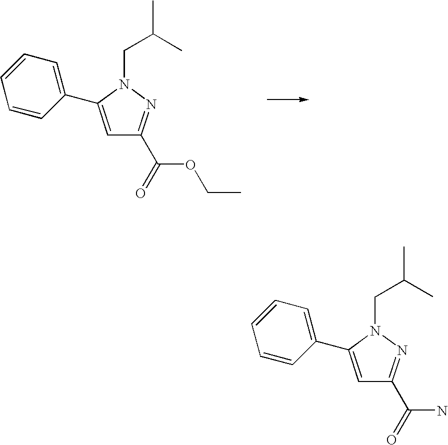

To Ethyl 1-isobutyl-5-phenyl-1H-pyrazole-3-carboxylate (3.85 g, 14.14 mmol) was added 7 M methanolic ammonia (100 mL, 700 mmol) in a steel reactor. The reaction was sealed and heated at 100° C. for 24 hours. The reaction was cooled to room temperature and the solvents were removed under reduced pressure to yield 1-isobutyl-5-phenyl-1H-pyrazole-3-carboxamide (3.00 g, 87%) as a pale yellow oil. LCMS (Table 1, Method b) Rₜ=1.95 min, m/z 244.21 (M+H)⁺; ¹H NMR (400 MHz, DMSO-d6) δ 7.45 (m, 6H), 7.20 (s broad, 1H), 6.65 (s, 1H), 3.93 (d, 2H), 2.02 (m, 1H), 0.66 (d, 6H).

### General Procedure V: Buchwald Coupling of an Amide to an Aryl Bromide

To a mixture of a primary amide (0.90-2 eq., e.g., 1.00 eq.), an aryl halide (e.g., an aryl bromide, aryl chloride or an aryl iodide) (0.7-3 eq., e.g., 1.1 eq.) and an inorganic base (e.g., KF, Na₂CO₃ or Cs₂CO₃) (2-8 eq., e.g., 1.2 eq.) in a degassed organic solvent (e.g., THF, DME, DMF, 1,4-dioxane, toluene) is added a palladium catalyst (e.g., tris(benzylideneacetone)dipalladium (0) and XANTPHOS, tetrakis(triphenylphosphine)palladium(0), bis(acetato)triphenylphosphinepalladium(II) (˜5% Pd) polymer-bound FibreCat™ or [1,1′-bis(diphenylphosphino)ferrocene]dichloropalladium(II), complex with dichloromethane, typically tris(benzylideneacetone)dipalladium (0) and xantphos (0.01-0.10 eq., typically 0.05 eq.). The reaction mixture is heated at about 40-150° C. (e.g., about 95° C.) for about 0.5-24 hours (e.g., about 2 hours) or at about 100-200° C. (e.g., 150° C.) for about 5-60 minutes (e.g., about 15 minutes) in a microwave under an inert atmosphere. The reaction mixture is allowed to cool and solvents are removed under reduced pressure to give the product that may be further purified by crystallization or chromatography.

### Exemplification of General Procedure V

### Example #4

### Preparation of 1-tert-Butyl-5-phenyl-1H-pyrazole-3-carboxylic acid (4-formyl-phenyl)-amide

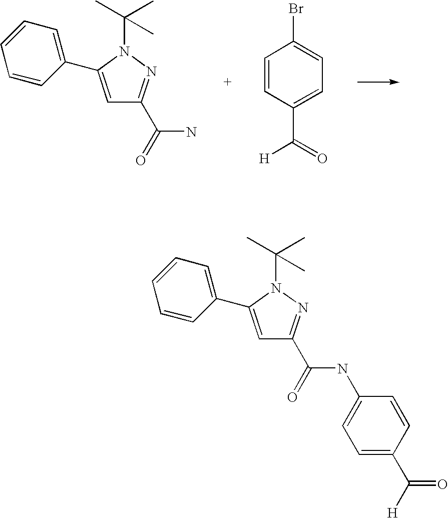

4-Bromobenzaldehyde (0.304 g, 1.644 mmol), 1-tert-butyl-5-(4-fluorophenyl)-1H-pyrazole-3-carboxamide (0.400 g, 1.644 mmol), cesium carbonate (0.625 g, 1.918 mmol), Zantphos (0.048 g, 0.082 mmol) and trisbenzylideneacetone)dipalladium(0) (0.025 g, 0.027 mmol) were combined neat under an atmosphere of nitrogen and were diluted with dioxane (3.0 mL). The mixture was further degassed with a stream of nitrogen for about 5 minutes, and then the mixture was heated at 100° C. overnight. The reaction was cooled to room temperature, diluted with ethyl acetate (5 mL) and filtered through silica gel (10 g), rinsing with ethyl acetate. Solvents were removed under reduced pressure and the crude was triturated with ether (20 mL), filtered and dried to yield 1-tert-butyl-5-phenyl-1H-pyrazole-3-carboxylic acid (4-formylphenyl)-amide (0.298 g, 52%) as an off-white solid. LCMS (Table 1, Method b) Rₜ=4.00 min, m/z 348.42 (M+H)⁺; ¹H NMR (400 MHz, DMSO-d6) δ 10.43 (s, 1H), 9.87 (s, 1H), 8.04 (d, 2H), 7.84 (d, 2H), 7.50-7.60 (m, 5H), 6.79 (s, 1H), 1.18 (s, 9H).

### General Procedure W: Deprotection of an Acetamide to an Amine

The acetate (1 eq.) was suspended in an a protic organic solvent (e.g., methanol). 1-5 eq. (e.g., 3.6 eq.) of sulfuric acid is added and the reaction heated to reflux for 2-24 hours (e.g., 16 hours). The reaction is cooled and concentrated under vacuum. The residue is partitioned between an organic solvent (e.g., ethyl acetate) and water. The organic layer is washed with water, dried over an anhydrous salt (e.g., sodium sulfate), filtered, and concentrated to afford the desired product that is used without further purification.

### Exemplification of General Procedure W

### Preparation of methyl 2-(4-amino-3-chlorophenylsulfonamido)acetate

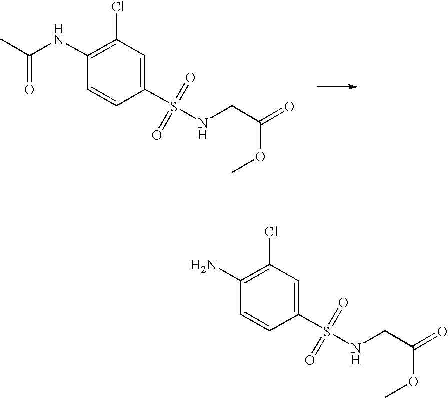

To a suspension of tert-butyl 2-(4-acetamido-3-chlorophenylsulfonamido)acetate (0.13 g, 0.358 mmol) in MeOH (6 mL) was added sulfuric acid (0.070 mL, 1.311 mmol). The reaction mixture was heated at reflux overnight and then allowed to cool down. The colorless solution was concentrated and the residue was partitioned between EtOAc (50 mL) and water (50 mL). The EtOAc layer was washed with water (3×20 mL). The organic layer was dried over Na₂SO₄, filtered and concentrated to afford methyl 2-(4-amino-3-chlorophenylsulfonamido)acetate (0.1 g, 0.358 mmol, 100% yield) as an orange oil. LCMS (Table 1, Method a) Rₜ=2.58 min, MS m/z: 279.01 (M+H)⁺; ¹H NMR (400 MHz, DMSO-d6) δ 7.87 (t, 1H, J=6.25 Hz), 7.54 (d, 1H, J=2.40 Hz), 7.39 (dd, 1H, J=2.15, 8.58 Hz), 6.83 (d, 1H, J=8.53 Hz), 6.20 (d, 2H, J=5.61 Hz) 3.62 (d, 2H, J=6.17 Hz), 3.54 (s, 3H).

### General Procedure X: Deprotection of a Silyl Protected Alkyne

The alkyne (1 eq.) is stirred in a protic organic solvent (e.g., methanol). Potassium carbonate (0.1 eq.) is added and the reaction stirred at ambient temperature for 10-60 minutes (e.g., 30 minutes). The reaction is concentrated under vacuum and the residue partitioned between an organic solvent (e.g., diethylether) and brine. The organic layer is separated and dried over an anhydrous salt (e.g., magnesium sulfate, filtered, and concentrated to give the desired product.

### Exemplification of General Procedure X

### Preparation of 2-(4-ethynylphenyl)-1,3-dioxolane

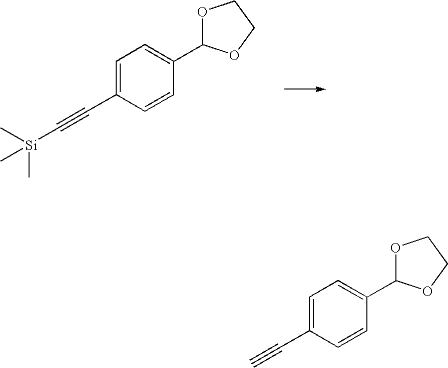

To a stirred solution of ((4-(1,3-dioxolan-2-yl)phenyl)ethynyl)trimethylsilane (11.31 g, 45.9 mmol) in methanol (54.0 mL) was added potassium carbonate (0.634 g, 4.59 mmol). The reaction was stirred at ambient temperature for 30 min. The crude reaction was then concentrated. The resulting residue was partitioned into brine and diethyl ether. The organic phase was separated, dried (MgSO₄) and concentrated to yield 2-(4-ethynylphenyl)-1,3-dioxolane (8.18 g, 47.0 mmol) as dark brown oil. ¹H NMR (400 MHz, CD₂Cl₂) δ ppm 7.51 (m, 2H), 7.45 (m, 2H), 5.78 (s, 1H), 4.04 (m, 4H), 3.17 (s, 1H).

### General Procedure Y: Michael Addition of a Amine to an α,β-Unsaturated Ester

To a solution of amine (1 eq.) in a suitable solvent (MeOH, EtOH, ACN, DMF) is added methyl acrylate (5-30 eq., e.g., 10 eq.) with or without a base (e.g., DBU, 0.5 eq). The mixture is heated at 100-180° C. by microwave (e.g., 120° C.) for 0.5-3 hours (e.g., 1 hour). The solvent is removed under reduced pressure. The residue is purified by flash column chromatography to give the desired product.

### Exemplification of General Procedure Y

### Preparation of methyl 3-(2-(4-(5-(3-chloro-4-(isopropylamino)phenyl)isoxazole-3-carboxamido)phenyl)propan-2-ylamino)propanoate

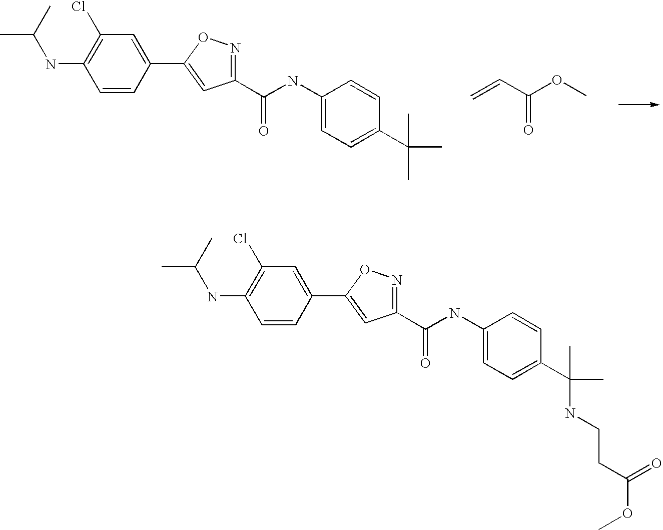

A 5 mL microwave vial equipped with a stir bar was charged with N-(4-(2-aminopropan-2-yl)phenyl)-5-(3-chloro-4-(isopropylamino)phenyl)isoxazole-3-carboxamide (220 mg, 0.533 mmol), methanol (2.0 mL) and methyl acrylate (0.480 mL, 5.33 mmol). The vial is sealed and heated to 120° C. for 60 minutes in the microwave. Solvent was removed and the residue was purified by flash chromatography (0-10% MeOH/DCM over 40 min; Redi-sep column, 40 g). Collected fractions and concentrated to dryness to give methyl 3-(2-(4-(5-(3-chloro-4-(isopropylamino)phenyl)isoxazole-3-carboxamido)phenyl)propan-2-ylamino)propanoate (0.254 g, 0.509 mmol, 96% yield) as a light yellow oil. %). LCMS (Method 6) Rₜ 1.84 min; m/z: (M+H)⁺ 499.24.

### General Procedure Z: Deprotection of a Boc-Protected Amine

To a solution of Boc-protected amine (1 eq.) in a suitable solvent (DCM, DCE) is added methyl acrylate (5-100 eq., e.g., 10-30 eq.) The mixture is stirred at room temperature for 0.25-24 hours (e.g., 1 hour). The solvent is removed under reduced pressure. The residue can be used as it is or be purified by flash column chromatography.

### Exemplification of General Procedure Z

### (R)-N-(4-(1-aminoethyl)phenyl)-5-(3-chloro-4-isopropoxyphenyl)isoxazole-3-carboxamide

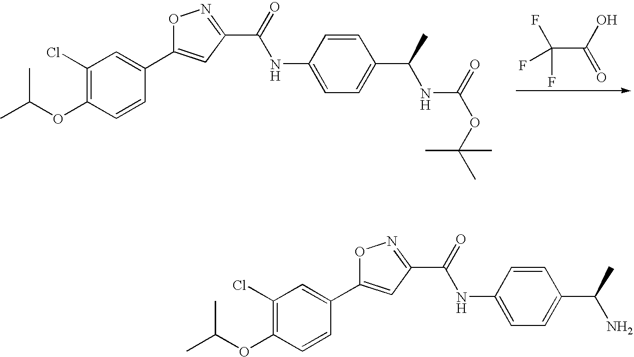

To a solution of (R)-tert-butyl 1-(4-(5-(3-chloro-4-isopropoxyphenyl)isoxazole-3-carboxamido)phenyl)ethylcarbamate (440 mg, 0.880 mmol) in DCM (8 mL) was added TFA (2 mL, 26.0 mmol). The solution was stirred for 1 hour. Solvent was removed under reduced pressure. The residue was purified by flash chromatography (0-10% MeOH/DCM over 30 min; Redi-Sep column, 12 g). Collected fractions and concentrated to give (R)—N-(4-(1-aminoethyl)phenyl)-5-(3-chloro-4-isopropoxyphenyl)isoxazole-3-carboxamide (350 mg, 0.875 mmol, 99% yield) as an off-white solid. LCMS (Method b) Rₜ 2.00 min; m/z: (M−H)⁻ 398.36.

### General Procedure AA: Cyclization of a Hydroxyamidine to an Oxadiazole

A hydroxyamidine compound is suspended in an organic solvent such as 1,2-dichloroethane at ambient temperature. An organic base such as pyridine (1-3 equivalents, e.g., 3 equivalents) is added dropwise and the resulting mixture is cooled to about 0° C. Ethyl 2-chloro-2-oxoacetate (1-3 equivalents, e.g., 1.5 equivalents) is added dropwise over a period of about 10 min. After stirring at 0° C. for about 1-4 hrs, e.g., 1 hr, the mixture is warmed to ambient temperature for about 0.5 hr and then heated to about 80° C. for 1-4 hrs, e.g., about 2 hrs. The reaction is cooled to ambient temperature and diluted with an organic solvent such as dichloromethane, ethyl acetate or toluene, typically dichloromethane. The organic phase is washed with aqueous acidic solution such as 1N HCl solution then washed with aqueous solvents to remove salts. The organic layer is dried, filtered and concentrated. The crude product may be used as is or further purified by crystallization or chromatography on silica gel.

### Exemplification of General Procedure AA

### Preparation of ethyl 3-(3-chloro-4-isopropoxyphenyl)-1,2,4-oxadiazole-5-carboxylate

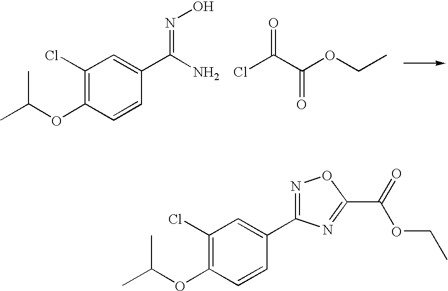

Pyridine (1.061 mL, 13.12 mmol) was added dropwise to a stirred suspension of (Z)-3-chloro-N′-hydroxy-4-isopropoxybenzimidamide (1.0 g, 4.37 mmol) in 1,2-dichloroethane (16.20 mL) at room temperature under an atmosphere of nitrogen. The mixture was cooled to 0° C. and ethyl 2-chloro-2-oxoacetate (0.730 mL, 6.56 mmol) was added dropwise over 10 min. After stirring at 0° C. for 1 hr, the mixture was warmed to room temperature for 0.5 hr and then to 80° C. and stirred for 2 hrs. Heating was stopped and the reaction was cooled to ambient temperature and diluted with DCM (50 mL) and washed with 1N HCl (2×50 mL), then water (50 mL), brine (50 mL), dried (MgSO₄) and concentrated in vacuo to give ethyl 3-(3-chloro-4-isopropoxyphenyl)-1,2,4-oxadiazole-5-carboxylate (1.35 g, 4.26 mmol) as colorless oil, later solidified to an off-white solid with the aid of a spatula. LCMS (Table 1, Method g) Rₜ=2.92 min, m/z 311.06 (M+H)⁺; ¹H NMR (400 MHz, CDCl₃) δ ppm 8.19 (d, J=2.10 Hz, 1H), 7.99 (dd, J=8.64, 2.08 Hz, 1H), 7.06-6.98 (m, 1H), 4.68 (td, J=12.14, 6.08 Hz, 1H), 4.57 (q, J=7.13 Hz, 2H), 1.49 (t, J=7.15 Hz, 3H), 1.43 (t sext., J=8.28, 7.19 Hz, 6H).

### General Procedure BB: Reduction of a Nitro Compound to an Amine

To a nitro compound dissolved in a suitable solvent (THF, MeOH, EtOAc) is added magnesium sulfate (1-5 eq, e.g., 2 eq)) and PtO₂ (0.05-0.5 eq, e.g., 0.1 eq). The mixture is degassed hydrogenated with 1 atm H₂ at room temperature for 8-24 hours. The mixture is filtered and washed with a suitable solvent and concentrated to dryness to the amine that can be purified by flash chromatography or used as it is.

### Exemplification of General Procedure BB

### tert-butyl 1-(4-aminophenyl)cyclopropylcarbamate

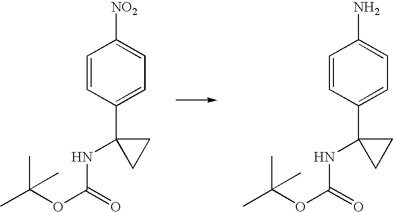

To a solution of tert-butyl 1-(4-nitrophenyl)cyclopropylcarbamate (400 mg, 1.437 mmol) in THF (15 mL) was added magnesium sulfate (346 mg, 2.87 mmol) and PtO₂ (32.6 mg, 0.144 mmol). The mixture was degassed, purged with N₂ and charged with H₂ at 1 atm. The mixture was stirred at room temperature overnight. Filtered and washed with THF, concentrated to dryness to give tert-butyl 1-(4-aminophenyl)cyclopropylcarbamate (360 mg, 1.450 mmol, 101% yield) as an colorless oil. LCMS (Table, 1, Method b) Rₜ=1.65 min, m/z 249.16 (M−H)⁻. ¹H NMR (400 MHz, DMSO-d6) δ 7.46 (s, 1H), 6.85 (d, J=8.2, 2H), 6.45 (d, J=8.5, 2H), 4.83 (s, 2H), 1.35 (s, 9H), 0.93 (dt, J=7.2, 11.7, 4H).

### Tables Utilizing General Procedures

TABLE A
Examples prepared following general procedures A, B, C, D.3 (Scheme 1)

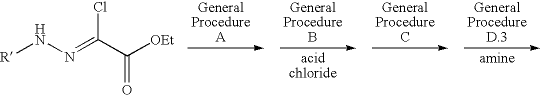

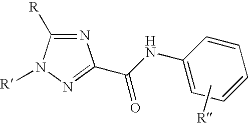

| | Acid chloride, | | HPLC Rₜ | |
|---|---|---|---|---|
| Ex. # | Amine | Product | (Method) | m/z |
| A1 | Trichloro-acetylchloride, 2-Chloroaniline | 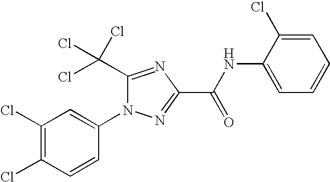 | 2.82 mins(a) | (M + H)484.97 |
| A2 | Trichloro-acetylchloride, 3-Chloroaniline | 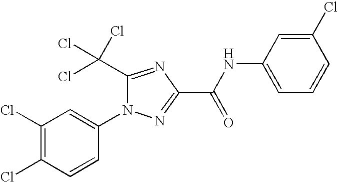 | 2.69 mins(a) | (M + H)484.97 |
| A3 | Trichloro-acetylchloride, m-Toluidine | 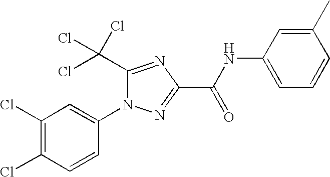 | 2.64 mins(a) | (M + H)462.98 |
| A4 | Trichloro-acetylchloride, 3-Methoxyaniline | 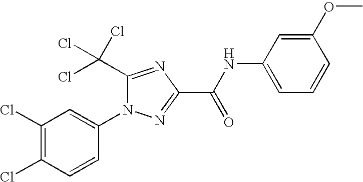 | 2.56 mins(a) | (M + H)482.97 |
| A5 | Trichloro-acetylchloride, 3-(Trifluoromethyl)-aniline | 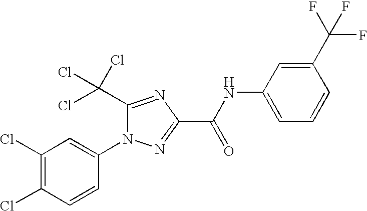 | 2.72 mins(a) | (M + H)518.95 |
| A6 | Trichloro-acetylchloride, Ethyl 3-aminobenzoate | 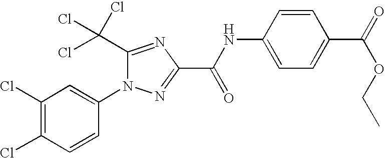 | 2.65 mins(a) | (M − H)521.07 |
| A7 | Trichloro-acetylchloride, 2,3-Dihydro-1H-inden-5-amine | 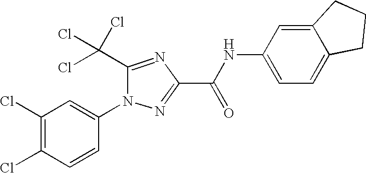 | 2.76 mins(a) | (M − H)489.04 |
| A8 | Trichloro-acetylchloride, Trichloro-acetyl chloride, 2-(Trifluoromethyl)-aniline | 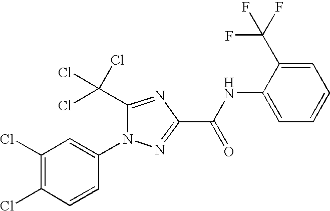 | 2.76 mins(a) | (M − H)517.01 |
| A9 | Trichloro-acetylchloride, 3-Methylpyridin-4-amine | 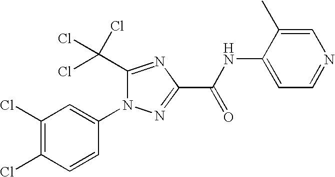 | 2.89 mins(a) | (M − H)466.23 |
| A10 | 1-(3-Chloro-phenyl)-5-trifluoromethyl-1H-[1,2,4]triazole-3-carboxylic acid | 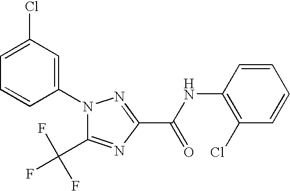 | 2.60 min(b) | 400.01(M − H)⁻ |
| A11 | 1-(3-Chloro-phenyl)-5-trichloromethyl-1H-[1,2,4]triazole-3-carboxylic acid | 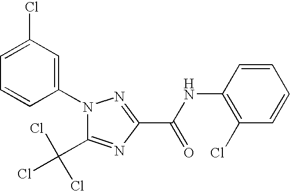 | 2.77 min(b) | 449.02(M − H)⁻ |

TABLE B
Examples prepared following general procedures A, B, C, D.1, G.1, H (Scheme II)

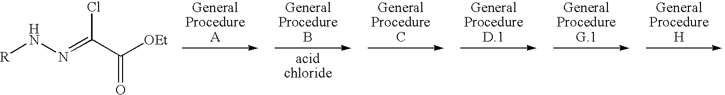

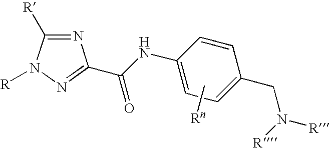

| | Starting material, | | HPLC Rₜ | |
|---|---|---|---|---|
| Ex. # | amine | Product | (Method) | m/z |
| B1 | 1-(3,4-dichlorophenyl)-5-(trichloromethyl)-1H-1,2,4-triazole-3-carboxylic acid(Bionet), 4-amino-3-chloro-benzonitrile | 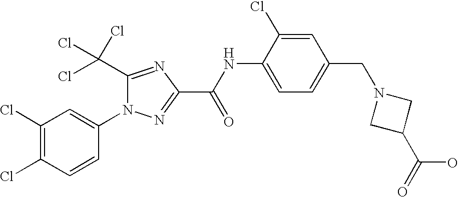 | 2.00 min(a) | 597.98(M + H)⁺ |
| B2 | (Z)-ethyl 2-chloro-2-(2-phenylhydrazono)acetate (Oakwood),4-amino-3-chlorobenzaldehyde | 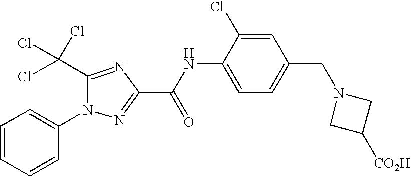 | 2.70 min(a) | 528.23(M − H)⁻ |
| B3 | (Z)-ethyl 2-chloro-2-(2-phenylhydrazono)acetate (Oakwood),4-amino-3-chlorobenzaldehyde | 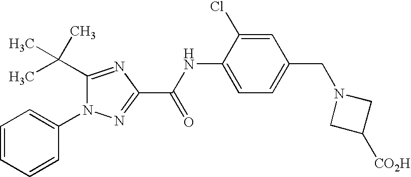 | 2.04 min(a) | 468.28(M + H)⁺ |

TABLE C
Examples prepared following general procedures I, J, H (Scheme III)

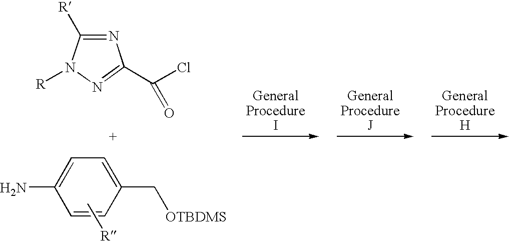

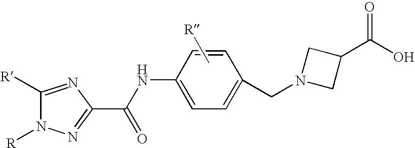

| | Starting material, | | HPLC Rₜ | |
|---|---|---|---|---|
| Ex. # | amine | Product | (Method) | m/z |
| C1 | 1-(3,4-dichlorophenyl)-5-(trichloromethyl)-1H-1,2,4-triazole-3-carboxylic acid(Bionet), 4-amino-benzonitrile | 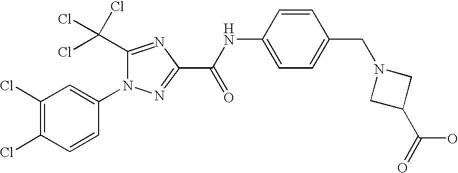 | 2.89 min(a) | 564.20(M + H)⁺ |
| C2 | 1-(3,4-dichlorophenyl)-5-(trichloromethyl)-1H-1,2,4-triazole-3-carboxylic acid(Bionet), 3-((tert-butyldimethylsilyl-oxy)methyl)aniline | 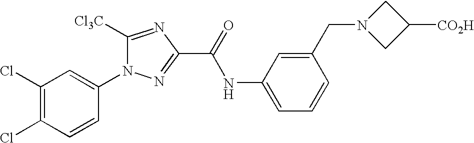 | 2.11 min(a) | 564.01(M + H)⁺ |
| C3 | (Z)-ethyl 2-chloro-2-(2-phenylhydrazono)acetate (Oakwood),4-((tert-butyldimethylsilyl-oxy)methyl)aniline | 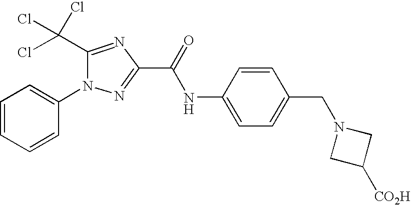 | 2.50 min(a) | 494.26(M − H)⁻ |
| C4 | (Z)-ethyl 2-chloro-2-(2-phenylhydrazono)acetate (Oakwood),4-((tert-butyldimethylsilyl-oxy)methyl)aniline | 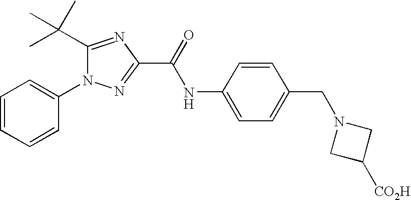 | 1.98 min(a) | 434.29(M − H)⁻ |
| C5 | 3-chloro-4-isopropoxyaniline(Matrix), 4-((tert-butyldimethylsilyl-oxy)methyl)aniline | 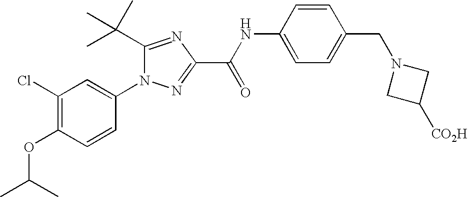 | 2.89 min(a) | 526.52(M + H)⁺ |

TABLE D
Examples prepared following general procedures L, K, X, M, D.4, N, H (Scheme IV)

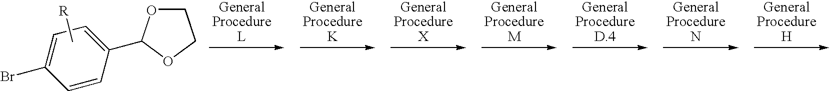

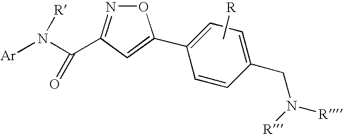

| | | | HPLC Rₜ | |
|---|---|---|---|---|
| Ex. # | Starting material | Product | (Method) | m/z |
| D1 | 2-(4-bromophenyl)-1,3-dioxolane | 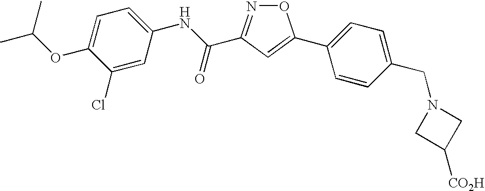 | 1.81 min(c) | 470.20(M + H)⁺ |
| D2 | 2-(4-bromophenyl)-1,3-dioxolane | 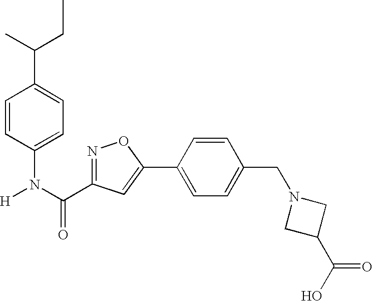 | 2.29 min(a) | 434 |
| D3 | 2-(4-bromophenyl)-1,3-dioxolane | 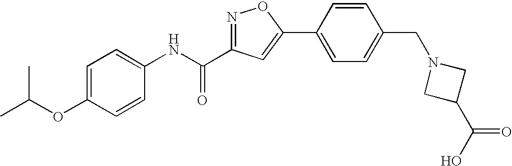 | 2.06 min(a) | 436 |
| D4 | 2-(4-bromophenyl)-1,3-dioxolane | 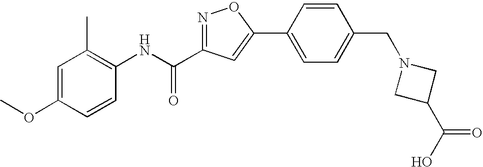 | 1.9 min (a) | 422 |
| D5 | 2-(4-bromophenyl)-1,3-dioxolane | 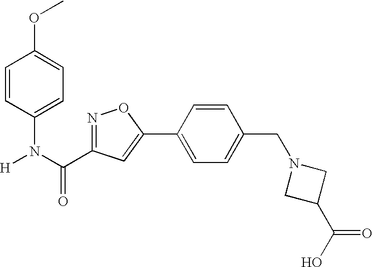 | 1.87 min(a) | 408 |
| D6 | 2-(4-bromophenyl)-1,3-dioxolane | 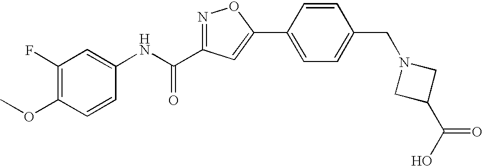 | 2.00 min(a) | 426 |
| D7 | 2-(4-bromophenyl)-1,3-dioxolane | 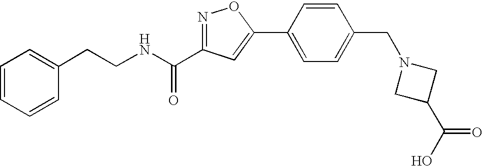 | 1.99 min(a) | 406 |
| D8 | 2-(4-bromophenyl)-1,3-dioxolane | 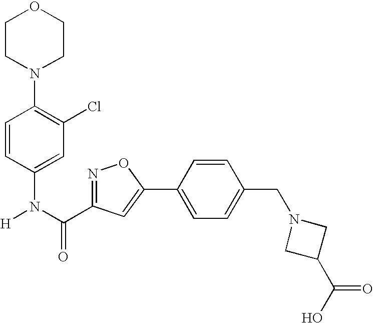 | 2.00 min(a) | 497 |
| D9 | 2-(4-bromophenyl)-1,3-dioxolane | 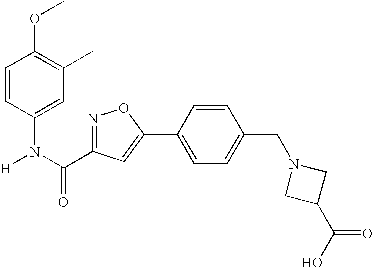 | 2.01 min(a) | 422 |
| D10 | 2-(4-bromophenyl)-1,3-dioxolane | 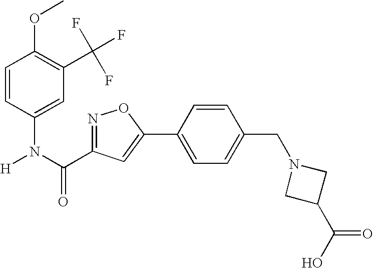 | 2.10 min(a) | 476 |
| D11 | 2-(4-bromophenyl)-1,3-dioxolane | 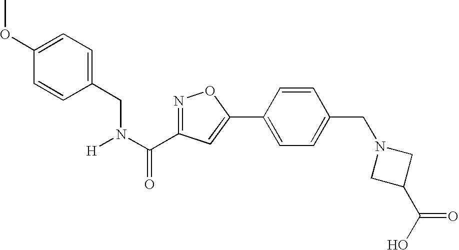 | 1.86 min(a) | 422 |
| D12 | 2-(4-bromophenyl)-1,3-dioxolane | 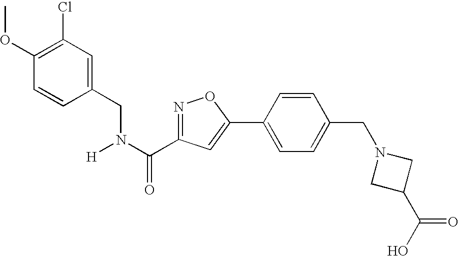 | 1.91 min(a) | 456 |
| D13 | 2-(4-bromophenyl)-1,3-dioxolane | 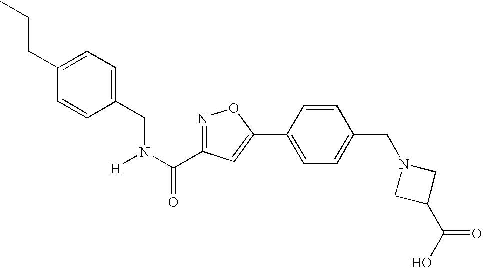 | 2.16 min(a) | 434 |
| D14 | 2-(4-bromophenyl)-1,3-dioxolane | 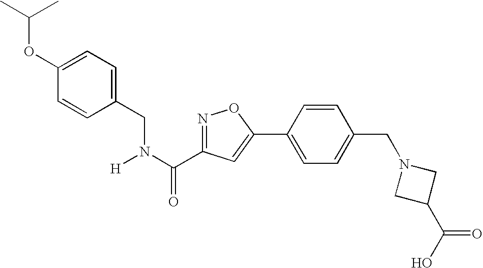 | 2.00 min(a) | 450 |
| D15 | 2-(4-bromophenyl)-1,3-dioxolane | 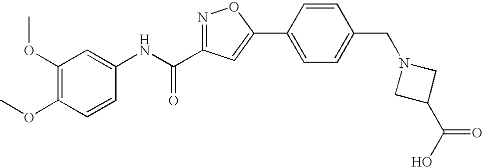 | 1.8 min (a) | 438 |
| D16 | 2-(4-bromophenyl)-1,3-dioxolane | 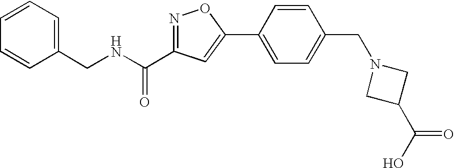 | 1.92 min(a) | 392 |
| D17 | 2-(4-bromo-3-chlorophenyl)-1,3-dioxolanePrep A LuII | 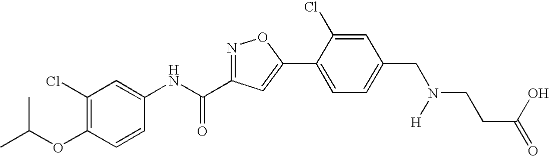 | 2.27 min(a) | 492 |
| D18 | 2-(4-bromo-3-chlorophenyl)-1,3-dioxolanePrep A LuII | 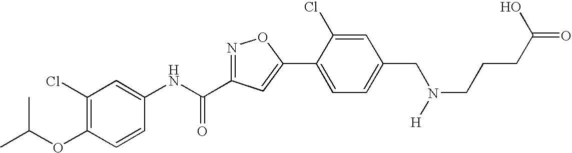 | 2.34 min(a) | 506 |
| D19 | 2-(4-bromo-3-chlorophenyl)-1,3-dioxolanePrep A LuII | 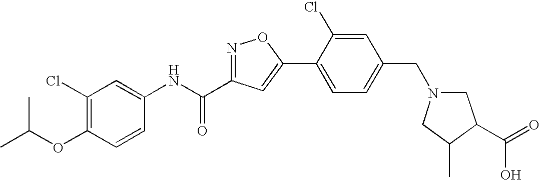 | 2.44 min(a) | 532 |
| D20 | 2-(4-bromo-3-chlorophenyl)-1,3-dioxolanePrep A LuII | 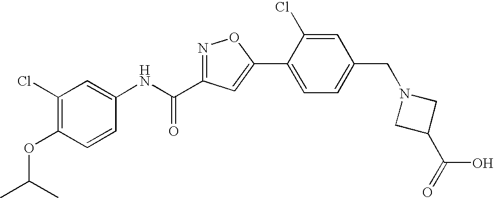 | 2.27 min(a) | 504 |
| D21 | 2-(4-bromo-3-chlorophenyl)-1,3-dioxolanePrep A LuII | 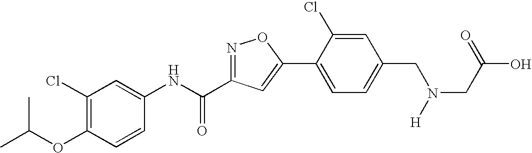 | 2.23 min(a) | 478 |
| D22 | 2-(4-bromo-3-chlorophenyl)-1,3-dioxolanePrep A LuII | 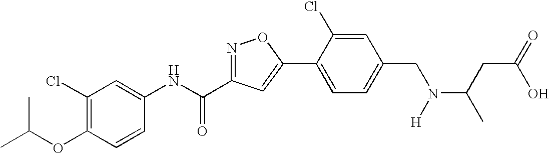 | 2.42 min(a) | 506 |
| D23 | 2-(4-bromo-3-chlorophenyl)-1,3-dioxolanePrep A LuII | 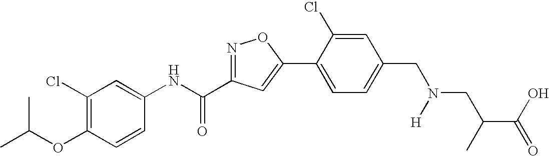 | 2.41 min(a) | 506 |
| D24 | 2-(4-bromo-3-chlorophenyl)-1,3-dioxolanePrep A LuII | 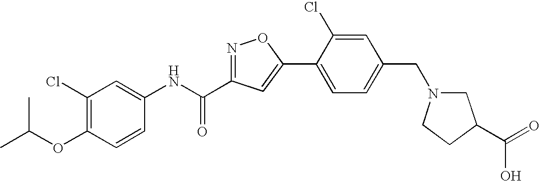 | 2.35 min(a) | 518 |
| D25 | 2-(4-bromo-3-chlorophenyl)-1,3-dioxolanePrep A LuII | 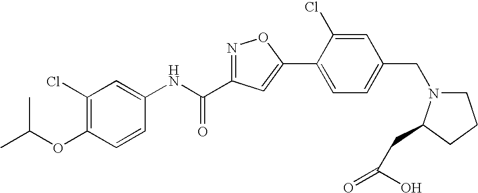 | 2.56 min(a) | 532 |
| D26 | 2-(4-bromo-3-chlorophenyl)-1,3-dioxolanePrep A LuII | 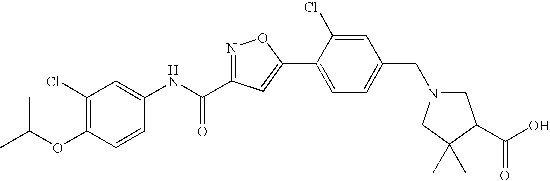 | 2.46 min(a) | 546 |
| D27 | 2-(4-bromo-3-chlorophenyl)-1,3-dioxolanePrep A LuII | 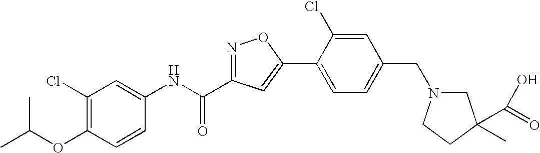 | 2.43 min(a) | 532 |
| D28 | 2-(4-bromo-3-chlorophenyl)-1,3-dioxolanePrep A LuII | 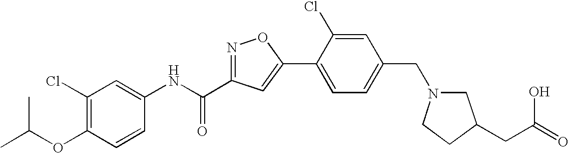 | 2.43 min(a) | 532 |
| D29 | 2-(4-bromo-3-chlorophenyl)-1,3-dioxolanePrep A LuII | 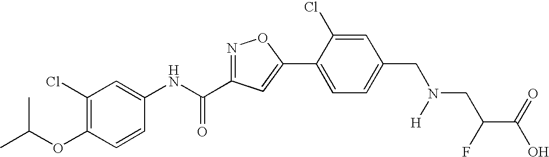 | 2.29 min(a) | 510 |
| D30 | 2-(4-bromo-3-chlorophenyl)-1,3-dioxolanePrep A LuII | 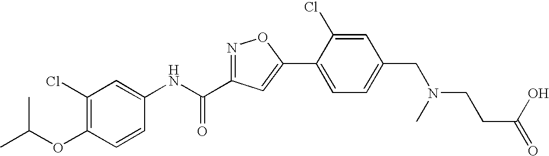 | 2.42 min(a) | 506 |
| D31 | 2-(4-bromo-3-chlorophenyl)-1,3-dioxolanePrep A LuII | 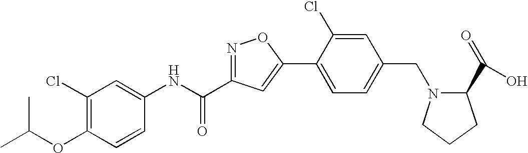 | 2.46 min(a) | 518 |
| D32 | 2-(4-bromo-3-chlorophenyl)-1,3-dioxolanePrep A LuII | 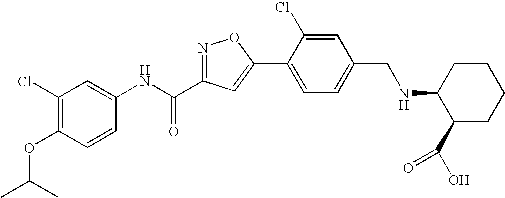 | 2.58 min(a) | 546 |
| D33 | 2-(4-bromo-3-chlorophenyl)-1,3-dioxolanePrep A LuII | 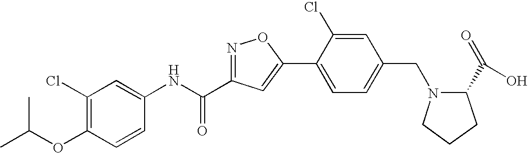 | 2.47 min(a) | 518 |
| D34 | 2-(4-bromo-3-chlorophenyl)-1,3-dioxolanePrep A LuII | 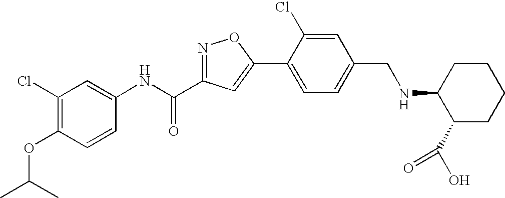 | 2.55 min(a) | 546 |
| D35 | 2-(4-bromo-3-chlorophenyl)-1,3-dioxolanePrep A LuII | 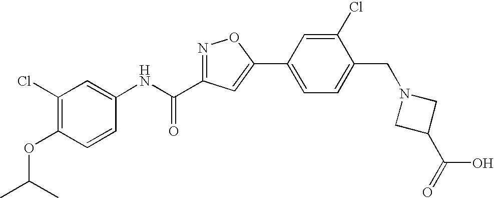 | 2.29 min(a) | 504 |

TABLE E
Examples prepared following general procedures D or F (Scheme VI)

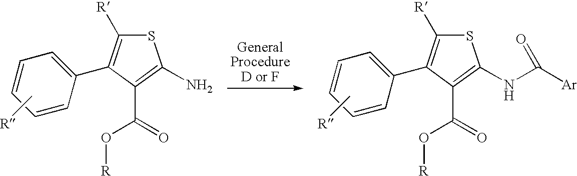

| | Amine, acid or | | HLPLC Rₜ | |
|---|---|---|---|---|
| Ex. # | acid chloride | Product | (Method) | m/z |
| E1 | ethyl 2-amino-4-phenylthiophene-3-carboxylate | 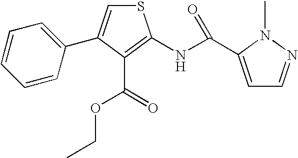 | 3.12 min(c) | 370.18(M + H)⁺ |
| E2 | ethyl 2-amino-4-phenylthiophene-3-carboxylate | 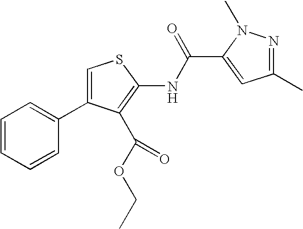 | 2.32 min(c) | 370.18(M + H)⁺ |
| E3 | ethyl 2-amino-4-phenylthiophene-3-carboxylate | 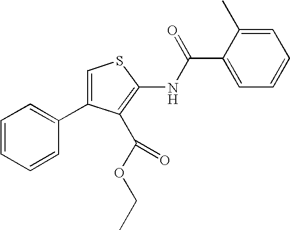 | 3.18 min(c) | 366.29(M + H)⁺ |
| E4 | ethyl 2-amino-4-phenylthiophene-3-carboxylate | 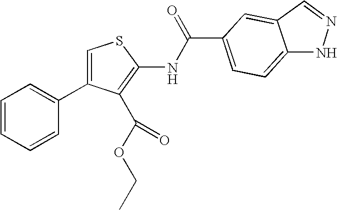 | 2.97 min(c) | 392.27(M + H)⁺ |
| E5 | ethyl 2-amino-4-(4-propoxyphenyl)-thiophene-3-carboxylate; 4-(1,3-dioxolan-2-yl)benzoic acid[prepared viageneral procedure Lfrom 4-formylbenzoic acid] | 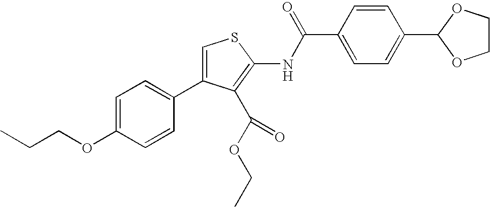 | 4.73 min(a) | 482.37(M + H)⁺ |
| E6 | 2-Amino-4-phenyl-thiophene-3-carboxylic acidethyl ester | 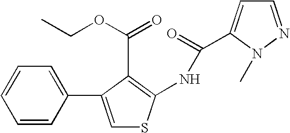 | 2.32 min(b) | 356.15(M + H)⁺ |

TABLE F
Examples prepared following general procedures D or F (Scheme VII)

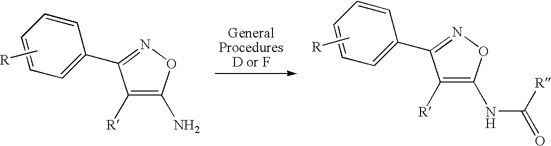

| | Amine, acid or | | HPLC Rₜ | |
|---|---|---|---|---|
| Ex. # | acid chloride | Product | (Method) | m/z |
| F1 | ethyl 5-amino-3-phenylisoxazole-4-carboxylate,benzoyl chloride | 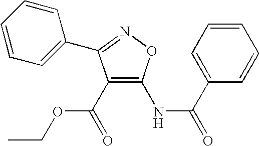 | 3.01 min(a) | 335.17(M − H)⁻ |
| F2 | 3-phenylisoxazol-5-amine; 4-cyanobenzoylchloride | 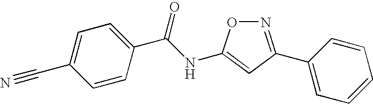 | 2.42 min(a) | 290.28(M + H)⁺ |
| F3 | 3-phenyl-isoxazol-5-ylamine, benzoylchloride | 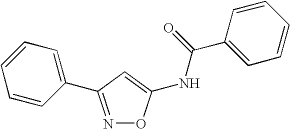 | 2.18 min(a) | 263.3(M + H)⁺ |
| F4 | 3-phenyl-isoxazol-5-ylamine, p-toluoyl chloride | 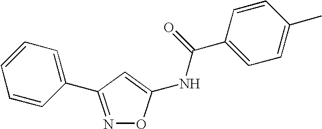 | 2.29 min(a) | 279.1(M + H)⁺ |
| F5 | 3-phenyl-isoxazol-5-ylamine, 4-fluorobenzoylchloride | 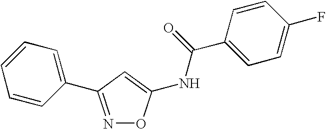 | 2.23 min(a) | 283.1(M + H)⁺ |
| F6 | 3-phenyl-isoxazol-5-ylamine, 4-methoxyobenzoylchloride | 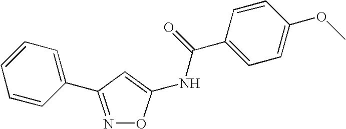 | 2.19 min(a) | 293.2(M − H)⁻ |
| F7 | 3-phenyl-isoxazol-5-ylamine, 4-chlorobenzoylchloride | 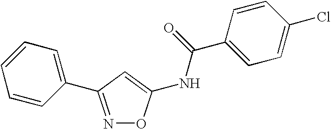 | 2.34 min(a) | 299.1(M + H)⁺ |
| F8 | 3-phenyl-isoxazol-5-ylamine, methyl4-chlorocarbonyl-benzoate | 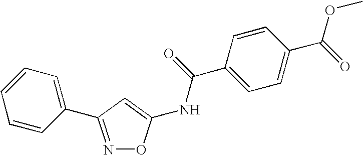 | 2.21 min(a) | 321.2(M − H)⁻ |
| F9 | 3-phenyl-isoxazol-5-ylamine, 3-methoxyobenzoylchloride | 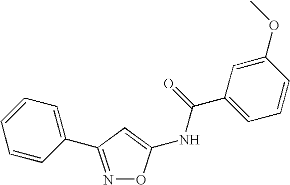 | 2.23 min(a) | 293.2(M − H)⁻ |
| F10 | 3-phenyl-isoxazol-5-ylamine,phenylacetylchloride | 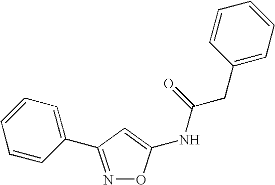 | 2.20 min(a) | 277.2(M − H)⁻ |
| F11 | 3-phenyl-isoxazol-5-ylamine, 4-methoxyphenyl-acetyl chloride | 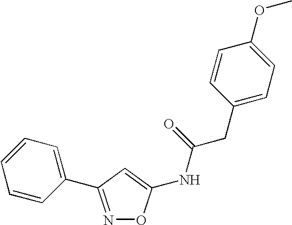 | 2.17 min(a) | 307.2(M − H)⁻ |
| F12 | 3-phenyl-isoxazol-5-ylamine, 1-naphthoyl chloride | 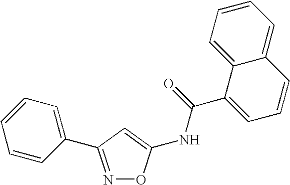 | 2.37 min(a) | 315.1(M + H)⁺ |
| F13 | 3-phenyl-isoxazol-5-ylamine, 2-naphthoyl chloride | 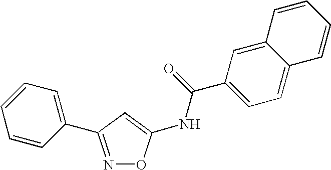 | 2.41 min(a) | 315.1(M + H)⁺ |
| F14 | 3-phenyl-isoxazol-5-ylamine,cyclohexanecarbon-yl chloride | 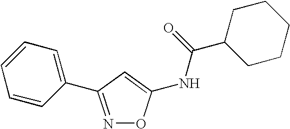 | 2.32 min(a) | 269.2(M − H)⁻ |
| F15 | 3-phenyl-isoxazol-5-ylamine, 2-thiophenecarbonylchloride | 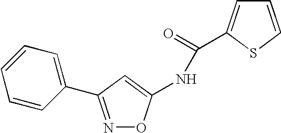 | 2.15 min(a) | 271.1(M + H)⁺ |
| F16 | 3-phenyl-isoxazol-5-ylamine,isonicotinoylchloride | 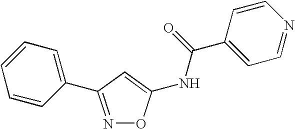 | 1.89 min(a) | 264.2(M − H)⁻ |
| F17 | 3-phenyl-isoxazol-5-ylamine,nicotinoyl chloride | 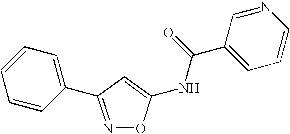 | 1.87 min(a) | 264.2(M − H)⁻ |
| F18 | 3-phenyl-isoxazol-5-ylamine, 2-chlorobenzoylchloride | 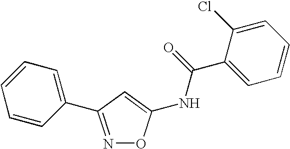 | 2.36 min(a) | 297.1(M − H)⁻ |
| F19 | 3-phenyl-isoxazol-5-ylamine, 3-chlorobenzoylchloride | 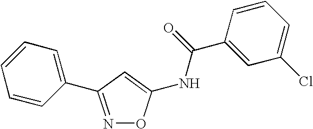 | 3.13 min(a) | 299.1(M + H)⁺ |
| F20 | 3-phenyl-isoxazol-5-ylamine, 2-fluorobenzoylchloride | 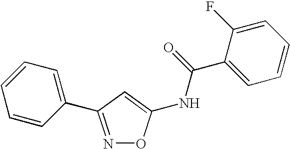 | 2.83 min(a) | 283.1(M + H)⁺ |
| F21 | 3-phenyl-isoxazol-5-ylamine, 3-fluorobenzoylchloride | 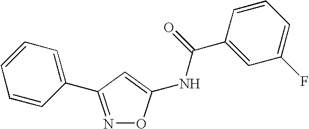 | 2.91 min(a) | 281.1(M − H)⁻ |
| F22 | 3-phenyl-isoxazol-5-ylamine, o-toluoyl chloride | 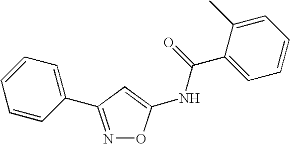 | 2.91 min(a) | 277.2(M − H)⁻ |
| F23 | 3-phenyl-isoxazol-5-ylamine, 3-(trifluoromethyl)-benzoyl chloride | 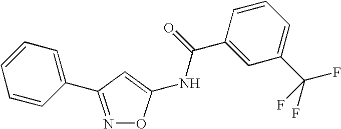 | 3.18 min(a) | 331.1(M − H)⁻ |

TABLE G
Examples prepared following general procedures E, F, I, J.1, H (Scheme IX)

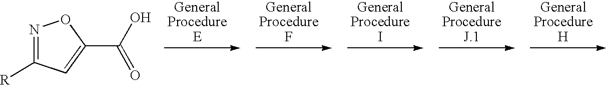

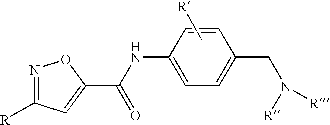

| | Isoxazole, | | HPLC Rₜ | |
|---|---|---|---|---|
| Ex. # | Aniline, amine | Product | (Method) | m/z |
| G1 | 3-phenylisoxazole-5-carboxylic acid | 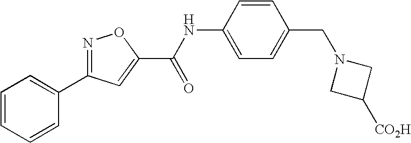 | 2.44 min(a) | 378.30(M + H)⁺ |
| G2 | 3-phenylisoxazole-5-carboxylic acid(Aldrich), 4-((tert-butyldimethylsilyl-oxy)methyl)aniline,Azetidine-3-carboxylic acid | 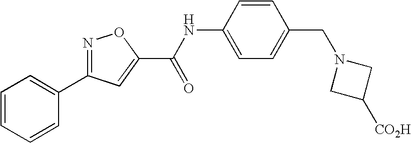 | 2.44 min(a) | 378.30(M + H)⁺ |
| G3 | 3-phenylisoxazole-5-carboxylic acid(Aldrich), 3-((tert-butyldimethylsilyl-oxy)methyl)aniline,Azetidine-3-carboxylic acid | 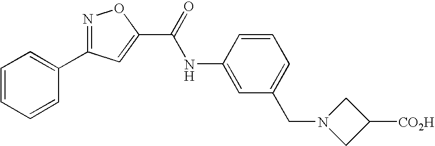 | 1.61 min(a) | 378.18(M + H)⁺ |

TABLE H
Examples prepared following general procedures F, I, J.2, H (Scheme X)

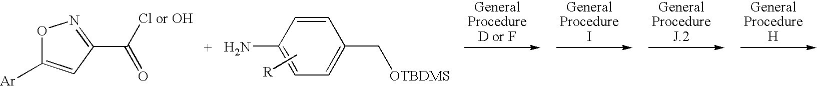

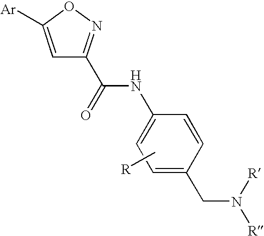

| | | | HPLC Rₜ | |
|---|---|---|---|---|
| Ex. # | | Product | (Method) | m/z |
| H1 | 5-phenylisoxazole-3-carboxylic acid | 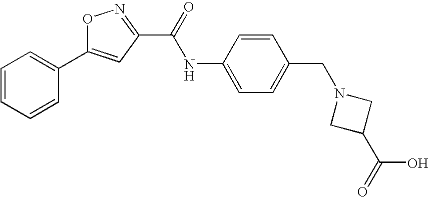 | 1.61 min(a) | 378.18(M + H)⁺ |
| H2 | 5-phenylisoxazole-3-carboxylic acid | 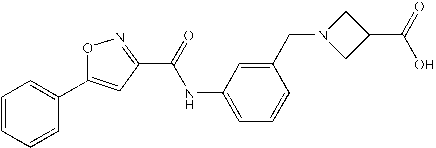 | 2.42 min(a) | 378.30(M + H)⁺ |
| H3 | 5-phenylisoxazole-3-carboxylic acid(Aldrich), 4-((tert-butyldimethylsilyl-oxy)methyl)aniline,2-aminoacetic acid(Aldrich) | 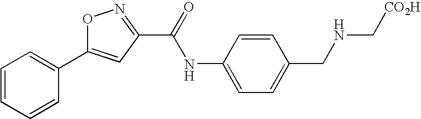 | 2.42 min(a) | 352.23(M + H)⁺ |
| H4 | 5-phenylisoxazole-3-carboxylic acid(Aldrich), 4-((tert-butyldimethylsilyl-oxy)methyl)aniline,3-aminopropanoicacid (Aldrich) | 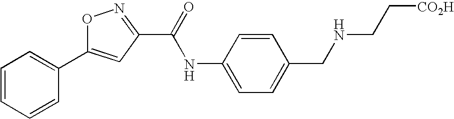 | 2.47 min(a) | 366.29(M + H)⁺ |
| H5 | 5-phenylisoxazole-3-carboxylic acid(Aldrich), 4-((tert-butyldimethylsilyl-oxy)methyl)aniline,4-aminobutanoicacid (Fluka) | 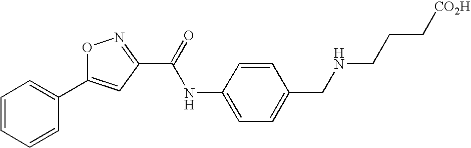 | 2.47 min(a) | 380.29(M + H)⁺ |

TABLE I
Examples prepared following general procedures R, S, C, E, F, I, J.1, H (Scheme XIV)

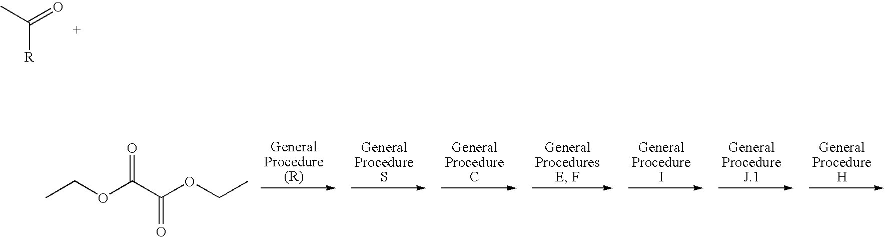

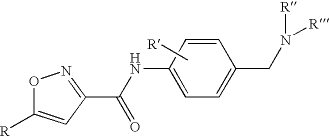

| | | | HPLC Rₜ | |
|---|---|---|---|---|
| Ex. # | | Product | (Method) | m/z |
| I1 | 1-(3-chloro-4-isopropoxyphenyl)-ethanone | 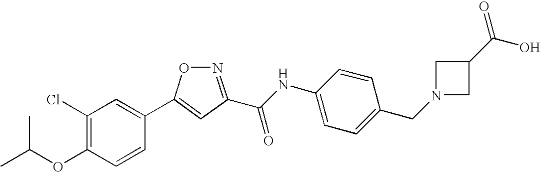 | 1.92 min(b) | 493.17(M + H)⁺ |
| I2 | 1-(3-chloro-4-isopropoxyphenyl)-ethanone | 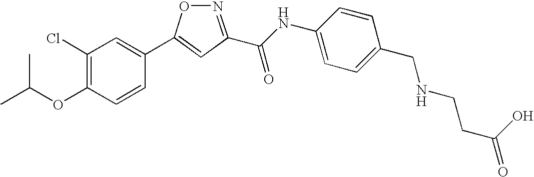 | 2.24 (a) | 456.17(M − H)− |
| I3 | 1-(3-chloro-4-isopropoxyphenyl)-ethanone | 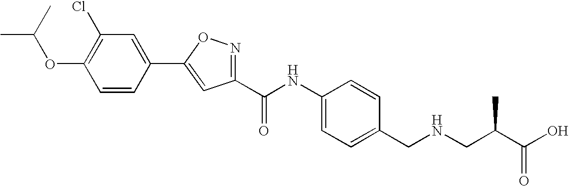 | 1.97 (a) | 470.19(M − H)− |
| I4 | 1-(3-chloro-4-isopropoxyphenyl)-ethanone | 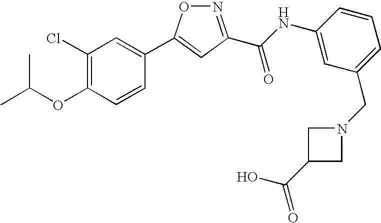 | 2.27 (a) | 468.18(M − H)− |
| I5 | 1-(3-chloro-4-isopropoxyphenyl)-ethanone | 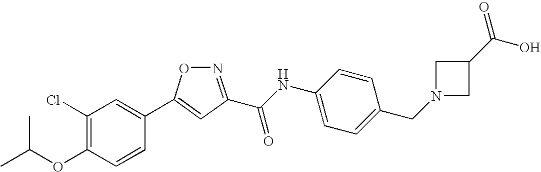 | 2.32 (a) | 468.18(M − H)− |
| I6 | 1-(4-isobutylphenyl)-ethanone | 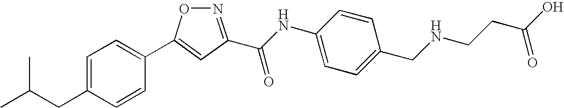 | 2.37 (a) | 420.28(M − H)− |
| I7 | 1-(4-isobutylphenyl)-ethanone | 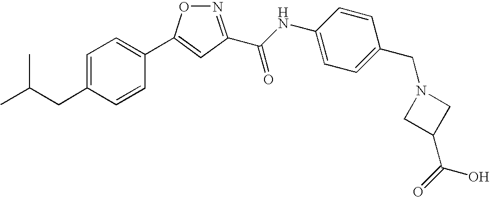 | 2.32 (a) | 432.25(M − H)− |
| I8 | 1-(3-chloro-4-isopropoxyphenyl)-ethanone | 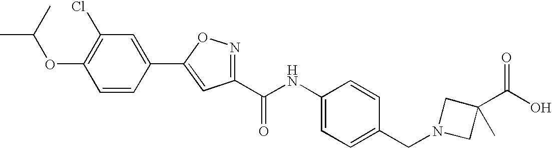 | 1.89 (a) | 482.18(M − H)− |

TABLE J
Examples prepared following general procedures R, T, U, V, HI (Scheme XVI)

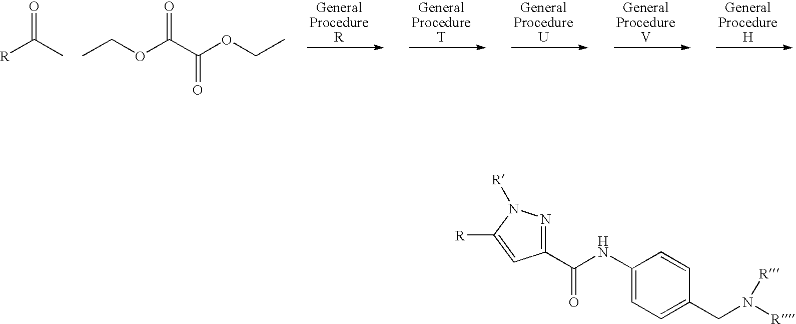

| | | | | HPLC Rₜ | |
|---|---|---|---|---|---|
| Ex. # | Acetophenone | hydrazine | Product | (Method) | m/z |
| J1 | 1-Phenylethanone | tert-Butyl-hydrazine;hydrochloride | 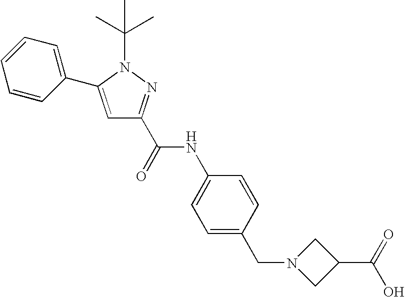 | 1.79min (a) | 433.28(M + H)⁺ |
| J2 | 1-Phenylethanone | tert-Butyl-hydrazine;hydrochloride | 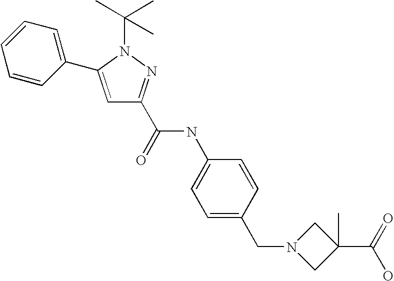 | 2.66min (a) | 447.33(M + H)⁺ |
| J3 | 1-(4-fluorophenyl)ethanone | tert-Butyl-hydrazine;hydrochloride | 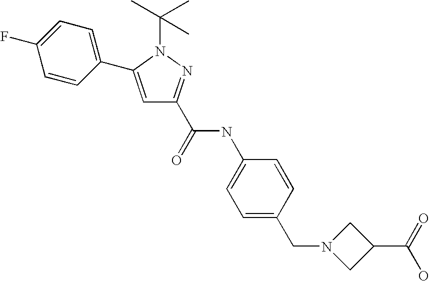 | 3.70min (a) | 451.32(M + H)⁺ |
| J4 | 1-(4-fluorophenyl)ethanone | tert-Butyl-hydrazine;hydrochloride | 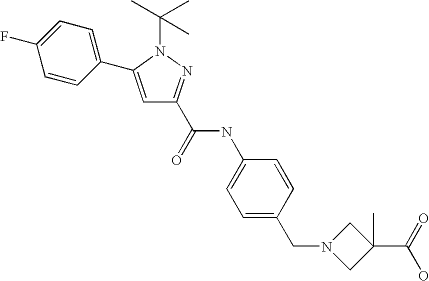 | 3.84min (a) | 465.43(M + H)⁺ |
| J5 | 1-Phenylethanone | isobutylhydrazinesulfate | 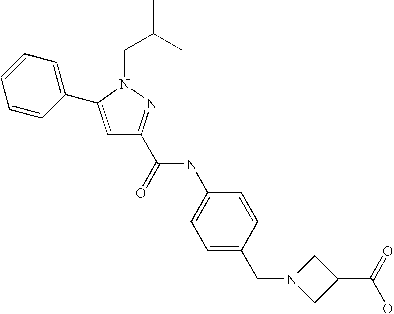 | 2.69min (a) | 433.29(M + H)⁺ |
| J6 | 3,3-Dimethyl-butan-2-one | Phenyl-hydrazinehydrochloride | 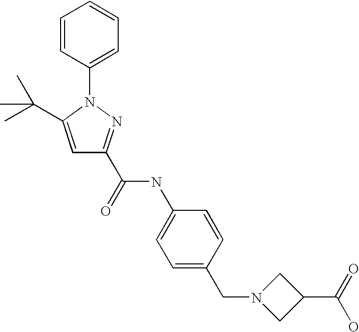 | 2.62min (a) | 433.54(M + H)⁺ |
| J7 | 1-(4-Benzyloxy-phenyl)-ethanone | tert-Butyl-hydrazine;hydrochloride | 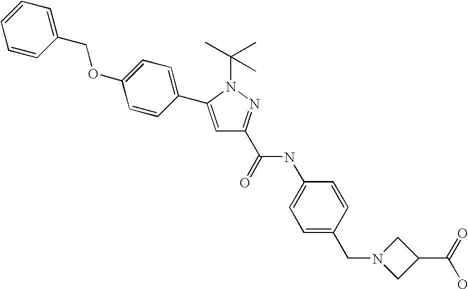 | 4.50min (l) | 539.50(M + H)⁺ |
| J8 | 1-Phenylethanone | tert-Butyl-hydrazine;hydrochloride | 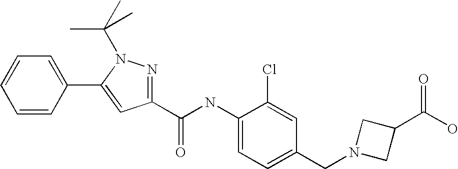 | 2.95min (a) | 467.294 69.29(M + H)⁺ |
| J9 | 1-(4-chlorophenyl)ethanone | tert-Butyl-hydrazine;hydrochloride | 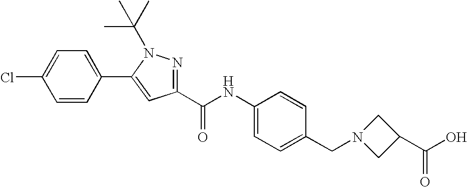 | 2.26min (a) | 467 |
| J10 | 1-Phenylethanone | tert-Butyl-hydrazine;hydrochloride | 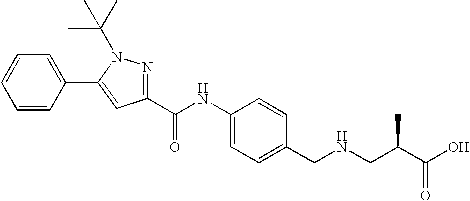 | 2.13min (a) | 435 |
| J11 | 1-Phenylethanone | tert-Butyl-hydrazine;hydrochloride | 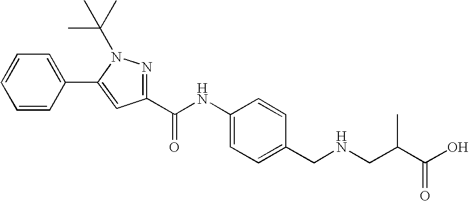 | 1.80min (a) | 435 |
| J12 | 1-Phenylethanone | tert-Butyl-hydrazine;hydrochloride | 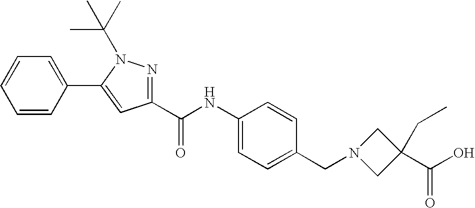 | 2.11min (a) | 461 |
| J13 | 1-Phenylethanone | tert-Butyl-hydrazine;hydrochloride | 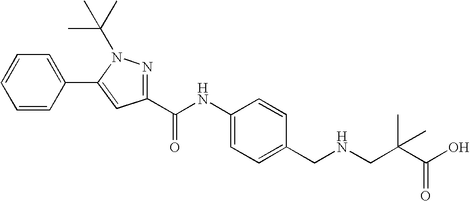 | 2.23min (a) | 449 |
| J14 | 1-(3-chloro-4-isopropoxyphenyl)ethanone | hydroxylamine;hydrochloride | 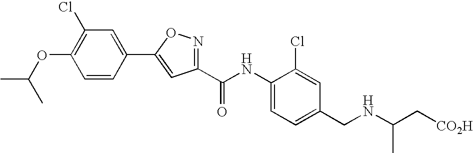 | 2.88min (a) | 506.22(M + H)⁺ |
| J15 | 1-Phenylethanone | methylhydrazine;hydrochloride | 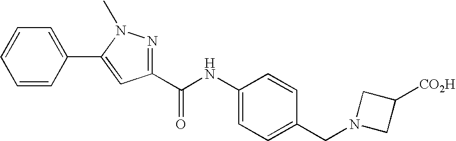 | 2.27min (a) | 391.18(M + H)⁺ |
| J16 | 1-Phenylethanone | methylhydrazine;hydrochloride | 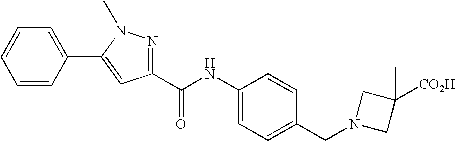 | 2.29min (a) | 405.22(M + H)⁺ |
| J17 | 1-Phenylethanone | methylhydrazine;hydrochloride | 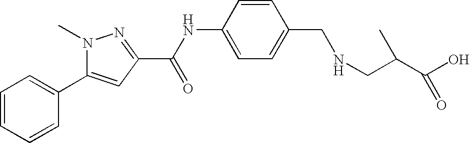 | 2.31min (a) | 393.18(M + H)⁺ |
| J18 | 1-Phenylethanone | methylhydrazine;hydrochloride | 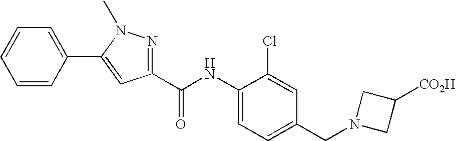 | 2.51min (a) | 425.24(M + H)⁺ |
| J19 | 1-(pyridin-2-yl)ethanone | tert-Butyl-hydrazine;hydrochloride | 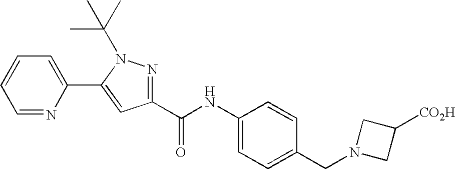 | 1.59min (g) | 434.22(M + H)⁺ |
| J20 | 1-(pyridin-4-yl)ethanone | tert-Butyl-hydrazine;hydrochloride | 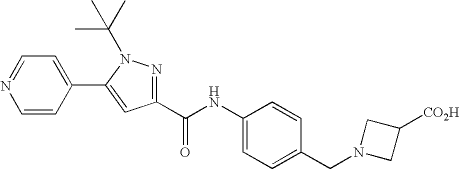 | 1.49min (g) | 434.29(M + H)⁺ |
| J21 | 1-(3-chloro-4-isopropoxyphenyl)ethanone | tert-Butyl-hydrazine;hydrochloride | 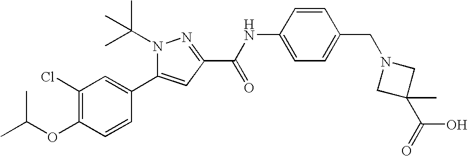 | 3.17min (a) | 539.36(M + H)⁺ |
| J22 | 1-(3-chloro-4-isopropoxyphenyl)ethanone | methylhydrazine;hydrochloride | 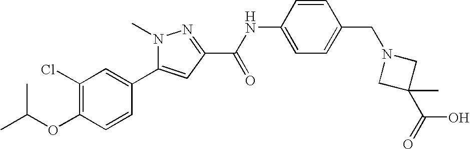 | 2.44min (a) | 495.37(M − H)⁻ |
| J23 | 1-(3-chloro-4-isopropoxyphenyl)ethanone | tert-Butyl-hydrazine;hydrochloride | 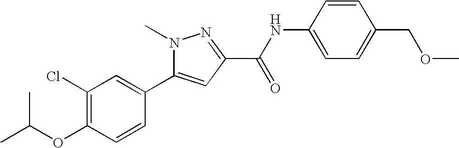 | 3.74min (a) | 414.13(M + H)⁺ |
| J24 | 1-(pyridin-3-yl)ethanone | tert-Butyl-hydrazine;hydrochloride | 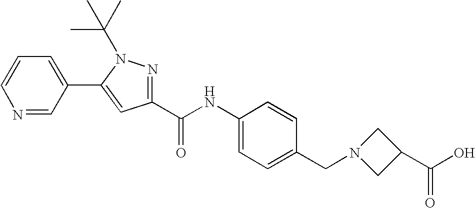 | 1.57min (g) | 434(M − H)⁻ |

TABLE K
Examples prepared following general procedures R, T, C, D.1, C (Scheme XVII)

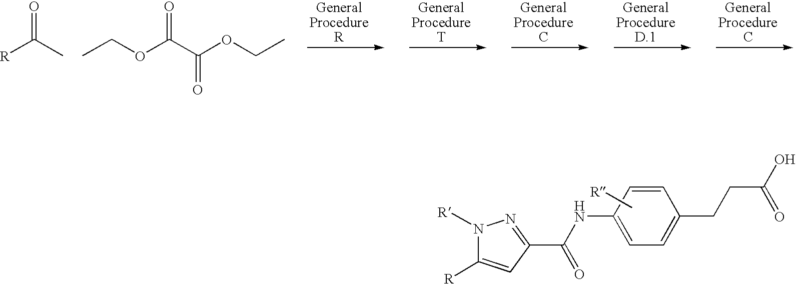

| | | | | HPLC Rₜ | |
|---|---|---|---|---|---|
| Ex. # | Carboxylic acid | amine | Product | (Method) | m/z |
| K1 | 1-tert-butyl-5-phenyl-1H-pyrazole-3-carboxylic acid | ethyl 3-(4-aminophenyl)propanoate | 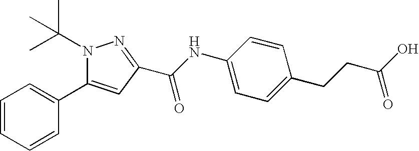 | 3.58min (a) | 392.44(M + H)⁺ |
| K2 | 1-tert-butyl-5-phenyl-1H-pyrazole-3-carboxylic acid | ethyl 3-(4-aminophenyl)propanoate | 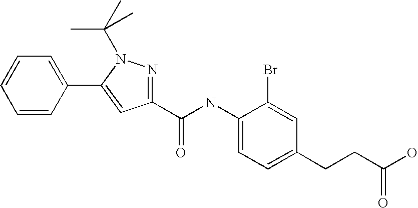 | 4.13min (a) | 472.39474.39(M + H)+ |
| K3 | 1-tert-butyl-5-phenyl-1H-pyrazole-3-carboxylic acid | ethyl 3-(4-aminophenyl)propanoate | 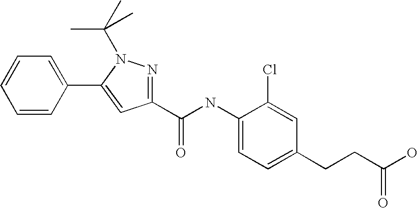 | 4.09min (a) | 424.43426.43(M − H)− |
| K4 | 1-tert-butyl-5-phenyl-1H-pyrazole-3-carboxylic acid | ethyl 3-(4-aminophenyl)propanoate | 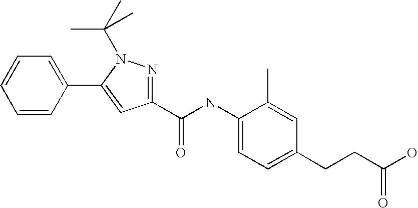 | 2.26min (b) | 406.33(M + H)+ |
| K5 | 5-tert-butyl-1-phenyl-1H-pyrazole-3-carboxylic acid | ethyl 3-(4-amino-3-chlorophenyl)propanoate | 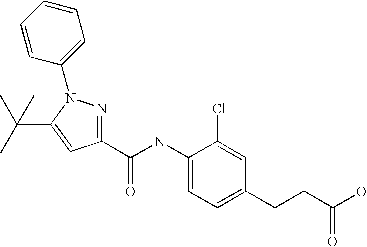 | 3.39min (a) | 424.43426.43(M − H)− |
| K6 | 5-tert-butyl-1-phenyl-1H-pyrazole-3-carboxylic acid | ethyl 3-(4-amino-3-chlorophenyl)propanoate | 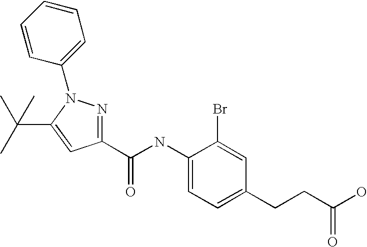 | 3.97min (a) | 468.32470.32(M − H)− |
| K7 | 5-tert-butyl-1-phenyl-1H-pyrazole-3-carboxylic acid | ethyl 3-(4-amino-3-chlorophenyl)propanoate | 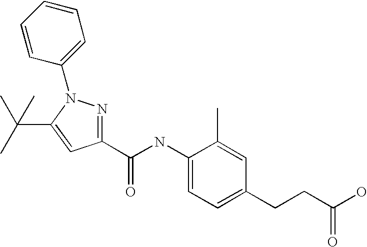 | 3.57min (a) | 404.48(M − H)− |
| K8 | 5-tert-butyl-1-phenyl-1H-pyrazole-3-carboxylic acid | ethyl 3-(4-amino-3-chlorophenyl)propanoate | 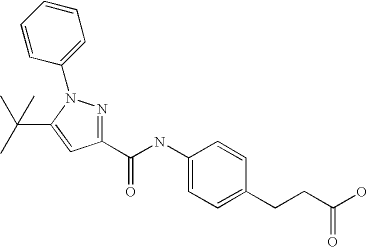 | 3.53min (a) | 390.51(M − H)− |
| K9 | 1-tert-butyl-4-methyl-5-phenyl-1H-pyrazole-3-carboxylic acid | ethyl 3-(4-aminophenyl)propanoate | 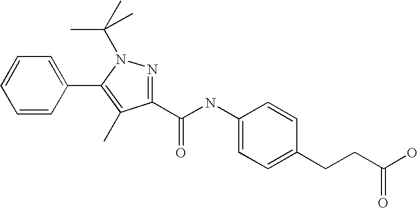 | 3.30min (a) | 404.17(M − H)− |
| K10 | 1-tert-butyl-4-methyl-5-phenyl-1H-pyrazole-3-carboxylic acid | ethyl 3-(4-amino-3-chlorophenyl)propanoate | 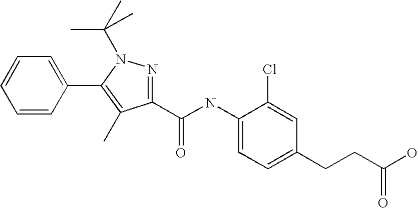 | 3.44min (a) | 438.15440.14(M − H)− |

TABLE L
Examples prepared following general procedures D or F (Scheme XVIII)

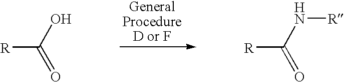

| | Acid or acid | | HPLC Rₜ | |
|---|---|---|---|---|
| Ex. # | chloride, amine | Product | (Method) | m/z |
| L1 | 5-(furan-2-yl)-1-phenyl-1H-pyrazole-3-carbonylchloride, 2-chloroaniline | 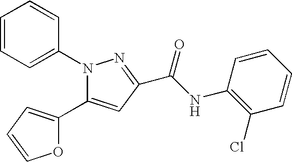 | 2.65min (b) | 364.19(M + H)⁺ |
| L2 | 3-phenylisoxazole5-carbonyl chloride,4-aminobenzonitrile | 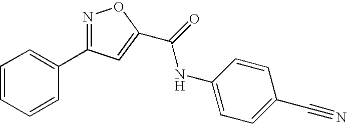 | 3.72min (a) | 288.27(M − H)⁻ |
| L3 | 3-phenylisoxazole-5-carbonyl chloride,3-chloro-4-methoxyaniline | 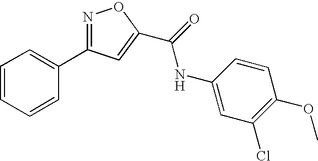 | 3.94min (a) | 329.25(M + H)⁺ |
| L4 | 5-phenylisoxazole-3-carbonyl chloride,2-(4-aminophenyl)aceticacid (Aldrich) | 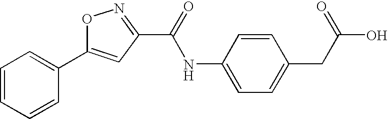 | 3.13min (a) | 323.19(M + H)⁺ |
| L5 | 5-phenylisoxazole-3-carbonyl chloride,methyl 4-aminobenzoate(Aldrich) | 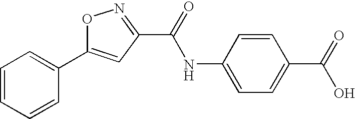 | 3.11min (a) | 307.28(M − H)⁻ |
| L6 | 3-phenyl-5-isoxazolecarbonylchloride, aniline | 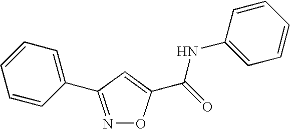 | 2.28min (a) | 265.1(M + H)⁺ |
| L7 | 3-phenyl-5-isoxazolecarbonylchloride, p-toluidine | 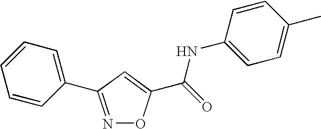 | 2.37min (a) | 279.1(M + H)⁺ |
| L8 | 3-phenyl-5-isoxazolecarbonylchloride, 4-fluoroaniline | 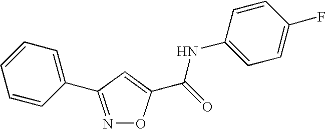 | 3.09min (a) | 283(M + H)⁺ |
| L9 | 3-phenyl-5-isoxazolecarbonylchloride, 4-methoxyaniline | 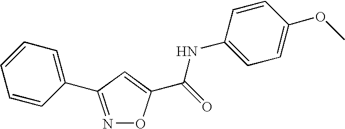 | 2.99min (a) | 295.1(M + H)⁺ |
| L10 | 3-phenyl-5-isoxazolecarbonylchloride, 4-chloroaniline | 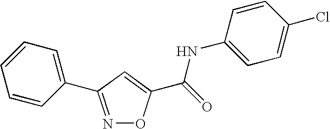 | 3.34min (a) | 299.1(M + H)⁺ |
| L11 | 3-phenyl-5-isoxazolecarbonylchloride, 3-methoxyaniline | 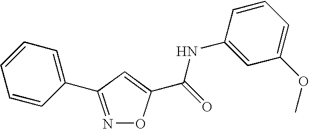 | 3.10min (a) | 295.1(M + H)⁺ |
| L12 | 3-phenyl-5-isoxazolecarbonylchloride, 2-methoxyaniline | 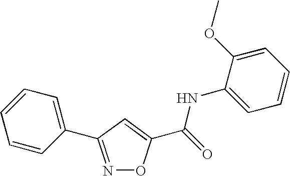 | 3.43min (a) | 295.1(M + H)⁺ |
| L13 | 3-phenyl-5-isoxazolecarbonylchloride, methyl 4-aminobenzoate | 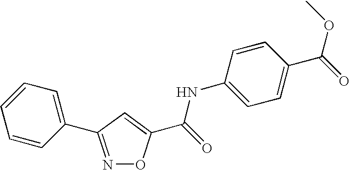 | 3.12min (a) | 321.2(M − H)⁻ |
| L14 | 3-phenyl-5-isoxazolecarbonylchloride,1,1′-biphenyl-4-amine | 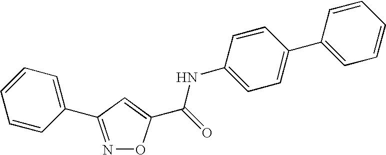 | 3.59min (a) | 341.2(M + H)⁺ |
| L15 | 3-phenyl-5-isoxazolecarbonylchloride,benzylamine | 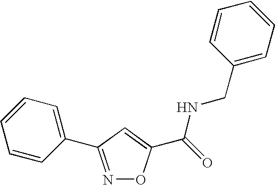 | 2.92min (a) | 279.1(M + H)⁺ |
| L16 | 3-phenyl-5-isoxazolecarbonylchloride,cyclohexylamine | 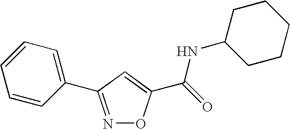 | 3.13min (a) | 293.2(M + H)⁺ |
| L17 | 3-phenyl-5-isoxazolecarbonylchloride,phenethylamine | 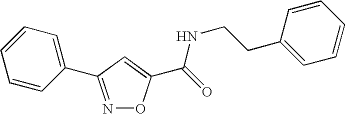 | 3.00min (a) | 293.2(M + H)⁺ |
| L18 | 3-phenyl-5-isoxazolecarbonylchloride, 2aminonaphthalene | 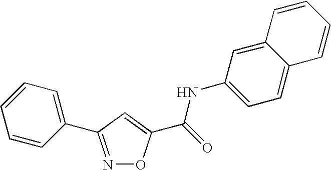 | 3.41min (a) | 315.1(M + H)⁺ |
| L19 | 3-phenyl-5-isoxazolecarbonylchloride, m-toluidine | 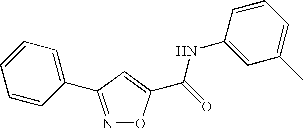 | 3.23min (a) | 279.1(M + H)⁺ |
| L20 | 3-phenyl-5-isoxazolecarbonylchloride, 3-aminobenzonitrile | 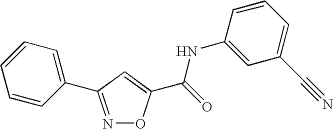 | 2.96min (a) | 288.2(M − H)⁻ |

TABLE M
Examples prepared following general procedures D or F (Scheme XIX)

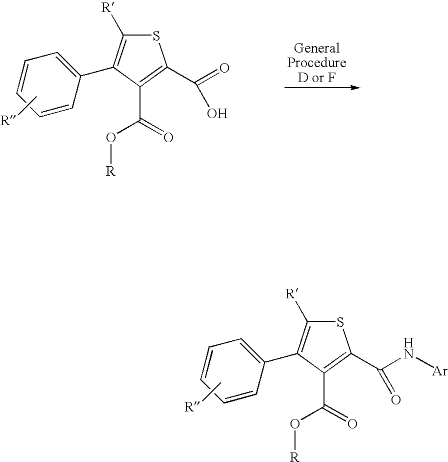

| | Acid or acid | | HPLC Rₜ | |
|---|---|---|---|---|
| Ex. # | chloride, Amine | Product | (Method) | m/z |
| M1 | 4-phenyl-5-(trifluoromethyl)thiophene-2-carbonylchloride, 1-ethyl-1H-pyrazol-5-amine | 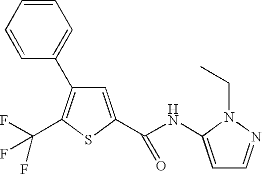 | 2.91min (a) | 366.10(M + H)⁺ |

TABLE N
Examples prepared following general procedures R, S, C, D.3, H

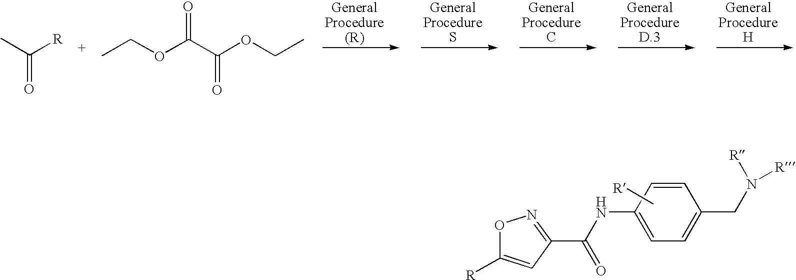

| | | | HPLC Rₜ | |
|---|---|---|---|---|
| Ex. # | Starting material | Product | (Method) | m/z |
| N1 | 1-(3-chloro-4-isopropoxyphenyl)ethanone | 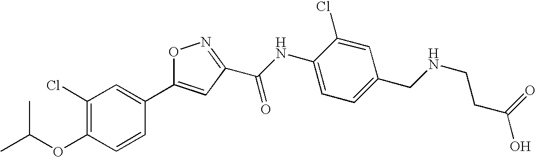 | 1.92min (b) | 490.15(M − H)⁻ |
| N2 | 1-(3-chloro-4-isopropoxyphenyl)ethanone | 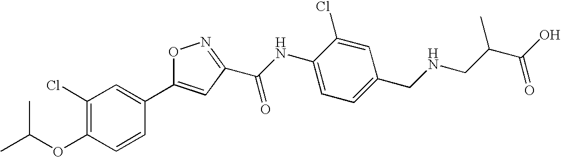 | 2.54min (a) | 504.16(M − H)⁻ |
| N3 | 1-(3-chloro-4-isopropoxyphenyl)ethanone | 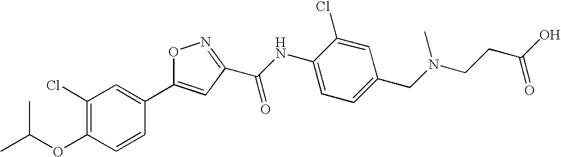 | 2.55min (a) | 504.16(M − H)⁻ |
| N4 | 1-(4-isobutylphenyl)ethanone | 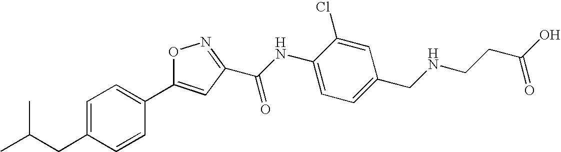 | 2.61min (a) | 454.21(M − H)⁻ |
| N5 | 1-(3-chloro-4-morpholinophenyl)ethanone | 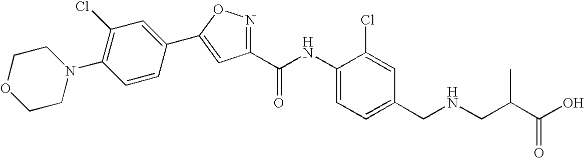 | 1.89min (a) | 531.15(M − H)⁻ |
| N6 | 1-(3-chloro-4-isopropoxyphenyl)ethanone | 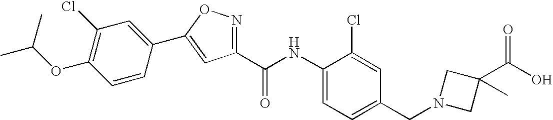 | 2.13min (a) | 516.16(M − H)⁻ |

TABLE O
Examples prepared following general procedures D, Y, C

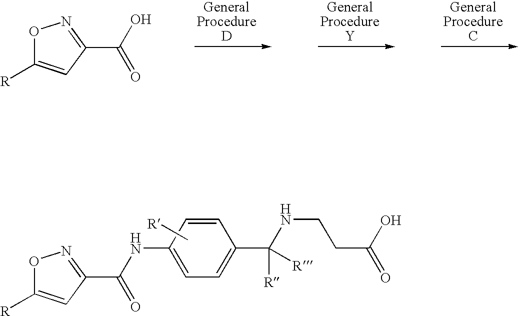

| | | | HPLC Rₜ | |
|---|---|---|---|---|
| Ex. # | Starting material | Product | (Method) | m/z |
| O1 | 5-(3-chloro-4-(isopropylamino)phenyl)isoxazole-3-carboxylic acid | 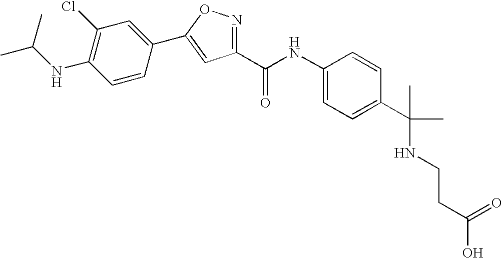 | 2.39min (a) | 483.19(M − H)− |
| O2 | 5-(3-chloro-4-isopropoxyphenyl)isoxazole-3-carboxylic acid | 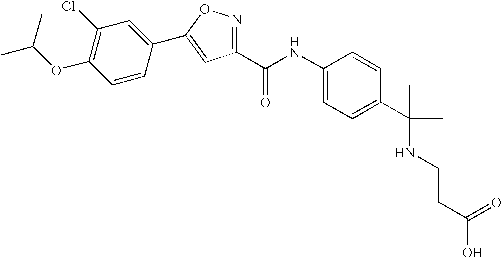 | 2.35min (a) | 484.22(M − H)− |
| O3 | 5-(3-chloro-4-morpholinophenyl)isoxazole-3-carboxylic acid | 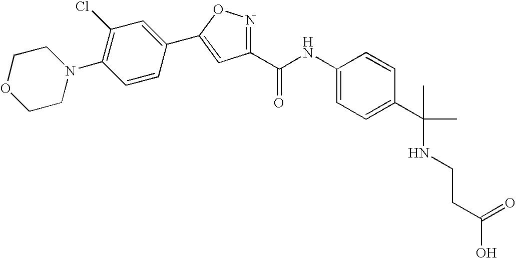 | 2.02min (a) | 511.23(M − H)− |
| O4 | 5-(3-chloro-4-isopropoxyphenyl)isoxazole-3-carboxylic acid | 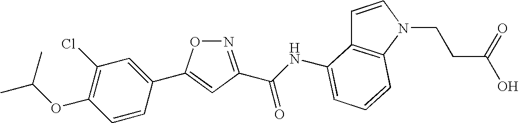 | 3.04min (a) | 366.17(M − H)− |
| O5 | 5-(3-chloro-4-isopropoxyphenyl)isoxazole-3-carboxylic acid | 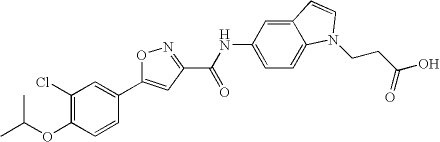 | 3.02min (a) | 466.18(M − H)− |
| O6 | 5-(4-ethylphenyl)isoxazole-3-carboxylic acid(ACB Blocks Ltd.) | 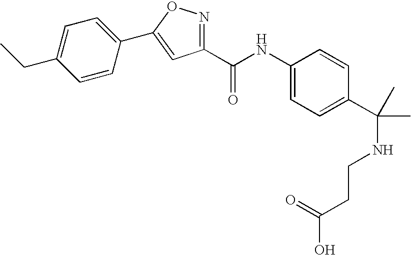 | 2.19min (a) | 422 |

TABLE P
Examples prepared following general procedures R, S, U, V, H

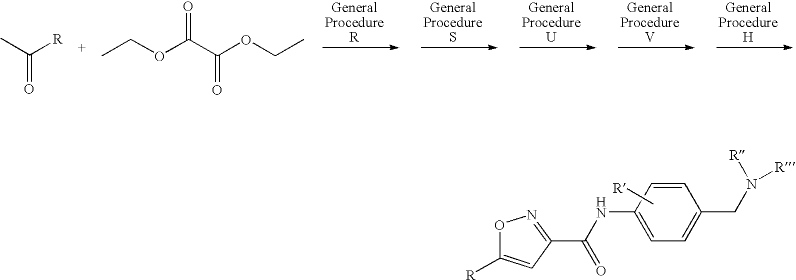

| | Acid or acid | | HPLC Rₜ | |
|---|---|---|---|---|
| Ex. # | chloride, Amine | Product | (Method) | m/z |
| P1 | 5-(3-chloro-4-(isopropylamino)phenyl)isoxazole-3-carboxylic acid | 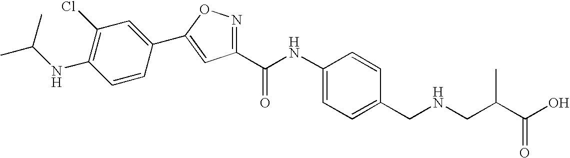 | 2.38min (a) | 469.20(M − H)⁻ |
| P2 | 5-(3-chloro-4-(diethylamino)phenyl)isoxazole-3-carboxylic acid | 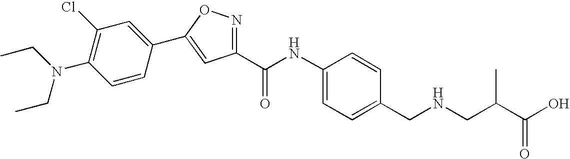 | 2.48min (a) | 483.23(M − H)⁻ |

TABLE Q
Examples prepared following general procedures R, S. C, E, F, N

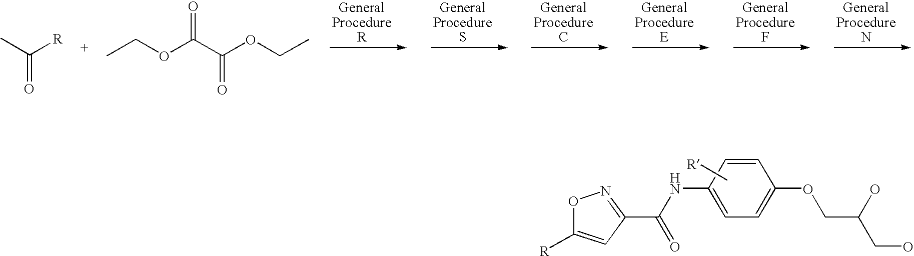

| | | | HPLC Rₜ | |
|---|---|---|---|---|
| Ex. # | Starting material | Product | (Method) | m/z |
| Q1 | 1-(3-chloro-4-isopropoxyphenyl)ethanone | 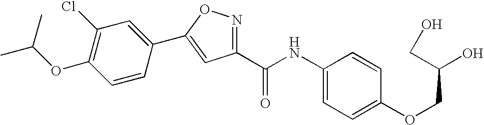 | 2.70min (a) | 455.20(M − H)− |
| Q2 | 1-(3-chloro-4-isobutylphenyl)ethanone | 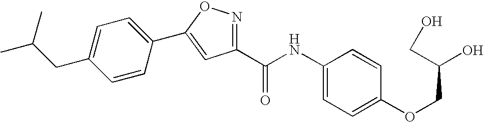 | 2.92min (a) | 409.25(M − H)− |

TABLE R
Examples prepared following general procedures E, F, Z, Y, C

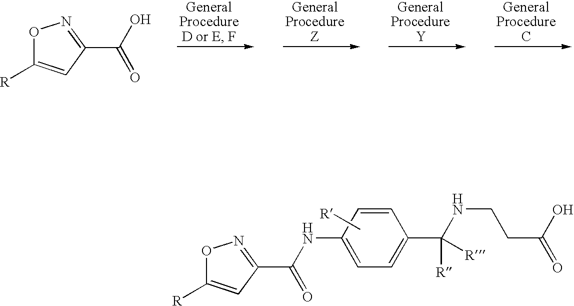

| | | | HPLC Rₜ | |
|---|---|---|---|---|
| Ex. # | Starting material | Product | (Method) | m/z |
| R1 | 5-(3-chloro-4-isopropoxyphenyl)isoxazole-3-carboxylic acid | 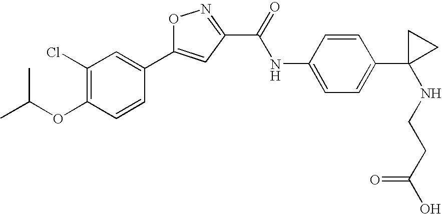 | 2.42min (a) | 482.17(M − H)⁻ |
| R2 | 5-(3-chloro-4-isopropoxyphenyl)isoxazole-3-carboxylic acid | 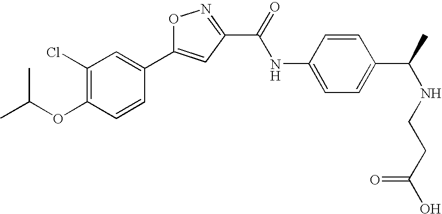 | 2.32min (a) | 470.18(M − H)⁻ |
| R3 | 5-(3-chloro-4-isopropoxyphenyl)isoxazole-3-carboxylic acid | 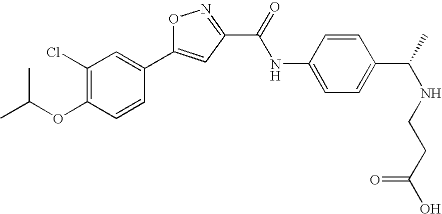 | 2.32min (a) | 470.20(M − H)⁻ |
| R4 | 5-(3-chloro-4-isopropoxyphenyl)isoxazole-3-carboxylic acid | 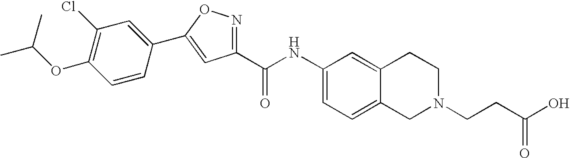 | 1.99min (a) | 482.20(M − H)⁻ |
| R5 | 5-(1H-indazol-5-yl)isoxazole-3-carboxylic acid | 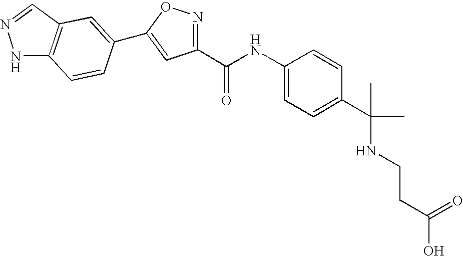 | 1.71min (a) | 432.23(M − H)⁻ |

TABLE S
Examples prepared following general procedures E, F

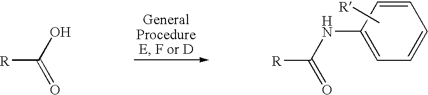

| | | | HPLC Rₜ | |
|---|---|---|---|---|
| Ex. # | Starting material | Product | (Method) | m/z |
| S1 | 5-(3-chloro-4-isopropoxyphenyl)isoxazole-3-carboxylic acid | 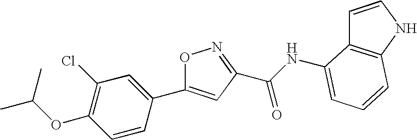 | 3.36min (a) | 396.18(M + H)⁺ |
| S2 | 5-(3-chloro-4-isopropoxyphenyl)isoxazole-3-carboxylic acid | 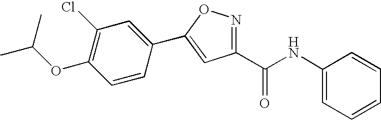 | 3.50min (a) | 355.14(M − H)⁻ |
| S3 | 5-(3-chloro-4-isopropoxyphenyl)isoxazole-3-carboxylic acid | 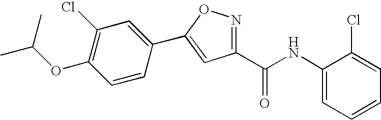 | 4.03min (a) | 389.12(M − H)⁻ |
| S4 | 5-(3-chloro-4-isopropoxyphenyl)isoxazole-3-carboxylic acid | 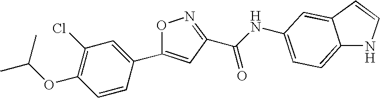 | 3.30min (a) | 394.20(M − H)⁻ |
| S5 | 4-(ethoxycarbonyl)-3-phenylisoxazole-5-carboxylic acid | 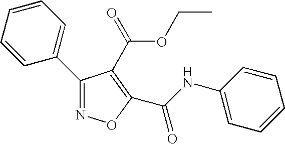 | 3.16min (a) | 337.14(M + H)⁺ |
| S6 | 4-phenyl-5-(trifluoromethyl)thiophene-2-carboxylic acid | 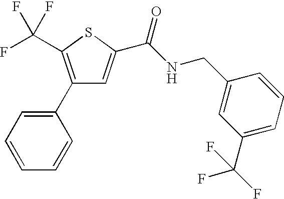 | 3.55min (a) | 428.15(M − H)⁻ |
| S7 | 4-phenyl-5-(trifluoromethyl)thiophene-2-carboxylic acid | 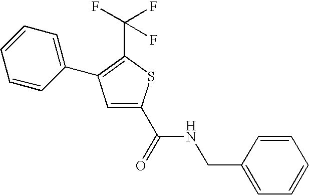 | 3.34min (a) | 360.19(M − H)⁻ |
| S8 | 5-(1H-Indazol-5-yl)isoxazole-3-carboxylic acid | 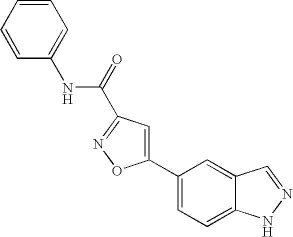 | 2.04min (b) | 303.18(M − H)⁻ |

TABLE T
Examples prepared following general procedures AA, U, V, H (Scheme XX)

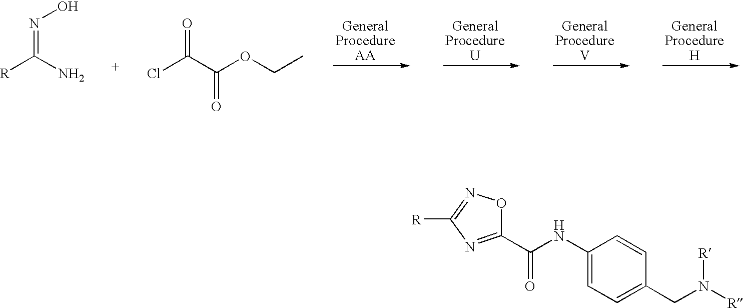

| | | | HPLC Rₜ | |
|---|---|---|---|---|
| Ex. # | Starting material | Product | (Method) | m/z |
| T1 | (Z)-3-chloro-N′-hydroxy-4-isopropoxy-benzimidamide | 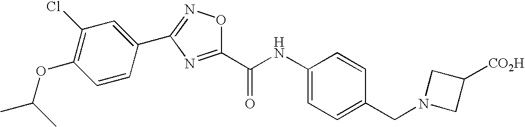 | 1.98min (g) | (M + H)471.22 |
| T2 | (Z)-3-chloro-N′-hydroxy-4-isopropoxy-benzimidamide | 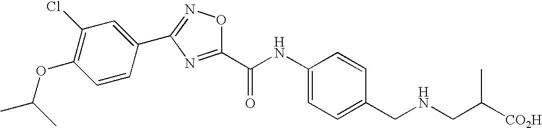 | 2.04min (g) | (M + H)473.21 |
| T3 | (Z)-3-chloro-N′-hydroxy-4-isopropoxy-benzimidamide | 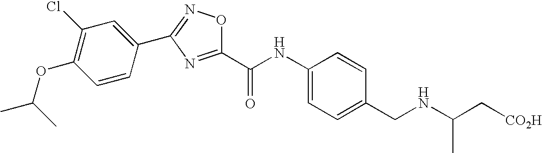 | 1.27min (k) | (M + H)473.28 |

TABLE U
Examples prepared following general procedures R, S, C, D.3, H (Scheme XXI)

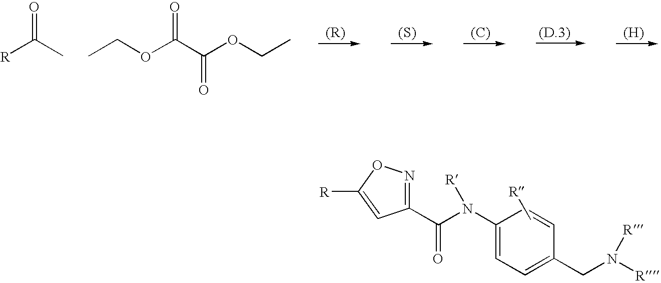

| | | | HPLC Rₜ | |
|---|---|---|---|---|
| Ex. # | Starting material | Product | (Method) | m/z |
| U1 | 1-(3-chloro-4-isopropoxyphenyl)ethanone | 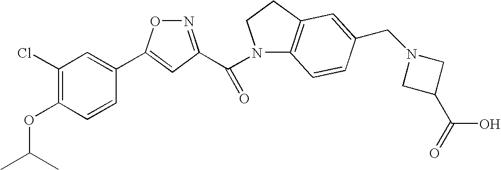 | 2.01min (g) | (M + H)⁺496 |
| U2 | 1-(3-chloro-4-isopropoxyphenyl)ethanone | 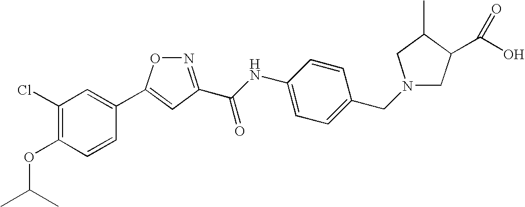 | 2.04min (g) | (M + H)⁺498 |
| U3 | 5-(4-ethylphenyl)isoxazole-3-carboxylic acid | 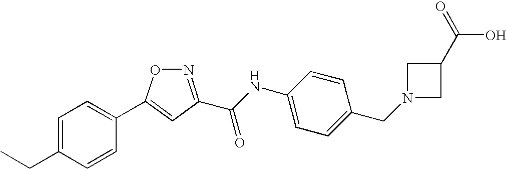 | 2.06min (a) | (M + H)⁺406 |
| U4 | 5-(4-cyclohexylphenyl)isoxazole-3-carboxylic acid | 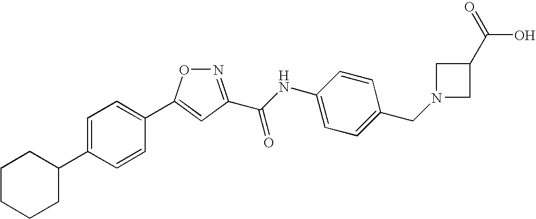 | 2.58min (a) | (M + H)⁺460 |
| U5 | 5-(4-butylphenyl)isoxazole-3-carboxylic acid | 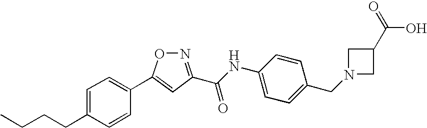 | 2.08min (a) | (M + H)⁺434 |
| U6 | 5-(4-tert-butylphenyl)isoxazole-3-carboxylic acid | 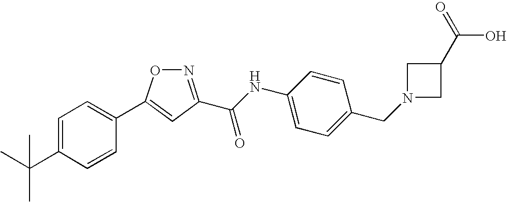 | 1.97min (a) | (M + H)⁺434 |

TABLE V
Examples prepared following general procedures Q, W, D.3, C (Scheme XII)

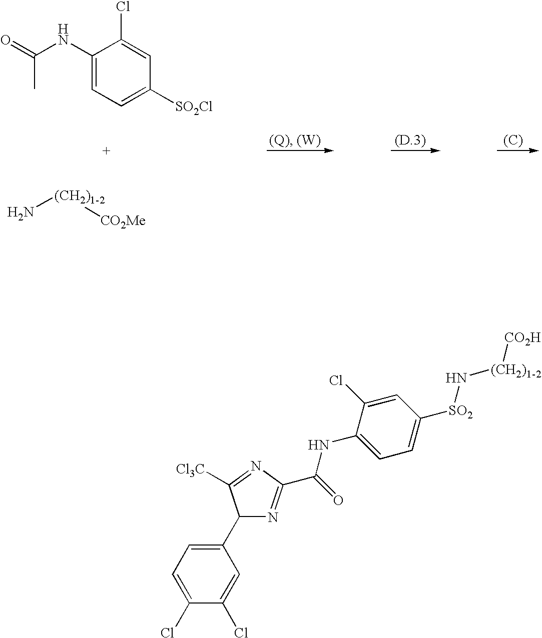

| | sulfonyl chloride, | | HPLC Rₜ | |
|---|---|---|---|---|
| Ex. # | amine, acid | Product | (Method) | m/z |
| V1 | 4-acetamido-3-chlorobenzene-1-sulfonyl chloride(Alfa Aesar), tert-butyl 2-aminoacetatehydrochloride(Aldrich), 1-phenyl-5-(trichloromethyl)-1H-1,2,4-triazole-3-carboxylic acid | 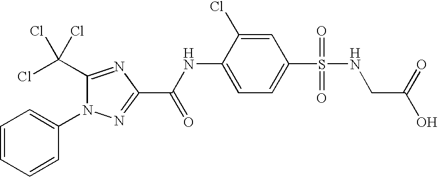 | 3.01min (a) | 553.96(M + H)⁺ |
| V2 | 4-acetamido-3-chlorobenzene-1-sulfonyl chloride(Alfa Aesar), 2-alanine methyl esterhydrochloride(Aldrich), 1-phenyl-5-(trichloromethyl)-1H-1,2,4-triazole-3-carboxylic acid | 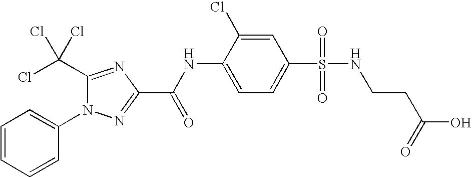 | 3.43min (a) | 567.95(M + H)⁺ |

TABLE W
General synthetic route to isoxazole amides following general procedures R, S,
C, E, F (Scheme XV)

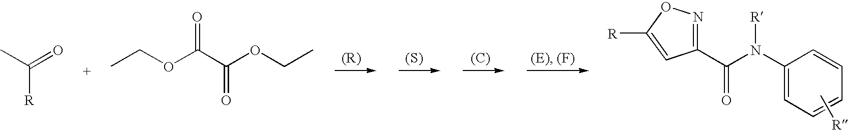

| | Acid or acid | | HPLC Rₜ | |
|---|---|---|---|---|
| Ex. # | chloride, Amine | Product | (Method) | m/z |
| W1 | 5-(3-chloro-4-isopropoxyphenyl)isoxazole-3-carbonyl chloridecarboxylate (prepared from 5-(3-chloro-4-isopropoxyphenyl)isoxazole-3-carboxylic acid viageneral procedure E; 4-aminophenol | 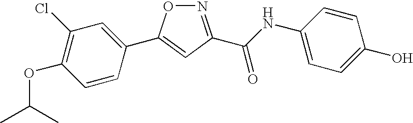 | 2.59 min (g) | 373.09(M + H)⁺ |
| W2 | 5-(3-chloro-4-isopropoxyphenyl)isoxazole-3-carbonyl chloridecarboxylate (prepared from 5-(3-chloro-4-isopropoxyphenyl)isoxazole-3-carboxylic acid viageneral procedure E; tert-butyl 4-aminopiperidine-1-carboxylate | 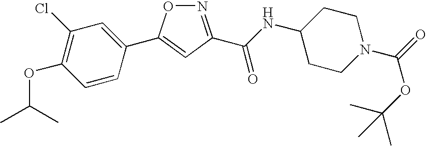 | 2.98 min (g) | 464.29(M + H)⁺ |
| W3 | 5-(3-chloro-4-isopropoxyphenyl)isoxazole-3-carbonyl chloridecarboxylate (prepared from 5-(3-chloro-4-isopropoxyphenyl)isoxazole-3-carboxylic acid viageneral procedure E, 1-(((1r, 4r)-4-aminocyclohexyl)methyl)azetidine-3-carboxylic acid | 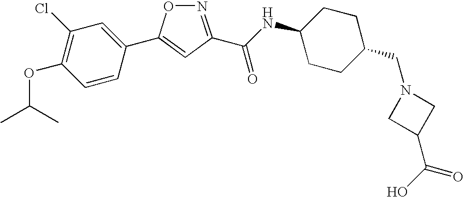 | 1.90 min (g) | 476.21(M + H)⁺ |
| W4 | 5-(3-chloro-4-isopropoxyphenyl)isoxazole-3-carbonyl chloridecarboxylate (prepared from 5-(3-chloro-4-isopropoxyphenyl)isoxazole-3-carboxylic acid viageneral procedure E); 1-(((1s, 4s)-4-aminocyclohexyl)methyl)acetidine-3-carboxylic acid | 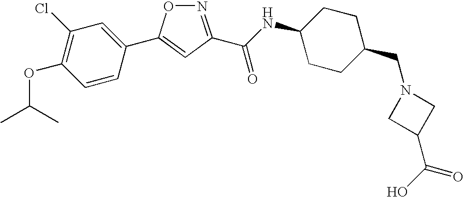 | 1.88 min (g) | 476.21(M + H)⁺ |

TABLE AA
Examples prepared following general procedure H: Reductive amination of an
aldehyde

| | | | HPLC Rₜ | |
|---|---|---|---|---|
| Ex. # | Aldehyde, Amine | Product | (Method) | m/z |
| AA1 | ethyl 2-(4-formylbenzamido)-4-(4-propoxyphenyl)thiophene-3-carboxylate(prepared viageneral proceduresD.2 and N fromethyl 2-amino-4-(4-propoxyphenyl)thio-phene-3-carboxylate[Otava] and 4-(1,3-dioxolan-2-yl)benzoic acid[prepared viageneral procedure Lfrom 4-formyl-benzoic acid]);azetidine-3-carboxylic acid | 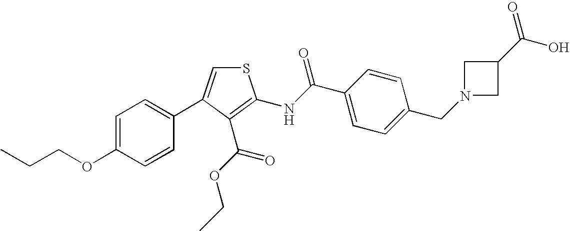 | 2.39 min (a) | 523.21(M + H)⁺ |

TABLE BB
Examples prepared following general procedure N: Deprotection of an acetal to
an aldehyde

| | | | HPLC Rₜ | |
|---|---|---|---|---|
| Ex. # | Acetal | Product | (Method) | m/z |
| BB1 | ethyl 2-(4-(1,3-dioxolan-2-yl)benzamido)-4-(4-propoxyphenyl)thiophene-3-carboxylate (preparedvia general procedure D.2 fromethyl 2-amino-4-(4-propoxyphenyl)thiophene-3-carboxylate [Otava] and4-(1,3-dioxolan-2-yl)benzoic acid[prepared via general procedure Lfrom 4-formylbenzoic acid] | 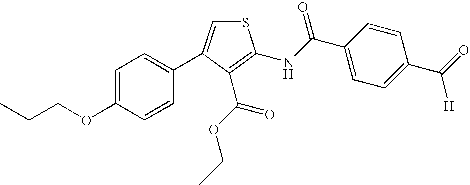 | 4.68 min (a) | 438.35(M + H)⁺ |

TABLE CC
Examples prepared following general procedure Z: Deprotection of a Boc-
protected amine

| | Boc-protected | | HPLC Rₜ | |
|---|---|---|---|---|
| Ex. # | Amine | Product | (Method) | m/z |
| CC1 | tert-butyl 4-(5-(1H-indazol-5-yl)isoxazole-3-carboxamido)piperidine-1-carboxylate (preparedvia general procedures D.5 from 5-(1H-indazol-5-yl)isoxazole-3-carboxylic acid and tert-butyl 4-aminopiperidine-1-carboxylate | 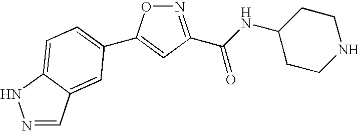 | 1.01 min (i) | 312.13(M + H)⁺ |
| CC2 | tert-butyl 4-(5-(3-chloro-4-isopropoxyphenyl)isoxazole-3-carboxamido)piperidine-1-carboxylate (prepared from 5-(3-chloro-4-isopropoxyphenyl)isoxazole-3-carboxylic acidand tert-butyl 4-aminopiperidine-1-carboxylatevia general procedure E and F) | 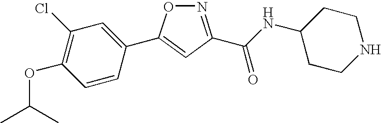 | 1.84 min (g) | 364.11(M + H)⁺ |

TABLE DD
Examples prepared following general procedure I: Deprotection of a silyl
protected alcohol

| | Silyl protected | | HPLC Rₜ | |
|---|---|---|---|---|
| Ex. # | alchol | Product | (Method) | m/z |
| DD1 | N-(4-((tert-butyldimethylsilyloxy)methyl)-2-methoxyphenyl)-5-(1H-indazol-5-yl)isoxazole-3-carboxamide [prepared via general procedureD.5 from 5-(1H-indazol-5-yl)isoxazole-3-carboxylic acid and 4-((tert-butyldimethyl-silyloxy)methyl)-2-methoxyaniline] | 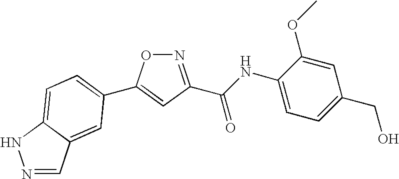 | 1.23 min (h) | 363.45(M + H)⁺ |

TABLE EE
Examples prepared following general procedures C, E, F, I, J.1, H
(Scheme XXII)

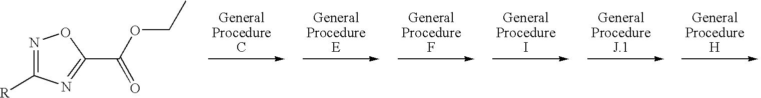

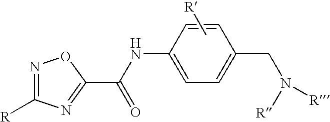

| | | | HPLC Rₜ | |
|---|---|---|---|---|
| Ex. # | Starting material | Product | (Method) | m/z |
| EE1 | ethyl 5-phenyl-1,2,4-oxadiazole-3-carboxylate(Bionet) | 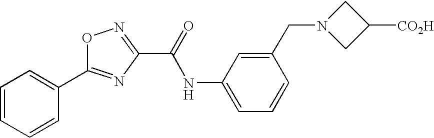 | 1.64 min(a) | 379.18(M + H)⁺ |
| EE2 | ethyl 5-phenyl-1,2,4-oxadiazole-3-carboxylate(Bionet) | 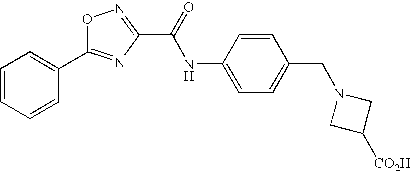 | 1.67 min(a) | 379.15(M + H)⁺ |

TABLE FF
Examples prepared from 1-tert-butyl-N-(4-formylphenyl)-5-phenyl-1H-
pyrazole-3-carboxamide

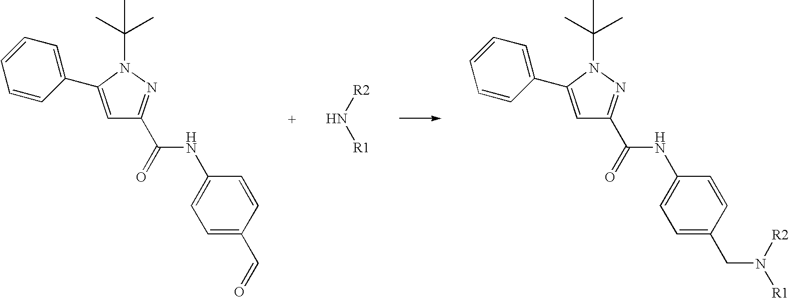

| | | | HPLC Rₜ | |
|---|---|---|---|---|
| Ex. # | amine | Product | (Method) | m/z |
| FF1 | Azetidine carboxylicacid | 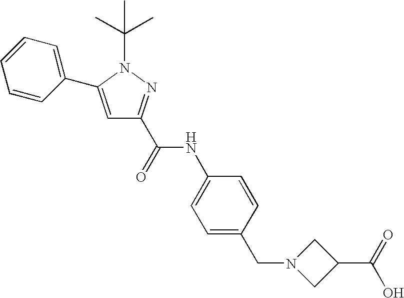 | 1.42 min (o) | 433.2(M + H)⁺ |
| FF2 | Piperidine-3-carboxylic acid | 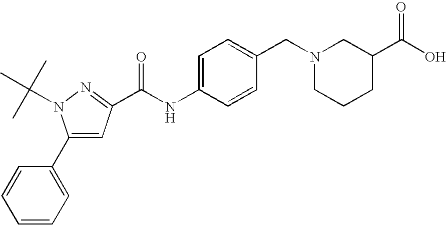 | 1.45 min (o) | 461.39(M + H)⁺ |
| FF3 | Piperidine-4-carboxylic acid | 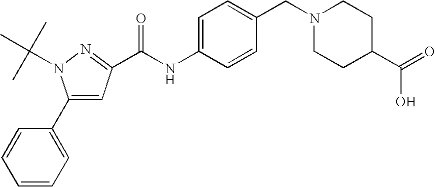 | 1.45 min (o) | 461.2(M + H)⁺ |
| FF4 | Hexahydro-cyclopenta[c]pyrrole-3a-carboxylic acid | 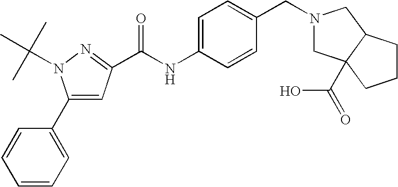 | 1.5 min (o) | 487.3(M + H)⁺ |
| FF5 | (1S,6S)-6-Amino-cyclohex-3-enecarboxylic acid | 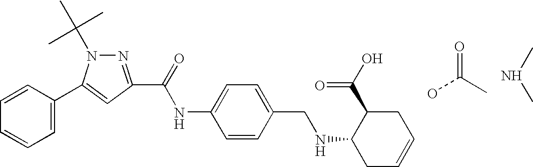 | 1.5 | 473.53(M + H)⁺ |
| FF6 | (R)-Amino-cyclopentyl-aceticacid | 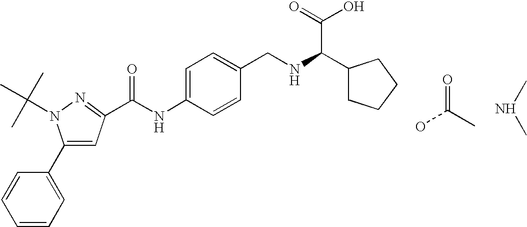 | 1.53 | 475.3(M + H)⁺ |
| FF7 | 3-Methylamino-propionic acid | 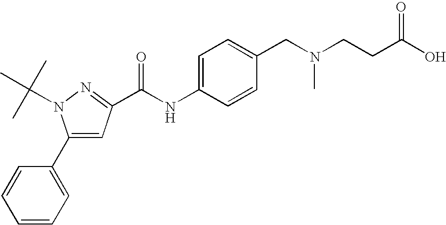 | 1.42 | 435.2(M + H)⁺ |
| FF8 | 4,4-Dimethyl-pyrrolidine-3-carboxylic acid | 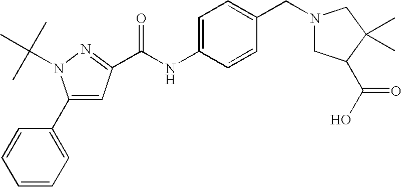 | 1.47 | 475.3(M + H)⁺ |
| FF9 | 3-Methyl-piperidine-4-carboxylic acid | 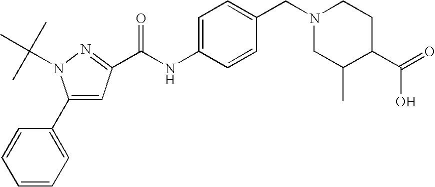 | 1.47 | 475.3(M + H)⁺ |
| FF10 | Pyrrolidine-3-carboxylic acid | 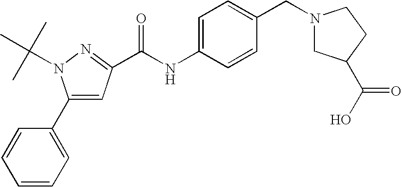 | 1.44 min (o) | 447.2(M + H)⁺ |

A 20 mL vial was charged with a solution of 1-tert-butyl-N-(4-formylphenyl)-5-phenyl-1H-pyrazole-3-carboxamide in 1.0 mL of MeOH (1.0 eq, 2.88 mmol, 25.64 mg) and a solution of pre-weighed 0.6 mmol amine monomer in DMA (1.20 eq, 2.0 mL DMA) followed by shaking for an hour at room temperature. A solution of acetic acid (3.0 eq, 8.64 mmol, 13.30 mg) in dichloromethane/MeOH (1:1 v/v) was then added along with resin bound macro porous cyanoborohydride (Biotage MP-BH₃CN, 3.0 eq, 2.44 mmol/g, 8.64 mmol) and placed on a heater/shaker overnight at 55° C. The crude mixture was then filtered and the solvent was removed in vacuo (Speed Vac), and redissolved in 1.4 mL DMSO/MeOH (1:1 v/v). The crude material was purified by HPLC method (n) and the solvents evaporated.

TABLE GG
Examples prepared from N-(4-formylphenyl)-1-isobutyl-5-phenyl-1H-
pyrazole-3-carboxamide

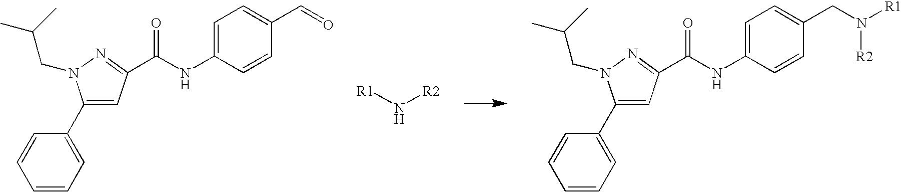

| | | | HPLC Rₜ | |
|---|---|---|---|---|
| Ex. # | Amine | Product | (Method) | m/z |
| GG1 | Furan-3-yl-methylamine | 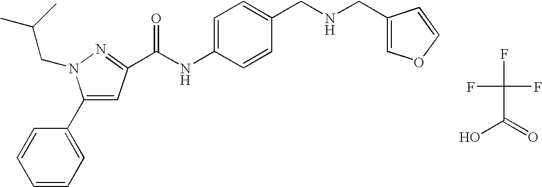 | 1.48 min (o) | 429.2(M + H)⁺ |
| GG2 | Pyridin-2-yl-methylamine | 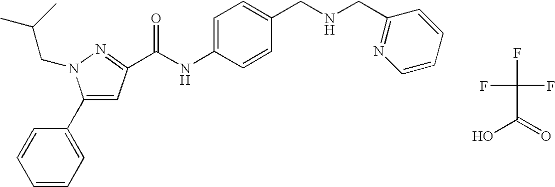 | 1.47 min (o) | 440.2(M + H)⁺ |
| GG3 | Thiazol-2-ylamine | 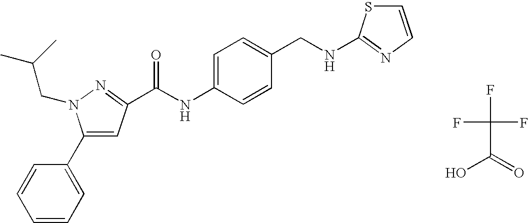 | 1.48 min (o) | 432.2(M + H)⁺ |

A 0.5 mL-2.0 mL microwave reaction vessel was charged with a solution of N-(4-formylphenyl)-1-isobutyl-5-phenyl-1H-pyrazole-3-carboxamide (1.0 eq, 2.85 mmol, 21.49 mg) in DCM/MeOH (1:1 v/v), a solution of pre-weighed 0.6 mmol amine monomer (1.20 eq, 2.0 mL DCM/MeOH (1:1 v/v)) in DCM/MeOH, a solution of acetic acid in DCM/MeOH, resin bound macro porous cyanoborohydride (Biotage, MP-BH₃CN, 2.44 mmol/g, 3.0 eq), and a microflea-teflon covered stir bar. The vessel was capped and placed to irradiate at 100° C. for 600 seconds. The mixture was filtered and dried under vacuum. The crude material was purified by HPLC method (n) and the solvents evaporated.

TABLE HH
Examples of amides prepared from 3-(2-chlorophenyl)isoxazole-5-carboxylic
acid

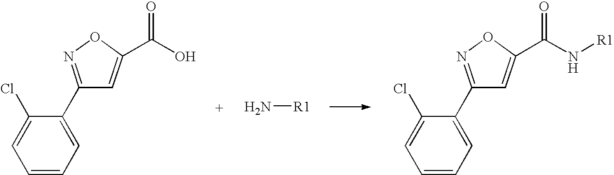

| | | | HPLC Rₜ | |
|---|---|---|---|---|
| Ex. # | Amine | Product | (Method) | m/z |
| HH1 | [2-(4-Aminomethyl-phenoxy)-ethyl]-dimethyl-amine | 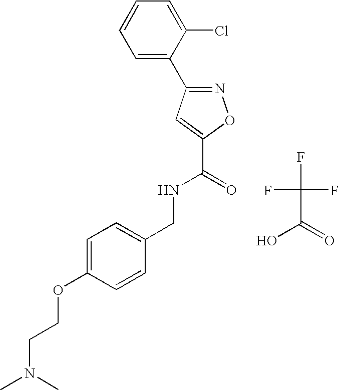 | 1.2 min (o) | 400.1(M + H)⁺ |
| HH2 | 6-(4-Methyl-piperazin-1-yl)-pyridin-3-ylamine | 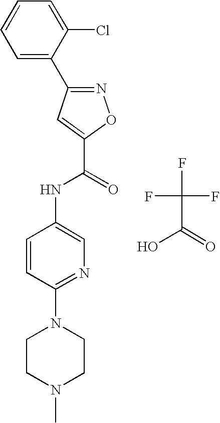 | 1.11 min (o) | 398.1(M + H)⁺ |
| HH3 | 3-Chloro-4-methoxy-phenylamine | 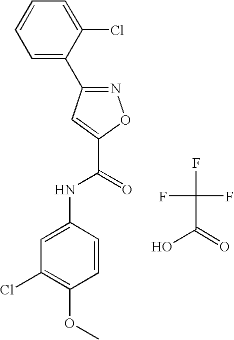 | 1.76 min (o) | 363 (M + H)⁺ |
| HH4 | 4-Isopropoxy-phenylamine | 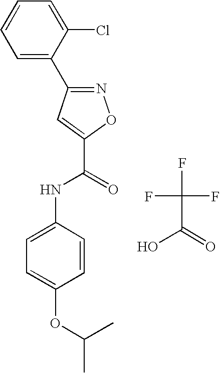 | 1.82 min (o) | 357.1(M + H)⁺ |
| HH5 | 3-Chloro-4-morpholin-4-yl-phenylamine | 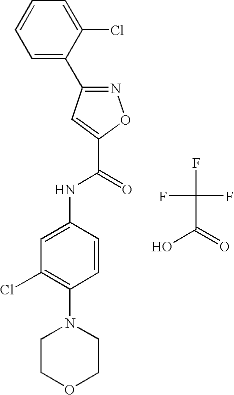 | 1.77 min (o) | 418.1(M + H)⁺ |
| HH6 | 6-Morpholin-4-yl-pyridin-3-ylamine | 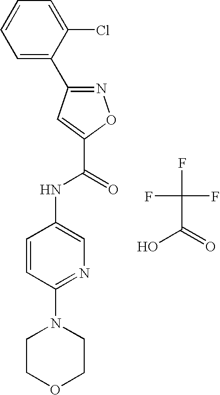 | 1.18 min (o) | 385.1(M + H)⁺ |

A 20 mL scintillation vial is charged with a solution of 3-(2-chlorophenyl)isoxazole-5-carboxylic acid in DMP (1.0 eq, 6.96 mmol, 35.31 mg), a solution of HATU in DMF (1.20 eq, 8.84 mmol), a solution of pre-weighed 0.6 mmol amine monomer in DMF (1.30 eq, 3.0 mL DMF), and a solution of N,N-diisopropylamine in DMF (2.0 eq, 14.74 mmol). The vial was capped and placed in a heater/shaker at 90° C. overnight. The mixture is then passed through a solid phase extraction column with silica carbonate medium using MeOH. The solvent was removed and the crude material was purified by HPLC method (n) and the solvents evaporated.

TABLE II
Examples of amides prepared from 4-phenyl-5-(trifluoromethyl)thiophen-3-
amine

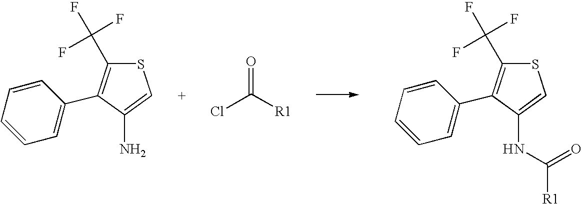

| | | | HPLC Rₜ | |
|---|---|---|---|---|
| Ex. # | Acid chloride | Product | (Method) | m/z |
| II1 | Benzoyl chloride | 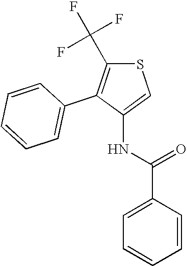 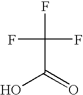 | 1.93 min (o) | 348 (M + H)⁺ |
| II2 | 2-methoxybenzoylchloride | 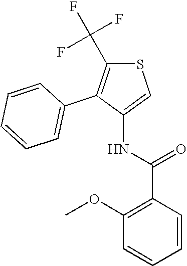 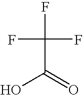 | 2.08 min (o) | 378.1(M + H)⁺ |
| II3 | 2-ethoxybenzoylchloride | 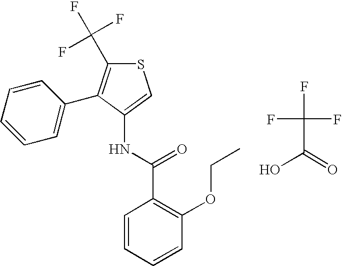 | 2.13 min (o) | 392.1(M + H)⁺ |
| II4 | 2,5-Dimethyl-2H-pyrazole-3-carbonylchloride | 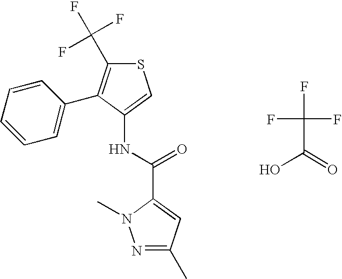 | 1.8 min (o) | 366.1(M + H)⁺ |
| II5 | 4-Acetylamino-benzoyl chloride | 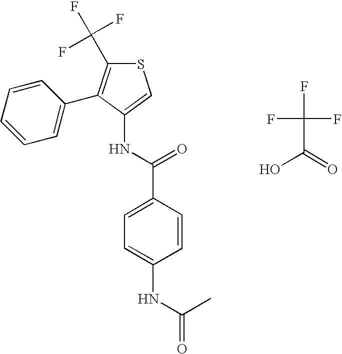 | 1.71 min (o) | 405.1(M + H)⁺ |

A 0.5-2.0 mL-microwave reaction vessel was charged with a microflea-teflon stirring bar, a solution of 4-phenyl-5-(trifluoromethyl)thiophen-3-amine in pyridine (1.0 eq, 6.17 mmol, 31.25 mg), and a solution of a pre-weighed 0.6 mmol acid chloride monomer in pyridine (1.20 eq, 2.0 mL pyridine). The vessel was capped and placed to irradiate at 150° C. for 1800 seconds. The solvent was then removed in vacuo and the crude material was purified by HPLC method (n) and the solvents evaporated.

TABLE JJ
Examples of amides prepared from 4-phenyl-5- (trifluoromethyl)thiophene-2-
carbonyl chloride

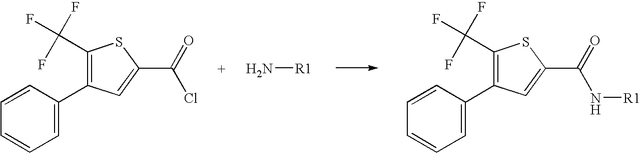

| | | | HPLC Rₜ | |
|---|---|---|---|---|
| Ex. # | Amine | Product | (Method) | m/z |
| JJ1 | 3-(4-Amino-phenyl)-propionicacid | 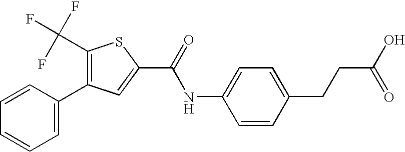 | 1.72 min (o) | 420.3(M + H)⁺ |
| JJ2 | (4-Amino-phenyl)-acetic acid | 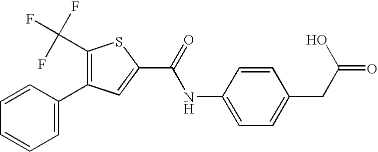 | 1.74 min (o) | 406.3(M + H)⁺ |
| JJ3 | (4-Amino-phenylsulfanyl)-acetic acid | 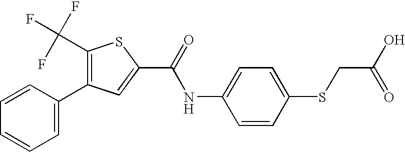 | 1.78 min (o) | 438.2(M + H)⁺ |
| JJ4 | (E)-3-(4-Amino-pyridin-3-yl)-acrylicacid | 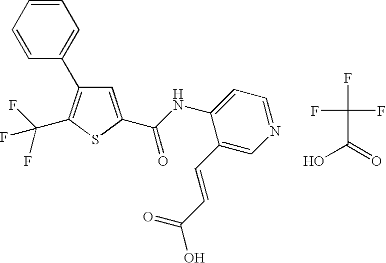 | 1.44 min (o) | 419.3(M + H)⁺ |
| JJ5 | 3-(3-Amino-phenyl)-propionicacid | 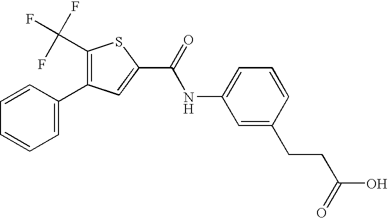 | 1.73 min (o) | 420.3(M + H)⁺ |
| JJ6 | (4-Amino-phenoxy)-acetic acid | 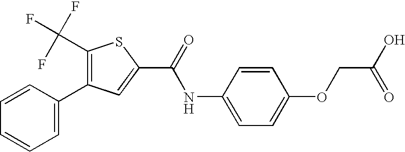 | 1.69 min (o) | 422.3(M + H)⁺ |
| JJ7 | 3-Aminomethyl-benzoic acid | 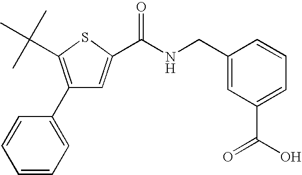 | 1.7 min (o) | 406.3(M + H)⁺ |
| JJ8 | (3-Amino-phenoxy)-acetic acid | 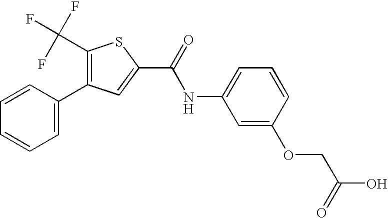 | 1.67 min (o) | 422.2(M + H)⁺ |
| JJ9 | 3-Amino-isonicotinic acid | 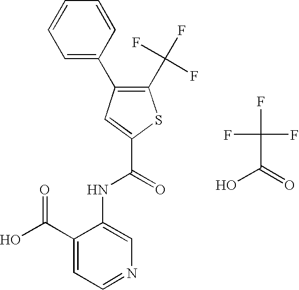 | 1.46 min (o) | 393.3(M + H)⁺ |
| JJ10 | (E)-3-(4-Amino-phenyl)-acrylic acid | 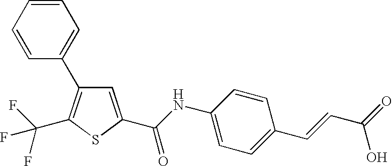 | 1.8 min (o) | 418.2(M + H)⁺ |
| JJ11 | 5-Amino-pyridine-2-carboxylic acid | 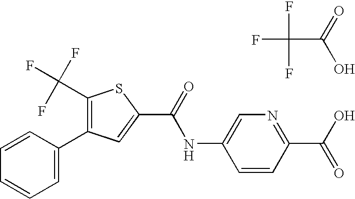 | Notdetected | 393 (M + H)⁺ |
| JJ12 | 3-Amino-thiophene-2-carboxylic acid | 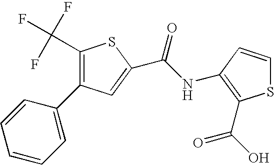 | Notdetected | 398 (M + H)⁺ |

A pre-weighed 4 mL scintillation vial containing 0.6 mmol of amine monomer (8.72 eq), was charged with a stir bar and 1M NaOH (aq) (11.0 eq, 750 μL). The vials were capped and stirred vigorously with a subsequent addition of a solution of 4-phenyl-5-(trifluoromethyl)thiophene-2-carbonyl chloride (20 mg in 250 μL in THF, 1.0 eq). After stirring for 3 hours at room temperature, the solvent was removed and 6 M HCl (aq) (150 μL, 13.0 eq) was added followed by DMSO/MeOH (1300 μL). The solution was placed to stir once more at room temperature and the mixture was then filtered using a syringe filter. The crude material was purified by HPLC method (n) and the solvents evaporated.

TABLE KK
Examples of amides prepared from 5-(1H-Indazol-5-yl)-isoxazole-3-carboxylic
acid

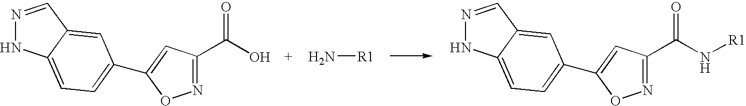

| | | | HPLC Rₜ | |
|---|---|---|---|---|
| Ex. # | Amine | Product | (Method) | m/z |
| KK1 | Cyclohexyl-methylamine | 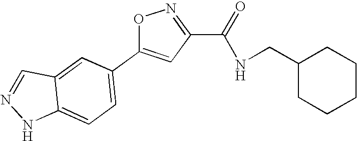 | 1.61 min (o) | 325.1(M + H)⁺ |
| KK2 | 3-Chloro-benzylamine | 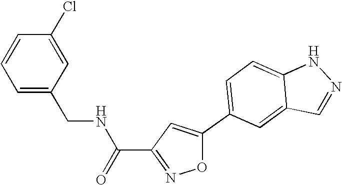 | 1.65 min (o) | 353 (M + H)⁺ |
| KK3 | 2-Ethoxy-phenylamine | 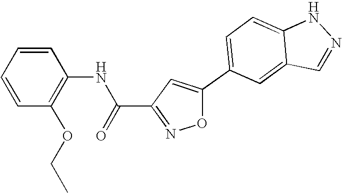 | 1.74 min (o) | 349.1(M + H)⁺ |
| KK4 | 6-Methoxy-pyridin-3-ylamine | 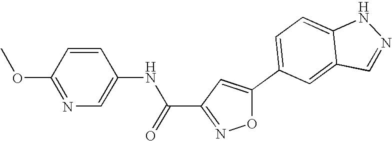 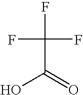 | 1.3 min (o) | 336.1(M + H)⁺ |
| KK5 | 2-Methoxy-phenylamine | 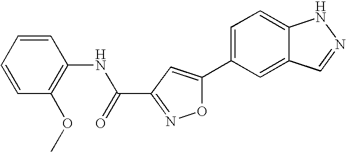 | 1.65 min (o) | 335.1(M + H)⁺ |
| KK6 | 4-chloroaniline | 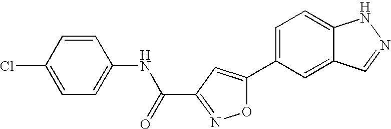 | 1.64 min (o) | 339 (M + H)⁺ |
| KK7 | 2-chlorobenzylamine | 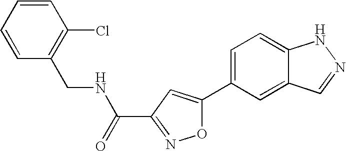 | 1.62 min (o) | 353 (M + H)⁺ |
| KK8 | 2-aminothiophene | 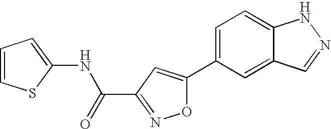 | 1.47 min (o) | 311.1(M + H)⁺ |
| KK9 | 3-chloro-4-isopropoxyaniline | 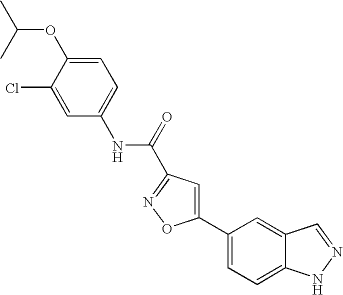 | 1.7 min (o) | 397 (M + H)⁺ |

A 20 mL scintillation vial was charged with 5-(1H-Indazol-5-yl)-isoxazole-3-carboxylic acid in DMF (1 eq, 6.43 mmol, 34.74 mg) followed by a solution of HATU in DMF (1.0 eq, 6.43 mmol). The amine monomers (1.10 eq, DMF solution) were then added followed by DIEA (2.0 eq, 12.86 mmol). The reaction mixture was placed on a heater/shaker at 40° C. for 3 hours. The solvent was then removed without using heat and the crude material was passed through a solid phase extraction column with silica carbonate media. Once the material that was passed through the column was recovered it was dried down once more and this material was then purified by HPLC method (n) and the solvents evaporated.

TABLE LL
Examples of amides prepared from 5-(4-Chloro-phenyl)-isoxazole-3-carboxylic acid

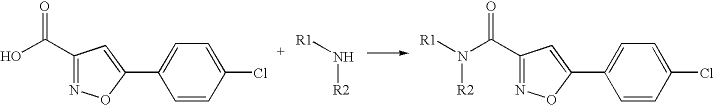

| | | | HPLC Rₜ | |
|---|---|---|---|---|
| Ex. # | Amine | Product | (Method) | m/z |
| LL1 | 1H-indol-4-amine | 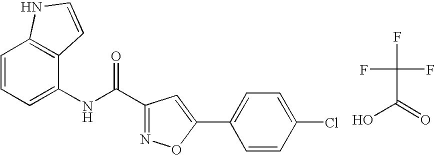 | 1.82 min (o) | 338(M + H)⁺ |
| LL2 | 1H-indol-5-amine | 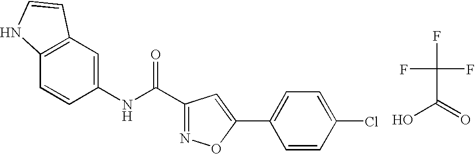 | 1.77 min (o) | 338(M + H)⁺ |

A 20 mL scintillation vial was charged with 5-(4-chloro-phenyl)-isoxazole-3-carboxylic acid in DMA (1.0 eq, 8.93 mmol, 35.78 mg) followed by a solution of HATU in DMA (1.10 eq, 9.83 mmol). This mixture was shaken briefly followed by the addition of triethylamine in a solution of DMA (2.20 eq, 19.65 mmol). The mixture is shaken again briefly at room temperature. A solution of amine monomer in DMA (1.4 eq, 3.0 mL DMA) was then added and the mixture was placed to shake overnight at room temperature. The solvent was then removed and the crude material was purified by HPLC method (n) and the solvents evaporated.

TABLE MM
Examples of amides prepared from 3-(4-Chloro-phenyl)-isoxazol-5-ylamine

| | | | HPLC Rₜ | |
|---|---|---|---|---|
| Ex. # | Acid chloride | Product | (Method) | m/z |
| MM1 | 4-methoxybenzoyl chloride | 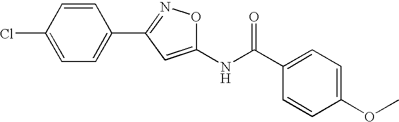 | 1.62 min (o) | 329 (M + H)⁺ |
| MM2 | 3-chloro-4-(methylsulfonyl)thiophene-2-carbonyl chloride | 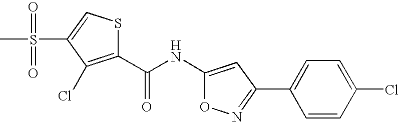 | 1.63 min (o) | 416.8(M + H)⁺ |
| MM3 | 3-chlorothiophene-2-carbonyl chloride | 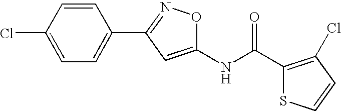 | 1.77 min (o) | 338.9(M + H)⁺ |

A Personal Chemistry 0.5 mL-2.0 mL microwave reaction vial was charged with 3-(4-Chloro-phenyl)-isoxazol-5-ylamine (1.0 eq, 7.60 mmol, 29.67 mg) dissolved in Pyridine along with a microflea-teflon coated stirring bar. To the solution was then added the acid chloride monomer dissolved in pyridine (1.5 eq, 0.23 mmol). The microwave reaction vessel was then capped and heated at 150° C. with stirring for 1200 seconds on a microwave optimizer. After cooling to ambient temperature, the vial was uncapped, an aliquot of the reaction solution was then removed for LCMS analysis (TFA+ion method) and the remaining solution was then evaporated in vacuo (Savant Speed Vac; medium heat). The residue was purified by HPLC method (n) and the solvents evaporated.

TABLE NN
Examples amide prepared from 4-(phenylsulfonyl)thiophen-3-amine

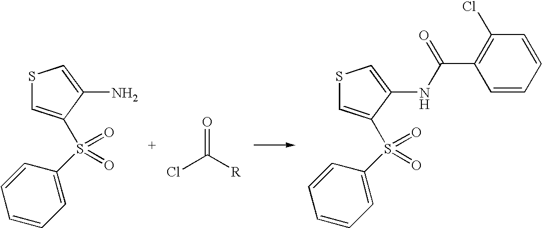

| | | | HPLC Rₜ | |
|---|---|---|---|---|
| Ex. # | Acid chloride | Product | (Method) | m/z |
| NN1 | 2-chlorobenzoyl chloride | 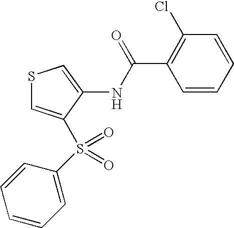 | 1.85 min (o) | 377.9(M + H)⁺ |

A 0.5 mL-2.0 mL microwave reaction vial was charged with a microflea-teflon coated stirring bar, a solution of 4-benzenesulfonyl-thiophen-3-ylamine in DMA (1.0 eq, 3.76 mmol, 17.68 mg) and pyridine (2.4 eq, 9.03 mmol). A solution of the acid chloride monomer in Chloroform (1.20 eq, 3.0 mL of CHCl₃) was then added and the vial was capped and was irradiated at 150° C. for 900 seconds. The solvent was removed from the mixture and the crude material was purified by HPLC method (n) and the solvents evaporated.

TABLE OO
Examples of amides prepared from 5-(furan-2-yl)-1-phenyl-1H-pyrazole-3-carboxylic acid

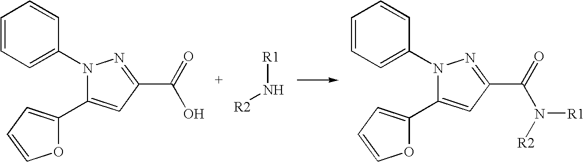

| | | | HPLC Rₜ | |
|---|---|---|---|---|
| Ex. # | Amine | Product | (Method) | m/z |
| OO1 | 2-methylaniline | 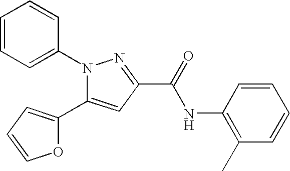 | 1.85 min (o) | 344(M + H)⁺ |
| OO2 | 2-chloroaniline | 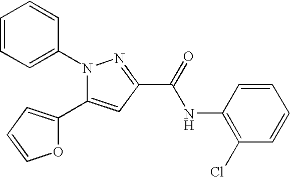 | 1.9 min (o) | 364(M + H)⁺ |
| OO3 | 4-(4-aminophenyl)butanoic acid | 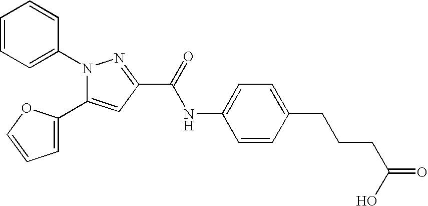 | 1.64 min (o) | 416(M + H)⁺ |
| OO4 | 3-(4-aminophenyl)propanoic acid | 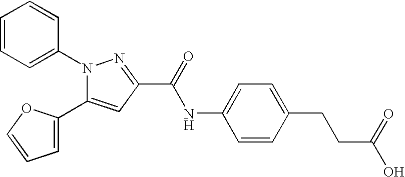 | 1.6 min (o) | 402(M + H)⁺ |
| OO5 | 1H-indazol-5-amine | 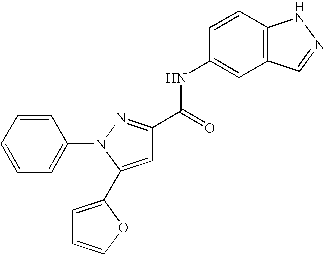 | 1.55 min (o) | 370(M + H)⁺ |
| OO6 | 2-methoxyaniline | 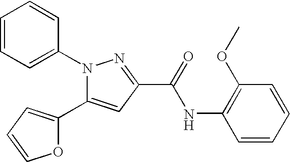 | 1.85 min (o) | 360(M + H)⁺ |
| OO7 | 3-methyl-1H-indazol-5-amine | 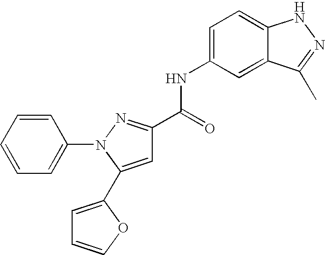 | 1.56 min (o) | 384(M + H)⁺ |
| OO8 | 7-bromo-1H-indazol-5-amine | 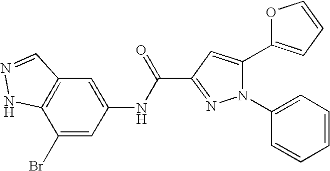 | 1.66 min (o) | 448(M + H)⁺ |
| OO9 | 2-(4-aminophenylsulfonamido)aceticacid | 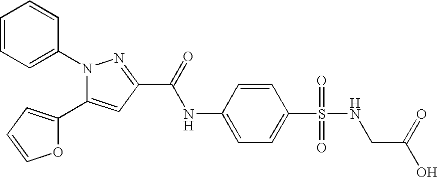 | 1.5 min (o) | 467(M + H)⁺ |
| OO10 | ethyl 5-amino-1H-indazole-3-carboxylate | 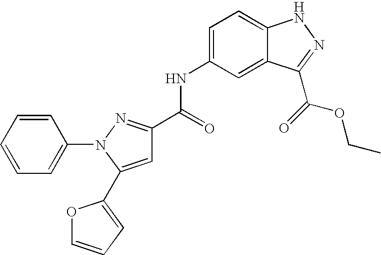 | 1.64 min (o) | 442(M + H)⁺ |
| OO11 | 6-methyl-2H-indazol-5-amine | 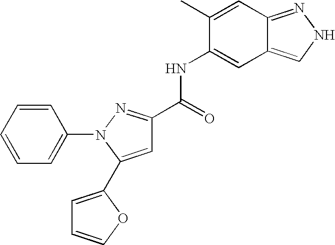 | 1.55 min (o) | 384(M + H)⁺ |
| OO12 | aniline | 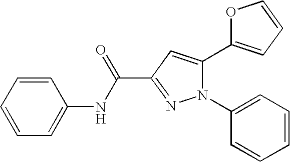 | 1.75 min (o) | 330(M + H)⁺ |
| OO13 | 6-chloro-2H-indazol-5-amine | 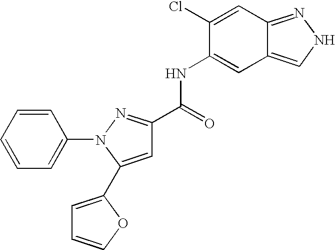 | 1.7 min (o) | 404(M + H)⁺ |
| OO14 | (E)-3-(4-aminophenyl)acrylic acid | 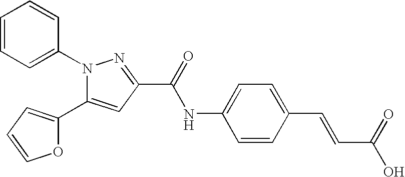 | 1.65 min (o) | 400(M + H)⁺ |

A microwave vial was charged with a stir bar and PS-TFP (2.0 eq.). To the vessel were added the 5-(furan-2-yl)-1-phenyl-1H-pyrazole-3-carboxylic acid (39 mg, 1 eq.) dissolved in dry THF and CCl3CN (1.2 eq) dissolved in dry THF. The reaction vessel was sealed and heated to 100° C. for 400 seconds. Then, the amine monomer (1.2 eq) dissolved in THF was added followed by DIEA (2 eq.) dissolved in THF. The mixture was heated to 100° C. for 600 seconds. After cooling the reaction mixture was filtered and products were collected and concentrated to dryness. The residues were dissolved in 1:1 DMSO/MeOH and the crude material was purified by HPLC method (n) and the solvents evaporated.

TABLE PP
Examples of amides prepared from 5-cyclopropylisoxazole-3-carboxylic acid

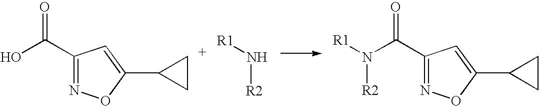

| | | | HPLC Rₜ | |
|---|---|---|---|---|
| Ex. # | Amine | Product | (Method) | m/z |
| PP1 | 2-chloroaninline | 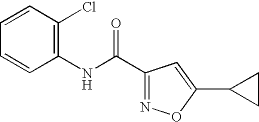 | 1.75 min (o) | 263(M + H)⁺ |
| PP2 | 4-(4-aminophenyl)butanoic acid | 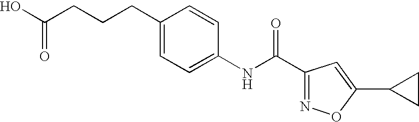 | 1.4 min (o) | 315(M + H)⁺ |
| PP3 | 1H-indazol-5-amine | 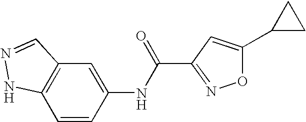 | 1.23 min (o) | 269(M + H)⁺ |
| PP4 | 7-methyl-2H-indazol-5-amine | 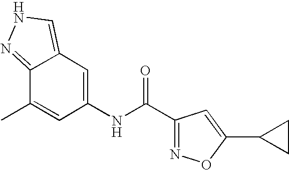 | 1.3 min (o) | 283(M + H)⁺ |
| PP5 | 3-methyl-1H-indazol-5-amine | 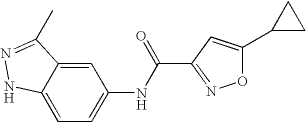 | 1.25 min (o) | 283(M + H)⁺ |
| PP6 | 6-chloro-2H-indazol-5-amine | 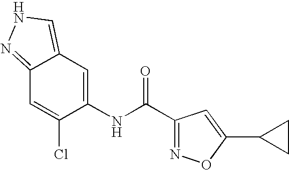 | 1.4 min (o) | 303(M + H)⁺ |

A microwave vial was charged with a stir bar and PS-TFP (2.0 eq.). To the vessel were added the 5-cyclopropylisoxazole-3-carboxylic acid (38 mg, 1 eq.) dissolved in dry THF and CCl₃CN (1.2 eq) dissolved in dry THF. The reaction vessel was sealed and heated to 100° C. for 400 seconds. Then, the amine monomer (1.2 eq) dissolved in THF was added followed by DIEA (2 eq.) dissolved in THF. The mixture was heated to 100° C. for 600 seconds. After cooling the reaction mixture was filtered and products were collected and concentrated to dryness. The residues were dissolved in 1:1 DMSO/MeOH and the crude material was purified by HPLC method (n) and the solvents evaporated.

TABLE QQ
Examples of amides prepared from 3-cyclohexylisoxazole-5-carboxylic acid

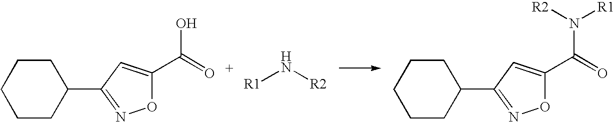

| | | | HPLC Rₜ | |
|---|---|---|---|---|
| Ex. # | Amine | Product | (Method) | m/z |
| QQ1 | 4-methyl-2-chloroaniline | 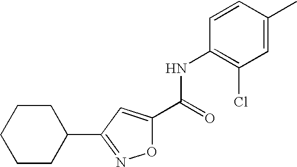 | 2.1 min (o) | 319(M + H)⁺ |
| QQ2 | 1H-indol-4-amine | 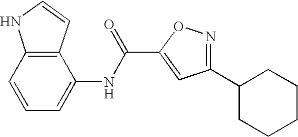 | 1.8 min (o) | 310(M + H)⁺ |
| QQ3 | 1H-indazol-5-amine | 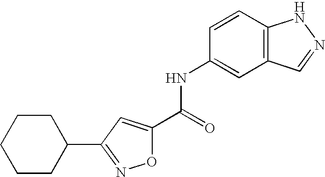 | 1.65 min (o) | 311(M + H)⁺ |
| QQ4 | 6-chloro-1H-indazol-5-amine | 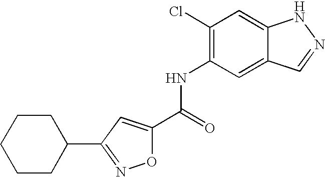 | 1.75 min (o) | 345(M + H)⁺ |

In a 20 ml vial a solution of 3-cyclohexylisoxazole-5-carboxylic acid (37 mg, 1 eq.) dissolved in DMA was added HATU (1.2 eq.) dissolved in DMA followed by TEA (4 eq.) dissolved in DMA. After some mixing, a solution of the amine monomer (1.4 eq.) was added dissolved in DMA. The mixture was shaken for 4 hours. After that the products were concentrated to dryness. The residues were dissolved in 1:1 DMSO/MeOH and the crude material was purified by HPLC method (n) and the solvents evaporated.

TABLE RR
Examples of amides prepared from 5-phenylisoxazole-3-carbonyl chloride

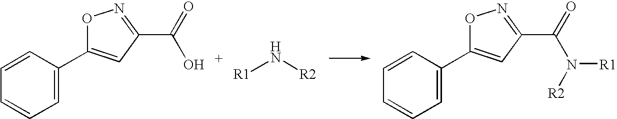

| | | | HPLC Rₜ | |
|---|---|---|---|---|
| Ex. # | Amine | Product | (Method) | m/z |
| RR1 | 4-isopropoxy-3-chloroaniline | 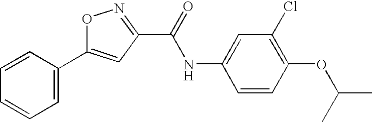 | 2.08 min (o) | 357(M + H)⁺ |
| RR2 | cyclohexylamine | 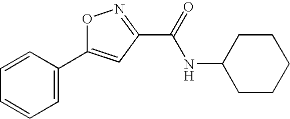 | 1.85 min (o) | 271(M + H)⁺ |
| RR3 | 2-chloroaniline | 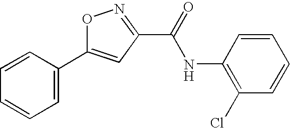 | 2 min (o) | 299(M + H)⁺ |
| RR4 | 4-methoxybenzylamine | 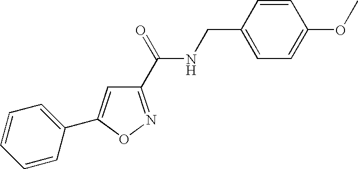 | 1.8 min (o) | 309(M + H)⁺ |
| RR5 | 3-aminobenzonitrile | 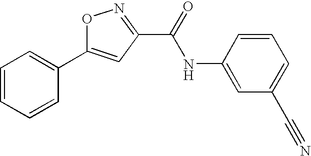 | 1.75 min (o) | 290(M + H)⁺ |
| RR6 | 4-methoxyaniline | 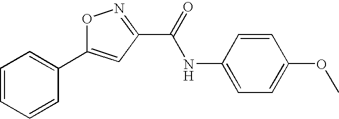 | 1.75 min (o) | 292(M + H)⁺ |

In a 20 mL vial a solution of 5-phenylisoxazole-3-carbonyl chloride (48 mg, 1 eq.) dissolved in dichloromethane was added followed by a solution of the amine monomer (1.2 eq), followed by TEA (2.4 eq.) dissolved in dichloromethane followed by DMAP (0.1 eq) dissolved in dichloromethane. The mixture was shaken overnight. Then the products were concentrated to dryness. The residues were dissolved in 1:1 DMSO/MeOH and the crude material was purified by HPLC method (n) and the solvents evaporated.

TABLE SS
Examples of amides prepared from 3-(2-chlorophenyl)isoxazol-5-amine

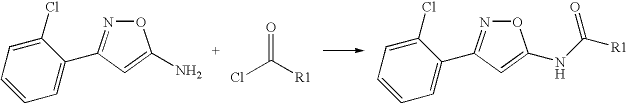

| | | | HPLC Rₜ | |
|---|---|---|---|---|
| Ex. # | Acid chloride | Product | (Method) | m/z |
| SS1 | 4-isopropoxybenzoyl chloride | 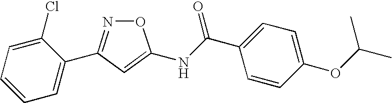 | 1.9 min (o) | 357(M + H)⁺ |
| SS2 | 4-methoxybenzoyl chloride | 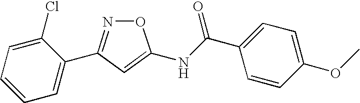 | 1.75 min (o) | 329(M + H)⁺ |
| SS3 | 4-ethoxybenzoyl chloride | 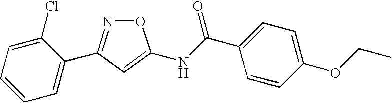 | 1.85 min (o) | 343(M + H)⁺ |
| SS4 | 2-phenylbutanoyl chloride | 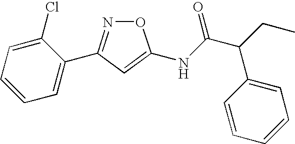 | 1.92 min (o) | 341(M + H)⁺ |
| SS5 | 4-trifluoromethoxybenzoyl chloride | 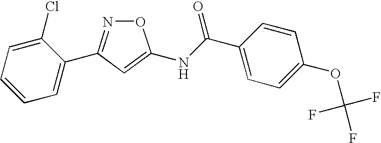 | 1.98 min (o) | 383(M + H)⁺ |
| SS6 | 3-methylthiophene-2-carbonyl chloride | 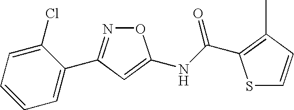 | 1.8 min (o) | 319(M + H)⁺ |
| SS7 | 4-methoxy-3-(trifluoromethyl)benzoyl chloride | 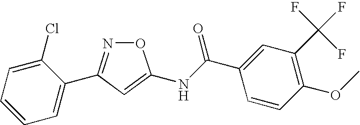 | 1.96 min (o) | 397(M + H)⁺ |

In a microwave vial a solution of 3-(2-chlorophenyl)isoxazol-5-amine (25 mg, 1 eq.), dissolved in pyridine was added followed by a solution of the acid chloride monomer (1.5 eq.) dissolved in pyridine. The mixture was heated to 150° C. for 1800 seconds. After cooling the reaction mixture was concentrated to dryness. The residues were dissolved in 1:1 DMSO/MeOH and the crude material was purified by HPLC method (n) and the solvents evaporated.

TABLE TT
Examples of amides prepared from 5-methyl-1-(3-(trifluoromethyl)phenyl)-1H-1,2,3-triazole-4-carboxylic acid

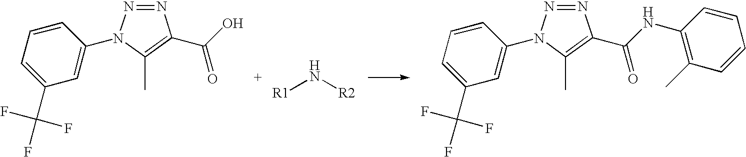

| | | | HPLC Rₜ | |
|---|---|---|---|---|
| Ex. # | Amine | Product | (Method) | m/z |
| TT1 | 2-methylaniline | 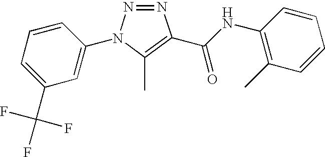 | 1.85 min (o) | 361(M + H)⁺ |
| TT2 | 2-chloroaniline | 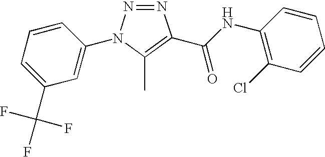 | 1.95 min (o) | 381(M + H)⁺ |
| TT3 | 1H-indazol-5-amine | 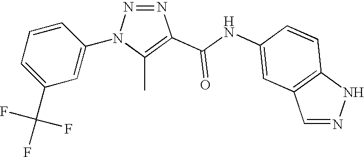 | 1.56 min (o) | 387(M + H)⁺ |
| TT4 | 2-methoxyaniline | 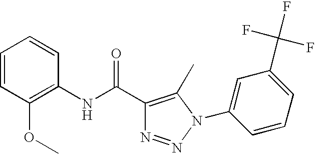 | 1.88 min (o) | 377(M + H)⁺ |
| TT5 | 2-(4-aminophenylsulfonamido)acetic acid | 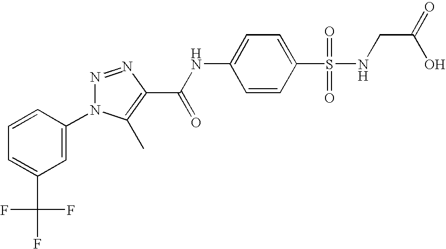 | 1.55 min (o) | 484(M + H)⁺ |

A microwave vial was charged with a stir bar and PS-TFP (2.0 eq.). To the vessel were added 5-methyl-1-(3-(trifluoromethyl)phenyl)-1H-1,2,3-triazole-4-carboxylic acid (40 mg, 1 eq.) dissolved in dry THF and CCl₃CN (1.2 eq) dissolved in dry THF. The reaction vessel was sealed and heated to 100° C. for 300 seconds. Then, the amine monomer (1.0 eq) dissolved in THF was added followed by DIEA (2 eq.) dissolved in THF. The mixture was heated to 100° C. for 600 seconds. After cooling the reaction mixture was filtered through a Si-Carbonate resin cartridge and products were collected and concentrated to dryness. The residues were dissolved in 1:1 DMSO/MeOH and the crude material was purified by HPLC method (n) and the solvents evaporated.

TABLE UU
Examples of amides prepared from 3-(pyridin-4-yl)isoxazol-5-amine

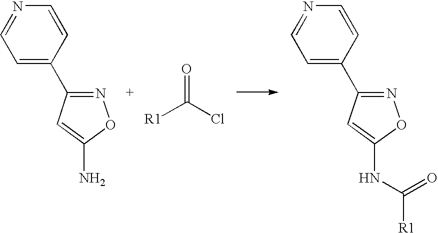

| | | | HPLC Rₜ | |
|---|---|---|---|---|
| Ex. # | Acid chloride | Product | (Method) | m/z |
| UU1 | 4-trifluromethoxybenzoyl chloride | 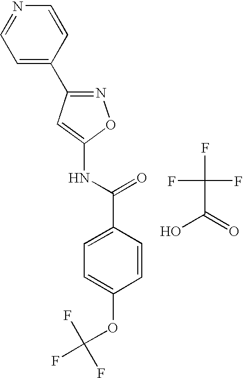 | 1.26 min (o) | 350(M + H)⁺ |
| UU2 | 4-isopropoxybenzoyl chloride | 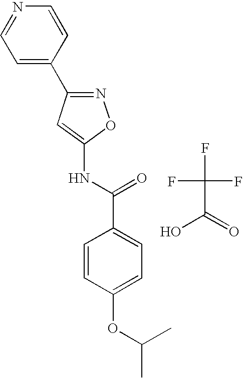 | 1.20 min (o) | 324(M + H)⁺ |
| UU3 | 4-ethoxybenzoyl chloride | 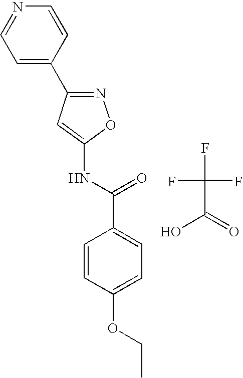 | 1.15 min (o) | 310(M + H)⁺ |
| UU4 | 2-phenylbutanoyl chloride | 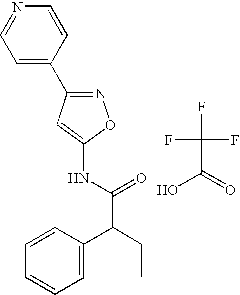 | 1.20 min (o) | 308 (M + H)⁺ |
| UU5 | 3-methylthiophene-2-carbonyl chloride | 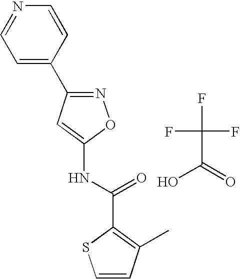 | 1.00 min (o) | 286(M + H)⁺ |
| UU6 | 4-methoxy-3-(trifluoromethyl)benzoyl chloride | 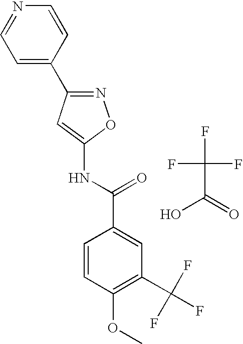 | 1.25 min (o) | 364(M + H)⁺ |
| UU7 | 6-(trifluoromethyl)nicotinoyl chloride | 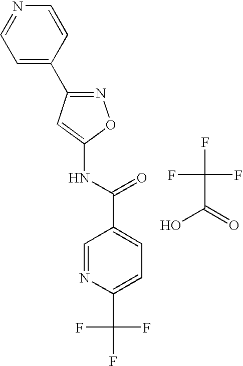 | 1.05 min (o) | 335(M + H)⁺ |

In a microwave vial a solution of 3-(pyridin-4-yl)isoxazol-5-amine (29 mg, 1 eq.), dissolved in pyridine was added followed by a solution of the acid chloride monomer (1.5 eq.) dissolved in pyridine. The mixture was heated to 150° C. for 1800 seconds. After cooling the reaction mixture was concentrated to dryness. The residues were dissolved in 1:1 DMSO/MeOH and the crude material was purified by HPLC method (n) and the solvents evaporated.

TABLE VV
Examples of amines prepared from 5-(3-methoxyphenyl)isoxazole-3-carboxylic acid

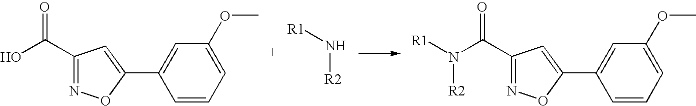

| | | | HPLC Rₜ | |
|---|---|---|---|---|
| Ex. # | Amine | Product | (Method) | m/z |
| VV1 | 2-chloroaniline | 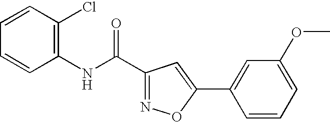 | 1.95 min (o) | 329(M + H)⁺ |
| VV2 | 1H-indazol-5-amine | 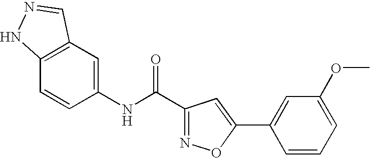 | 1.50 min (o) | 335(M + H)⁺ |
| VV3 | 6-chloro-2H-indazol-5-amine | 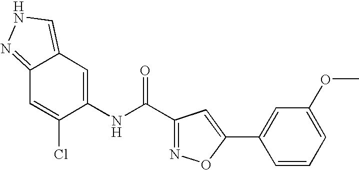 | 1.65 min (o) | 369(M + H)⁺ |

A microwave Vial was charged with a stir bar and PS-TFP (2.0 eq.). To the vessel were added 5-(3-methoxyphenyl)isoxazole-3-carboxylic acid (40 mg, 1 eq.) dissolved in dry THF and CCl₃CN (1.2 eq) dissolved in dry THF. The reaction vessel was sealed and heated to 100° C. for 400 seconds. Then, the amine monomer (1.2 eq) dissolved in THF was added followed by DIEA (2 eq.) dissolved in THF. The mixture was heated to 100° C. for 600 seconds. After cooling the reaction mixture was filtered and products were collected and concentrated to dryness. The residues were dissolved in 1:1 DMSO/MeOH and the crude material was purified by HPLC method (n) and the solvents evaporated.

TABLE WW
Examples of amides prepared from 3-phenylisoxazol-5-amine

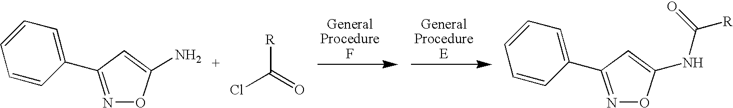

| | | | HPLC Rₜ | |
|---|---|---|---|---|
| Ex. # | Amine, acid or acid chloride | Product | (Method) | m/z |
| WWI | 3-phenyl-isoxazol-5-ylamine,methyl 3-chlorocarbonylbenzoate | 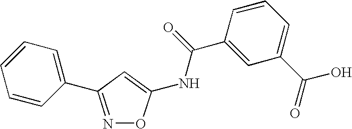 | 2.14 min (a) | 309.1(M + H)⁺ |
| WWII | 3-phenyl-isoxazol-5-ylamine,methyl 4-chlorocarbonylbenzoate | 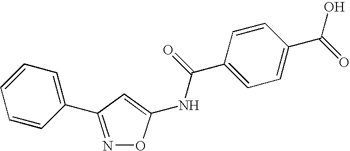 | 2.09 min (a) | 309.1(M + H)⁺ |

### Preparation of 3-phenyl-isoxazole-5-carbonyl chloride

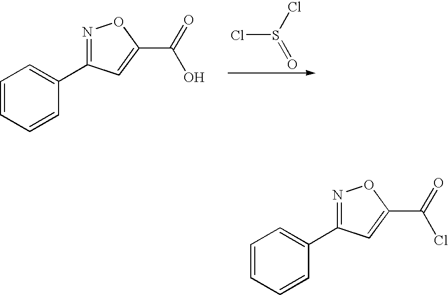

3-phenylisoxazole-5-carboxylic acid (1 g, 5.29 mmol) was dissolved in thionyl chloride (10 mL, 137 mmol) to give a pale yellow suspension. The reaction mixture was heated at 50° C. for about 96 h giving a clear yellow solution. After cooling to ambient temperature the thionyl chloride was removed under vacuum (Genevac) to provide 3-phenylisoxazole-5-carbonyl chloride (1.02 g, 4.91 mmol, 93% yield) as a pale yellow solid: ¹H NMR (400 MHz, CDCl₃) δ ppm 7.88-7.80 (m, 2H), 7.54-7.48 (m, 3H), 7.00 (s, 1H).

### Preparation of 4-(2-aminopropan-2-yl)aniline hydrochloride

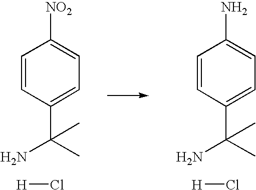

A solution of 2-(4-nitrophenyl)propan-2-amine hydrochloride (500 mg, 2.308 mmol) (Sinova) in MeOH (50 μL) was passed through a Pd/C cartridge on H-cube (Thales Nano) with a flow rate of 1 mL/min. The solvent was removed and the residue was triturated with ether to give 4-(2-aminopropan-2-yl)aniline hydrochloride (348 mg, 1.864 mmol, 81% yield) as an off-white solid: ¹H NMR (400 MHz, DMSO-d6) δ ppm 8.16 (s, 3H), 7.19 (m, 2H), 6.56 (m, 2H), 5.15 (s, 2H), 1.55 (s, 6H).

### Preparation of 3-((tert-butyldimethylsilyloxy)methyl)aniline

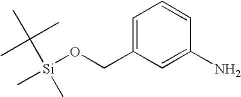

In a 100 mL round-bottomed flask was combined (3-aminophenyl)methanol (2.0 g, 16.24 mmol), tert-butylchlorodimethylsilane (2.69 g, 17.86 mmol), DMAP (0.655 g, 5.36 mmol) and DMF (50 mL). Triethylamine (2.72 mL, 19.49 mmol) was added in one portion. The mixture was stirred at room temperature overnight. The crude reaction was poured into water and extracted with EtOAc. The organic layer was concentrated to dryness to give a black liquid which was purified via flash chromatography (0-50% EtOAc/heptane over 50 min; RS-120 Si column) to give 3-((tert-butyldimethylsilyloxy)methyl)aniline (3.58 g, 15.08 mmol, 93% yield) as an oil: LCMS (Table 1, Method b) Rₜ=2.63 min, m/z 238.19 (M+H)⁺.

### Preparation of 4-((tert-butyldimethylsilyloxy)methyl)aniline

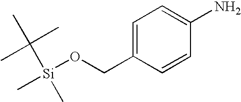

To a solution of (4-aminophenyl)methanol (3 g, 24.36 mmol) in DMF (85 mL) was added DMAP (0.982 g, 8.04 mmol) and triethylamine (4.07 mL, 29.2 mmol). Tertbutylchloro-dimethylsilane (4.04 g, 26.8 mmol) was added. The reaction mixture was stirred at ambient temperature overnight. The reaction mixture was filtered to remove the salt. The filtrate was concentrated to afford 7.9 g of a red solid, which was dissolved in EtOAc (200 mL), washed with water (100 mL), saturated ammonium chloride (2×50 mL), water (2×50 mL) and brine (50 mL), the organic layer was dried over Na₂SO₄. Filtration and concentration afforded 4-((tertbutyldimethylsilyloxy)methyl)aniline (5.66 g, 23.84 mmol, 98% yield) as a red oil. LCMS (Table 1, Method a) Rₜ=3.10 min, m/z 238.19 (M+H)⁺; ¹H NMR (400 MHz, DMSO-d6) δ ppm 6.94 (d, 2H), 6.51 (d, 2H), 4.95 (s, 2H), 4.49 (s, 2H), 0.87 (s, 9H), 0.03 (s, 6H).

### Preparation of 1-(3,4-dichlorophenyl)-N-(4-(hydroxymethyl)phenyl)-5-(trichloromethyl)-1H-1,2,4-triazole-3-carboxamide

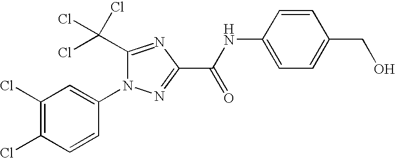

To a red solution of 4-((tert-butyldimethylsilyloxy)methyl)aniline (0.309 g, 1.300 mmol) in THF (20 mL) was added triethylamine (0.217 mL, 1.560 mmol). 1-(3,4-Dichlorophenyl)-5-(trichloromethyl)-1H-1,2,4-triazole-3-carbonyl chloride (0.512 g, 1.3 mmol) was added in one portion. The reaction mixture was stirred at ambient temperature for 10 min until TLC showed no starting material. A solution of TBAF in THF (1M, 2.60 mL, 2.60 mmol) was added dropwise. The reaction mixture was stirred at ambient temperature overnight and then concentrated. The resulting residue was partitioned between EtOAc (100 mL) and HCl (5%, 50 mL). The organic layer was washed by HCl (10%, 50 mL), sat. NaHCO₃ (50 mL), water (50 mL), the ethyl acetate was dried over Na₂SO₄. Filtration and concentration afforded 1-(3,4-dichlorophenyl)-N-(4-(hydroxymethyl)phenyl)-5-(trichloromethyl)-1H-1,2,4-triazole-3-carboxamide (0.664 g, 1.105 mmol, 85% yield) as an orange solid. LCMS (Table 1, Method a) Rₜ=3.70 min, m/z 481.11 (M+H)⁺; ¹H NMR (400 MHz, DMSO-d6) δ ppm 10.71 (s, 1H), 8.10 (d, 1H), 8.05 (d, 1H), 7.78-7.81 (m, 3H), 7.35 (d, 2H), 5.19 (t, 1H), 4.51 (d, 2H).

### Preparation of 1-(3-(3-(ethoxycarbonyl)-4-phenylthiophen-2-ylcarbamoyl)benzyl)azetidine-3-carboxylic acid

### Example #5

### Step A. Preparation of ethyl 2-(3-(1,3-dioxolan-2-yl)benzamido)-4-phenylthiophene-3-carboxylate

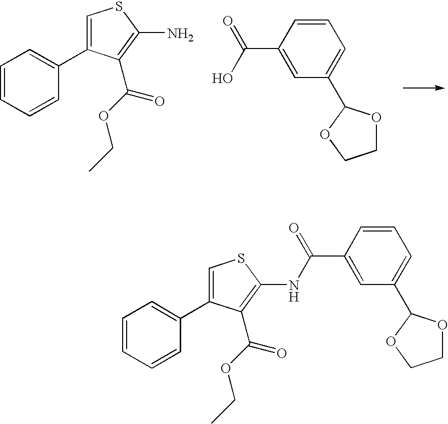

A 5 mL microwave vial equipped with a stirring bar was charged with 3-(1,3-dioxolan-2-yl)benzoic acid (306 mg, 1.577 mmol), ethyl 2-amino-4-phenylthiophene-3-carboxylate (300 mg, 1.213 mmol), BEMP (1.369 mL, 4.73 mmol), HBTU (598 mg, 1.577 mmol), and acetonitrile (3.5 mL). The vessel was capped and the reaction heated to 140° C. for 30 min under microwave irradiation (Biotage Optimizer, 300 W). Solvent was removed under reduced pressure, and the crude material purified by flash column chromatography (Analogix System; 80 g column; 100% heptane ramping to 50% ethyl acetate in heptane over 30 min) to give ethyl 2-(3-(1,3-dioxolan-2-yl)benzamido)-4-phenylthiophene-3-carboxylate (138 mg, 26.6%). LCMS (Table 1, Method c) Rₜ=3.03 min, m/z=422.38 (M−H)⁻; ¹H NMR (400 MHz, DMSO-d6) δ ppm 11.98 (s, 1H), 8.03 (s, 1H), 8.01-7.95 (m, 1H), 7.76 (d, J=7.69 Hz, 1H), 7.68 (t, J=7.66 Hz, 1H), 7.42-7.28 (m, 5H), 7.01 (s, 1H), 5.88 (s, 1H), 4.16-4.10 (m, 2H), 4.10-3.98 (m, 4H), 0.96 (t, J=7.11 Hz, 3H).

### Step B. Preparation of ethyl 2-(3-formylbenzamido)-4-phenylthiophene-3-carboxylate

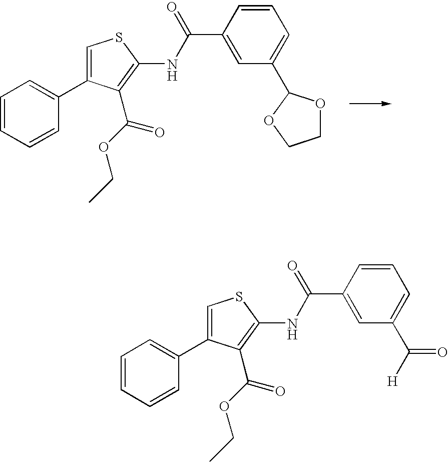

A 50 mL round bottom flask equipped with a stirring bar was charged with ethyl 2-(3-(1,3-dioxolan-2-yl)benzamido)-4-phenylthiophene-3-carboxylate (125 mg, 0.295 mmol) and THF (8 mL). 2M aqueous HCl (4 mL, 8.00 mmol) was then added and the reaction stirred at ambient temperature for 18 h. Product precipitated from reaction mixture and was collected by filtration and washed with a 1:1 mixture of water and THF (3×10 mL). The solid was then dried in a vacuum oven overnight to give ethyl 2-(3-formylbenzamido)-4-phenylthiophene-3-carboxylate (96.3 mg, 77%). LCMS (Table 1, Method a) Rₜ=3.64 min, m/z=378.16 (M−H)⁻; 1H NMR (400 MHz, DMSO-d6) δ ppm 12.03 (s, 1H), 10.15 (s, 1H), 8.46 (s, 1H), 8.29-8.24 (m, 1H), 8.23 (d, J=7.64 Hz, 1H), 7.89 (t, J=7.69 Hz, 1H), 7.36 (m, 5H), 7.04 (s, 1H), 4.13 (q, J=7.11 Hz, 2H), 0.96 (t, J=7.11 Hz, 3H).

### Step C. Preparation of 1-(3-(3-(ethoxycarbonyl)-4-phenylthiophen-2-ylcarbamoyl)benzyl)azetidine-3-carboxylic acid

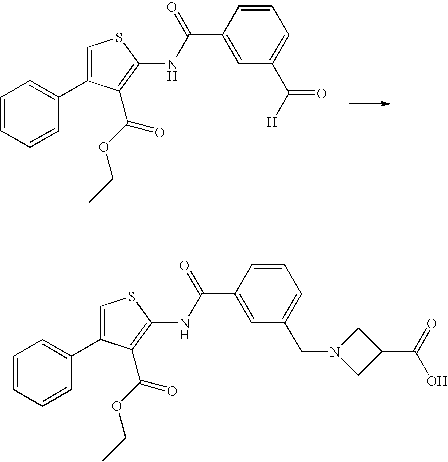

A 20 mL vial equipped with a stirring bar was charged with ethyl 2-(3-formylbenzamido)-4-phenylthiophene-3-carboxylate (83 mg, 0.219 mmol), azetidine-3-carboxylic acid (22.12 mg, 0.219 mmol), and methanol (2 mL). After stirring for a few minutes at ambient temperature, sodium cyanoborohydride (13.75 mg, 0.219 mmol) was added in one portion and the reaction sealed and stirred at ambient temperature overnight. Solvent was removed under reduced pressure and the crude product was purified by RP-HPLC (A=50 mM ammonium acetate, B=acetonitrile; 30-80% B over 30.0 min (21.0 mL/min flow rate); 21.2×250 mm Thermo Hyperprep® C18 column, 8 μm particles) to give 1-(3-(3-(ethoxycarbonyl)-4-phenylthiophen-2-ylcarbamoyl)benzyl)azetidine-3-carboxylic acid (20.3 mg; 19.8%). LCMS (Table 1, Method c) Rₜ=1.86 min, m/z 465.23 (M+H)⁺; ¹H NMR (400 MHz, DMSO-d6) δ ppm 11.97 (s, 1H), 7.86 (d, J=16.76 Hz, 2H), 7.60 (s, 2H), 7.35 (d, J=5.43 Hz, 5H), 7.00 (s, 1H), 4.21-4.03 (m, 2H), 3.71 (s, 2H), 3.47 (d, J=5.15 Hz, 2H), 3.28 (t, J=9.83 Hz, 3H), 0.96 (t, J=6.99 Hz, 3H).

### Preparation of ethyl 5-amino-3-phenylisoxazole-4-carboxylate

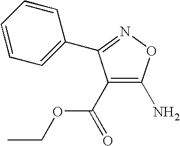

Into a round bottom flask was added benzoyl chloride (2.5 mL, 0.022 mol), benzene (100 mL, 1 mol) and cyanoacetic acid ethyl ester (1.1 mL, 0.011 mol) (prepared according to the procedure of Gilman, H., and A. H. Blatt, Organic Syntheses, 1941, 1, 254-256). Triethylamine (3.0 mL, 0.022 mol) was added and the reaction turned orange and thick. The reaction was stirred at ambient temperature for 48 hours. The reaction was diluted with water and separated. The organic layer was dried over Na₂SO₄, and filtered and concentrated to leave a orange oil. The oil was purified by flash chromatography on silica gel (gradient 0-40% over 60 min, ethyl acetate/heptane). The fraction containing the correct mass by LCMS was concentrated to dryness, dissolved in DCM (100 mL, 2 mol) and placed into a round bottom flask. To the solution was added phosphoryl chloride (1 mL, 0.01 mol). Triethylamine (3 mL, 0.02 mol) was added dropwise to the solution while stirring. The reaction changed from yellow to dark red upon heating the mixture to reflux. The reaction was refluxed overnight. The reaction was cooled and extracted with HCl (5 M). The solvent was evaporated under reduced pressure to leave an orange/brown tar. The tar was dissolved in ether (50 mL) and washed with HCl (5 M) and sodium bicarbonate solution. The organic phase was collected, dried over Na₂SO₄, filtered and the solvent was evaporated under reduced pressure. The residue was purified by flash chromatography on silica gel using a 0-50% ethyl acetate/heptane gradient over 60 min to afford a residue to which 1 equivalent of hydroylamine-hydrochloride in 1 mL of 10% NaOH was added. The reaction was stirred for 3 h at ambient temperature. Water was added to the reaction and then it was extracted with DCM (3×50 mL). The organic extracts were combined, dried over Na₂SO₄, filtered and concentrated to dryness. The residue was purified by flash chromatography on silica gel using a 0-40% ethyl acetate/heptane gradient over 35 min to afford ethyl 5-amino-3-phenylisoxazole-4-carboxylate (498 mg, 2 mmol) as a white powder. LCMS (Table 1, Method a) Rₜ=2.46 min, m/z 233.1 (M+H)⁺.

### Preparation of 1-(4-(4-phenyl-5-(trifluoromethyl)thiophene-2-carboxamido)benzyl)azetidine-3-carboxylic acid

### Example #6

### Step A. Preparation of N-(4-cyanophenyl)-4-phenyl-5-(trifluoromethyl)thiophene-2-carboxamide

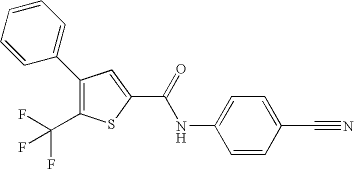

To a suspension of 4-phenyl-5-(trifluoromethyl)thiophene-2-carbonyl chloride (0.750 g, 2.58 mmol) in dry toluene (5.16 mL) was added 4-aminobenzonitrile (0.305 g, 2.58 mmol), followed by triethylamine (0.539 mL, 3.87 mmol) dropwise. The resulting mixture was heated to 110° C. overnight. Heating was stopped and the reaction mixture was diluted with DCM (150 mL). The organic layer was washed with saturated bicarbonate solution, then 0.6 M HCl solution. The organic layer was washed with brine (150 mL), dried MgSO₄ and concentrated to yield light brown solid. The solid was triturated with EtOAc/Heptane mixture to remove trace impurity. The mixture was filtered and dried to yield N-(4-cyanophenyl)-4-phenyl-5-(trifluoromethyl)thiophene-2-carboxamide (827 mg, 2.22 mmol) as an off-white solid. ¹H NMR (400 MHz, CDCl₃) δ ppm 7.83 (d, J=0.5 Hz, 1H), 7.76 (m, 2H), 7.68 (m, 2H), 7.58 (m, 1H), 7.46 (m, 5H).

### Step B. Preparation of N-(4-formylphenyl)-4-phenyl-5-(trifluoromethyl)thiophene-2-carboxamide

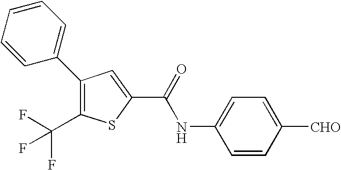

N-(4-Cyanophenyl)-4-phenyl-5-(trifluoromethyl)thiophene-2-carboxamide (0.951 g, 2.55 mmol) was dissolved in a mixture of pyridine (33.6 mL), acetic acid (16.80 mL) and water (16.80 mL), then cooled to 0° C. in an ice bath. Sodium dihydrogen phosphate monohydrate (4.60 g, 43.4 mmol) was added as a solid, followed by the addition of raney nickel (0.42 g per 1.4 mmol nitrile) as a water slurry. The mixture was heated to 60° C. under an atmosphere of nitrogen for 1.5 hrs. The reaction mixture was cooled to ambient temperature and filtered through Celite®. The Celite® cake was washed with ethanol (20 mL) and EtOAc (20 mL). The filtrate was concentrated in vacuo to give crude product as a green liquid. The crude material was taken up in EtOAc (150 mL) and water (150 mL). The organic layer was washed with 5% aqueous citric acid (150 mL), saturated NaHCO₃ solution (150 mL), and brine (150 mL). The organic layer was dried (MgSO₄) and concentrated to yield crude solid. The residue was dissolved in EtOAc and purified via Analogix FC system using RediSep® RS 40 g column, with a gradient of 0-100% EtOAc/Heptane over 30 min. at 30 mL/min. to give N-(4-formylphenyl)-4-phenyl-5-(trifluoromethyl)thiophene-2-carboxamide (698 mg, 1.86 mmol) as an off-white solid. ¹H NMR (400 MHz, CDCl3) δ ppm 9.97 (s, 1H), 7.93 (d, J=8.6 Hz, 2H), 7.82 (t, J=10.9 Hz, 3H), 7.58 (d, J=1.2 Hz, 1H), 7.44 (d, J=9.1 Hz, 5H).

### Step C. Preparation of 1-(4-(4-phenyl-5-(trifluoromethyl)thiophene-2-carboxamido)benzyl)azetidine-3-carboxylic acid

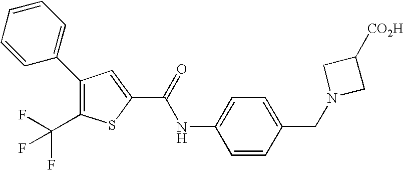

N-(4-Formylphenyl)-4-phenyl-5-(trifluoromethyl)thiophene-2-carboxamide (0.150 g, 0.400 mmol) was suspended in a mixture of MeOH (5.00 mL) and DCE (5.00 mL) to give clear solution. To this was added azetidine-3-carboxylic acid (0.042 g, 0.420 mmol) as solid, followed shortly by acetic acid (0.2 mL, 3.49 mmol). The resulting mixture was stirred at ambient temperature for 30 min. under the atmosphere of nitrogen, then sodium cyanoborohydride (0.013 g, 0.200 mmol) was added in one portion. The reaction was stirred at ambient temperature overnight. The resulting white suspension was filtered. The collected solid was washed with water and cold MeOH, and air-dried to yield 1-(4-(4-phenyl-5-(trifluoromethyl)thiophene-2-carboxamido)benzyl)azetidine-3-carboxylic acid (149 mg, 0.32 mmol). LCMS (Table 1, Method c) Rₜ=1.98 min, m/z 461.27 (M+H)⁺. ¹H NMR (400 MHz, DMSO-d6) δ ppm 11.96-12.45 (m, 1H), 10.45 (s, 1H), 8.19 (d, J=1.2 Hz, 1H), 7.66 (d, J=8.5 Hz, 2H), 7.52 (m, 5H), 7.27 (d, J=8.5 Hz, 2H), 3.51 (s, 2H), 3.35 (m, 2H), 3.19 (t, J=6.4 Hz, 3H).

### Preparation of (R)-N-(3-chloro-4-isopropoxyphenyl)-5-(4-(2,3-dihydroxypropoxy)phenyl)isoxazole-3-carboxamide

### Example #7

### Step A. Preparation of 1-(4-((2,2-dimethyl-1,3-dioxolan-4-yl)methoxy)phenyl)ethanone

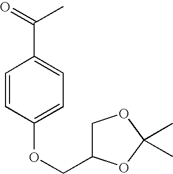

Into a 250 mL round-bottomed was added triphenylphosphine (3.13 g, 11.92 mmol) and THF (120 mL) to give a colorless solution. The solution was cooled to 0° C. Diisopropyl azodicarboxylate (2.317 mL, 11.92 mmol) was added dropwise over 10 minutes. The reaction mixture was stirred at 0° C. for 30 min. Then a colorless solution of 1-(4-hydroxyphenyl)ethanone (1.623 g, 11.92 mmol) and (2,2-dimethyl-1,3-dioxolan-4-yl)methanol (1.407 mL, 11.35 mmol) was added to the mixture over 30 minutes. The mixture was stirred for 2 hours at 0° C. and then overnight at ambient temperature. The reaction mixture was concentrated to dryness and the residue triturated with ether. The white solid was filtered off and the filtrate was concentrated to afford an yellow viscous oil which was purified via Analogix (0-40% EtOAc/Heptane over 40 minutes; RS-120 Si column) to give 1-(4-((2,2-dimethyl-1,3-dioxolan-4-yl)methoxy)phenyl)ethanone (2.352 g, 83% yield) as a white solid. LCMS (Table 1, Method b) Rₜ=1.97 min (no ionization); ¹H NMR (400 MHz, DMSO-d6) δ 7.93 (d, 2H), 7.06 (d, 2H), 4.40-4.46 (m, 1H), 4.05-4.15 (m, 3H), 3.75-3.78 (m, 1H), 2.52 (s, 3H), 1.36 (s, 3H), 1.31 (s, 3H).

### Step B. Preparation of ethyl 4-(4-((2,2-dimethyl-1,3-dioxolan-4-yl)methoxy)phenyl)-2,4-dioxobutanoate

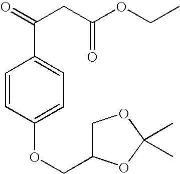

Sodium (0.648 g, 28.2 mmol) was added to ethanol (30 mL) and the mixture was stirred until the sodium completely dissolved. 1-(4-((2,2-dimethyl-1,3-dioxolan-4-yl)methoxy)phenyl)ethanone (2.352 g, 9.40 mmol) and diethyl oxalate (1.914 mL, 14.10 mmol) were added to the solution. The mixture was heated to reflux for 2 hours. After cooling to ambient temperature, the mixture was diluted with EtOAc and washed with 2M HCl solution. The organic layer was concentrated to dryness and purified via Analogix (5-55% EtOAc/heptane over 30 minutes) to give 4-(4-((2,2-dimethyl-1,3-dioxolan-4-yl)methoxy)phenyl)-2,4-dioxobutanoate as a light yellow solid (2.074 g, 63%). LCMS (Table 1, Method b) Rₜ=2.31 min, m/z 351.29 (M+H)⁺; ¹H NMR (400 MHz, DMSO-d6) δ 8.08 (d, 2H), 7.13 (d, 2H), 4.44 (m, 1H), 4.32 (q, 2H), 4.09-4.20 (m, 3H), 3.77 (m, 1H), 1.36 (s, 3H), 1.32 (s, 3H), 1.31 (t, 3H).

### Step C. Preparation of ethyl 5-(4-(2,3-dihydroxypropoxy)phenyl)isoxazole-3-carboxylate

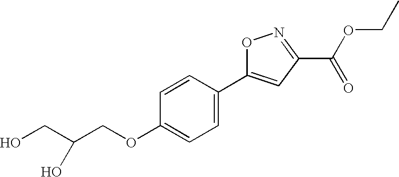

A mixture of ethyl 4-(4-((2,2-dimethyl-1,3-dioxolan-4-yl)methoxy)phenyl)-2,4-dioxobutanoate (2.074 g, 5.92 mmol) and hydroxylamine (HCl salt, 1.224 g, 17.76 mmol) in ethanol (20 mL) was heated to reflux for 1 hour. After cooling to ambient temperature, the crude reaction was diluted with EtOAc (200 mL) and washed with water (200 mL). The aqueous layer was back extracted with EtOAc (3×20 mL). Organic layers were combined, dried over Na₂SO₄ and concentrated to dryness to give ethyl 5-(4-(2,3-dihydroxypropoxy)phenyl)isoxazole-3-carboxylate (1.9 g, 104%) as a white solid. LCMS (Table 1, Method b) Rₜ=1.74 min (no ionization).

### Step D. Preparation of ethyl 5-(4-((2,2-dimethyl-1,3-dioxolan-4-yl)methoxy)phenyl)isoxazole-3-carboxylate

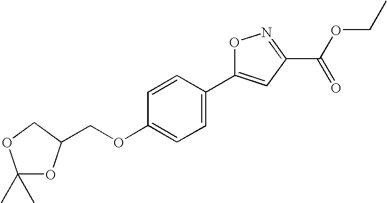

A solution of ethyl 5-(4-(2,3-dihydroxypropoxy)phenyl)isoxazole-3-carboxylate (1.9 g, 6.18 mmol) and toluene-4-sulfonic acid hydrate (0.118 g, 0.618 mmol) in 2,2-dimethoxypropane (19.01 mL, 155 mmol) was stirred at ambient temperature for 2 hours. The precipitate was collected by filtration and washed with ether to give 0.85 g of (S)-ethyl-5-(4-((2,2-dimethyl-1,3-dioxolan-4-yl)methoxy)phenyl)isoxazole-3-carboxylate as a white solid. The filtrate was concentrated and purified via Analogix (0-50% EtOAc/heptane over 30 minutes; RS-120 Si column) to give 1.1 g more of (S)-ethyl 5-(4-((2,2-dimethyl-1,3-dioxolan-4-yl)methoxy)phenyl)isoxazole-3-carboxylate as light yellow solid (90.8% combined yield). LCMS (Table 1, Method b) Rₜ=2.31 min, m/z 348.21 (M+H)⁺; ¹H NMR (400 MHz, DMSO-d6) δ 7.90 (d, 2H), 7.35 (s, 1H), 7.13 (d, 2H), 4.32 (q, 2H), 4.42 (m, 3H), 4.11 (m, 3H), 3.77 (m, 1H), 1.34 (m, 9H).

### Step E. Preparation of 5-(4-((2,2-dimethyl-1,3-dioxolan-4-yl)methoxy)phenyl)isoxazole-3-carboxylic acid

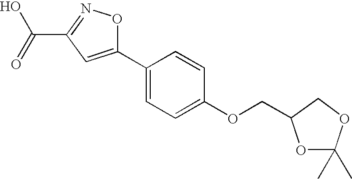

To a solution of ethyl 5-(4-((2,2-dimethyl-1,3-dioxolan-4-yl)methoxy)phenyl)isoxazole-3-carboxylate (1 g, 2.88 mmol) in THF (20 mL) and MeOH (5.00 mL) was added 2M potassium hydroxide (7.20 mL, 14.39 mmol). The mixture was heated at 60° C. for 2 hours. The pH of the mixture was adjusted to 3-4 with 2M HCl and then extracted with DCM (3×50 mL). Organic layers were combined, dried over Na₂SO₄ and concentrated to dryness to give (S)-5-(4-((2,2-dimethyl-1,3-dioxolan-4-yl)methoxy)phenyl)isoxazole-3-carboxylic acid (819 mg, 89%) as a light yellow solid. LCMS (Table 1, Method b) Rₜ=1.59 min, m/z 320.17 (M+H)⁺.

### Step F. Preparation of N-(3-chloro-4-isopropoxyphenyl)-5-(4-((2,2-dimethyl-1,3-dioxolan-4-yl)methoxy)phenyl)isoxazole-3-carboxamide

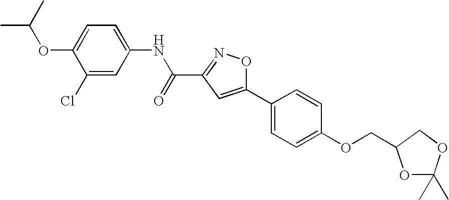

To a solution of 5-(4-((2,2-dimethyl-1,3-dioxolan-4-yl)methoxy)phenyl)-isoxazole-3-carboxylic acid (200 mg, 0.626 mmol) in DMF (5 mL) was added DIEA (0.328 mL, 1.879 mmol). The mixture was stirred at ambient temperature for 5 minutes and then HATU (357 mg, 0.940 mmol) and 3-chloro-4-isopropoxyaniline (128 mg, 0.689 mmol) were added. The mixture was stirred at ambient temperature for 30 minutes and then diluted with EtOAc (100 mL), washed with water. The organic layer was concentrated to afford a residue that was purified via Analogix (0-50% EtOAc/heptane over 30 minutes; RS-80 Si column) to give (S)-N-(3-chloro-4-isopropoxyphenyl)-5-(4-((2,2-dimethyl-1,3-dioxolan-4-yl)methoxy)phenyl)isoxazole-3-carboxamide as a white solid (210 mg, 68.9%). LCMS (Table 1, Method b) Rₜ=2.49 min, m/z 485.26 (M−H)⁻; ¹H NMR (400 MHz, DMSO-d6) δ 10.74 (s, 1H), 7.94 (d, 1H), 7.90 (d, 2H), 7.69 (dd, 1H), 7.34 (s, 1H), 7.19 (d, 1H), 7.15 (d, 1H), 4.62 (m, 1H), 4.44 (m, 1H), 4.11 (m, 3H), 3.78 (m, 1H), 1.37 (s, 3H), 1.32 (s, 3H), 1.29 (d, 6H).

### Step G. Preparation of (R)-N-(3-chloro-4-isopropoxyphenyl)-5-(4-(2,3-dihydroxypropoxy)phenyl)isoxazole-3-carboxamide

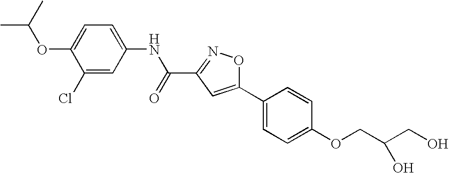

A round bottom flask was charged with (S)-N-(3-chloro-4-isopropoxyphenyl)-5-(4-((2,2-dimethyl-1,3-dioxolan-4-yl)methoxy)phenyl)isoxazole-3-carboxamide (210 mg, 0.431 mmol) and toluene-4-sulfonic acid hydrate (16.41 mg, 0.086 mmol) in MeOH (5.0 mL) to give a white suspension. The reaction mixture was heated to 70° C. for 6 hours. After cooling to ambient temperature the mixture was filtered and the filtrate was concentrated, then purified via HPLC to give (R)-N-(3-chloro-4-isopropoxyphenyl)-5-(4-(2,3-dihydroxypropoxy)phenyl)-isoxazole-3-carboxamide (85.1 mg, 0.190 mmol, 44.2% yield) as a white solid. LCMS (Table 1, Method a) Rₜ=2.72 min, m/z 447.21 (M+H)⁺; ¹H NMR (400 MHz, DMSO-d6) δ 10.74 (s, 1H), 7.94 (d, 1H), 7.90 (d, 2H), 7.68 (dd, 1H), 7.32 (s, 1H), 7.19 (d, 1H), 7.12 (d, 2H), 4.99 (d, 1H), 4.70 (t, 1H), 4.63 (m, 1H), 4.10 (dd, 1H), 3.96 (dd, 1H), 3.82 (m, 1H), 3.46 (t, 2H), 1.28 (d, 6H).

### Preparation of 2-(3-chloro-4-(1-phenyl-5-(trichloromethyl)-1H-1,2,4-triazole-3-carboxamido)phenylsulfonamido)acetic acid

### Example #8

### Step A. Preparation of methyl 2-(4-acetamido-3-chlorophenylsulfonamido)acetate

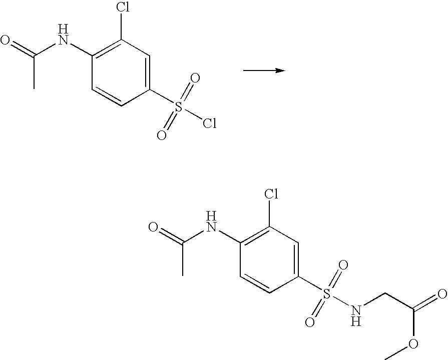

To a suspension of amino-acetic acid methyl ester hydrochloride (0.937 g, 7.46 mmol) in DCM (37.3 mL) was added triethylamine (2.183 mL, 15.66 mmol). 4-Acetamido-3-chlorobenzene-1-sulfonyl chloride (1 g, 3.73 mmol) was added in one portion and the reaction mixture was stirred at ambient temperature overnight. The reaction mixture was washed with HCl (10%, 25 mL). The aqueous layer was extracted with DCM (20 mL). The combined DCM layers was washed with water (20 mL) and brine (20 mL). The organic layer was dried over MgSO₄, filtered and concentrated to afford methyl 2-(4-acetamido-3-chlorophenylsulfonamido)acetate (0.93 g, 2.61 mmol, 70.0% yield) as a pale yellow solid. LCMS (Table 1, Method a) Rₜ=2.46 min, MS m/z: 321.06 (M+H)⁺; ¹H NMR (400 MHz, DMSO-d6) δ 9.72 (s, 1H), 8.30 (t, 1H, J=6.20 Hz), 8.05 (d, 1H, J=8.62 Hz), 7.84 (d, 1H, J=2.06 Hz), 7.70 (dd, 1H, J=8.47, 2.03 Hz), 3.76 (d, 2H, J=6.21 Hz), 3.53 (s, 3H), 2.16 (s, 3H).

### Step B. Preparation of methyl 2-(4-amino-3-chlorophenylsulfonamido)acetate

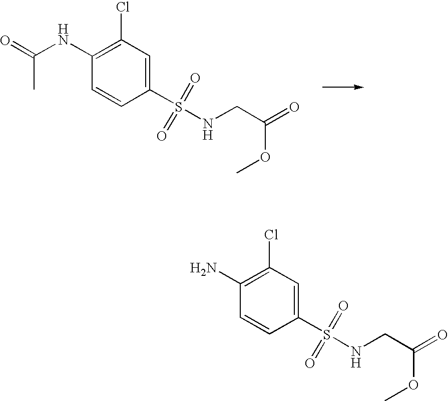

To a suspension of tert-butyl 2-(4-acetamido-3-chlorophenylsulfonamido)acetate (0.13 g, 0.358 mmol) in MeOH (6 mL) was added sulfuric acid (0.070 mL, 1.311 mmol). The reaction mixture was heated at reflux overnight and then allowed to cool down. The colorless solution was concentrated and the residue was partitioned between EtOAc (50 mL) and water (50 mL). The EtOAc layer was washed with water (3×20 mL). The organic layer was dried over Na₂SO₄, filtered and concentrated to afford methyl 2-(4-amino-3-chlorophenylsulfonamido)acetate (0.1 g, 0.358 mmol, 100% yield) as an orange oil. LCMS (Table 1, Method a) Rₜ=2.58 min, MS m/z: 279.01 (M+H)⁺; ¹H NMR (400 MHz, DMSO-d6) δ 7.87 (t, 1H, J=6.25 Hz), 7.54 (d, 1H, J=2.40 Hz), 7.39 (dd, 1H, J=2.15, 8.58 Hz), 6.83 (d, 1H, J=8.53 Hz), 6.20 (d, 2H, J=5.61 Hz) 3.62 (d, 2H, J=6.17 Hz), 3.54 (s, 3H).

### Step C. Preparation of methyl 2-(3-chloro-4-(1-phenyl-5-(trichloromethyl)-1H-1,2,4-triazole-3-carboxamido)phenylsulfonamido)acetate

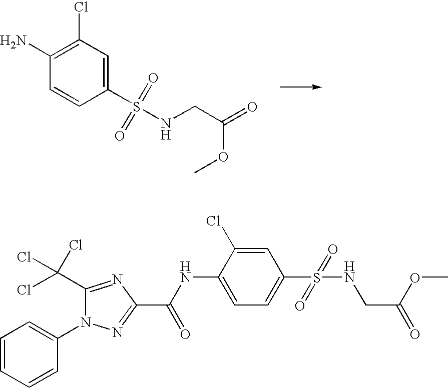

To a suspension of 1-phenyl-5-(trichloromethyl)-1H-1,2,4-triazole-3-carboxylic acid (0.194 g, 0.631 mmol) in pyridine (3.4 mL) was added methyl 2-(4-amino-3-chlorophenylsulfonamido)acetate (0.16 g, 0.574 mmol). The reaction mixture was cooled in a dry ice-ethylene glycol bath (−10° C.). Phosphorus oxychloride (0.107 mL, 1.148 mmol) was added dropwise over 5 min. The reaction mixture was stirred at −10° C. for 1 h. Ice-cold water (1 mL) was added to quench the reaction. The reaction mixture was partitioned between DCM (30 mL) and HCl (10%, 30 mL). The aqueous layer was extracted with DCM (30 mL) and the combined organic layers washed with HCl (10%, 20 mL) and water (20 mL) and concentrated to afford an orange oil, which was purified via silica gel chromatography (12 g, 50% EtOAc:Heptane) to afford methyl 2-(3-chloro-4-(1-phenyl-5-(trichloromethyl)-1H-1,2,4-triazole-3-carboxamido)phenylsulfonamido)-acetate (0.26 g, 0.458 mmol, 80% yield) as a white solid. LCMS (Table 1, Method a) Rₜ=3.96 min, MS m/z: 567.97 (M+H)⁺; ¹H NMR (400 MHz, DMSO-d6) δ 10.40 (s, 1H), 8.42 (t, 1H, J=6.15 Hz), 8.10-8.15 (m, 1H), 7.95 (d, 1H, J=2.05 Hz), 7.76-7.83 (m, 3H), 7.63-7.71 (m, 3H), 3.81 (d, 2H, J=5.91 Hz), 3.54 (s, 3H).

### Step D. Preparation of 2-(3-chloro-4-(1-phenyl-5-(trichloromethyl)-1H-1,2,4-triazole-3-carboxamido)phenylsulfonamido)acetic acid

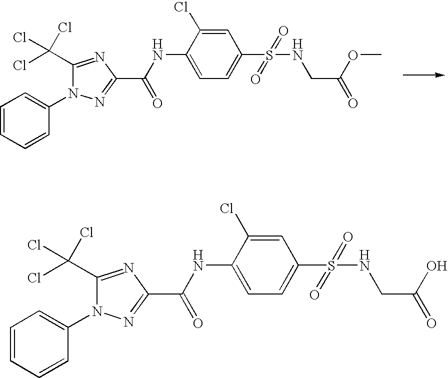

To a solution of methyl 2-(3-chloro-4-(1-phenyl-5-(trichloromethyl)-1H-1,2,4-triazole-3-carboxamido)phenylsulfonamido)acetate (0.255 g, 0.450 mmol) in MeOH (9 mL) was added dropwise a solution of sodium hydroxide (0.294 g, 7.35 mmol) in water (9 mL). The reaction mixture was stirred at ambient temperature overnight. Methanol was removed under reduced pressure and the aqueous solution was acidified with HCl (10%) to pH=2. The resulting solid was collected by vacuum filtration and washed with water to afford 0.14 g of a white solid, which was purified via semi-Prep HPLC to afford 2-(3-chloro-4-(1-phenyl-5-(trichloromethyl)-1H-1,2,4-triazole-3-carboxamido)phenyl-sulfonamido)acetic acid (54 mg, 0.098 mmol, 21.8% yield) as a white solid. LCMS (Table 1, Method a) Rₜ=3.01 min, MS m/z: 553.96 (M+H)⁺; ¹H NMR (400 MHz, DMSO-d6) δ 8.14 (d, 1H, J=8.52 Hz), 7.98 (d, 1H, J=2.06 Hz), 7.83 (dd, 1H, J=2.08, 8.52 Hz), 7.75-7.78 (m, 2H), 7.61-7.71 (m, 3H), 3.30 (s, 2H).

### Preparation of N-(2-chlorophenyl)-1-(3,4-dichlorophenyl)-5-(trifluoromethyl)-1H-pyrazole-3-carboxamide

### Example #9

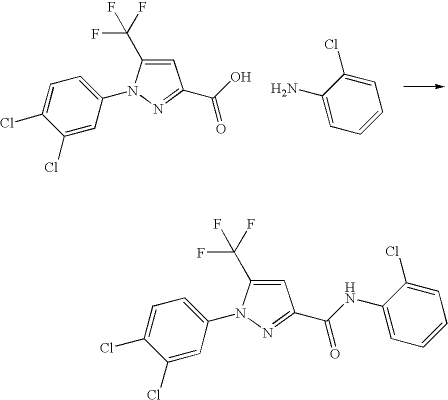

1-(3,4-Dichlorophenyl)-5-(trifluoromethyl)-1H-pyrazole-3-carboxylic acid (0.100 g, 0.308 mmol) (ABCR) was dissolved in DMF (1.5 mL) open to air. To this was added DIEA (0.204 mL, 1.168 mmol) dropwise. The mixture was stirred for a few minutes then HATU (0.222 g, 0.584 mmol) was added. The resulting mixture was stirred for 15 mins, then 2-chloroaniline (0.041 mL, 0.390 mmol) was added dropwise. The reaction was stirred at ambient temperature for 16 hours then heated at 60° C. overnight. The crude reaction was diluted with DCM (100 mL). The organic phase was washed with aqueous 1M HCl solution (75 mL), then aqueous 1M NaOH solution (75 mL), washed with brine (75 mL), dried MgSO₄) and concentrated to yield crude brown oil. The residue was purified via Analogix FC system using RediSep® RS 40 g column, with a gradient of 0-50% EtOAc/Heptane over 50 min. at 30 mL/min. to give N-(2-chlorophenyl)-1-(3,4-dichlorophenyl)-5-(trifluoromethyl)-1H-pyrazole-3-carboxamide (40 mg, 0.08 mmol) as an off-white solid. LCMS (Table 1, Method c) Rₜ=3.00 min, m/z 434.04 (M+H)⁺. ¹H NMR (400 MHz, CD₂Cl₂) δ ppm 8.44 (d, J=8.1 Hz, 1H), 8.07 (s, 2H), 7.65 (m, 2H), 7.46 (dd, J=1.3, 8.0 Hz, 1H), 7.36 (ddd, J=1.8, 6.1, 8.6 Hz, 2H), 7.15 (dt, J=1.4, 7.9, 7.9 Hz, 1H).

### Preparation of 4-methyl-5-o-tolyl-2-(4-(trifluoromethyl)phenyl)thiazole

### Example #10

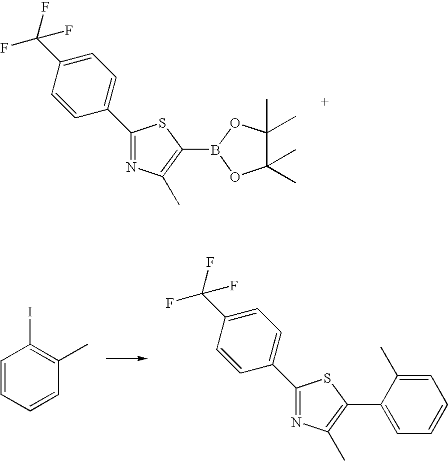

4-methyl-5-(4,4,5,5-tetramethyl-1,3,2-dioxaborolan-2-yl)-2-(4-(trifluoromethyl)phenyl)thiazole (186 mg, 0.505 mmol), Pd Tetrakis (31.8 mg, 0.028 mmol), Cesium carbonate (448 mg, 1.376 mmol) and 1-iodo-2-methylbenzene (100 mg, 0.459 mmol) were weighed into a vial, to which DME (5 mL) and Water (2.5 mL) were added. The mixture was heated at 80° C. for 3 hours. The mixture was diluted with DCM and washed with water (3×10 mL). The organic layer was concentrated to dryness to give an yellow oil. The residue purified by flash chromatography (0-100% DCM/Heptane; RS-12 Si column) to give 4-methyl-5-o-tolyl-2-(4-(trifluoromethyl)phenyl)thiazole (93.6 mg, 0.281 mmol, 61.2% yield) as an oil. LCMS (Table, 1, Method a) Rₜ=4.38 min, m/z 334.12 (M+H)⁺. ¹H NMR (400 MHz, DMSO-d₆) δ 8.16 (d, J=8.7, 2H), 7.87 (d, J=8.2, 2H), 7.40 (d, J=1.3, 1H), 7.39 (d, J=1.5, 1H), 7.37-7.28 (m, 2H), 2.25 (s, 3H), 2.23 (s, 3H).

### Preparation of N-(5-phenyl-1,2,4-oxadiazol-3-yl)benzamide

### Example # 11

### Step A. Preparation of (Z)-methyl N-cyanobenzimidate

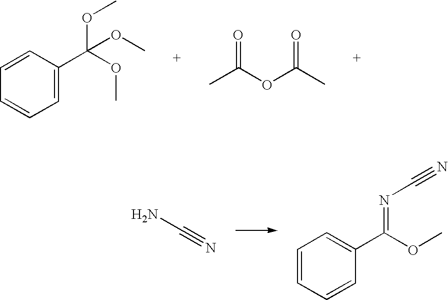

To a mixture of (trimethoxymethyl)benzene (3.44 mL, 20 mmol) and cyanamide (0.842 g, 20 mmol) was added acetic anhydride (3.78 mL, 40 mmol). The mixture was heated to 135° C. for 45 min. Acetic acid produced in the reaction was removed under reduced pressure. The residue was purified with flash chromatography with 0-40% ethyl acetate/heptane over 20 minutes, then kept 40% ethyl acetate/heptane for 5 minutes to give (Z)-methyl N-cyanobenzimidate colorless oil (2.54 g, 79.2% yield). ¹H NMR (400 MHz, CDCl₃) δ 8.00-7.95 (m, 2H), 7.76-7.70 (m, 1H), 7.66-7.60 (m, 2H), 4.03 (m, 3H).

### Step B. Preparation of 5-phenyl-1,2,4-oxadiazol-3-amine

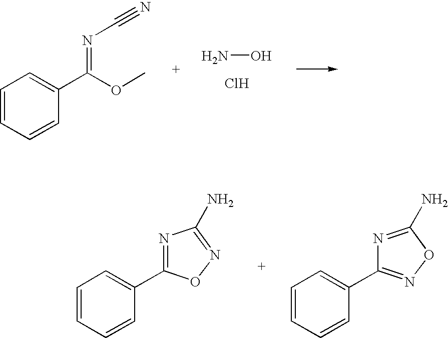

To a solution of (Z)-methyl N-cyanobenzimidate (2.52 g, 15.73 mmol) in methanol (30 mL) was added hydroxylamine hydrochloride (1.093, 15.73 mmoL) followed by triethylamine (2.193 mL, 15.73 mmol). The clear solution turned warm after ˜5 minutes of stirring. Continued stirring at room temperature for 1 hour and then white precipitate was formed. Solvent was removed and water was added to the residue. The solid was collected by filtration and washed with water. The crude product was purified by flash chromatography with 15-45% ethyl acetate/heptane over 40 minutes. Two peaks were collected separately. The early eluting peak was concentrated to give 5-phenyl-1,2,4-oxadiazol-3-amine (0.95 g, 37.5% yield). ¹H NMR (400 MHz, DMSO-d₆) δ 8.03-7.95 (m, 2H), 7.69-7.63 (m, 1H), 7.63-7.56 (m, 2H), 6.40 (s, 2H). The second peak was concentrated to give 3-phenyl-1,2,4-oxadiazol-5-amine (0.15 g, 5.9% yield). ¹H NMR (400 MHz, DMSO-d₆) δ 7.92 (s, 2H), 7.90-7.84 (m, 2H), 7.55-7.46 (m, 3H).

### Step C. Preparation of N-(5-phenyl-1,2,4-oxadiazol-3-yl)benzamide

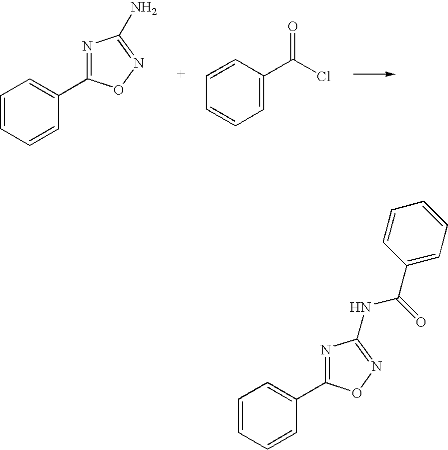

To a solution of 5-phenyl-1,2,4-oxadiazol-3-ylamine (32 mg, 0.2 mmol) in pyridine (1 mL) in a CEM microwave was added benzoyl chloride (0.046 mL, 0.4 mmol). The mixture was heated at 150° C. for 10 minutes. Solvent was removed under reduced pressure and the residue was purified by mass triggered HPLC to give N-(5-phenyl-1,2,4-oxadiazol-3-yl)benzamide (40 mg, 74.5% yield). LCMS (Table, 1, Method a) Rₜ=2.43 min, m/z 266.12 (M+H)⁺. ¹H NMR (400 MHz, DMSO-d₆) δ 8.15-8.10 (m, 2H), 8.07-8.02 (m, 2H), 7.77-7.71 (m, 1H), 7.70-7.62 (m, 3H), 7.59-7.52 (m, 2H).

### Preparation of N-(3-phenyl-1,2,4-oxadiazol-5-yl)benzamide

### Example #12

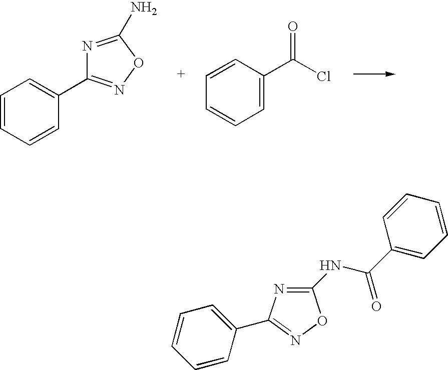

To a solution of 3-phenyl-1,2,4-oxadiazol-5-ylamine (16.1 mg, 0.1 mmol) in pyridine (0.5 mL) in a CEM microwave was added benzoyl chloride (0.023 mL, 0.2 mmol). The mixture was heated at 150° C. for 10 minutes. Solvent was removed under reduced pressure and the residue was purified by mass triggered HPLC to give N-(3-phenyl-1,2,4-oxadiazol-5-yl)benzamide (4 mg, 15.5% yield). LCMS (Table, 1, Method a) Rₜ=2.57 min, m/z 266.13 (M+H)⁺. ¹H NMR (400 MHz, DMSO-d₆) δ 8.15-8.10 (m, 2H), 8.08-8.02 (m, 2H), 7.78-7.71 (m, 1H), 7.70-7.61 (m, 3H), 7.59-7.52 (m, 2H).

### Preparation of 3-(3-chloro-4-(5-(3-chloro-4-isopropoxyphenyl)isoxazole-3-carboxamido)benzylamino)-2-methylpropanoic acid

### Example #13

### Step A. Preparation of 1-(3-chloro-4-isopropoxyphenyl)ethanone

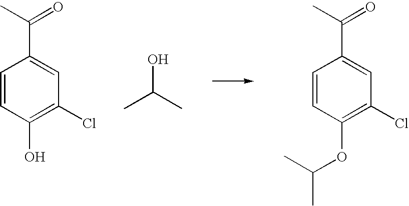

A solution of 1-(3-chloro-4-hydroxyphenyl)ethanone (6.658 g, 39.0 mmol) and triphenylphosphine (16.38 g, 62.4 mmol) in THF (20 mL) was cooled to 0° C., to which DIAD (12.14 mL, 62.4 mmol) was added dropwise. The mixture was stirred for 5 minutes and then propan-2-ol (2.93 g, 48.8 mmol) was added dropwise. The mixture was stirred at rt over the weekend. Solvent was removed under reduced pressure. The residue was triturated with heptane. The white solid was filtered off and washed with ether. The filtrate was concentrated to dryness to give an oil which was purified by flash chromatography (0-50% EtOAc/heptane over 30 min; RS-120 Si column) to give 1-(3-chloro-4-isopropoxyphenyl)ethanone (6.9 g, 32.4 mmol, 83% yield) as a white solid. LCMS (Table, 1, Method b) Rₜ=2.31 min, m/z 213.12 (M+H)⁺. ¹H NMR (400 MHz, DMSO-d₆) δ 7.97 (d, J=2.2, 1H), 7.90 (dd, J=2.2, 8.7, 1H), 7.28 (d, J=8.7, 1H), 4.83 (hept, J=6.1, 1H), 2.53 (s, 3H), 1.33 (d, J=6.0, 6H).

### Step B. Preparation of ethyl 4-(3-chloro-4-isopropoxyphenyl)-2,4-dioxobutanoate

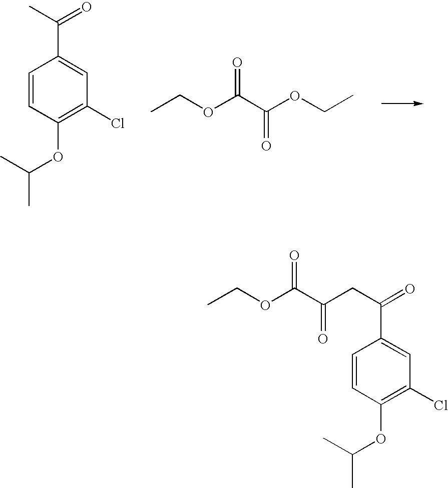

Sodium (1.913 g, 83 mmol) was added to ethanol (90 mL) and the mixture was stirred until the metal completely dissolved. 1-(3-chloro-4-isopropoxyphenyl)ethanone (5.9 g, 27.7 mmol) and diethyl oxalate (5.65 mL, 41.6 mmol) were added to the solution. The mixture was heated to reflux for 2 hours. Cooled down to room temperature and the crude mixture was diluted with EtOAc, washed with water. Organic layer was concentrated to dryness and purified by flash chromatography (0-40% EtOAc/heptane over 40 min; RS-120 Si column) to give ethyl 4-(3-chloro-4-isopropoxyphenyl)-2,4-dioxobutanoate (8.65 g, 27.7 mmol, 100% yield) as a brown oil. LCMS (Table, 1, Method b) Rₜ=2.60 min, m/z 313.19 (M+H)⁺. ¹H NMR (400 MHz, DMSO-d₆) δ 8.13 (d, J=2.2, 1H), 8.06 (dd, J=2.2, 8.8, 11H), 7.33 (d, J=8.9, 1H), 7.12 (s, 1H), 4.93-4.82 (m, 1H), 4.32 (q, J=7.1, 2H), 1.35 (d, J=6.1, 6H), 1.31 (t, J=7.1, 3H).

### Step C. Preparation of ethyl 5-(3-chloro-4-isopropoxyphenyl)isoxazole-3-carboxylate

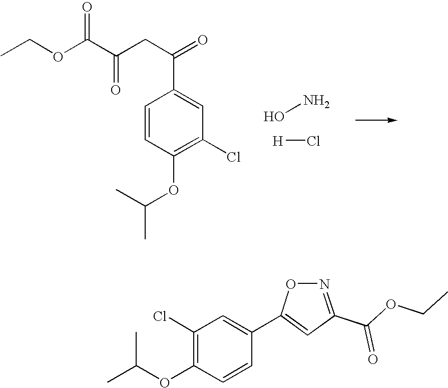

Ethyl 4-(3-chloro-4-isopropoxyphenyl)-2,4-dioxobutanoate (8.65 g, 27.7 mmol) and hydroxylamine hydrochloride (5.77 g, 83 mmol) in Ethanol (90 mL) was heated to reflux for 1 hour. Cooled down to room temperature and the crude reaction was partitioned between EtOAc and water. Organic layer was concentrated to dryness to give a light brown solid which was purified by flash chromatography (0-40% EtOAc/heptane over 40 min) to give ethyl 5-(3-chloro-4-isopropoxyphenyl)isoxazole-3-carboxylate (7.3 g, 23.57 mmol, 85% yield) as a white solid. LCMS (Table, 1, Method b) Rₜ=2.54 min, m/z 310.19 (M+H)⁺. ¹H NMR (400 MHz, DMSO-d₆) δ 8.06 (d, J=2.1, 1H), 7.88 (dd, J=2.1, 8.7, 1H), 7.47 (s, 1H), 7.35 (d, J=8.8, 1H), 4.87-4.76 (m, 1H), 4.39 (q, J=7.1, 2H), 1.35 (t, J=7.3, 3H), 1.32 (d, J=5.9, 6H).

### Step D. Preparation of 5-(3-chloro-4-isopropoxyphenyl)isoxazole-3-carboxylic acid

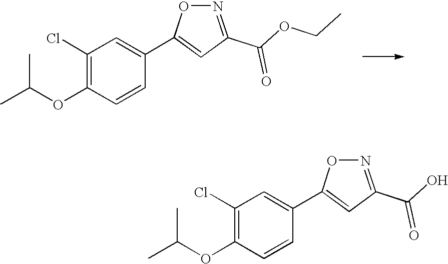

To a solution of ethyl 5-(3-chloro-4-isopropoxyphenyl)isoxazole-3-carboxylate (1 g, 3.23 mmol) in THF (24 mL) was added 2.0 M sodium hydroxide solution (8.07 mL, 16.14 mmol). The mixture was heated at 65° C. for 2 hours. Organic solvent was removed under reduced pressure. The aqueous solution was acidified with 1M HCl. Extracted with EtOAc (3×25 mL). Organic layer was dried over Na₂SO₄ and concentrated to dryness to give 5-(3-chloro-4-isopropoxyphenyl)isoxazole-3-carboxylic acid (0.874 g, 3.10 mmol, 96% yield) as a white solid. LCMS (Table, 1, Method b) Rₜ=1.66 min, m/z 282.14 (M+H)⁺. ¹H NMR (400 MHz, DMSO-d₆) δ 8.03 (d, J=2.2, 1H), 7.86 (dd, J=2.2, 8.7, 1H), 7.38 (s, 1H), 7.34 (d, J=8.9, 1H), 4.86-4.76 (m, 1H), 1.33 (d, J=6.0, 6H).

### Step E. Preparation of N-(2-chloro-4-formylphenyl)-5-(3-chloro-4-isopropoxyphenyl)isoxazole-3-carboxamide

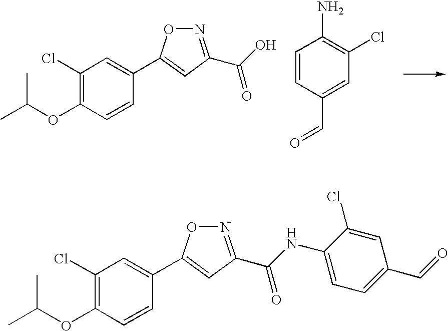

5-(3-chloro-4-isopropoxyphenyl)isoxazole-3-carboxylic acid (300 mg, 1.065 mmol) and 4-amino-3-chlorobenzaldehyde (151 mg, 0.968 mmol) in anhydrous pyridine (3 mL) was cooled to −15° C. with a ice-acetone bath. To the reaction mixture was added phosphorus oxychloride (0.186 mL, 2.000 mmol) dropwise with vigorous stirring. The reaction mixture was stirred at −15° C. for another 1 hour and was then quenched with ice water. Extracted with ethyl acetate (25×3 mL). Organic layers were combined and washed with 1N HCl and brine, dried over sodium sulfate and evaporated to dryness to give an orange solid. The residue was triturated with a small amount of ether to give N-(2-chloro-4-formylphenyl)-5-(3-chloro-4-isopropoxyphenyl)isoxazole-3-carboxamide (226 mg, 55.7% yield) as a light brown solid. LCMS (Table, 1, Method b) Rₜ=2.80 min, m/z 417.09 (M−H)⁻. ¹H NMR (400 MHz, DMSO-d₆) δ 10.35 (s, 1H), 9.98 (s, 1H), 8.14 (d, J=8.3, 1H), 8.11 (d, J=1.5, 1H), 8.08 (d, J=2.1, 1H), 7.96 (dd, J=1.6, 8.3, 1H), 7.91 (dd, J=2.0, 8.6, 1H), 7.54 (s, 1H), 7.37 (d, J=8.7, 1H), 4.88-4.77 (m, 1H), 1.34 (d, J=5.9, 6H).

### Step F. Preparation of 3-(3-chloro-4-(5-(3-chloro-4-isopropoxyphenyl)isoxazole-3-carboxamido)benzylamino)-2-methylpropanoic acid

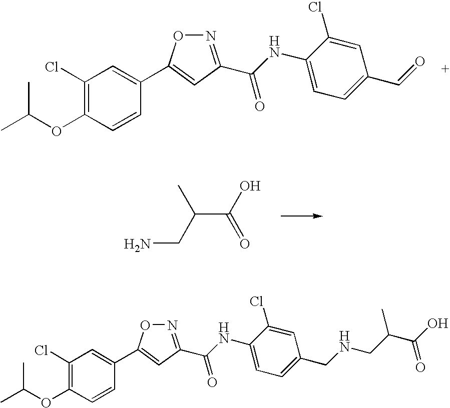

A mixture of N-(2-chloro-4-formylphenyl)-5-(3-chloro-4-isopropoxyphenyl)-isoxazole-3-carboxamide (30 mg, 0.072 mmol) and 3-aminobutanoic acid (7.4 mg, 0.072 mmol), AcOH (0.020 mL, 0.358 mmol) in DCM (3 mL) and MeOH (3.00 mL) was stirred at 40° C. overnight. Sodium cyanoborohydride (4.50 mg, 0.072 mmol) was added. The mixture was stirred at room temperature for 8 hours. Solvent was removed under reduced pressure. Product was purified by mass triggered HPLC to give 3-(3-chloro-4-(5-(3-chloro-4-isopropoxyphenyl)isoxazole-3-carboxamido)benzylamino)butanoic acid (13.2 mg, 36.4% yield). LCMS (Table, 1, Method a) Rₜ=2.54 min, m/z 504.16 (M−H)⁻. ¹H NMR (400 MHz, DMSO-d₆) δ 10.28 (s, 1H), 8.06 (d, J=2.1, 1H), 7.89 (dd, J=1.8, 8.5, 1H), 7.65 (d, J=8.2, 1H), 7.56 (d, J=1.0, 1H), 7.47 (s, 1H), 7.36 (d, J=9.0, 2H), 4.82 (dt, J=5.9, 12.1, 1H), 4.82 (dt, J=5.9, 12.1, 1H), 3.78 (s, 2H), 2.70-2.60 (m, 2H), 2.73-2.39 (m, 1H), 1.34 (d, J=6.0, 6H), 1.04 (d, J=7.0, 3H).

### Preparation of 1-(3-chloro-4-(5-(3-chloro-4-isopropoxyphenyl)isoxazole-3-carboxamido)benzyl)azetidine-3-carboxylic acid

### Example #14

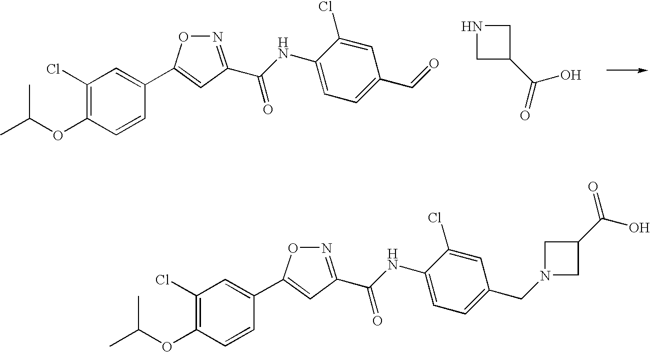

To a suspension of N-(2-chloro-4-formylphenyl)-5-(3-chloro-4-isopropoxyphenyl)isoxazole-3-carboxamide (100 mg, 0.239 mmol) (Example #13, Step E) in MeOH (4.00 mL) and DCE (4 mL) was added AcOH (0.068 mL, 1.193 mmol) followed by azetidine-3-carboxylic acid (48.2 mg, 0.477 mmol). The mixture was stirred at 40° C. for 4 hours and sodium cyanoborohydride (14.99 mg, 0.239 mmol) was added in one portion. The mixture was stirred at room temperature overnight. Solvent was removed under reduced pressure and the residue was purified via HPLC (10-95% ACN/50 mM ammonium acetate over 30 min) to give 1-(3-chloro-4-(5-(3-chloro-4-isopropoxyphenyl)isoxazole-3-carboxamido)benzyl)azetidine-3-carboxylic acid (75 mg, 0.149 mmol, 62.3% yield) as a white powder. LCMS (Table, 1, Method a) Rₜ=2.51 min, m/z 502.17 (M−H)⁻. ¹H NMR (400 MHz, DMSO-d₆) δ 10.26 (s, 1H), 8.06 (d, J=2.1, 1H), 7.89 (dd, J=2.2, 8.6, 1H), 7.63 (d, J=8.3, 1H), 7.47 (s, 1H), 7.45 (d, J=1.5, 1H), 7.36 (d, J=8.8, 1H), 7.29 (dd, J=1.1, 7.9, 1H), 4.82 (dt, J=5.9, 12.0, 1H), 3.56 (s, 2H), 3.45-3.38 (m, 2H), 3.24-3.16 (m, 3H), 1.34 (d, J=6.0, 6H).

### Preparation of (R)-N-(3-chloro-4-isopropoxyphenyl)-5-(4-(2,3-dihydroxy-propoxy)phenyl)isoxazole-3-carboxamide

### Example #15

### Step A. Preparation of 1-(4-((2,2-dimethyl-1,3-dioxolan-4-yl)methoxy)-phenyl)ethanone

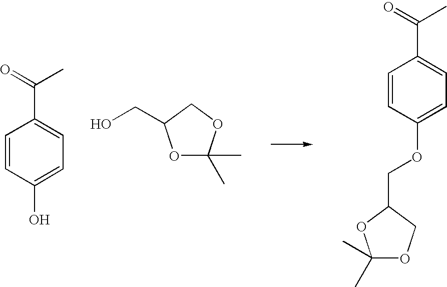

Into a 250 mL round-bottomed flask was added triphenylphosphine (3.13 g, 11.92 mmol) in THF (120 mL) to give a colorless clear solution. The solution was cooled to 0° C. with ice-water bath. diisopropyl azodicarboxylate (2.317 mL, 11.92 mmol) was added dropwise over 10 min. The reaction mixture turned into off white suspension in the process of adding. The reaction mixture was stirred at 0° C. for 30 min. A mixture of 1-(4-hydroxyphenyl)ethanone (1.623 g, 11.92 mmol) and (2,2-dimethyl-1,3-dioxolan-4-yl)methanol (1.407 mL, 11.35 mmol) was added to the mixture over 30 min. Stirred at 0° C. for 2 hours and then room temperature overnight. The reaction mixture was concentrated and the residue was triturated with ether. The white solid was filtered off and the filtrate was concentrated and purified by flash chromatography (0-40% EtOAc/Heptane; RS-120 Si column) to give 1-(4-((2,2-dimethyl-1,3-dioxolan-4-yl)methoxy)phenyl)ethanone (2.352 g, 9.40 mmol, 83% yield) as a white solid. LCMS (Table, 1, Method b) Rₜ=1.97 min, m/z 251.17 (M+H)⁺.

### Step B. Preparation of ethyl 4-(4-((2,2-dimethyl-1,3-dioxolan-4-yl)methoxy)phenyl)-2,4-dioxobutanoate

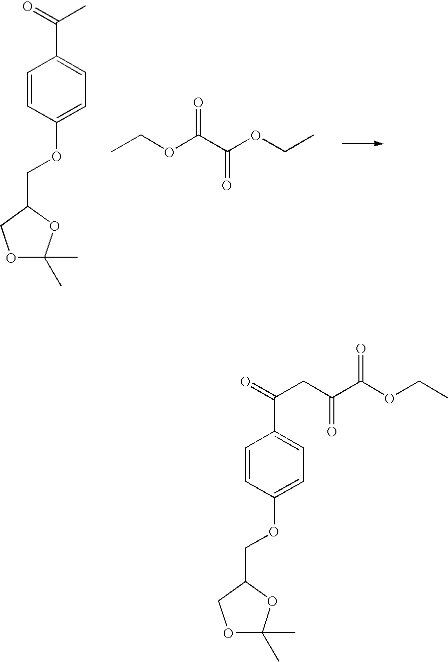

Sodium (0.648 g, 28.2 mmol) was added to ethanol (30 mL) and the mixture was stirred until the metal completely dissolved. 1-(4-((2,2-dimethyl-1,3-dioxolan-4-yl)methoxy)phenyl)ethanone (2.352 g, 9.40 mmol) and diethyl oxalate (1.914 mL, 14.10 mmol) were added to the solution. The mixture was heated to reflux for 2 hours. Cooled down to room temperature and the crude mixture was diluted with EtOAc, washed with 2M HCl solution. Organic layer was concentrated to dryness and purified by flash chromatography (5-55% EtOAc/heptane over 30 minutes) to give a ethyl 4-(4-((2,2-dimethyl-1,3-dioxolan-4-yl)methoxy)phenyl)-2,4-dioxobutanoate (2.074 g, 63% yield) as a light yellow solid. LCMS (Table, 1, Method b) Rₜ=2.31 min, m/z 351.21 (M+H)⁺. ¹H NMR (400 MHz, DMSO-d6) δ 8.08 (d, J=9.0, 2H), 7.13 (d, J=9.0, 2H), 7.09 (s, 1H), 4.44 (qd, J=4.3, 6.4, 1H), 4.23-4.07 (m, 3H), 3.78 (dd, J=6.3, 8.4, 1H), 1.36 (s, 3H), 1.31 (s, 3H).

### Step C. Preparation of ethyl 5-(4-(2,3-dihydroxypropoxy)phenyl)isoxazole-3-carboxylate

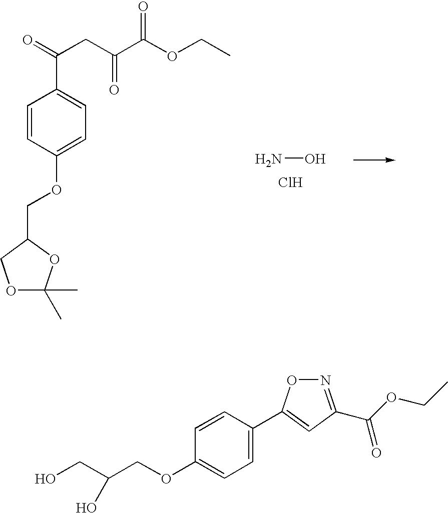

Ethyl 4-(4-((2,2-dimethyl-1,3-dioxolan-4-yl)methoxy)phenyl)-2,4-dioxobutanoate (2.074 g, 5.92 mmol) and hydroxylamine hydrochloride (1.224 g, 17.76 mmol) in ethanol (20 mL) was heated to reflux for 1 h. LCMS indicated the reaction was complete. Cooled down to room temperature and the crude reaction was diluted with EtOAc and washed with water. Aqueous layer was back extracted with EtOAc (3×20 mL). Organic layers were combined, dried over Na2SO4 and concentrated to dryness to give ethyl 5-(4-(2,3-dihydroxypropoxy)phenyl)isoxazole-3-carboxylate (1.9 g, 104% yield) as a white solid. LCMS (Table, 1, Method b) Rₜ=1.74 min, m/z (no ionization).

### Step D. Preparation of (S)-ethyl 5-(4-((2,2-dimethyl-1,3-dioxolan-4-yl)methoxy)phenyl)isoxazole-3-carboxylate

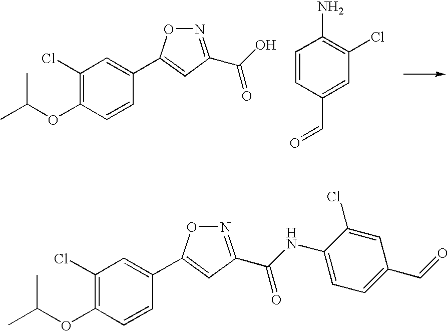

A solution of ethyl 5-(4-(2,3-dihydroxypropoxy)phenyl)isoxazole-3-carboxylate (1.9 g, 6.18 mmol) and Toluene-4-sulfonic acid hydrate (0.118 g, 0.618 mmol) in 2,2-dimethoxypropane (19.01 mL, 155 mmol) was stirred at room temperature for 2 hours. Solvent was removed and the residue was purified by flash chromatography (0-50% EtOAc/heptane over 30 min; RS-120 Si column) to give (S)-ethyl 5-(4-((2,2-dimethyl-1,3-dioxolan-4-yl)methoxy)phenyl)isoxazole-3-carboxylate (1.95 g, 3.17 mmol, 90.8% yield) as light yellow solid. LCMS (Table, 1, Method b) Rₜ=2.31 min, m/z 348.19 (M+H)⁺. ¹H NMR (400 MHz, DMSO-d6) δ 7.90 (d, J=8.8, 2H), 7.34 (s, 1H), 7.13 (d, J=8.9, 2H), 4.45-4.41 (m, 1H), 4.15-4.04 (m, 3H), 3.77 (dd, J=6.3, 8.4, 1H), 1.36 (s, 3H), 1.32-1.30 (s, 3H).

### Step E. Preparation of (S)-5-(4-((2,2-dimethyl-1,3-dioxolan-4-yl)methoxy)phenyl)isoxazole-3-carboxylic acid

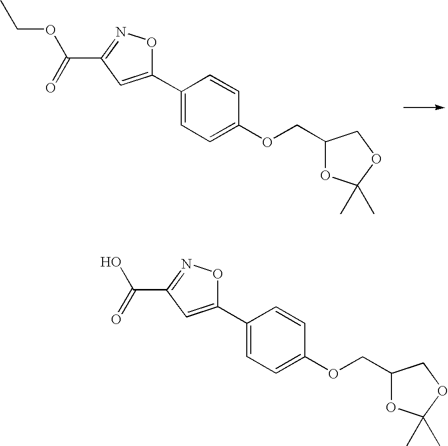

To a solution of (S)-ethyl 5-(4-((2,2-dimethyl-1,3-dioxolan-4-yl)methoxy)phenyl)isoxazole-3-carboxylate (1 g, 2.88 mmol) in THF (20 mL) and MeOH (5.00 mL) was added 2.0 M potassium hydroxide (7.20 mL, 14.39 mmol). The mixture was heated at 60° C. for 2 hours. Adjusted the pH to 34 with 2M HCl solution—extracted with DCM. Organic layers were combined, dried over Na₂SO₄ and evaporated to dryness to give (S)-5-(4-((2,2-dimethyl-1,3-dioxolan-4-yl)methoxy)phenyl)isoxazole-3-carboxylic acid (819 mg, 2.56 mmol, 89% yield) as a light yellow solid. LCMS (Table, 1, Method b) Rₜ=1.59 min, ni/z 320.17 (M+H)⁺.

### Step F. Preparation of (S)-N-(3-chloro-4 isopropoxyphenyl)-5-(4-((2,2-dimethyl-1,3-dioxolan-4-yl)methoxy)phenyl)isoxazole-3-carboxamide

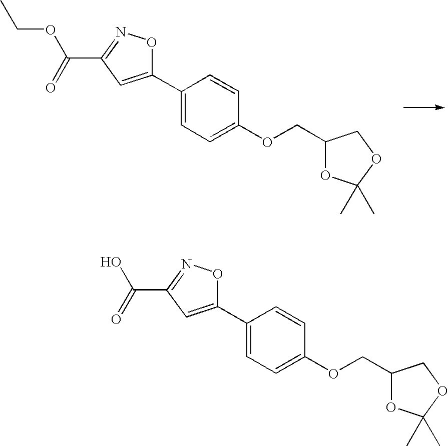

To a suspension of (S)-5-(4-((2,2-dimethyl-1,3-dioxolan-4-yl)methoxy)phenyl)isoxazole-3-carboxylic acid (100 mg, 0.313 mmol) in DCM (2 mL) was added oxalyl chloride (0.188 mL, 0.376 mmol) followed by a drop of DMF. The mixture was stirred at rt for 1. The mixture was concentrated to dryness and then DCM (4 mL) was added. 3-chloro-4-isopropoxyaniline (64.0 mg, 0.344 mmol) and DIEA (0.109 mL, 0.626 mmol) were added to the solution in one portion. The mixture was stirred at rt for 15 minutes and then was concentrated to dryness. The brown residue was triturated with a small amount of methanol. The precipitate was collected by filtration, washed with methanol and ether to give (S)-N-(3-chloro-4-isopropoxyphenyl)-5-(4-((2,2-dimethyl-1,3-dioxolan-4-yl)methoxy)phenyl)isoxazole-3-carboxamide (113 mg, 0.225 mmol, 71.9% yield) as an off-white solid. LCMS (Table, 1, Method a) Rₜ=3.61 min, m/z (no ionization).

### Step G. Preparation of (R)-N-(3-chloro-4-isopropoxyphenyl)-5-(4-(2,3-dihydroxypropoxy)phenyl)isoxazole-3-carboxamide

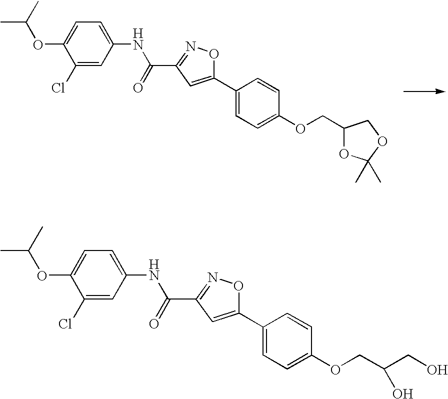

A round bottom flash was charged with (S)-N-(3-chloro-4-isopropoxyphenyl)-5-(4-((2,2-dimethyl-1,3-dioxolan-4-yl)methoxy)phenyl)isoxazole-3-carboxamide (210 mg, 0.431 mmol) and toluene-4-sulfonic acid hydrate (16.41 mg, 0.086 mmol) in MeOH (5.0 mL) to give a white suspension. The reaction mixture was heated at 70° C. for 6 hours. After cooled down, white precipitate was filtered off. Filtrate was concentrated and purified via HPLC to give (R)-N-(3-chloro-4-isopropoxyphenyl)-5-(4-(2,3-dihydroxypropoxy)phenyl)isoxazole-3-carboxamide (85.1 mg, 0.190 mmol, 44.2% yield) as a white solid. LCMS (Table, 1, Method a) Rₜ=2.72 min, m/z 445.23 (M−H)⁻. ¹H NMR (400 MHz, DMSO-d₆) δ 10.74 (s, 1H), 7.93 (d, J=2.6, 1H), 7.90 (d, J=8.9, 2H), 7.68 (dd, J=2.6, 9.0, 1H), 7.32 (s, 1H), 7.19 (d, J=9.3, 1H), 7.12 (d, J=9.0, 2H), 5.00 (d, J=5.2, 1H), 4.70 (t, J=5.7, 1H), 4.62 (dq, J=6.0, 11.9, 1H), 4.10 (dd, J=4.0, 10.0, 1H), 3.96 (dd, J=6.2, 10.0, 1H), 3.86-3.77 (m, 1H), 3.46 (t, J=5.7, 2H), 1.29 (d, J=6.0, 6H).

### Preparation of 4-bromo-3-chloro-benzaldehyde (prep a luII)

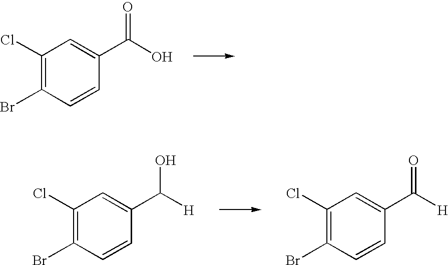

1M Borane/THF solution (150 mL, 150 mmol) was added to a solution of 4-bromo-3-chlorobenzoic acid (15 g, 63.7 mmol) in THF (150 mL) at 0° C. dropwisely. The reaction was allowed to warm to room temperature and stirred at room temperature for two hours. The reaction was quenched by dropwise addition of a mixture of THF and water (20 mL, 1:1), followed by addition of water (20 mL). The reaction was extracted with EtOAc (100 mL) three times. The organic layer was combined and washed with brine, dried with anhydrous sodium sulfate and concentrated to yield solid intermediate. LCMS (Method b) Rₜ=1.96 min; m/z: 221 (M+H)⁺.

PCC (20.60 g, 96 mmol) was added slowly to the solution of intermediate in DCM (150 mL) at 0° C. The reaction was allowed to warm to room temperature and stirred at room temperature for five hours. The crude was filtered through celite and concentrated. The residue was purified through silica gel column with 0-30% EtOAc/Hept to yield 4-bromo-3-chlorobenzaldehyde (12.0 g, 54.7 mmol) in solid form. LCMS (Method b) Rₜ=2.22 min; m/z: 220 (M+H)⁺.

### Preparation of 3-(3-chloro-4-isopropoxy-phenyl)-isoxazole-5-carboxylic acid (prep b luII)

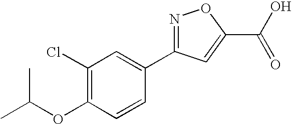

Hydroxylamine hydrochloride (1.719 g, 24.74 mmol) and 3-chloro-4-isopropoxybenzaldehyde (4.68 g, 23.56 mmol) were dissolved in a mixture of t-BuOH (30 mL) and Water (30.0 mL) to give a clear solution. 1 N NaOH (24.74 mL, 24.74 mmol) was added to the reaction mixture. The mixture was stirred for 30 min before Chloramine-T (6.08 g, 24.74 mmol) was added. The mixture was stirred for about another 5 min, followed by addition of cupric sulfate (0.031 mL, 0.707 mmol), copper (0.225 g, 3.53 mmol) and propiolic acid (1.650 g, 23.56 mmol). 2 N NaOH (15 mL) was added to the mixture to adjust pH to 6. The mixture was stirred at RT over night. Ice water (100 mL) was added to the mixture, followed by 5 N HCl (10 mL) to turn pH to 1. The reaction mixture was extracted with EtOAc (100 mL) three times. The organic layer was combined and concentrated. Purified by prep HPLC to give 3-(3-chloro-4-isopropoxyphenyl)isoxazole-5-carboxylic acid (1.43 g, 5.08 mmol) as white solid. LCMS (Method a) Rₜ=1.85 min; m/z: 280 (M−H)⁻.

### Preparation of 3-(3-chloro-4-(3-(3-chloro-4-isopropoxyphenyl)isoxazole-5-carboxamido)benzylamino)butanoic acid

### Example #16

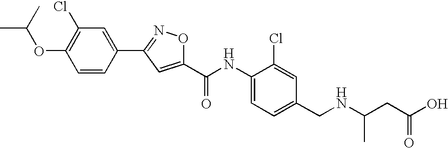

Prepared from 3-(3-chloro-4-isopropoxyphenyl)isoxazole-5-carboxylic acid following procedures D.3 and H to give 3-(3-chloro-4-(3-(3-chloro-4-isopropoxyphenyl)isoxazole-5-carboxamido)benzylamino)butanoic acid (0.05 g, 37%) as a white solid: LCMS (Method a) Rₜ=2.37 min; m/z: 506 (M−H)⁻.

### Preparation of 3-(3-chloro-4-(3-(3-chloro-4-isopropoxyphenyl)isoxazole-5-carboxamido)benzylamino)-2-methylpropanoic acid

### Example #17

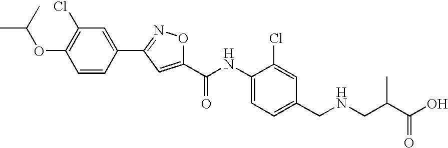

Prepared from 3-(3-chloro-4-isopropoxyphenyl)isoxazole-5-carboxylic acid following procedures D.3 and H to give 3-(3-chloro-4-(3-(3-chloro-4-isopropoxyphenyl)isoxazole-5-carboxamido)benzylamino)-2-methylpropanoic acid (0.038 g, 28%) as a white solid: LCMS (Method a) Rₜ=2.39 min; m/z: 506.13 (M−H)⁻.

### Preparation of 1-(3-chloro-4-(3-(3-chloro-4-isopropoxyphenyl)isoxazole-5-carboxamido)benzyl)azetidine-3-carboxylic acid

### Example #18

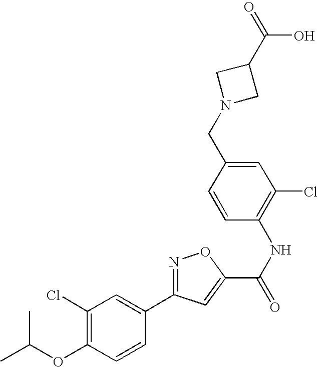

Prepared from 3-(3-chloro-4-isopropoxyphenyl)isoxazole-5-carboxylic acid following procedures D.3 and H to give 1-(3-chloro-4-(3-(3-chloro-4-isopropoxyphenyl)isoxazole-5-carboxamido)benzyl)azetidine-3-carboxylic acid (55 mg, 25%) as a white solid: LCMS (Method a) Rₜ=1.96 min; mm/z: 504.15 (M−H)⁻.

### Preparation of 3-(2-(4-(3-(3-chloro-4-isopropoxyphenyl)isoxazole-5-carboxamido)phenyl)propan-2-ylamino)propanoic acid

### Example #19

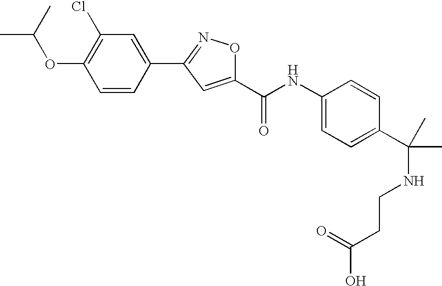

Prepared from 3-(3-chloro-4-isopropoxyphenyl)isoxazole-5-carboxylic acid following procedures D.4, Y, O to give 3-(2-(4-(3-(3-chloro-4-isopropoxyphenyl)isoxazole-5-carboxamido)phenyl)propan-2-ylamino)propanoic acid (13 mg, 20%) as a white solid: LCMS (Method a) Rₜ=2.29 min; m/z: 486.17 (M−H)⁻.

### Preparation of 1-(3-chloro-4-isopropoxy-phenyl)-1H-[1,2,3]triazole-4-carboxylic acid ethyl ester

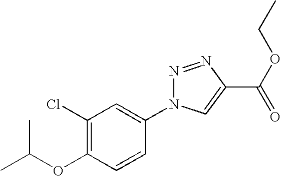

t-Butyl nitrite (0.535 mL, 4.05 mmol) was added to a solution of 3-chloro-4-isopropoxyaniline (0.501 g, 2.70 mmol) in Acetonitrile (9 mL) at 0° C., followed by TMS-N3 (0.430 mL, 3.24 mmol) dropwise. The resulting mixture was stirred at room temperature for 2 hours. Propynoic acid ethyl ester (0.413 mL, 4.05 mmol), a solution of copper (II) sulfate pentahydrate (0.067 g, 0.270 mmol) in water (6 mL) and sodium ascorbate (0.535 g, 2.70 mmol) was added. The mixture was stirred at room temperature for another 4 hours. The reaction was cooled to 0° C. and quenched with water (10 mL). The solution was filtered to give ethyl 1-(3-chloro-4-isopropoxyphenyl)-1H-1,2,3-triazole-4-carboxylate (0.771 g, 2.295 mmol) as dark brown solid. LCMS (Method b) Rₜ=1.96 min; nm/z: 310 (M+H)⁺.

### Preparation of 3-(4-(1-(3-chloro-4-isopropoxyphenyl)-1H-1,2,3-triazole-4-carboxamido)benzylamino)-2-methylpropanoic acid

### Example #20

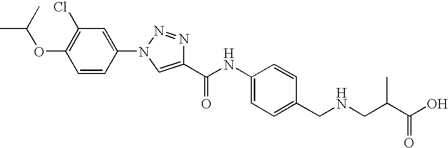

Prepared from 1-(3-chloro-4-isopropoxy-phenyl)-1H-[1,2,3]triazole-4-carboxylic acid ethyl ester following procedures U, V, H to give 3-(4-(1-(3-chloro-4-isopropoxyphenyl)-1H-1,2,3-triazole-4-carboxamido)benzylamino)-2-methylpropanoic acid (116 mg, 43%) as a white solid: LCMS (Method a) Rₜ=2.14 min; m/z: 470.2 (M−H)⁻.

### Preparation of 3-(4-(1-(3-chloro-4-isopropoxyphenyl)-1H-1,2,3-triazole-4-carboxamido)benzylamino)butanoic acid

### Example #21

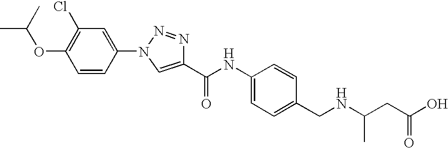

Prepared from 1-(3-chloro-4-isopropoxy-phenyl)-1H-[1,2,3]triazole-4-carboxylic acid ethyl ester following procedures U, V, H to give 3-(4-(1-(3-chloro-4-isopropoxyphenyl)-1H-1,2,3-triazole-4-carboxamido)benzylamino)butanoic acid (102 mg, 41%) as a white solid: LCMS (Method a) Rₜ=2.14 min; m/z: 470.25 (M−H)⁻.

### Preparation of 4-{4-[3-(3-chloro-4-isopropoxy-phenylcarbamoyl)-isoxazol-5-yl]-phenoxy}-butyric acid methyl ester (prep d luII)

### Example #22

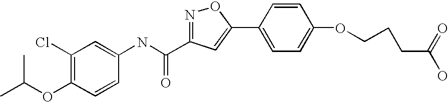

To a solution of N-(3-chloro-4-isopropoxyphenyl)-5-(4-hydroxyphenyl)isoxazole-3-carboxamide (0.080 g, 0.215 mmol) in DMA (3 mL) in microwave tube was added K₂CO₃ (0.059 g, 0.429 mmol) followed by methyl 4-bromobutanoate (0.039 g, 0.215 mmol). The reaction mixture was heated in a biotage microwave at 160° C. for 10 min. The reaction mixture was filtered and concentrated to give intermediate methyl 4-(4-(3-(3-chloro-4-isopropoxyphenylcarbamoyl)isoxazol-5-yl)phenoxy)butanoate (0.083 g, 0.176 mmol). LCMS (Method b) Rₜ=2.60 min; m/z: 471 (M−H)⁻. Methyl-4-(4-(3-(3-chloro-4-isopropoxyphenylcarbamoyl)isoxazol-5-yl)phenoxy)butanoate (0.060 g, 0.127 mmol) was dissolved in THF (0.846 mL) and MeOH (0.423 mL), followed by addition of NaOH (0.015 g, 0.381 mmol). The reaction mixture was stirred at room temperature for 2 hour. Solvent removed under vacuum. The reaction mixture was extracted between 1 N HCl (10 mL) and DCM (5 mL). The inorganic layer was washed with DCM (5 mL) twice. The organic layer was combined and concentrated and purified by prep HPLC to give 4-(4-(3-(3-chloro-4-isopropoxyphenylcarbamoyl)isoxazol-5-yl)phenoxy)butanoic acid (0.023 g, 0.050 mmol). LCMS (Method b) Rₜ=3.06 min; m/z: 457 (M−H)⁻.

### Preparation of N-(3-chloro-4-isopropoxyphenyl)-5-(4-formylphenyl)-N-methylisoxazole-3-carboxamide

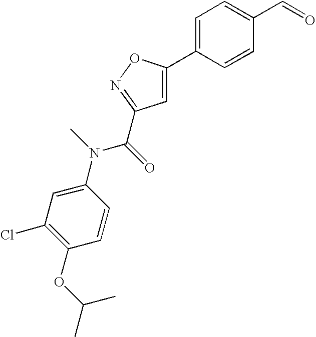

To the flask containing 5-(4-(1,3-dioxolan-2-yl)phenyl)-N-(3-chloro-4-isopropoxyphenyl)isoxazole-3-carboxamide (0.235 g, 0.548 mmol) in DMF (4 mL) was added NaOH (0.055 g, 1.370 mmol) at room temperature. After stirring for 15 minutes at room temperature, methyl iodide (0.069 mL, 1.096 mmol) was added in one portion. The reaction was stirred at room temperature for about 16 hours. The reaction was extracted with DCM (10 mL) and saturated sodium bicarbonate (10 mL). The aqueous phase was washed with DCM (10 mL) two times. The organic phase was combined, dried. The residue was purified via Analogix system with a gradient of 0-50% EtOAc/Heptane. Fractions containing product were combined and concentrated to yield the intermediate (0.046 g). The intermediate was dissolved in THF (2 mL). 5 N HCl (0.4 mL) was added. The mixture was stirred at room temperature over night. The reaction was extracted with DCM (10 mL) and saturated sodium bicarbonate (10 mL). The aqueous phase was washed with DCM (10 mL) two times. The organic phase was combined, dried to give N-(3-chloro-4-isopropoxyphenyl)-5-(4-formylphenyl)-N-methylisoxazole-3-carboxamide (0.020 g, 0.050 mmol) as brown solid. LCMS (Method a) Rₜ=3.11 min; m/z: 399 (M+H)⁺.

### Preparation of 1-(4-(3-((3-chloro-4-isopropoxyphenyl)(methyl)carbamoyl)-isoxazol-5-yl)benzyl)azetidine-3-carboxylic acid

### Example #23

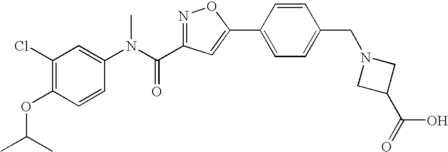

Prepared from N-(3-chloro-4-isopropoxyphenyl)-5-(4-formylphenyl)-N-methylisoxazole-3-carboxamide following general procedures N, H to give 1-(4-(3-((3-chloro-4-isopropoxyphenyl)(methyl)carbamoyl)isoxazol-5-yl)benzyl)azetidine-3-carboxylic acid (16 mg, 66%): LCMS (Method a) Rₜ=2.09 min; m/z: 484 (M−H)⁻.

### Preparation of 3-(2-(4-(1-tert-butyl-5-phenyl-1H-pyrazole-3-carboxamido)-phenyl)propan-2-ylamino)propanoic acid

### Example #24

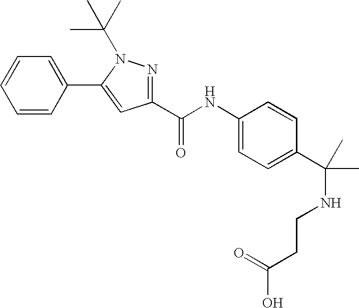

Prepared from 1-(pyridin-3-yl)ethanone following general procedures R, T, D4, Y to give 3-(2-(4-(1-tert-butyl-5-phenyl-1H-pyrazole-3-carboxamido)phenyl)propan-2-ylamino)propanoic acid (144 mg, 69%) as a white solid: LCMS (Method a) Rₜ=2.19 min; m/z: 449.28 (M+H)⁺.

### Preparation of 3-(5-(3-(cyclohexylcarbamoyl)isoxazol-5-yl)-1H-indazol-1-yl)propanoic acid

### Example #25

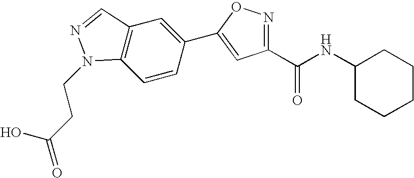

Prepared from 5-(1H-indazol-5-yl)isoxazole-3-carboxylic acid following general procedures K, X, M, D.4, Y to give 3-(5-(3-(cyclohexylcarbamoyl)isoxazol-5-yl)-1H-indazol-1-yl)propanoic acid (12 mg, 62%) as a white solid: LCMS (Method a) Rₜ=2.05 min; m/z: 383 (M+H)⁺.

### Preparation of 1-(4-(3-(3-chloro-4-isopropoxyphenyl)-1H-pyrazole-5-carboxamido)benzyl)azetidine-3-carboxylic acid

### Example #26

### General Synthetic Route

### General Procedures R, S, C, E, F, I, J.1, H

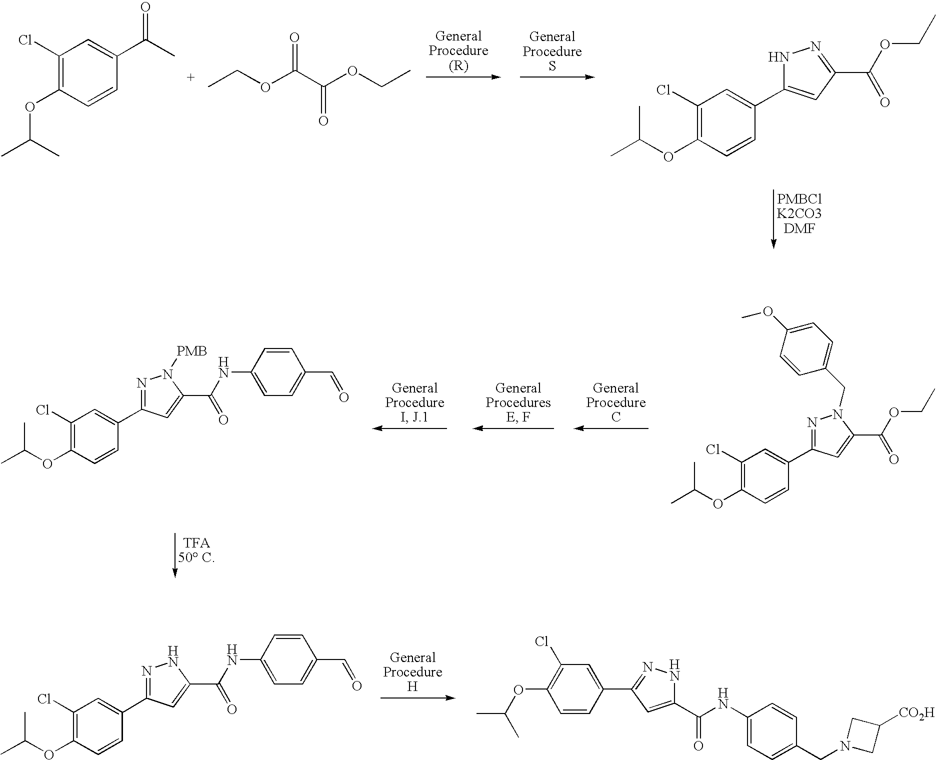

### Ethyl 3-(3-chloro-4-isopropoxyphenyl)-1-(4-methoxybenzyl)-1H-pyrazole-5-carboxylate

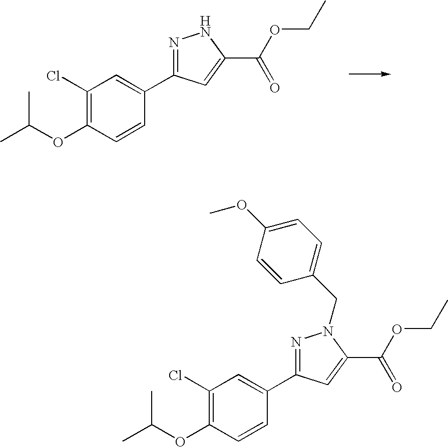

Ethyl 5-(3-chloro-4-isopropoxyphenyl)-1H-pyrazole-3-carboxylate (0.28 g, 0.907 mmol) (Prepared by general procedures R, S), potassium carbonate (0.627 g, 4.53 mmol) and 1-(chloromethyl)-4-methoxybenzene (0.568 g, 3.63 mmol) were added in DMF (9.07 mL) to give a deep red solution. The reaction mixture was heated in Microwave (Biotage™ Initiator 2.0) at about 120° C. for 25 min. Cool down, the reaction mixture was partitioned between EtOAc (100 mL) and hydrochloric acid (5%, 100 mL), the organic layer was washed by hydrochloric acid (10%, 50 mL), water (50 mL) and brine (30 mL), concentration afforded a red oil, which was purified via silica gel flash column chromatography (40 g, 40% EtOAc/heptane) to afford ethyl 3-(3-chloro-4-isopropoxyphenyl)-1-(4-methoxybenzyl)-1H-pyrazole-5-carboxylate (0.38 g, 0.886 mmol, 98% yield) as yellow oil.: LCMS (Table 1, Method g) Rₜ=3.35 min.; MS m/z: 429.23 (M+H)⁺; ¹H NMR (400 MHz, d-DMSO) d 7.91 (d, 1H, J=2.0), 7.78 (dd, 1H, J=2.1, 8.6), 7.41 (s, 1H), 7.19 (m, 3H), 6.89 (d, 2H, J=8.4), 5.66 (s, 2H), 4.71 (dt, 1H, J=6.1, 11.9), 4.31 (q, 2H, J=7.1), 3.71 (s, 3H), 1.30 (m, 9H).

### 3-(3-chloro-4-isopropoxyphenyl)-N-(4-formylphenyl)-1H-pyrazole-5-carboxamide

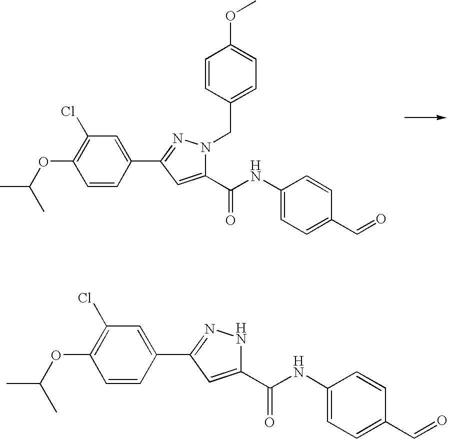

A solution of 3-(3-chloro-4-isopropoxyphenyl)-N-(4-formylphenyl)-1-(4-methoxybenzyl)-1H-pyrazole-5-carboxamide (0.245 g, 0.486 mmol) (Prepared from ethyl 3-(3-chloro-4-isopropoxyphenyl)-1-(4-methoxybenzyl)-1H-pyrazole-5-carboxylate following general procedures C, E, F, I, J.1)) in TFA (5 mL, 64.9 mmol) was heated at 50° C. for 1 h. Cool down, the reaction mixture was diluted with ethyl acetate (100 mL), washed by saturated sodium bicarbonate (3×50 mL), water (30 mL), brine (30 mL), dried over sodium sulfate, filtration and concentration afforded 3-(3-chloro-4-isopropoxyphenyl)-N-(4-formylphenyl)-1H-pyrazole-5-carboxamide (0.180 g, 0.516 mmol, 99% yield) as pale yellow solid. LCMS (Table 1, Method g) Rₜ=2.51 min.; MS m/z: 384.06 (M+H)⁺.

### Preparation of 1-(4-(3-(3-chloro-4-isopropoxyphenyl)-1H-pyrazole-5-carboxamido)benzyl)azetidine-3-carboxylic acid

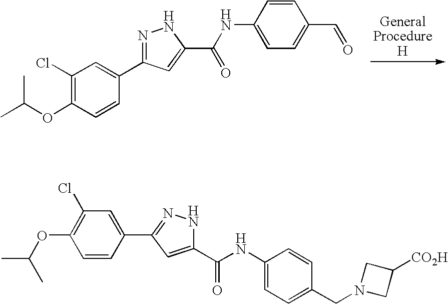

Prepared from 3-(3-chloro-4-isopropoxyphenyl)-N-(4-formylphenyl)-1H-pyrazole-5-carboxamide following general procedure H to give 1-(4-(3-(3-chloro-4-isopropoxyphenyl)-1H-pyrazole-5-carboxamido)benzyl)azetidine-3-carboxylic acid (70 mg, 57%) as a white solid: LCMS (Method g) Rₜ=1.78 min.; MS m/z: 468.92 (M+H)⁺.

### 3-(3-chloro-4-isopropoxyphenyl)-5-(1-methyl-1H-pyrazol-5-yl)-1,2,4-oxadiazole

### Example #27

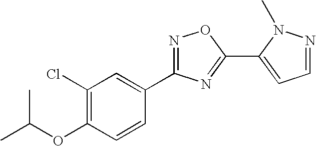

A suspension of 1-methyl-1H-pyrazole-5-carboxylic acid (200 mg, 1.586 mmol) in thionyl chloride (6 mL, 82 mmol) was heated at 70° C. for 16 h. The reaction mixture was concentrated to afford 120 mg pale yellow solid, which was combined with (Z)-3-chloro-N′-hydroxy-4-isopropoxybenzimidamide (120 mg, 0.525 mmol) to be dissolved in pyridine (3 mL). The reaction mixture was heated in Microwave (Biotage™ Initiator 2.0) at about 200° C. for 20 min. Cool down, the reaction mixture was poured into HCl (10%, 30 mL), the suspension was extracted by DCM (2×30 mL). The combined DCM solution was concentrated to afford an orange oil, which was purified via automated silica gel chromatography (12 g, 10% EtOAc:Heptane) to afford 3-(3-chloro-4-isopropoxyphenyl)-5-(1-methyl-1H-pyrazol-5-yl)-1,2,4-oxadiazole (111 mg, 0.348 mmol, 66.4% yield) as colorless oil. LCMS (Table 1, Method a) Rₜ=3.08 min.; MS m/z: 319.25 (M+H)⁺; ¹H NMR (400 MHz, d-DMSO) δ 8.07 (d, J=2.11 Hz, 1H), 8.01 (dd, J=8.64, 2.14 Hz, 1H), 7.72 (d, J=2.11 Hz, 1H), 7.39 (d, J=8.80 Hz, 1H), 7.22 (d, J=2.11 Hz, 1H), 4.83 (sept., J=6.11 Hz, 1H), 4.30 (s, 3H), 1.35 (d, J=6.02 Hz, 6H).

### Preparation of N-(2-chloro-4-hydroxyphenyl)-1-phenyl-5-(trichloromethyl)-1H-1,2,4-triazole-3-carboxamide

### Example #28

### Step A. Preparation of 4-amino-3-chlorophenyl tert-butyl carbonate

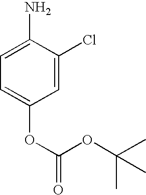

1-methyl-1H-imidazole (0.041 g, 0.500 mmol) was added to a gray suspension of 4-amino-3-chloro-phenol hydrochloride (1.800 g, 10 mmol) and BOC-Anhydride (3.30 mL, 14.20 mmol) in water (10 mL). TEA (1.394 mL, 10.00 mmol) was added drop wise to the stirring mixture. The reaction mixture was stirred at 32° C. for 7 hr, the resultant mixture was partitioned between EtOAc (75 mL) and water (75 mL), the organic layer was washed by water (3×50 mL), concentration afforded 3.35 g red oil, which was purified via automated silica gel chromatography (40 g, 30% EtOAc:Heptane) to afford 4-amino-3-chlorophenyl tert-butyl carbonate (2.5 g, 10.29 mmol, 100% yield) as deep red oil. LCMS (Table 1, Method a) Rₜ=3.66 min.; MS m/z: 244.07 (M+H)⁺; ¹H NMR (400 MHz, d-DMSO) δ 7.08 (d, J=2.6, 1H), 6.86 (dd, J=2.7, 8.8, 1H), 6.77 (d, J=8.8, 1H), 5.32 (s, 2H), 1.47 (s, 9H).

### Step B. Preparation of tert-butyl 3-chloro-4-(1-phenyl-5-(trichloromethyl)-1H-1,2,4-triazole-3-carboxamido)phenyl carbonate

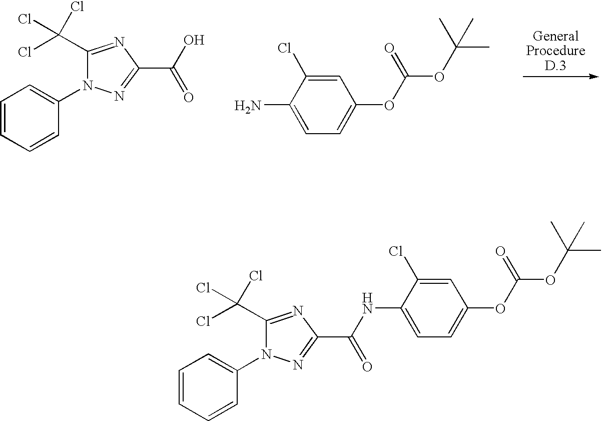

Prepared from 4-amino-3-chlorophenyl tert-butyl carbonate and 1-phenyl-5-(trichloromethyl)-1H-1,2,4-triazole-3-carboxylic acid via General Procedure D.3 to give tert-butyl-3-chloro-4-(1-phenyl-5-(trichloromethyl)-1H-1,2,4-triazole-3-carboxamido)phenyl carbonate (2.52 g, 69%) as a pale yellow solid: LCMS (Table 1, Method a) Rₜ=4.59 min.; MS m/z: 533.05 (M+H)⁺; ¹H NMR (400 MHz, d-DMSO) δ 10.33 (s, 1H), 7.82-7.73 (m, 3H), 7.72-7.61 (m, 3H), 7.55 (dd, J=1.2, 2.7, 1H), 7.28 (ddd, J=1.2, 2.7, 8.8, 1H), 1.51 (s, 9H).

### Step C. Preparation of N-(2-chloro-4-hydroxyphenyl)-1-phenyl-5-(trichloromethyl)-1H-1,2,4-triazole-3-carboxamide

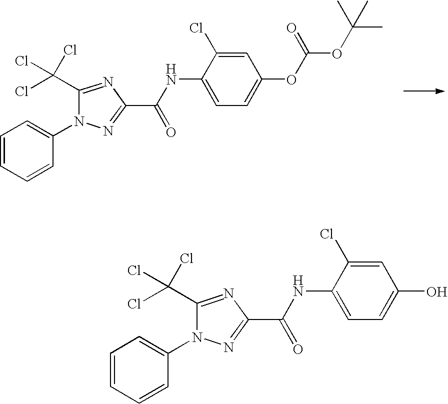

To a stirring yellow solution of tert-butyl 3-chloro-4-(1-phenyl-5-(trichloromethyl)-1H-1,2,4-triazole-3-carboxamido)phenyl carbonate (2.5 g, 4.70 mmol) in dioxane (70.2 mL) was added drop wise aqueous hydrochloric acid (4M, 70.5 mL, 282 mmol) over 15 minutes. The reaction mixture was heated at 100° C. for 17 hr. Cool down, the resulting precipitate was filtered, washed by water (3×20 mL) and dried to afford N-(2-chloro-4-hydroxyphenyl)-1-phenyl-5-(trichloromethyl)-1H-1,2,4-triazole-3-carboxamide (1.858 g, 4.30 mmol, 92% yield) as a pale yellow powder. LCMS (Table 1, Method a) Rₜ=3.67 min.; MS m/z: 432.96 (M+H)⁺; ¹H NMR (400 MHz, d-DMSO) δ 10.07 (s, 1H), 9.94 (s, 1H), 7.79-7.72 (m, 2H), 7.71-7.59 (m, 3H), 7.46 (t, J=8.7, 1H), 6.92 (d, J=2.6, 1H), 6.79 (dd, J=2.7, 8.7, 1H).

### Preparation of N-(2-chloro-4-((2,2-dimethyl-1,3-dioxolan-4-yl)methoxy)-phenyl)-1-phenyl-5-(trichloromethyl)-1H-1,2,4-triazole-3-carboxamide

### Example #29

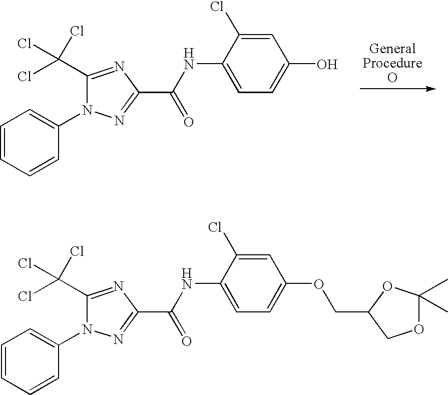

Prepared from N-(2-chloro-4-hydroxyphenyl)-1-phenyl-5-(trichloromethyl)-1H-1,2,4-triazole-3-carboxamide (Example #28, Step C) via General Procedure 0 to give N-(2-chloro-4-((2,2-dimethyl-1,3-dioxolan-4-yl)methoxy)phenyl)-1-phenyl-5-(trichloro-methyl)-1H-1,2,4-triazole-3-carboxamide (527 mg, 83%) as a white solid: Yield 83%. LCMS (Table 1, Method a) Rₜ=4.37 min.; MS m/z: 545.29 (M−H)⁻; ¹H NMR (400 MHz, d-DMSO) δ 10.17 (s, 1H), 7.79-7.73 (m, 2H), 7.72-7.61 (m, 3H), 7.58 (d, J=8.94 Hz, 1H), 7.20 (d, J=2.83 Hz, 1H), 7.01 (dd, J=8.89, 2.82 Hz, 1H), 4.41 (dq, J=6.39, 4.39 Hz, 1H), 4.16-3.97 (m, 3H), 3.75 (dd, J=8.39, 6.30 Hz, 1H), 3.31 (s, 4H), 1.34 (d, J=21.84 Hz, 6H).

### Preparation of N-(2-chloro-4-(2,3-dihydroxypropoxy)phenyl)-1-phenyl-5-(trichloromethyl)-1H-1,2,4-triazole-3-carboxamide

### Example #30

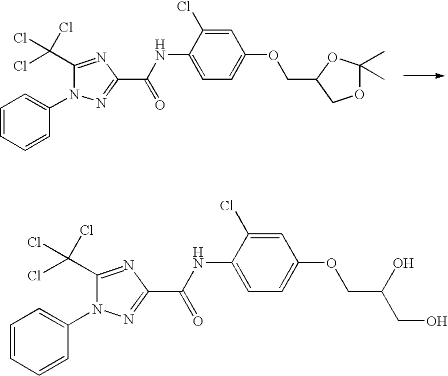

To a suspension of N-(2-chloro-4-((2,2-dimethyl-1,3-dioxolan-4-yl)methoxy)phenyl)-1-phenyl-5-(trichloromethyl)-1H-1,2,4-triazole-3-carboxamide (0.15 g, 0.275 mmol) (Example #29) in MeOH (2.0 mL) and water (2.0 mL) was added drop wise aqueous HCl (6M, 2.0 mL, 12 mmol) over 1 minute. The reaction mixture was heated in Microwave (Biotage™ Initiator 2.0) at about 100° C. for 25 min. Cool down, the resulting precipitate was filtered, washed by water (3×20 mL) and dried to afford N-(2-chloro-4-(2,3-dihydroxy-propoxy)phenyl)-1-phenyl-5-(trichloromethyl)-1H-1,2,4-triazole-3-carboxamide (0.133 g, 0.263 mmol, 96% yield) as white solid. LCMS (Table 1, Method a) Rₜ=3.35 min.; MS m/z: 507.00 (M+H)⁺; ¹H NMR (400 MHz, d-DMSO) δ 10.16 (s, 1H), 7.79-7.73 (m, 2H), 7.71-7.61 (m, 3H), 7.58 (t, J=8.99 Hz, 1H), 7.15 (t, J=3.78 Hz, 1H), 6.99 (dd, J=8.88, 2.81 Hz, 1H), 4.98 (d, J=5.14 Hz, 1H), 4.71-4.65 (m, 1H), 4.05 (dd, J=10.05, 4.06 Hz, 1H), 3.95-3.87 (m, 1H), 3.84-3.74 (m, 1H), 3.44 (t, J=5.81 Hz, 2H).

### Preparation of Ethyl 3-(4-(5-tert-butyl-1-phenyl-1H-1,2,4-triazole-3-carboxamido)-3-chlorophenyl)propanoate

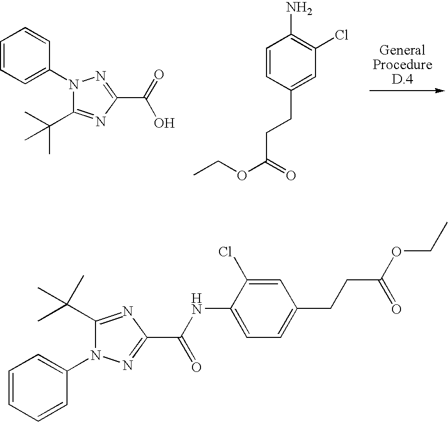

Prepared from 5-tert-butyl-1-phenyl-1H-1,2,4-triazole-3-carboxylic acid and ethyl 3-(4-amino-3-chlorophenyl)propanoate via general procedure D.4 to give ethyl 3-(4-(5-tert-butyl-1-phenyl-1H-1,2,4-triazole-3-carboxamido)-3-chlorophenyl)propanoate (124 mg, 44.6%) as a colorless oil: LCMS (Table 1, Method a) Rₜ=3.90 min.; MS m/z: 455.31 (M+H)⁺.

### 3-(4-(5-tert-butyl-1-phenyl-1H-1,2,4-triazole-3-carboxamido)-3-chloro-phenyl)propanoic acid

### Example #31

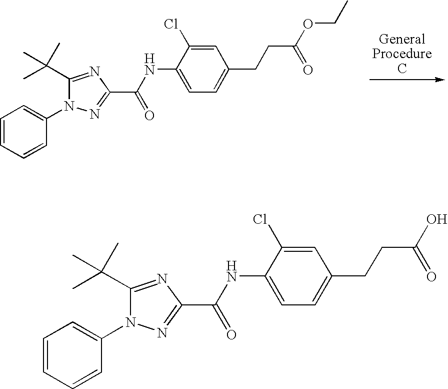

Prepared from ethyl 3-(4-(5-tert-butyl-1-phenyl-1H-1,2,4-triazole-3-carboxamido)-3-chlorophenyl)propanoate via General Procedure C to give 3-(4-(5-tert-butyl-1-phenyl-1H-1,2,4-triazole-3-carboxamido)-3-chlorophenyl)propanoic acid (105 mg, 90%) as a white solid: LCMS (Table 1, Method a) Rₜ=3.34 min.; MS m/z: 427.17 (M+H)⁺; ¹H NMR (400 MHz, d-DMSO) δ 12.17 (s, 1H), 9.86 (s, 1H), 7.88 (d, J=8.29 Hz, 1H), 7.70-7.57 (m, 5H), 7.44 (d, J=1.61 Hz, 1H), 7.26 (dd, J=8.36, 1.66 Hz, 1H), 2.83 (t, J=7.50 Hz, 2H), 2.57 (t, J=7.53 Hz, 2H), 1.25 (s, 9H).

### Preparation of (Z)-methyl 2-chloro-2-(2-(3-chloro-4-isopropoxyphenyl)hydrazono)acetate

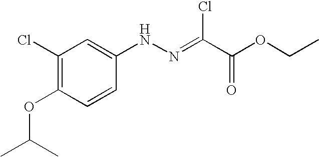

To a solution of 3-chloro-4-isopropoxyaniline (0.299 g, 1.611 mmol) in MeOH (1.5 mL) was added water (0.5 mL) under nitrogen. The reaction was cooled to about 0-5° C. in an ice bath. Hydrochloric acid (0.049 mL, 1.611 mmol) was added dropwise. A ice-cold solution of sodium nitrite (0.111 g, 1.611 mmol) in water (0.5 μL) was added dropwise over 7 min. It was stirred for 30 min. Sodium acetate (0.661 g, 8.05 mmol) was added portionwise to adjust pH to 5. A colorless solution of methyl 2-chloro-3-oxobutanoate (0.196 mL, 1.611 mmol) in MeOH (1.5 mL) was added dropwise. The ice-bath was removed after 1 hr, the reaction mixture was stirred at room temperature for 18 hr. The reaction mixture was partitioned between ether (25 mL) and sat. NaHCO₃ (20 mL), the ether layer was washed by saturated sodium bicarbonate (20 mL) and dried over sodium sulfate, filtration and concentration afforded 0.56 g black residue, which was purified by silica gel flash column chromatography (40 g, 1:1 EtOAc/heptane) to afford (Z)-methyl 2-chloro-2-(2-(3-chloro-4-isopropoxyphenyl)hydrazono)acetate (0.32 g, 1.049 mmol, 65.1% yield) as yellow solid.: LCMS (Table 1, Method a) Rₜ=3.58 min.; MS m/z: 304.84 (M−H)⁺; ¹H NMR (400 MHz, d-DMSO) δ 10.55 (s, 1H), 7.39 (d, J=2.61 Hz, 1H), 7.26 (dd, J=8.96, 2.63 Hz, 1H), 7.16 (d, J=8.93 Hz, 1H), 4.59-4.50 (m, 1H), 3.83 (s, 3H), 1.27 (d, J=6.04 Hz, 6H).

### Preparation of 5-(3-chloro-4-isopropoxyphenyl)isoxazole-3-carboxamide

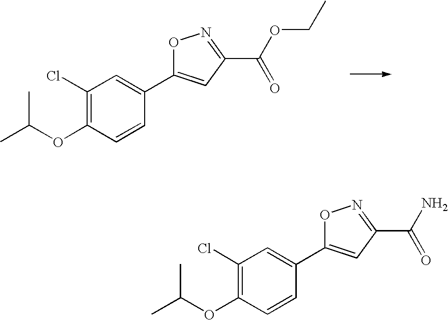

Prepared from ethyl 5-(3-chloro-4-isopropoxyphenyl)isoxazole-3-carboxylate via General Procedure U to give 5-(3-chloro-4-isopropoxyphenyl)isoxazole-3-carboxamide (1.79 g, 100%) as a yellow solid: LCMS (Table 1, Method e) Rₜ=2.78 min.; MS m/z: 281.07 (M+H)⁺; ¹H NMR (400 MHz, d-DMSO) δ 8.12 (s, 1H), 8.00 (d, J=1.27 Hz, 1H), 7.89-7.79 (m, 2H), 7.34 (d, J=8.63 Hz, 1H), 7.28 (d, J=0.70 Hz, 1H), 4.81 (sept., J=6.25 Hz, 1H), 1.33 (d, J=5.86 Hz, 6H).

### Preparation of N-(2-chloro-4-formylphenyl)-5-(3-chloro-4-isopropoxyphenyl)isoxazole-3-carboxamide

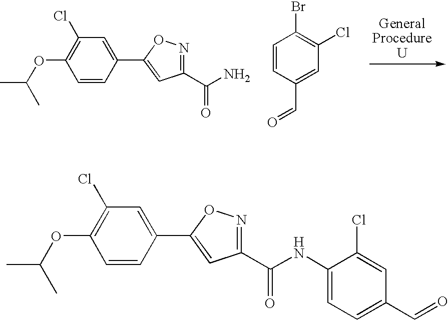

Prepared from 5-(3-chloro-4-isopropoxyphenyl)isoxazole-3-carboxamide and 4-bromo-3-chlorobenzaldehyde via General Procedure V to give N-(2-chloro-4-formylphenyl)-5-(3-chloro-4-isopropoxyphenyl)isoxazole-3-carboxamide (1.28 g, 60%) as a yellow solid: LCMS (Table 1, Method a) Rₜ=4.48 min.; MS m/z: 417.12 (M−H)⁻; ¹H NMR (400 MHz, d-DMSO) δ 10.35 (s, 1H), 9.98 (s, 1H), 8.18-8.05 (m, 3H), 8.00-7.87 (m, 2H), 7.54 (d, J=1.10 Hz, 1H), 7.37 (d, J=8.55 Hz, 1H), 4.89-4.77 (m, 1H), 1.34 (d, J=5.47 Hz, 6H).

### 1-(3-chloro-4-(5-(3-chloro-4-isopropoxyphenyl)thiazole-2-carboxamido)benzyl)azetidine-3-carboxylic acid

### Example #32

### Step A. Preparation of 5-(3-chloro-4-isopropoxyphenyl)thiazole

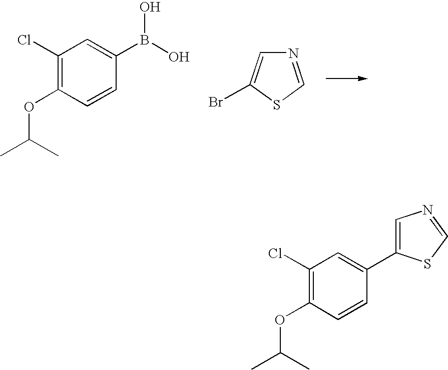

Sodium carbonate (5.00 mL, 6.00 mmol), 3-chloro-4-isopropoxyphenylboronic acid (0.236 g, 1.100 mmol) and 5-bromothiazole (0.164 g, 1 mmol) was added in DMF (20.00 mL) to give a yellow heterogeneous suspension. The reaction mixture was vacuumed and refilled with Nitrogen 4 times. Tetrakis(triphenylphosphine)palladium(0) (0.116 g, 0.100 mmol) was added, the reaction mixture was heated at about 85° C. under Nitrogen for 16 hr. Cool down, the reaction mixture was partitioned between EtOAc (200 mL) and hydrochloric acid (1M, 100 mL), the organic layer was washed by HCl (1M, 50 mL), brine (50 mL), dried over sodium sulfate, filtration and concentration afforded 0.54 g red syrup, which was purified via silica gel flash column chromatography (40 g, 40% EtOAc/heptane) to afford 5-(3-chloro-4-isopropoxyphenyl)thiazole (0.254 g, 1.001 mmol, 100% yield) as yellow syrup. LCMS (Table 1, Method a) Rₜ=3.59 min.; MS m/z: 253.87 (M+H)⁺; ¹H NMR (400 MHz, d-DMSO) δ 9.05 (s, 1H), 8.28 (s, 1H), 7.79 (dd, J=2.29, 1.01 Hz, 1H), 7.62-7.52 (m, 1H), 7.24 (d, J=8.50 Hz, 1H), 4.73 (sept., J=6.01 Hz, 1H), 1.31 (dd, J=6.02, 0.72 Hz, 6H).

### Step B. Preparation of 5-(3-chloro-4 isopropoxyphenyl)thiazole-2-carboxylic acid

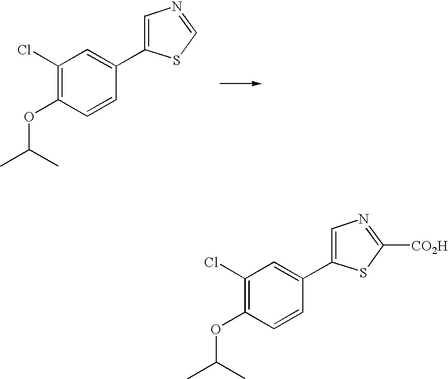

A solution of n-butyllithium (1.6 M, 6 mL, 9.60 mmol) in hexane was added dropwise to anhydrous ether (30 mL) at −78° C. A solution of 5-(3-chloro-4-isopropoxyphenyl)thiazole (1.6 g, 6.31 mmol) in ether (6 mL) was added dropwise to the reaction mixture over 15 min at −78° C., the reaction mixture was stirred at −78° C. for about 50 min. The dry-ice acetone cooling bath was replaced by dry-ice acetonitrile bath. The reaction mixture was stirred at 40° C. for 50 min. The reaction mixture was cooled at −78° C. again, at which point a gas stream of carbon dioxide was bubbled through the reaction mixture for 2 hr min at −78° C. The reaction mixture was then allowed to warm to room temperature over 30 min. The reaction mixture was bubbled through a gas stream of carbon dioxide for 5 hr at room temperature. The reaction mixture was partitioned between ether (100 mL) and hydrochloric acid (1M, 50 mL), the organic layer was washed by hydrochloric acid (1 M, 2×20 mL), brine (50 mL), dried over magnesium sulfate, filtration and concentration afforded 5-(3-chloro-4-isopropoxyphenyl)thiazole-2-carboxylic acid (1.14 g, 3.83 mmol, 60% yield). LCMS (Table 1, Method g) Rₜ=1.69 min.; MS m/z: 297.91 (M+H)⁺.

### Step C. Preparation of N-(2-chloro-4-formylphenyl)-5-(3-chloro-4-isopropoxyphenyl)thiazole-2-carboxamide

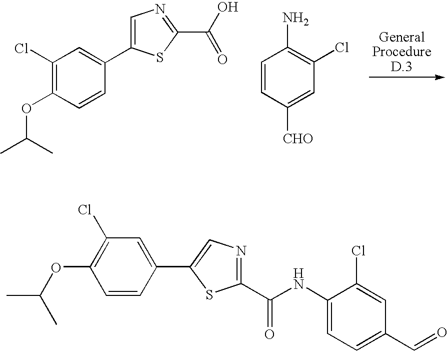

Prepared from 5-(3-chloro-4-isopropoxyphenyl)thiazole-2-carboxylic acid and via General Procedure D.3 to give N-(2-chloro-4-formylphenyl)-5-(3-chloro-4-isopropoxyphenyl)thiazole-2-carboxamide (0.14 g, 47%) as a yellow solid: LCMS (Table 1, Method g) Rₜ=3.51 min.; MS m/z: 433.36 (M−H)⁻; ¹H NMR (400 MHz, d-DMSO) δ 10.25 (s, 1H), 9.96 (s, 1H), 8.54 (s, 1H), 8.40 (d, J=8.37 Hz, 1H), 8.12 (d, J=1.77 Hz, 1H), 8.00-7.94 (m, 2H), 7.72 (dd, J=8.62, 2.33 Hz, 1H), 7.30 (d, J=8.71 Hz, 1H), 4.84-4.73 (m, 1H), 1.33 (d, J=6.02 Hz, 6H).

### Step D. Preparation of 1-(3-chloro-4-(5-(3-chloro-isopropoxyphenyl)thiazole-2-carboxamido)benzyl)azetidine-3-carboxylic acid

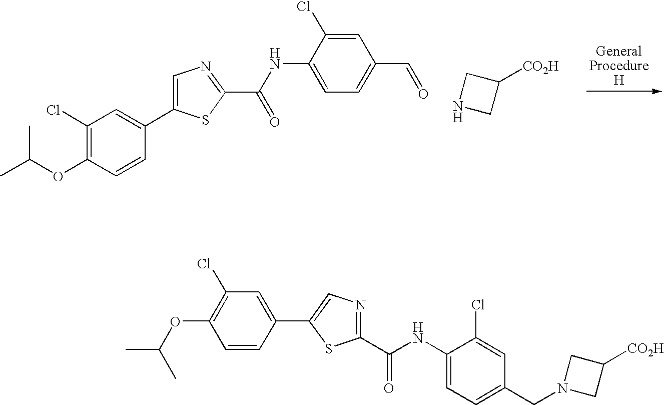

Prepared from N-(2-chloro-4-formylphenyl)-5-(3-chloro-4-isopropoxyphenyl)thiazole-2-carboxamide and azetidine carboxylic acid via General Procedure H to give 1-(3-chloro-4-(5-(3-chloro-4-isopropoxyphenyl)thiazole-2-carboxamido)benzyl)azetidine-3-carboxylic acid (55 mg, 70%) as a yellow solid: LCMS (Table 1, Method g) Rₜ=2.17 min.; MS m/z: 521.99 (M+H)⁺; ¹H NMR (400 MHz, d-DMSO) δ 10.12 (s, 1H), 8.47 (s, 1H), 7.94 (d, J=2.28 Hz, 1H), 7.86 (d, J=8.23 Hz, 1H), 7.68 (dd, J=8.67, 2.17 Hz, 1H), 7.44 (s, 1H), 7.32-7.22 (m, 2H), 4.82-4.70 (m, 1H), 3.54 (s, 2H), 3.42-3.35 (m, 2H), 3.23-3.16 (m, 3H), 1.31 (d, J=6.03 Hz, 6H).

### Preparation of 5-(3-chloro-4-isopropoxyphenyl)-N-(4-(oxiran-2-ylmethoxy)phenyl)isoxazole-3-carboxamide

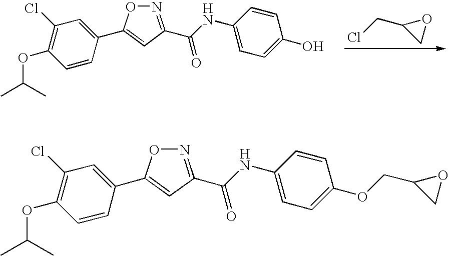

To a mixture of 5-(3-chloro-4-isopropoxyphenyl)-N-(4-hydroxyphenyl)isoxazole-3-carboxamide (0.300 g, 0.805 mmol) (Ex. Z1) in isopropanol (10 mL) was added NaOH (2.68 mL, 8.05 mmol) followed by epichlorohydrin (0.214 mL, 2.74 mmol). The reaction was stirred under an atmosphere of nitrogen at ambient temperature for 18 h. The resulting solid was collected by vacuum filtration. Solid was washed with isopropanol (10 mL), 1:1 water:isopropanol (15 mL), and dried in a vacuum oven overnight at 60° C. to provide 5-(3-chloro-4-isopropoxyphenyl)-N-(4-(oxiran-2-ylmethoxy)phenyl)isoxazole-3-carboxamide as a white solid (0.071 g, 20.6%). LCMS (Table 1, Method g) Rₜ=1.49 min.; MS m/z: 427.36. ¹H NMR (400 MHz, DMSO) δ ppm 10.62 (s, 1H), 8.04 (d, J=2.09 Hz, 1H), 7.88 (dd, J=8.69, 2.08 Hz, 1H), 7.70 (t, J=6.12 Hz, 2H), 7.42 (s, 1H), 7.40-7.31 (m, 1H), 6.98 (d, J=9.02 Hz, 2H), 4.82 (m, 1H), 4.32 (dd, J=11.36, 2.65 Hz, 1H), 3.83 (dd, J=11.36, 6.50 Hz, 1H), 3.34 (m, 1H), 2.85 (t, J=4.66 Hz, 1H), 2.71 (dd, J=5.04, 2.65 Hz, 1H), 1.34 (d, J=6.00 Hz, 6H).

### Preparation of N-(4-(3-amino-2-hydroxypropoxy)phenyl)-5-(3-chloro-4-isopropoxyphenyl)isoxazole-3-carboxamide

### Example #33

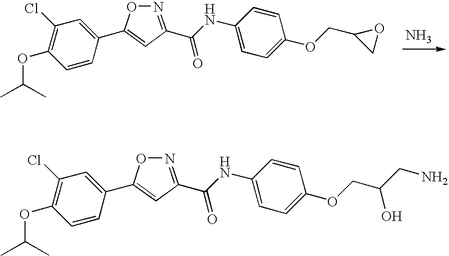

A mixture of 5-(3-chloro-4-isopropoxyphenyl)-N-(4-(oxiran-2-ylmethoxy)phenyl)isoxazole-3-carboxamide (0.067 g, 0.156 mmol), ammonia (1.7 mL, 11.90 mmol, 7 M in MeOH) and tetrahydrofuran (3.44 mL) was stirred at 60° C., under an atmosphere of nitrogen overnight. Heating was stopped and reaction left to cool to ambient temperature. Solid was collected by vacuum filtration, washed with THF (2×10 mL), and dried in a vacuum oven overnight at 60° C. to provide N-(4-(3-amino-2-hydroxypropoxy)phenyl)-5-(3-chloro-4-isopropoxyphenyl)isoxazole-3-carboxamide as a white solid (0.017 g, 24%). LCMS (Table 1, Method g) Rₜ=1.18 min.; MS m/z: 446.13 (M+H)⁺. ¹H NMR (400 MHz, DMSO) δ ppm 10.60 (s, 1H), 8.04 (d, J=1.79 Hz, 1H), 7.88 (dd, J=8.60, 1.71 Hz, 1H), 7.69 (d, J=8.86 Hz, 2H), 7.42 (s, 1H), 7.36 (d, J=8.72 Hz, 1H), 6.95 (d, J=8.91 Hz, 2H), 5.04-4.86 (m, 1H), 4.86-4.75 (m, 1H), 3.94 (dd, J=9.69, 4.76 Hz, 1H), 3.85 (m, 1H), 3.70 (td, J=10.24, 5.14 Hz, 1H), 2.69 (dd, J=12.83, 4.76 Hz, 1H), 2.58 (dd, J=12.81, 6.40 Hz, 1H), 1.82-1.46 (m, 2H), 1.34 (d, J=5.97 Hz, 6H).

### Preparation of 5-(3-chloro-4-isopropoxyphenyl)-N-(4-(2-hydroxy-3-(2-hydroxyacetamido)propoxy)phenyl)isoxazole-3-carboxamide

### Example #34

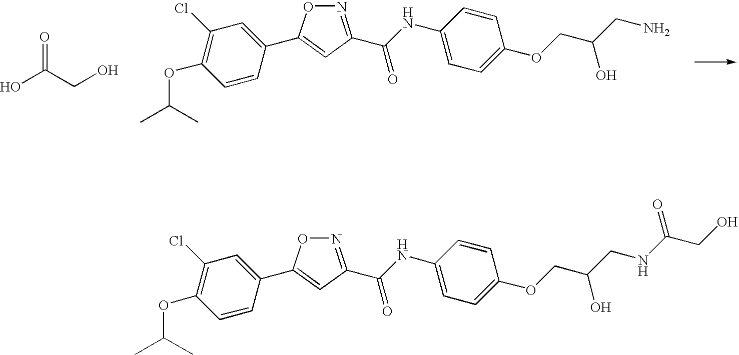

A 25 mL round bottom flask was charged with N-(4-(3-amino-2-hydroxypropoxy)phenyl)-5-(3-chloro-4-isopropoxyphenyl)isoxazole-3-carboxamide (0.015 g, 0.034 mmol), hydroxy-acetic acid (2.014 μL, 0.034 mmol), HOBT (0.0052 mg, 0.034 mmol), EDC (0.00645 g, 0.034 mmol), and DMF (2.0 mL). The reaction mixture was stirred overnight, at ambient temperature, under an atmosphere of nitrogen. Solvent was removed under reduced pressure and the crude material was purified by RP-HPLC (A=50 mM ammonium acetate, B=acetonitrile; 10-90% B over 9.0 min (22.50 mL/min flow rate); Waters: Atlantis Prep T3 OBD, 5 um particle size to give 5-(3-chloro-4-isopropoxyphenyl)-N-(4-(2-hydroxy-3-(2-hydroxyacetamido)propoxy)phenyl)isoxazole-3-carboxamide as a white solid (0.08 g, 72%). LCMS (Table 1, Method g) Rₜ=2.24 min.; MS m/z: 504.29 (M+H)⁺. ¹H NMR (600 MHz, DMSO) δ ppm 10.59 (s, 1H), 8.04 (d, J=2.13 Hz, 1H), 7.88 (dd, J=8.64, 2.13 Hz, 1H), 7.74-7.65 (m, 3H), 7.42 (s, 1H), 7.35 (d, J=8.88 Hz, 1H), 6.94 (d, J=9.02 Hz, 2H), 5.82-4.93 (m, 2H), 4.82 (sept., J=6.08 Hz, 1H), 3.93-3.84 (m, 3H), 3.82 (s, 2H), 3.37 (m, 1H), 3.22-3.18 (m, 1H), 1.34 (d, J=6.02 Hz, 6H).

### 3-Ethoxy-N-(3-(pyridin-4-yl)isoxazol-5-yl)benzamide

### Example #35

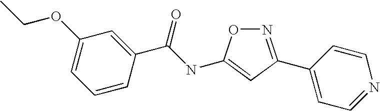

A mixture of 3-ethoxybenzoyl chloride (115 mg, 0.620 mmol), 3-(pyridin-4-yl)isoxazol-5-amine (BetaPharma, 100 mg, 0.620 mmol) and pyridine was stirred at 150° C. in a Biotage microwave for 15 min. The residue was diluted with acetonitrile (7.6 mL), separated in to 4 fractions (2.4 mL) and subjected to purification by RP-LCMS. The combined fractions were evaporated to dryness and dried in vacuo at 60° C. for 6 hours. This afforded 3-ethoxy-N-(3-(pyridin-4-yl)isoxazol-5-yl)benzamide (51 mg, 0.165 mmol, 26.6% yield) as an off-white solid. LCMS (Purity QC) Rₜ=2.45 min; MS m/z 310.14 (M+H)⁺.

### N-(3-(pyridin-4-yl)isoxazol-5-yl)-4-(trifluoromethoxy)benzamide

### Example #36

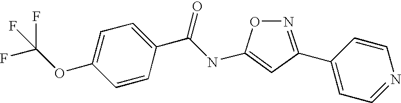

A mixture of 4-(trifluoromethoxy)benzoyl chloride (139 mg, 0.620 mmol), 3-(pyridin-4-yl)isoxazol-5-amine (BetaPharma, 100 mg, 0.620 mmol) and pyridine was stirred at 150° C. in a Biotage microwave for 15 min. The residue was diluted with acetonitrile (7.6 mL), separated in to 4 fractions (2.4 mL) and subjected to purification by RP-LCMS. The combined fractions were evaporated to dryness and dried in vacuo at 60° C. for 6 hours. This afforded N-(3-(pyridin-4-yl)isoxazol-5-yl)-4-(trifluoromethoxy)benzamide (76 mg, 0.218 mmol, 35.1% yield) as an off-white solid. LCMS (Purity QC) Rₜ=2.61 min; MS m/z 348.16 (M−H)⁻.

### Propyl 2-benzamido-4-phenylthiophene-3-carboxylate

### Example #37

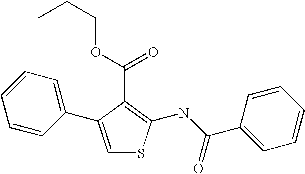

A mixture of propyl 2-amino-4-phenylthiophene-3-carboxylate (Chembridge, 200 mg, 0.765 mmol), benzoyl chloride (108 mg, 0.765 mmol) and pyridine was stirred at 140° C. in a Biotage microwave for 30 min with cooling. The residue was diluted with acetonitrile (7.6 mL), separated in to 4 fractions and subjected to purification by RP-LCMS. The combined fractions were evaporated to dryness and dried in vacuo at 60° C. for 6 hours. This afforded propyl 2-benzamido-4-phenylthiophene-3-carboxylate (105 mg, 0.282 mmol, 36.8% yield) as a yellow solid. LCMS (Purity QC) Rₜ=3.73 min; MS m/z 366.12 (M+H)⁺; H NMR (400 MHz, DMSO-d6) δ 12.09 (s, 1H), 7.98 (m, 2H), 7.72 (m, 1H), 7.66 (m, 2H), 7.35 (m, 5H), 6.99 (s, 1H), 4.03 (t, 2H, J=6.4), 1.31 (m, 2H), 0.54 (t, 3H, J=7.4).

### Ethyl 2-benzamido-4-(4-methoxyphenyl)thiophene-3-carboxylate

### Example #38

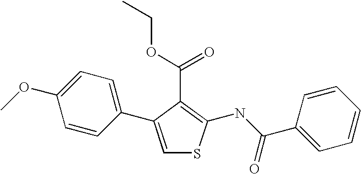

A mixture of ethyl 2-amino-4-(4-methoxyphenyl)thiophene-3-carboxylate (Maybridge, 200 mg, 0.721 mmol), benzoyl chloride (101 mg, 0.721 mmol) and pyridine was stirred at 140° C. in a Biotage microwave for 30 min with cooling. The residue was diluted with acetonitrile (7.6 mL), separated in to 4 fractions and subjected to purification by RP-LCMS. The combined fractions were evaporated to dryness and dried in vacuo at 60° C. for 6 hours. This afforded ethyl 2-benzamido-4-(4-methoxyphenyl)thiophene-3-carboxylate (60 mg, 0.140 mmol, 19.41% yield) as a white solid. LCMS (Purity QC) Rₜ=3.41 min; MS m/z 382.12 (M+H)⁺.

### Ethyl 2-benzamido-4-(4-ethylphenyl)thiophene-3-carboxylate

### Example #39

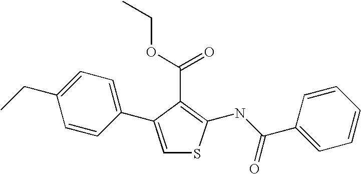

A mixture of ethyl 2-amino-4-(4-ethylphenyl)thiophene-3-carboxylate (Chembridge, 200 mg, 0.726 mmol), benzoyl chloride (102 mg, 0.726 mmol) and pyridine was stirred at 140° C. in a Biotage microwave for 30 min with cooling. The residue was diluted with acetonitrile (7.6 mL), separated in to 4 fractions and subjected to purification by RP-LCMS. The combined fractions were evaporated to dryness and dried in vacuo at 60° C. for 6 hours. This afforded ethyl 2-benzamido-4-(4-ethylphenyl)thiophene-3-carboxylate (91 mg, 0.240 mmol, 33.0% yield) as a white solid. LCMS (Purity QC) Rₜ=3.95 min; MS m/z 380.14 (M+H)⁺.

### Methyl 2-benzamido-4-(4-chlorophenyl)thiophene-3-carboxylate

### Example #40

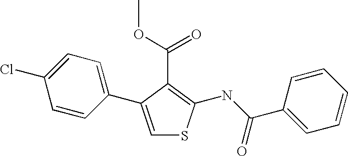

A mixture of methyl 2-amino-4-(4-chlorophenyl)thiophene-3-carboxylate (Chembridge, 200 mg, 0.747 mmol), benzoyl chloride (105 mg, 0.747 mmol) and pyridine was stirred at 140° C. in a Biotage microwave for 30 min with cooling. The residue was diluted with acetonitrile (7.6 mL), separated in to 4 fractions and subjected to purification by RP-LCMS. The combined fractions were evaporated to dryness and dried in vacuo at 60° C. for 6 hours. This afforded methyl 2-benzamido-4-(4-chlorophenyl)thiophene-3-carboxylate (66 mg, 0.156 mmol, 20.91% yield) as an off-white solid. LCMS (Purity QC) Rₜ=3.53 min; MS m/z 372.05 (M+H)⁺.

### N-(3-(4-Chlorophenyl)isoxazol-5-yl)-3-ethoxybenzamide

### Example #41

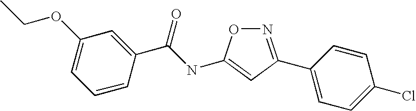

A mixture of 3-ethoxybenzoyl chloride (95 mg, 0.514 mmol), 3-(4-chlorophenyl)isoxazol-5-amine (100 mg, 0.514 mmol) and DMA (2 mL) was stirred at 150° C. in a Biotage microwave for 15 min. Analysis by LCMS showed that starting material was still present and so the mixture was heated at for an additional 15 minutes. The residue was diluted with acetonitrile (7.6 mL), separated in to 4 fractions (2.4 mL) and subjected to purification by RP-LCMS. The combined fractions were evaporated to dryness and dried in vacuo at 60° C. for 6 hours. This afforded N-(3-(4-chlorophenyl)isoxazol-5-yl)-3-ethoxybenzamide (32 mg, 0.093 mmol, 18.17% yield) as an off-white solid. LCMS (Purity QC) Rₜ=3.25 min; MS m/z 343.10 (M+H)⁺.

### N-(3-(2-chlorophenyl)isoxazol-5-yl)-3-ethoxybenzamide

### Example #42

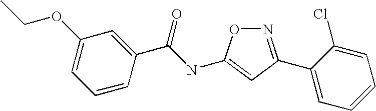

A mixture of 3-ethoxybenzoyl chloride (95 mg, 0.514 mmol), 3-(2-chlorophenyl)isoxazol-5-amine (100 mg, 0.514 mmol) and DMA (2 mL) was stirred at 150° C. in a Biotage microwave for 30 min. The residue was diluted with acetonitrile (7.6 mL), separated in to 4 fractions (2.4 mL) and subjected to purification by RP-LCMS. The combined fractions were evaporated to dryness and dried in vacuo at 60° C. for 6 hours. This afforded N-(3-(2-chlorophenyl)isoxazol-5-yl)-3-ethoxybenzamide (45 mg, 0.131 mmol, 25.5% yield) as an off-white solid. LCMS (Purity QC) Rₜ=2.94 min; MS m/z 343.09 (M+H)⁺.

### N-(3-(4-methoxyphenyl)isoxazol-5-yl)-2-methylbenzamide

### Example #43

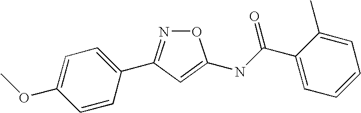

A mixture of 3-(4-methoxyphenyl)isoxazol-5-amine (200 mg, 1.052 mmol), 2-methylbenzoyl chloride (163 mg, 1.052 mmol) and pyridine was stirred at 150° C. in a Biotage microwave for 15 min. The residue was diluted with acetonitrile (7.6 mL), separated in to 4 fractions (2.4 mL) and subjected to purification by RP-LCMS. The combined fractions were evaporated to dryness and dried in vacuo at 60° C. for 6 hours. This afforded N-(3-(4-methoxyphenyl)isoxazol-5-yl)-2-methylbenzamide (175 mg, 0.568 mmol, 54.0% yield) as an off-white solid. LCMS (Purity QC) Rₜ=2.68 min; MS m/z 309.13 (M+H)⁺.

### 2-Chloro-N-(3-(4-methoxyphenyl)isoxazol-5-yl)benzamide

### Example #44

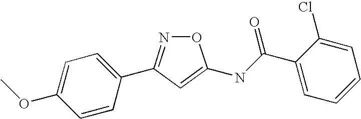

A mixture of 3-(4-methoxyphenyl)isoxazol-5-amine (200 mg, 1.052 mmol), 2-chlorobenzoyl chloride (184 mg, 1.052 mmol) and pyridine was stirred at 150° C. in a Biotage microwave for 15 min. The residue was diluted with acetonitrile (7.6 mL), separated in to 4 fractions (2.4 mL) and subjected to purification by RP-LCMS. The combined fractions were evaporated to dryness and dried in vacuo at 60° C. for 6 hours. This afforded 2-chloro-N-(3-(4-methoxyphenyl)isoxazol-5-yl)benzamide (200 mg, 0.608 mmol, 57.9% yield) as an off-white solid. LCMS (Purity QC) Rₜ=2.56 min; MS m/z 329.07 (M+H)⁺.

### N-(3-(4-Methoxyphenyl)isoxazol-5-yl)-2-(trifluoromethyl)benzamide

### Example #45

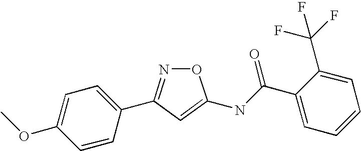

A mixture of 3-(4-methoxyphenyl)isoxazol-5-amine (200 mg, 1.052 mmol), 2-(tri-fluoromethyl)benzoyl chloride (219 mg, 1.052 mmol) and pyridine was stirred at 150° C. in a Biotage microwave for 15 min. The residue was diluted with acetonitrile (7.6 mL), separated in to 4 fractions (2.4 mL) and subjected to purification by RP-LCMS. The combined fractions were evaporated to dryness and dried in vacuo at 60° C. for 6 hours. This afforded N-(3-(4-methoxyphenyl)isoxazol-5-yl)-2-(trifluoromethyl)benzamide (220 mg, 0.608 mmol, 60.8% yield) as an off-white solid. LCMS (Purity QC) Rₜ=2.65 min; MS m/z 363.09 (M+H)⁺.

### N-(1-Ethyl-1H-pyrazol-5-yl)-4-phenyl-5-(trifluoromethyl)thiophene-2-carboxamide

### Example #46

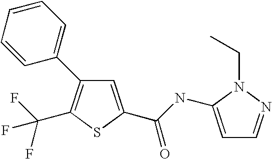

A mixture of 4-phenyl-5-(trifluoromethyl)thiophene-2-carbonyl chloride (100 mg, 0.344 mmol), 1-ethyl-1H-pyrazol-5-amine (38.2 mg, 0.344 mmol) and pyridine was stirred at 150° C. in a Biotage microwave for 15 min. The residue was diluted with acetonitrile (7.6 mL), separated in to 4 fractions (2.4 mL) and subjected to purification by RP-LCMS. The combined fractions were evaporated to dryness and dried in vacuo at 60° C. for 6 hours. This afforded N-(1-ethyl-1H-pyrazol-5-yl)-4-phenyl-5-(trifluoromethyl)thiophene-2-carboxamide (78 mg, 0.199 mmol, 57.7% yield) as an off-white solid. LCMS (Purity QC) Rₜ=2.81 min; MS m/z 366.10 (M+H)⁺; H NMR (400 MHz, DMSO-d6) δ 10.56 (s, 1H), 8.16 (s, 1H), 7.52 (m, 5H), 7.47 (d, 1H, J=1.8), 6.26 (d, 1H, J=1.8), 4.04 (q, 2H, J=7.1), 1.32 (t, 3H, J=7.2).

### N-(2-Ethylphenyl)-4-phenyl-5-(trifluoromethyl)thiophene-2-carboxamide

### Example #47

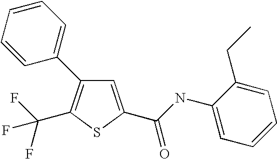

A mixture of 4-phenyl-5-(trifluoromethyl)thiophene-2-carbonyl chloride (100 mg, 0.344 mmol), 2-ethylaniline (41.7 mg, 0.344 mmol) and pyridine was stirred at 150° C. in a Biotage microwave for 15 min. The residue was diluted with acetonitrile (7.6 mL), separated in to 4 fractions (2.4 mL) and subjected to purification by RP-LCMS. The combined fractions were evaporated to dryness and dried in vacuo at 60° C. for 6 hours. This afforded (2-ethylphenyl)-4-phenyl-5-(trifluoromethyl)thiophene-2-carboxamide (61 mg, 0.162 mmol, 47.2% yield) as an off-white solid. LCMS (Purity QC) Rₜ=3.52 min; MS m/z 376.10 (M+H)⁺.

### 4-Phenyl-N-o-tolyl-5-(trifluoromethyl)thiophene-2-carboxamide

### Example #48

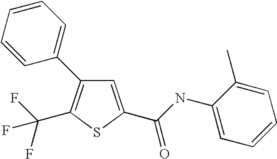

A mixture of 4-phenyl-5-(trifluoromethyl)thiophene-2-carbonyl chloride (100 mg, 0.344 mmol), o-toluidine (36.9 mg, 0.344 mmol) and pyridine was stirred at 150° C. in a Biotage microwave for 15 min. The residue was diluted with acetonitrile (7.6 mL), separated in to 4 fractions (2.4 mL) and subjected to purification by RP-LCMS. The combined fractions were evaporated to dryness and dried in vacuo at 60° C. for 6 hours. This afforded 4-phenyl-N-o-tolyl-5-(trifluoromethyl)thiophene-2-carboxamide (73 mg, 0.186 mmol, 54.0% yield) as an off-white solid. LCMS (Purity QC) Rₜ=3.31 min; MS m/z 362.09 (M+H)⁺.

### N-(1,3-Dimethyl-1H-pyrazol-5-yl)-4-phenyl-5-(trifluoromethyl)thiophene-2-carboxamide

### Example #49

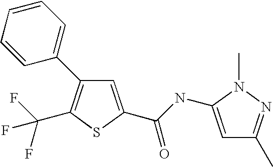

A mixture of 4-phenyl-5-(trifluoromethyl)thiophene-2-carbonyl chloride (100 mg, 0.344 mmol), 1,3-dimethyl-1H-pyrazol-5-amine (38.2 mg, 0.344 mmol) and pyridine was stirred at 150° C. in a Biotage microwave for 15 min. The residue was diluted with acetonitrile (7.6 mL), separated in to 4 fractions (2.4 mL) and subjected to purification by RP-LCMS. The combined fractions were evaporated to dryness and dried in vacuo at 60° C. for 6 hours. This afforded N-(1,3-dimethyl-1H-pyrazol-5-yl)-4-phenyl-5-(trifluoromethyl)thiophene-2-carboxamide (37 mg, 0.099 mmol, 28.8% yield) as an off-white solid. LCMS (Purity QC) Rₜ=2.76 min; MS m/z 366.09 (M+H)⁺.

### Ethyl 2-benzamido-4-(4-propylphenyl)thiophene-3-carboxylate

### Example #50

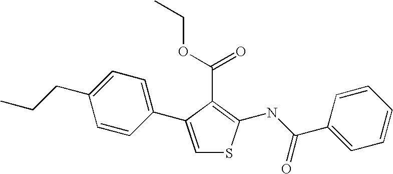

A mixture of ethyl 2-amino-4-(4-propylphenyl)thiophene-3-carboxylate (200 mg, 0.691 mmol), benzoyl chloride (97 mg, 0.691 mmol) and pyridine was stirred at 140° C. in a Biotage microwave for 30 min with cooling. The residue was diluted with acetonitrile (7.6 mL), separated in to 4 fractions and subjected to purification by RP-LCMS. The combined fractions were evaporated to dryness and dried in vacuo at 60° C. for 6 hours. This afforded ethyl 2-benzamido-4-(4-propylphenyl)thiophene-3-carboxylate (87 mg, 0.221 mmol, 32.0% yield) as an off-white solid. LCMS (Purity QC) Rₜ=4.18 min; MS m/z 394.15 (M+H)⁺.

### Ethyl 2-benzamido-4-(4-methoxyphenyl)-5-methylthiophene-3-carboxylate

### Example #51

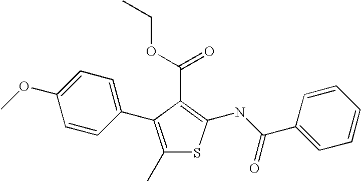

A mixture of ethyl 2-amino-4-(4-methoxyphenyl)-5-methylthiophene-3-carboxylate (200 mg, 0.686 mmol), benzoyl chloride (96 mg, 0.686 mmol) and pyridine was stirred at 140° C. in a Biotage microwave for 30 min with cooling. The residue was diluted with acetonitrile (7.6 mL), separated in to 4 fractions and subjected to purification by RP-LCMS. The combined fractions were evaporated to dryness and dried in vacuo at 60° C. for 6 hours. This afforded ethyl 2-benzamido-4-(4-methoxyphenyl)-5-methylthiophene-3-carboxylate (140 mg, 0.354 mmol, 51.6% yield) as a yellow solid. LCMS (Purity QC) Rₜ=3.68 min; MS m/z 396.3 (M+H)⁺.

### Preparation of 4-Formyl-N-hexylbenzamide

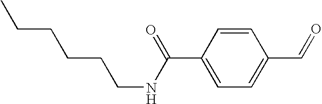

A mixture of 4-carboxybenzaldehyde (0.5 g, 3.3 mmol), fluoro-N,N,N′,N′-tetramethyl-formamidinium hexafluorophosphate (0.88 g, 3.3 mmol), DIPEA (0.58 mL, 3.3 mmol) and NMP (10 mL) was stirred at ambient temperature for 30 min. Hexylamine (0.44 mL, 3.3 mmol) in NMP (6 mL) was added and the mixture stirred for a further 30 min. Water was added (20 mL) and the mixture extracted with DCM (2×30 mL). The combined organic layers were dried (Na₂SO₄), filtered and evaporated to dryness. This gave a yellow/gold residue. The residue was diluted with 1:1 DMSO:acetonitrile (24 mL) and subjected to purification by RP-LCMS. The combined fractions were evaporated to dryness. This afforded 4-formyl-N-hexylbenzamide (410 mg, 58% yield) as a white solid. LCMS (Purity QC) Rₜ=2.65 min; MS m/z 234.24 (M+H)⁺.

### 1-(4-(Hexylcarbamoyl)benzyl)azetidine-3-carboxylic acid

### Example #52

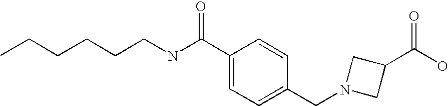

A mixture of 4-formyl-N-hexylbenzamide (390 mg, 1.7 mmol), azetidine carboxylic acid (169 mg, 1.7 mmol), MP-sodium cyanoborohydride (Biotage, 1.5 g, 3.4 mmol, 2.3 mmol/g loading), methanol (10 mL) and acetic acid (12 drops) was stirred at ambient temperature for 24 h. The mixture was filtered and evaporated to dryness. This gave a white solid on standing. The residue was subjected to purification by RP-LCMS. The combined fractions were evaporated to dryness and dried in vacuo at 60° C. overnight. This afforded 1-(4-(hexylcarbamoyl)benzyl)azetidine-3-carboxylic acid (470 mg, 88% yield) as a white solid. LCMS (table 1, Method a) Rₜ=1.67 min; MS m/z 319.34 (M+H)⁺; ¹H NMR (400 MHz, DMSO-d6) δ 7.38 (d, 2H, J=8.0), 7.15 (d, 2H, J=8.0), 4.08 (d, 2H, J=9.4), 3.95 (m, 4H), 3.34 (m, 1H), 2.95 (t, 2H, J=6.9), 1.18 (m, 2H), 0.90 (m, 6H), 0.42 (t, 3H, J=6.9).

### Preparation of 1-(5-iodo-1H-indazol-1-yl)ethanone

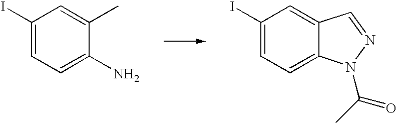

A solution of 4-iodo-2-methylaniline (50.0 g, 215 mmol) in toluene (500 mL) was cooled to 0-5° C., with stirring, before the dropwise addition of acetic anhydride (46.6 mL, 493 mmol) in toluene (2 mL) over 10 mms. The reaction was stirred for 30 mins. The acetylated, purple material precipitated. To the suspension isoamyl nitrite (57.8 mL, 429 mmol) and potassium acetate (6.32 g, 64.4 mmol) were added and the reaction mixture was stirred at 80° C. for 18 hr.

The reaction was cooled to ambient temperature before the dark yellow solution was basified by the addition of 1N NaOH (50 mL). The organic layer was separated and the basic aqueous phase was washed with toluene (3×100 mL). The extracts and original organic layer were combined, washed with water (3×300 mL), dried over MgSO₄, filtered and solvent removed in vacuo to yield a tan solid 56.5 g that still contained a minor amount of toluene. The residue was triturated with 30-60° C. pet/ether (56 mL), solid collected, washed with 30-60° C. pet/ether (2×15 mL) and dried to yield a light tan solid 53.55 g. The solid was stirred with 30-60° C. pet/ether (150 mL), solid collected, washed with 30-60° C. pet/ether (2×50 mL) and dried to yield 1-(5-iodo-1H-indazol-1-yl)ethanone (52.20 g, 90%) as a light tan solid: LCMS (Method b) Rt=1.99 min; MS m/z 243.02 (M-acetate)⁻.

### Preparation of 1-(5-((trimethylsilyl)ethynyl)-1H-indazol-1-yl)ethanone

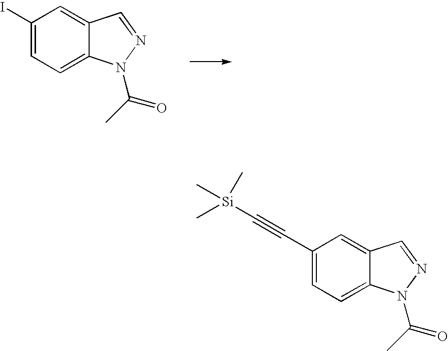

Nitrogen was bubbled through a suspension of 1-(5-iodo-1H-indazol-1-yl)ethanone (52.2 g, 182 mmol), bis(triphenylphosphine)palladium(II) chloride (6.53 g, 9.31 mmol) and copper (1) iodide (1.668 g, 8.76 mmol) in triethylamine (453 mL, 3252 mmol) for 10 mins at room temperature. TMS-Acetylene (21.90 g, 223 mmol) was added and the reaction was sealed, under an atmosphere of nitrogen, then stirred and heated at 60° C. for 18 hr.

The solvent was removed in vacuo. The resulting residue was dissolved in DCM (300 mL) and to the dark brown solution was added H₂O (250 mL). The mixture was stirred for 10 minutes and passed through a Celite® pad. The pad was washed with DCM (3×150 mL). The aqueous phase was extracted with DCM (2×60 mL). The organic extracts were combined and washed with H₂O (3×200 mL), dried over MgSO₄, filtered and solvent removed to yield 1-(5-((trimethylsilyl)ethynyl)-1H-indazol-1-yl)ethanone (57.87 g, 124%) as a dark yellow solid: LCMS (Method b) Rt=2.38 min; MS m/z 256.16 (M+H)⁺.

### Preparation of 5-ethynyl-1H-indazole

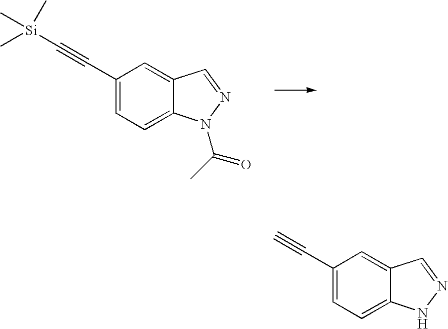

The 1-(5-((trimethylsilyl)ethynyl)-1H-indazol-1-yl)ethanone (57.6 g, 225 mmol) was dissolved in MeOH (609 mL) and to this water (115 mL) followed by potassium hydroxide (27.7 g, 494 mmol). The brown solution was stirred at 20-25° C. for 3 h. The solvent was removed in vacuo and the brown residue was diluted with H₂O (400 mL). The product was partitioned between the basic aqueous layer and EtOAc (3×250 mL). The extracts were combined, washed with H₂O (3×100 mL), dried over MgSO₄, filtered and solvent removed in vacuo to yield the 5-ethynyl-1H-indazole (28.65 g, 88%) as a brown coloured solid: LCMS (Method b) Rt=1.85 min; MS m/z 143.07 (M+H)+

### Preparation of ethyl 5-(1H-indazol-5-yl)isoxazole-3-carboxylate

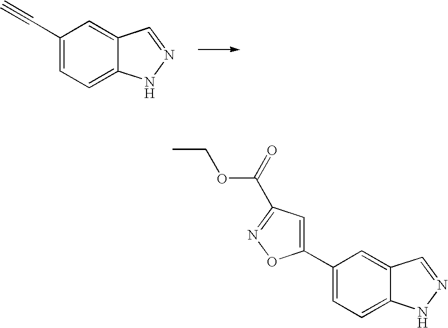

A solution of ethyl chlorooximidoacetate (24.31 g, 160 mmol) in Toluene (504 mL) was added dropwise, extremely slowly (over 2.5 hr) to a solution of 5-ethynyl-1H-indazole (19.00 g, 134 mmol) and triethylamine (20.49 mL, 147 mmol) in toluene (700 mL) at 90° C. After the addition was complete the reaction mixture was heated and stirred at 90° C. for 1 hr.

Solvent removed and the residue was diluted with water (400 mL), stirred for 15 min. Solid collected and washed with H₂O (3×100 mL) and washed with IPA (2×40 mL), followed by MeCN (2×25 mL) and dried in vacuo to yield 5-(1H-indazol-5-yl)isoxazole-3-carboxylate (23.59 g, 68%) as a tan powdery solid: LCMS (Method b) Rt=1.98 min; MS m/z 258.12 (M+H)⁺.

### Preparation of 5-(1H-indazol-5-yl)isoxazole-3-carboxylic acid

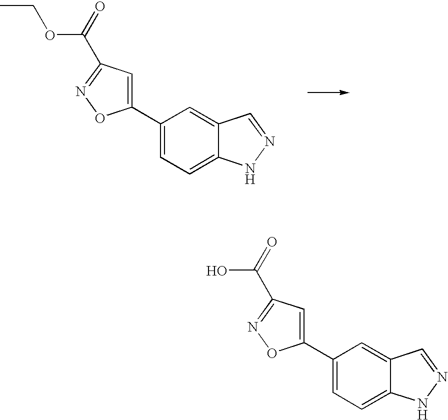

The ethyl 5-(1H-indazol-5-yl)isoxazole-3-carboxylate (23.15 g, 90 mmol) was dissolved in THF (235 mL), and to the solution were added MeOH (23.51 mL), water (23.51 mL) and potassium hydroxide (10.10 g, 180 mmol) and the reaction mixture was stirred at ambient temperature for 3 h. The THF was removed in vacuo and 1N HCl (157 mL) was added to the basic aqueous phase and the carboxylic acid was collected and washed with water (3×50 mL), followed by IPA (2×25 mL) and finally acetonitrile (3×50 mL). The solid was dried in a vacuum oven at 40° C. overnight to afford crude 5-(1H-indazol-5-yl)isoxazole-3-carboxylic acid (23.54 g). The solid was triturated with boiling acetonitrile (250 mL), cooled to room temperature, and the solid collected and washed with acetonitrile (2×25 mL) and dried. The solid was stirred at room temperature with MeOH (100 mL), filtered and dried in vacuo at 40° C. to yield 5-(1H-indazol-5-yl)isoxazole-3-carboxylic acid (19.76 g, 93%) as a tan powdery solid: LCMS (Method b) Rt=1.28 min; MS m/z 228.16 (M−H)⁻.

### Preparation of 1-(4-(2-(5-(3-chloro-4-isopropoxyphenyl)isoxazol-3-yl)-2-hydroxyethyl)benzyl)azetidine-3-carboxylic acid

### Example #53

### Step A. Preparation of (5-(3-chloro-4-isopropoxyphenyl)isoxazol-3-yl)methanol

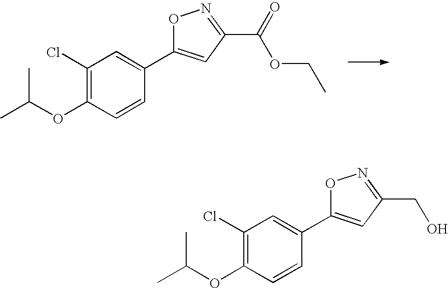

Ethyl 5-(3-chloro-4-isopropoxyphenyl)isoxazole-3-carboxylate (0.1 g, 0.323 mmol) was dissolved in THF (6.46 mL) under nitrogen to give a colorless solution. A solution of lithium aluminum hydride (0.161 mL, 0.323 mmol) was added carefully and the reaction stirred for about 2 h. The reaction was quenched by careful addition of water (12 μL) and the reaction stirred for 30 min. 10% NaOH (36 μL) was added and the reaction stirred for about 30 min. Finally, water (12 μL) was added and the reaction stirred for about 30 min. The resulting precipitate was removed by filtering through a syringe filter. The solvents were removed under reduced pressure. The residue was purified by flash column chromatography (12 g Redi-Sep) eluting with ethyl acetate/heptane (20-50%) and the product reactions combined. The solvents were removed under reduced pressure to provide (5-(3-chloro-4-isopropoxyphenyl)isoxazol-3-yl)methanol (0.077 g, 0.288 mmol, 89% yield) as a yellow oil: LCMS (Method a) Rₜ=3.01 min.; MS m/z: 267.95, 269.87 (M+H)⁺.

### Step B. Preparation of 5-(3-chloro-4-isopropoxyphenyl)isoxazole-3-carbaldehyde

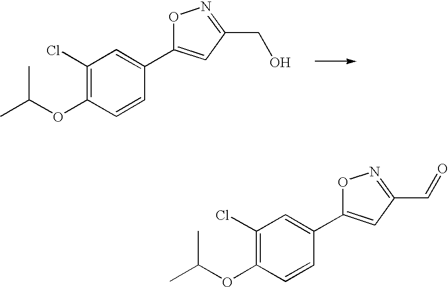

(5-(3-chloro-4-isopropoxyphenyl)isoxazol-3-yl)methanol (1.267 g, 4.73 mmol) was dissolved in dichloromethane (47.3 mL) in a sealed vial to give a yellow solution. Dess-Martin periodinane (2.208 g, 5.21 mmol) was added and the reaction stirred for about 2 h. TLC in 1:1 EtOAc/heptane showed (uv light visualization) reaction complete. The reaction was quenched by addition of saturated sodium bicarbonate (50 mL). The aqueous layer was extracted with methylene chloride (2×50 mL). The combined extracts were washed with brine, dried over sodium sulfate, filtered, and evaporated to a colorless oil that solidified on standing. The residue was purified by flash column chromatography (80 g Redi-Sep) eluting with 1:1 EtOAc/heptane and the product fractions combined. The solvents were removed under reduced pressure to provide 5-(3-chloro-4-isopropoxyphenyl)isoxazole-3-carbaldehyde (1.16 g, 4.37 mmol, 92% yield) as a white solid: LCMS (Method m) Rₜ=2.35 min.; MS m/z: 266.11 (M+H)⁺.

### Step C. Preparation of 4-(2-(5-(3-chloro-4 isopropoxyphenyl)isoxazol-3-yl)-2-hydroxyethyl)benzonitrile

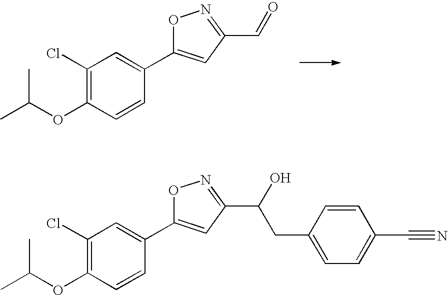

5-(3-chloro-4-isopropoxyphenyl)isoxazole-3-carbaldehyde (0.5 g, 1.882 mmol) was dissolved in THF (37.6 mL) under nitrogen to give a colorless solution. The reaction was cooled to about −45° C. in a dry ice/acetonitrile bath. A solution of 4-cyano-benzylzinc bromide (7.53 mL, 3.76 mmol) was added slowly and the reaction stirred for about 30 min. TLC in 1:1 EtOAc/heptane showed (uv light visualization) no reaction. The cooling was removed, the reaction allowed to warm to ambient temperature and stirred for about 18 h. TLC showed some product forming with much baseline material. The reaction was quenched by addition of saturated sodium bicarbonate (15 mL). Ethyl acetate (100 mL) and water (15 mL) were added and the layers separated. The aqueous layer was extracted with ethyl acetate (2×25 mL). The combined extracts were washed with brine, dried over sodium sulfate, filtered, and evaporated to a yellow solid. The residue was purified by flash column chromatography (80 g Redi-Sep) eluting with 10-50% ethyl acetate/heptane and the product fractions combined. The solvents were removed under reduced pressure to give two products, the less polar spot was the desired product 4-(2-(5-(3-chloro-4-isopropoxyphenyl)isoxazol-3-yl)-2-hydroxyethyl)benzo-nitrile (0.163 g, 0.426 mmol, 22.62% yield): LCMS (Method g) Rₜ=2.65 min.; MS m/z: 383.13 (M+H)⁺.

### Step D. Preparation of 4-(2-(5-(3-chloro-4-isopropoxyphenyl)isoxazol-3-yl)-2-hydroxyethyl)benzaldehyde

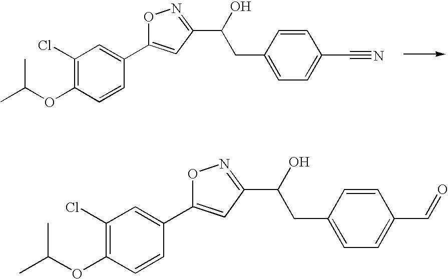

4-(2-(5-(3-chloro-4-isopropoxyphenyl)isoxazol-3-yl)-2-hydroxyethyl)benzonitrile (0.163 g, 0.426 mmol) was dissolved in dichloromethane (8.52 mL) under nitrogen to give a colorless solution. The reaction was cooled to about −45° C. in a dry ice/acetonitrile bath. A solution of DIBAL-H (1.277 mL, 1.277 mmol) (Aldrich) was added slowly and the reaction stirred for about 2 h. The reaction was quenched by addition of 10% potassium sodium tartrate (Rochelle's salt) (5 mL) and the mixture was removed from the cooling bath and stirred rapidly overnight. The layers were separated. The aqueous layer was extracted with ethyl acetate (2×10 mL). The combined extracts were washed with 0.1 N HCl (1×10 mL). The combined extracts were washed with brine, dried over sodium sulfate, filtered, and evaporated to give 4-(2-(5-(3-chloro-4-isopropoxyphenyl)isoxazol-3-yl)-2-hydroxyethyl)benzaldehyde (0.142 g, 0.368 mmol, 86% yield) as an off-white solid: LCMS (Method g) Rₜ=2.60 min.; MS m/z: 386.13, 388.05 (M+H)⁺.

### Step E. Preparation of 1-(4-(2-(5-(3-chloro-4-isopropoxyphenyl)isoxazol-3-yl)-2-hydroxyethyl)benzyl)azetidine-3-carboxylic acid

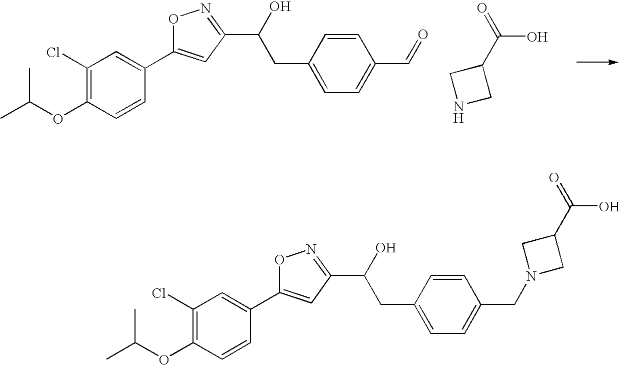

4-(2-(5-(3-chloro-4-isopropoxyphenyl)isoxazol-3-yl)-2-hydroxyethyl)benzaldehyde (0.142 g, 0.368 mmol), azetidine-3-carboxylic acid (0.045 g, 0.442 mmol) (Synchem), and acetic acid (0.084 mL, 1.472 mmol) were combined in methanol (7.36 mL) in a sealed vial to give a colorless solution. MP-cyanoborohydride (0.514 g, 1.104 mmol) (Argonaut) was added and the reaction stirred for about 72 h. The mixture was filtered through celite and washed through with methanol containing ammonium hydroxide. The solvents were removed under reduced pressure. The residue was purified by flash column chromatography (0.5″×6″ of silica) eluting with 1:1 EtOAc/(6:3:1 CHCl₃/MeOH/NH₄OH) and the product fractions combined. The solvents were removed under reduced pressure. The residue was triturated with ether. The resulting solid was collected by vacuum filtration and washed with ether to provide 1-(4-(2-(5-(3-chloro-4-isopropoxyphenyl)isoxazol-3-yl)-2-hydroxyethyl)benzyl)azetidine-3-carboxylic acid (0.098 g, 0.208 mmol, 56.5% yield) as a white solid on drying under vacuum at 60° C.: LCMS (Method g) Rₜ=1.86 min.; MS m/z: 471.05, 473.08 (M+H)⁺.

### Preparation of 1-(4-((5-(3-chloro-4-isopropoxyphenyl)isoxazol-3-yl)methylamino)benzyl)azetidine-3-carboxylic acid

### Example #54

### Step A. Preparation of 1-(4-(benzyloxycarbonylamino)benzyl)azetidine-3-carboxylic acid

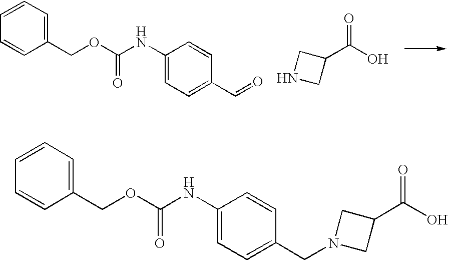

Azetidine-3-carboxylic acid (3.49 g, 34.6 mmol) was dissolved in acetic acid (15.07 mL, 263 mmol) and a few mL of methanol. This was added to a solution of benzyl 4-formylphenyl-carbamate (8.4 g, 32.9 mmol) (J. Am. Chem. Soc. 2005, 127, 2717-2724) in methanol (823 mL). MP-cyanoborohydride (15.31 g, 32.9 mmol) (Argonaut) was added and the reaction stirred for about 7 days. A heavy percipitate had formed that was not forming initially but formed on the long reaction/stir time. The precipitate and resin were collected by vacuum filtration and wash with methanol. Wash product off of resin with methanol and ammonium hydroxide. Concentrate and chromatograph over silica gel in 6:3:1 CHCl₃/MeOH/NH₄OH. Product containing fractions were combined and concentrated. Some methanol was added and let stand resulting in a precipitate. The resulting solid was collected by vacuum filtration and washed with methanol to provide 1-(4-(benzyloxycarbonylamino)benzyl)azetidine-3-carboxylic acid (5.15 g, 15.13 mmol, 46.0% yield) as a off-white solid: LCMS (Method a) Rₜ=2.34 min.; MS m/z: 341.20 (M+H)⁺.

### Step C. Preparation of 1-(4-aminobenzyl)azetidine-3-carboxylic acid

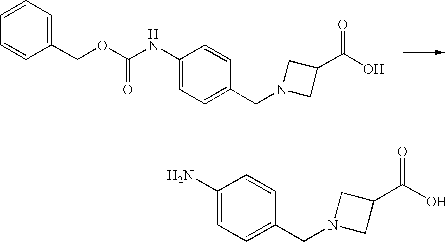

1-(4-(benzyloxycarbonylamino)benzyl)azetidine-3-carboxylic acid (4 g, 11.75 mmol) was stirred in ethanol (235 mL). Palladium on carbon (1.251 g, 1.175 mmol) was added and the reaction was flushed with hydrogen gas stirred rapidly for about 48 h. The starting material was slow to dissolve necessitating the long reaction time. Filter through celite and wash through with methanol followed by rotovaporization. Wash celite with methanol/ammonium hydroxide until TLC shows no more product eluting. The residue was purified by flash column chromatography (1″×7″ of silica) eluting with 6:3:1 CHCl₃/MeOH/NH OH and the product fractions combined. Rotovap to a tan solid. Dry under vacuum overnight. Add ether and scrape off flask. The resulting solid was collected by vacuum filtration and washed with ether to provide 1-(4-aminobenzyl)azetidine-3-carboxylic acid (1.526 g, 7.40 mmol, 63.0% yield) as a off-white solid: LCMS (Method a) Rₜ=0.57 min.; MS m/z: 207.09 (M+H)⁺ and MS m/z: 413.28 (2M+H)⁺.

### Step D. Preparation of 1-(4-((5-(3-chloro-4-isopropoxyphenyl)isoxazol-3-yl)methylamino)benzyl)azetidine-3-carboxylic acid

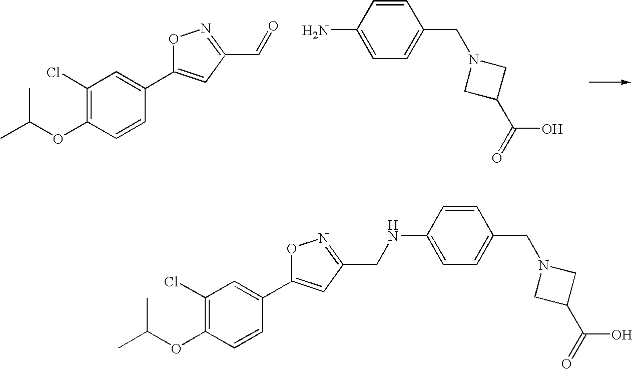

5-(3-chloro-4-isopropoxyphenyl)isoxazole-3-carbaldehyde (0.1 g, 0.376 mmol), 1-(4-aminobenzyl)azetidine-3-carboxylic acid (0.093 g, 0.452 mmol), and acetic acid (0.086 mL, 1.506 mmol) (VWR) were combined in methanol (3.76 mL) in a sealed vial to give a colorless suspension. The mixture was warmed with a heat gun to facilitate dissolution and the solution stirred for about 45 min. MP-cyanoborohydride (0.525 g, 1.129 mmol) (Argonaut) was added and the reaction stirred for about 2 h. A heavy white precipitate had formed in the reaction. Additional methanol (4 mL) was added and the reaction stirred for about 16 h. The precipitate was collected by vacuum filtration (buchner funnel, Whatman #1 filter paper) and washed with methanol. The collection flask was changed and the product separated from the MP-cyanoborohydride by washing it through the filter with mixture of methanol and ammonium hydroxide. The filtrate was evaporated and triturated with ether and methanol to give a white solid that was collected by vacuum filtration and washed with ether. NMR shows a minor impurity. TLC in 6:3:1 CHCl₃/MeOH/NH₄OH showed (uv light visualization) a small amount of a more polar impurity. The product was purified by flash column chromatography (0.5″×7″ of silica) eluting with 1:1 EtOAc/(6:3:1 CHCl₃/MeOH/NH₄OH) and the product fractions combined. The solvents were removed under reduced pressure. The residue was triturated with methanol only. The resulting solid was collected by vacuum filtration and washed with methanol and pentane to provide 1-(4-((5-(3-chloro-4-isopropoxyphenyl)isoxazol-3-yl)methylamino)benzyl)azetidine-3-carboxylic acid (0.0704 g, 0.154 mmol, 41.0% yield) as a white solid on drying under vacuum: LCMS (Method g) Rₜ=1.90 min.; MS m/z: 454.50 (M−H)⁻.

### 1-(4-((5-(3-chloro-4-isopropoxyphenyl)isoxazol-3-yl)methoxy)benzyl)-azetidine-3-carboxylic acid

### Example #55

### Step A. Preparation of 4-((5-(3-chloro-4-isopropoxyphenyl)isoxazol-3-yl)methoxy)benzonitrile

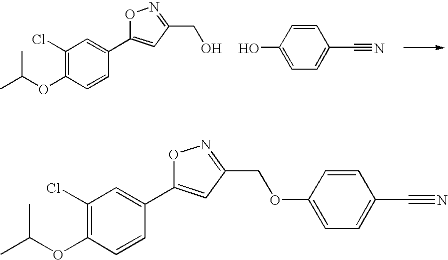

(5-(3-chloro-4-isopropoxyphenyl)isoxazol-3-yl)methanol (0.267 g, 0.997 mmol), 4-hydroxybenzonitrile (0.178 g, 1.496 mmol), and triphenylphosphine resin bound (0.831 g, 2.493 mmol) were combined in THF (9.97 mL) under nitrogen to give a brown suspension. Molecular sieves 4 A (0.45 g, 0.997 mmol) were added and the reaction stirred for about 30 min. Di-tert-butyl azodicarboxylate (0.344 g, 1.496 mmol) was added and the reaction stirred for about 18 h. The mixture was filtered through celite and washed through with ethyl acetate. The solvents were removed under reduced pressure. The residue was purified by flash column chromatography (40 g Redi-Sep) eluting with ethyl acetate/heptane and the product fractions combined. The solvents were removed under reduced pressure. NMR showed reduced DBAD. The residue was dissolve in methylene chloride (5 mL). Trifluoroacetic acid (5 mL) was added and the reaction stirred for about 40 h. The solvents were removed under reduced pressure. methylene chloride (20 mL) and saturated sodium bicarbonate (15 mL) were added and the layers separated. The aqueous layer was extracted with methylene chloride (2×10 mL). The combined extracts were washed with brine, dried over sodium sulfate, filtered, and evaporated to a yellow oil. The residue was purified by flash column chromatography (40 g Redi-Sep) eluting with 20-50% ethyl acetate/heptane and the product fractions combined. The solvents were removed under reduced pressure to provide 4-((5-(3-chloro-4-isopropoxyphenyl)isoxazol-3-yl)methoxy)benzonitrile (0.118 g, 0.320 mmol, 32.1% yield) as an off-white solid: LCMS (Method g) Rₜ=2.92 min.; MS m/z: 369.10 (M+H)⁺.

### Step B. Preparation of 4-((5-(3-chloro-4-isopropoxyphenyl)isoxazol-3-yl)methoxy)benzaldehyde

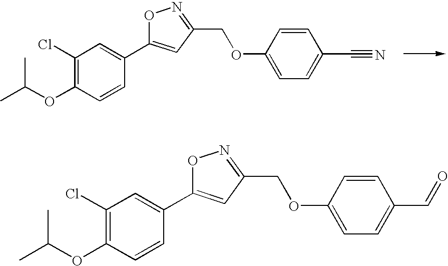

4-((5-(3-chloro-4-isopropoxyphenyl)isoxazol-3-yl)methoxy)benzonitrile (0.118 g, 0.320 mmol) was dissolved in dichloromethane (6.40 mL) under nitrogen to give a colorless solution. The reaction was cooled to about −45° C. in a dry ice/acetonitrile bath. A solution of diisobutylaluminum hydride (0.640 mL, 0.640 mmol) was added dropwise and the reaction stirred for about 30 min. The reaction was quenched by addition of 10% potassium sodium tartrate (Rochelle's salt) (5 mL) and stirred rapidly for 1 h. The layers were separated. The aqueous layer was extracted with methylene chloride (2×5 mL). The combined extracts were washed with brine, dried over sodium sulfate, filtered, and evaporated to an off-white solid. NMR showed that the aldehyde did not integrate enough and there were other peaks present indicating incomplete imine hydrolysis. Redissolve the solid in about 10 mL of methylene chloride and stir rapidly with about 10 mL of 1 N HCl. Separate the layers. The aqueous layer was extracted with methylene chloride (2×10 mL). The combined extracts were washed with brine, dried over sodium sulfate, filtered, and evaporated to provide 4-((5-(3-chloro-4-isopropoxyphenyl)isoxazol-3-yl)methoxy)benzaldehyde (0.09 g, 0.242 mmol, 76% yield) as a white solid: LCMS (Method g) Rₜ=2.89 min.; MS m/z: 372.09 (M+H)⁺.

### Step C. Preparation of 1-(4-((5-(3-chloro-4-isopropoxyphenyl)isoxazol-3-yl)methoxy)benzyl)azetidine-3-carboxylic acid

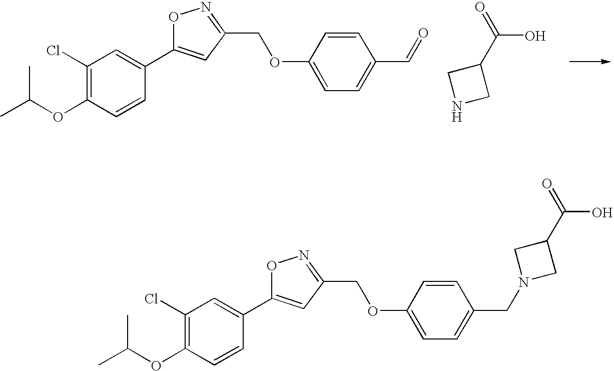

4-((5-(3-chloro-4-isopropoxyphenyl)isoxazol-3-yl)methoxy)benzaldehyde (0.09 g, 0.242 mmol), azetidine-3-carboxylic acid (0.029 g, 0.290 mmol) (Synchem), and acetic acid (0.055 mL, 0.968 mmol) were combined in methanol (2.421 mL) in a sealed vial to give a yellow solution. MP-cyanoborohydride (0.338 g, 0.726 mmol) (Argonaut) was added and the reaction stirred for about 18 h. LCMS shows product formation. Add several drops of concentrated ammonium hydroxide and 2-3 mL of methanol to solubilize the precipitate. The mixture was filtered through buchner funnel and washed through with methanol with added ammonium hydroxide. The solvents were removed under reduced pressure. The residue was purified by flash column chromatography (0.5″×6″ of silica) eluting with 1:1 EtOAc/(6:3:1 CHCl₃/MeOH/NH₄OH) and the product fractions combined. The solvents were removed under reduced pressure to a colorless oil that partially crystallized on standing. Crystallization was completed with addition of a few mL of methanol. The solvents were removed under reduced pressure. Add pentane to aid in transfer of solid from flask and collect by vacuum filtration. The resulting solid was collected by vacuum filtration and washed with pentane to provide 1-(4-((5-(3-chloro-4-isopropoxyphenyl)isoxazol-3-yl)methoxy)benzyl)azetidine-3-carboxylic acid (0.0859 g, 0.188 mmol, 78% yield) as a white solid on drying under vacuum at 60° C.: LCMS (Method g) Rₜ=1.98 min.; MS m/z: 457.11, 459.04 (M+H)⁺.

### Preparation of 1-(4-(4-(3-chloro-4-isopropoxyphenylcarbamoyl)-1H-1,2,3-triazol-1-yl)benzyl)azetidine-3-carboxylic acid

### Example #56

### Step A. Preparation of 2-(4-azidophenyl)-1,3-dioxolane

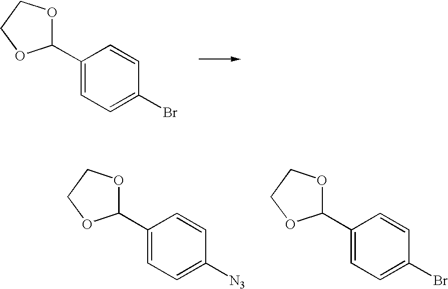

Obtained as a mixture 2-(4-bromophenyl)-1,3-dioxolane (5 g, 21.83 mmol) (synthesis reference: U.S. Pat. No. 5,157,149), sodium azide (1.537 mL, 43.7 mmol), and sodium ascorbate (0.649 g, 3.27 mmol) were combined in EtOH (30.6 mL) and water (13.10 mL) under nitrogen to give a yellow solution. Copper(I) iodide (0.416 g, 2.183 mmol) was added and the reaction stirred for about 16 h at reflux. The reaction was allowed to cool to room temperature. Methylene chloride (150 mL) and water (100 mL) were added and the layers separated. Extract with methylene chloride (3×50 mL). The combined extracts were washed with brine, dried over sodium sulfate, filtered, and evaporated to a yellow oil. NMR showed a 3:2 mixture of starting material to the azide. Used in next reaction as is.

### Step B. Preparation of ethyl 1-(4-(1,3-dioxolan-2-yl)phenyl)-1H-1,2,3-triazole-4-carboxylate

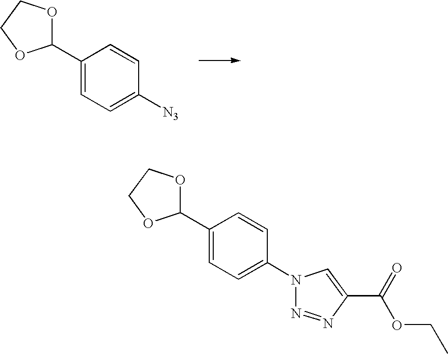

2-(4-azidophenyl)-1,3-dioxolane (1.9 g, 9.94 mmol) and propynoic acid ethyl ester (1.012 mL, 9.94 mmol) (Alfa Aesar) were combined in t-butanol (19.88 mL) and water (19.88 mL) open to the air to give a cloudy yellow solution. A solution of sodium ascorbate (0.994 mL, 0.994 mmol) was added in one portion and the reaction stirred for about 10 min. A solution of copper (II) sulfate pentahydrate (0.099 mL, 0.099 mmol) was added in one portion and the reaction stirred for about 16 h. Methylene chloride (50 mL) and water (25 mL) were added and the layers separated. Extract with methylene chloride (3×20 mL). The combined extracts were washed with brine, dried over sodium sulfate, filtered, and evaporated to a yellow oil. The residue was purified by flash column chromatography (80 g Redi-Sep) eluting with ethyl acetate/heptane and the product fractions combined. Remove solvent under reduced pressure to provide ethyl 1-(4-(1,3-dioxolan-2-yl)phenyl)-1H-1,2,3-triazole-4-carboxylate (0.348 g, 1.203 mmol, 12.10% yield) as a yellow solid. The resulting solid was stirred with ether, collected by vacuum filtration, and washed with ether to provide ethyl 1-(4-(1,3-dioxolan-2-yl)phenyl)-1H-1,2,3-triazole-4-carboxylate (0.348 g, 1.203 mmol, 12.10% yield) as a yellow solid: LCMS (Method a) Rₜ=2.49 min.; MS m/z: 290.16 (M+H)⁺.

### Step C. Preparation of 1-(4-(1,3-dioxolan-2-yl)phenyl)-1H-1,2,3-triazole-4-carboxylic acid

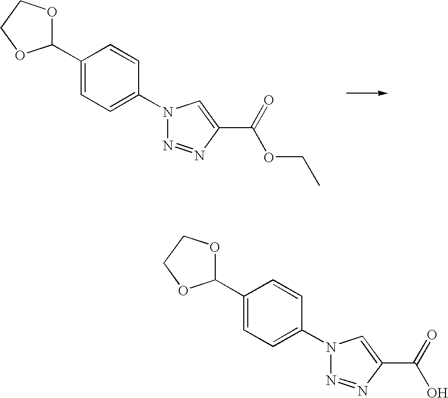

Ethyl 1-(4-(1,3-dioxolan-2-yl)phenyl)-1H-1,2,3-triazole-4-carboxylate (0.348 g, 1.203 mmol) was dissolved in EtOH (10.83 mL) and water (1.203 mL) open to the air to give a yellow solution. Potassium hydroxide (0.135 g, 2.406 mmol) was added and the reaction heated at about 95° C. for about 4 h. The reaction was allowed to cool to ambient temperature. Add conc. HCl until pH˜1. Ethyl acetate (100 mL) and water (25 mL) were added and the layers separated. Extract with ethyl acetate (2×25 mL). The combined extracts were washed with brine, dried over sodium sulfate, filtered, and evaporated to give 1-(4-(1,3-dioxolan-2-yl)phenyl)-1H-1,2,3-triazole-4-carboxylic acid (0.295 g, 1.129 mmol, 94% yield) as an orange solid: LCMS (Method a) Rₜ=1.46 min.; MS m/z: 262.18 (M+H)⁺.

### Step D. Preparation of 1-(4-(1,3-dioxolan-2-yl)phenyl)-N-(3-chloro-4-isopropoxyphenyl)-1H-1,2,3-triazole-4-carboxamide

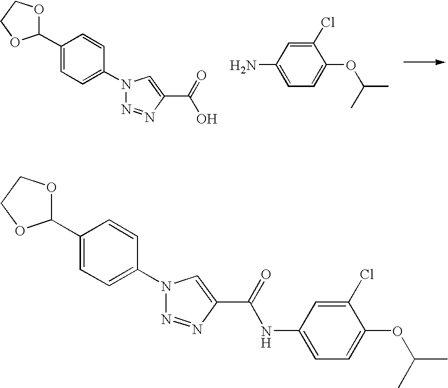

1-(4-(1,3-dioxolan-2-yl)phenyl)-1H-1,2,3-triazole-4-carboxylic acid (0.295 g, 1.129 mmol), 3-chloro-4-isopropoxyaniline (0.231 g, 1.242 mmol) (Matrix), and HATU (0.644 g, 1.694 mmol) were combined in DMF (11.29 mL) in a sealed vial to give a orange suspension. N,N-diisopropylethylamine (0.586 mL, 3.39 mmol) was added and the reaction stirred for about 20 h. Ethyl acetate (100 mL) and water (50 mL) were added and the layers separated. Extract with ethyl acetate (2×25 mL). The combined extracts were washed with 5% LiCl soln. (3×25 mL). The combined extracts were then washed with brine, dried over magnesium sulfate, filtered, and evaporated to a light brown oil. The residue was purified by flash column chromatography (40 g Redi-Sep) eluting with ethyl acetate/heptane and the product fractions combined. Remove solvent under reduced pressure to provide 1-(4-(1,3-dioxolan-2-yl)phenyl)-N-(3-chloro-4-isopropoxyphenyl)-1H-1,2,3-triazole-4-carboxamide (0.386 g, 0.900 mmol, 80% yield) as a tan solid: LCMS (Method a) Rₜ=3.08 min.; MS m/z: 429.17 (M+H)⁺.

### Step E. Preparation of N-(3-chloro-4 isopropoxyphenyl)-1-(4-formylphenyl)-1H-1,2,3-triazole-4-carboxamide

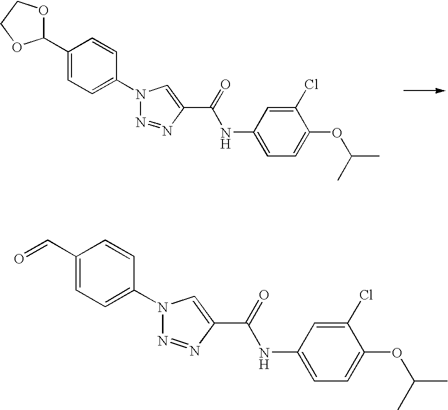

1-(4-(1,3-dioxolan-2-yl)-phenyl)-N-(3-chloro-4-isopropoxyphenyl)-1H-1,2,3-triazole-4-carboxamide (0.38 g, 0.886 mmol) was dissolved in acetone (18 mL) and equipped with a reflux condensor to give a colorless solution. A solution of HCl (2 mL, 2.000 mmol) was added in one portion and the reaction heated at about 65° C. for about 16 h. Remove solvents under reduced pressure—add some water. Collect solid by vacuum filtration and wash with water. Methylene chloride (25 mL) and brine (10 mL) were added and the layers separated. The combined extracts were washed with brine, dried over sodium sulfate, filtered, and evaporated to provide N-(3-chloro-4-isopropoxyphenyl)-1-(4-formylphenyl)-1H-1,2,3-triazole-4-carboxamide (0.299 g, 0.777 mmol, 88% yield) as a tan solid: LCMS (Method a) Rₜ=3.86 min.; MS m/z: 385.13 (M+H)⁺.

### Step F. Preparation of 1-(4-(4-(3-chloro-4-isopropoxyphenylcarbamoyl)-1H-1,2,3-triazol-1-yl)benzyl)azetidine-3-carboxylic acid

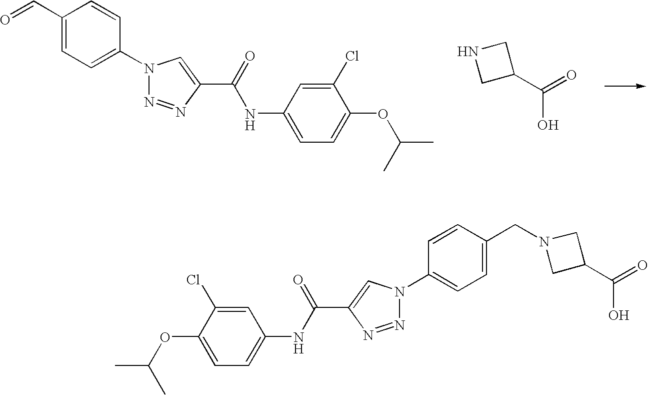

N-(3-chloro-4-isopropoxyphenyl)-1-(4-formylphenyl)-1H-1,2,3-triazole-4-carboxamide (0.299 g, 0.777 mmol), azetidine-3-carboxylic acid (0.079 g, 0.777 mmol) (Synchem), and MP-cyanoborohydride (1.084 g, 2.331 mmol) (Argonaut) were combined in methanol (15.54 mL) in a sealed vial to give a tan suspension. Acetic acid (0.178 mL, 3.11 mmol) was added and the reaction stirred for about 72 h. Filter through buchner funnel and wash through with a mixture of methanol and ammonium hydroxide. Concentrate and triturate with ether. Collect solid by vacuum filtration and wash with ether. Solid was dissolved in water with ammonium hydroxide. Filter through syringe filter. Rotovap to remove excess ammonium hydroxide—a heavy ppt. forms. The resulting solid was collected by vacuum filtration and washed with water to provide 1-(4-(4-(3-chloro-4-isopropoxyphenylcarbamoyl)-1H-1,2,3-triazol-1-yl)benzyl)azetidine-3-carboxylic acid (0.071 g, 0.151 mmol, 19.45% yield) as a white solid: LCMS (Method a) Rₜ=2.68 min.; MS m/z: 470.29, 472.22 (M+H)⁺.

### Preparation of methyl 1-(3-chloro-4-(5-(3-chloro-4-isopropoxyphenyl)-isoxazole-3-carboxamido)benzyl)azetidine-3-carboxylate

### Example #57

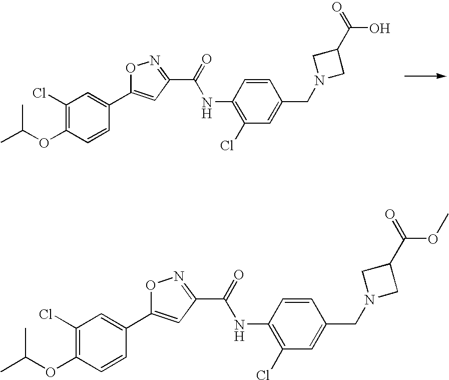

1-(3-chloro-4-(5-(3-chloro-4-isopropoxyphenyl)isoxazole-3-carboxamido)benzyl)azetidine-3-carboxylic acid (0.075 g, 0.149 mmol) and benzenesulfonic acid (0.0252 g, 0.159 mmol) were combined in acetonitrile (4 mL) in a sealed vial to give a white suspension. The mixture was heated but not everything dissolved. A heavy white precipitate formed. The mixture was allowed to cool to ambient temperature. The mixture was redissolved in methanol and additional benzene sulfonic acid added and the mixture reheated. The precipitate dissolved to give a solution. Remove methanol under vacuum. LCMS showed that the ester had formed. The residue was purified by flash column chromatography (0.5″×5″ of silica) eluting with EtOAc and the product fractions combined. The solid was triturated with heptane. The resulting solid was collected by vacuum filtration and washed with heptane to provide methyl 1-(3-chloro-4-(5-(3-chloro-4-isopropoxyphenyl)isoxazole-3-carboxamido)benzyl)azetidine-3-carboxylate (0.0255 g, 0.049 mmol, 33.1% yield) as a white solid: LCMS (Method d) Rₜ=1.97 min.; MS m/z: 518.17 (M+H)⁺.

### Preparation of 1-(4-(5-(3-chloro-4-isopropoxyphenyl)thiazole-2-carbox-amido)benzyl)azetidine-3-carboxylic acid

### Example #58

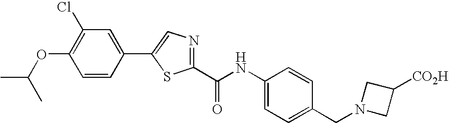

Prepared from 5-(3-chloro-4-isopropoxyphenyl)thiazole-2-carboxylic acid and 4-((tertbutyldimethylsilyloxy)methyl)aniline (Example #32, step B) following general procedures E, F, I, J.1, H to give 1-(4-(5-(3-chloro-4-isopropoxyphenyl)thiazole-2-carboxamido)benzyl)azetidine-3-carboxylic acid (65 mg, 78%) as a pale yellow solid: LCMS (Table 1, Method a) Rₜ=2.60 min.; MS m/z: 486.19 (M+H)⁺.

### Preparation of 3-(4-(5-(3-chloro-4-isopropoxyphenyl)thiazole-2-carbox-amido)benzylamino)-2-methylpropanoic acid

### Example #59

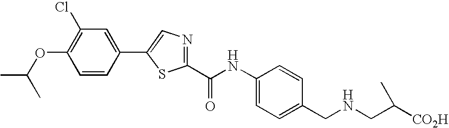

Prepared from 5-(3-chloro-4-isopropoxyphenyl)thiazole-2-carboxylic acid and 4-((tertbutyldimethylsilyloxy)methyl)aniline (Example #32, step B) following general procedures E, F, I, J.1, H to give 3-(4-(5-(3-chloro-4-isopropoxyphenyl)thiazole-2-carboxamido)benzylamino)-2-methylpropanoic acid (47 mg, 64%) as a white solid: LCMS (Table 1, Method g) Rₜ=2.08 min.; MS m/z: 490.08 (M+H)⁺.

### Preparation of N-(2-chloro-4-formylphenyl)-5-(3-chloro-4-isopropoxy-phenyl)isoxazole-3-carboxamide

### Example #60

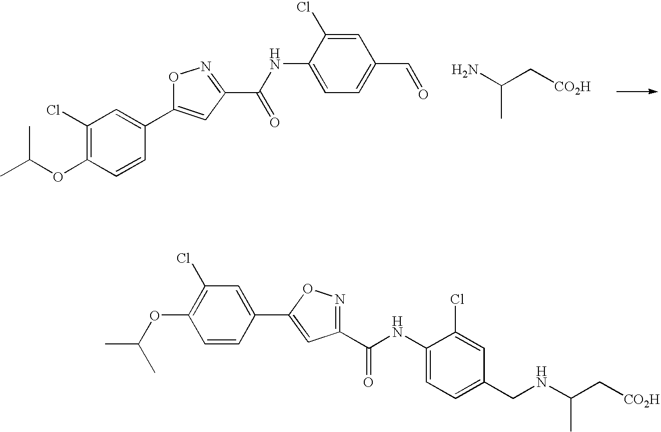

N-(2-chloro-4-formylphenyl)-5-(3-chloro-4-isopropoxyphenyl)isoxazole-3-carboxamide (1.28 g, 3.05 mmol) (Example #13, Step E) and 3-aminobutanoic acid (0.693 g, 6.72 mmol) were combined in methanol (28.0 mL) to give a yellow suspension. Acetic acid (0.874 mL, 15.27 mmol) was added dropwise, the reaction mixture was heated at 40° C. for 1 h. A solution of sodium cyanoborohydride (0.192 g, 3.05 mmol) in methanol (2 mL) was added dropwise over 3 min. The reaction was stirred at 40° C. overnight. The reaction mixture was a milky pale yellow suspension. The mixture was filtered without cooling and the filter cake was washed with cold methanol (90 mL) and then water (30 mL). The cake was stirred in water (100 mL), filtered, and was washed with water (100 mL), and then dried in a vacuum oven overnight to afford N-(2-chloro-4-formylphenyl)-5-(3-chloro-4-isopropoxyphenyl)isoxazole-3-carboxamide (1.28 g, 3.05 mmol) as a off-white solid: LCMS (table 1, Method a) Rₜ=2.88 min.; MS m/z: 506.22 (M+H)⁺. ¹H NMR (400 MHz, DMSO-d6) δ 10.3 (s, 1H), 8.08 (s, 1H), 7.89 (d, 1H), 7.68 (d, 1H), 7.61 (s, 1H), 7.48 (s, 1H), 7.4 (m, 2H), 4.84 (m, 1H), 3.85 (dd, 2H), 3.05 (m, 1H), 2.29 (d, 2H), 1.35 (d, 6H), 1.12 (d, 3H).

### Preparation of 1-(4-(1-tert-butyl-5-phenyl-1H-pyrazole-3-carboxamido)-3-chlorobenzyl)azetidine-3-carboxylic acid

### Example #61

### Step A. Preparation of 4-bromo-3-chlorobenzaldehyde

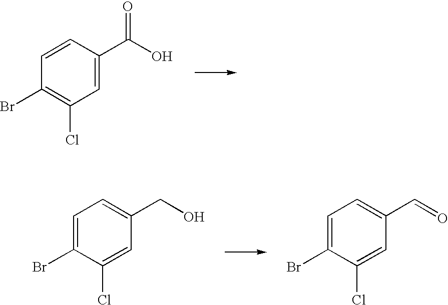

4-bromo-3-chlorobenzoic acid (5.0 g, 21.23 mmol) was dissolved in THF (25 mL) and cool to 0° C. Borane tetrahydrofuran complex (42.5 mL, 42.5 mmol) was added at such a rate as to maintain reaction temperature below 5° C. The reaction was allowed to warm to room temperature. The reaction was then heated at reflux for about 2 hours. The reaction was then cooled to 0° C. and quenched by the careful addition of methanol (50 mL). The solvents were removed under reduced pressure. The residue was dissolved in chloroform (90 mL) and washed once with saturated sodium bicarbonate and once with 2 N HCl. The organic layer was dried over sodium sulfate, filtered, and the filter cake rinsed with chloroform (10 mL). The product was used without further purification.

Crude (4-bromo-3-chlorophenyl)methanol (4.70 g, 21.22 mmol) was dissolved in chloroform (100 mL). Manganese dioxide (5.53 g, 63.7 mmol) was added and the mixture heated at reflux and then heated overnight at 60° C. The reaction was cooled to room temperature and filtered through celite that was rinsed with chloroform (2×20 mL). The solvents were removed under vacuum to give an oil which solidified on standing. The solid was purified via flash column chromatography (9:1 heptane/ethyl acetate). The product fractions were combined, solvent removed, and dried under vacuum to give 4-bromo-3-chlorobenzaldehyde (3.0 g, 64%) as a white solid: ¹H NMR (400 MHz, DMSO) ppm 9.99 (s, 1H), 8.12 (d, J=1.90 Hz, 1H), 8.04 (d, J=8.20 Hz, 1H), 7.78 (dd, J=8.20, 1.92 Hz, 1H).

### Step B. Preparation of lithium (Z)-1-ethoxy-1,4-dioxo-4-phenylbut-2-en-2-olate

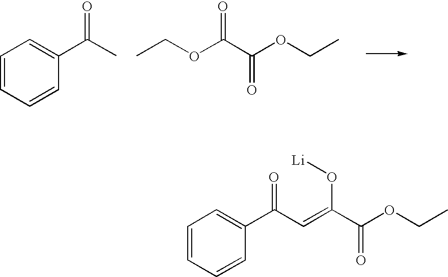

A solution of lithium bis(trimethylsilyl)amide (5.73 g, 34.2 mmol) in Et₂O (165 mL) was cooled to −78° C. under nitrogen. Acetophenone (5.00 g, 41.6 mmol) in Et₂O (33 mL) was added 30 dropwise while maintaining the reaction temperature below −75° C. The reaction was stirred for 30 minutes at −78° C. and then diethyl oxalate (5.00 g, 34.2 mmol) was added in one portion. The reaction was allowed to warm to room temperature and the solution became turbid and solid product began to form. The reaction was stirred for 4 hours at room temperature. The resulting precipitate was collected by vacuum filtration and washed with ether (3×40 mL) to provide lithium (Z)-1-ethoxy-1,4-dioxo-4-phenylbut-2-en-2-olate (7.1 g, 92%) as a pale yellow product on drying under vacuum: ¹H NMR (400 MHz, DMSO) ppm 7.83 (d, J=6.50 Hz, 2H), 7.49-7.39 (m, 3H), 6.43 (s, 1H), 4.15 (q, J=7.10 Hz, 2H), 1.26 (t, J=7.10 Hz, 3H).

### Step C. Preparation of ethyl 1-tert-butyl-5-phenyl-1H-pyrazole-3-carboxylate

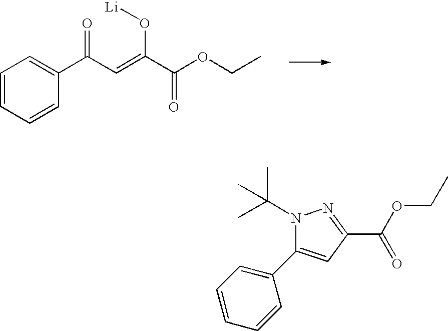

Lithium (Z)-1-ethoxy-1,4-dioxo-4-phenylbut-2-en-2-olate (3.72 g, 16.45 mmol) was dissolved in of ethanol (93 mL) while tert-butylhydrazine hydrochloride (2.050 g, 16.45 mmol) was dissolved in of ethanol (56 mL) and of DMF (15 mL). The solutions were combined and stirred for 48 h. The solution was placed in an oil bath heated to 70° C. overnight. The solvents were removed under vacuum and the residue was dissolved in methylene chloride. This organic layer was then washed twice with water and then diluted with heptane. The methylene chloride was removed under vacuum and crystals began to appear. The precipitate was collected by vacuum filtration and washed with heptane to provide a white solid. A second crop was obtained in the same way from the filtrate. The solids were combined, purified by flash chromatography, and the fractions evaporated to give ethyl 1-tert-butyl-5-phenyl-1H-pyrazole-3-carboxylate (2.3 g, 51%) as a solid: LCMS (table 1, Method a) Rₜ=3.62 min.; MS m/z: 273.76 (M+H)⁺; ¹H NMR (400 MHz, DMSO) δ ppm 7.55-7.37 (m, 5H), 6.61 (s, 1H), 4.28 (q, J=7.08 Hz, 2H), 1.41 (s, 9H), 1.29 (t, J=7.07 Hz, 3H).

### Step D. Preparation of 1-tert-butyl-5-phenyl-1H-pyrazole-3-carboxamide

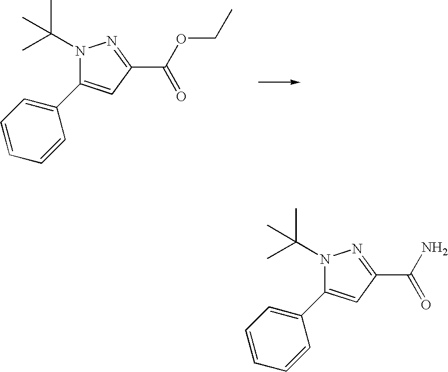

To ethyl 1-tert-butyl-5-phenyl-1H-pyrazole-3-carboxylate (4.00 g, 14.69 mmol) was added methanolic ammonia (100 mL, 700 mmol) in a Parr reactor. The reaction was sealed and heated at 100° C. for about 24 hours. The reaction was cooled on ice and the resulting precipitate was collected by vacuum filtration to provide 1-tert-butyl-5-phenyl-1H-pyrazole-3-carboxamide (3.10 g, 87%) as a white solid: LCMS (table 1, Method a) Rₜ=2.95 min; ¹H NMR (400 MHz, DMSO) δ ppm 7.52-7.35 (m, 6H), 7.24-7.19 (m, 1H), 6.49 (s, 1H), 1.41 (m, 9H).

### Step E. Preparation of 1-tert-butyl-N-(2-chloro-4-formylphenyl)-5-phenyl-1H-pyrazole-3-carboxamide

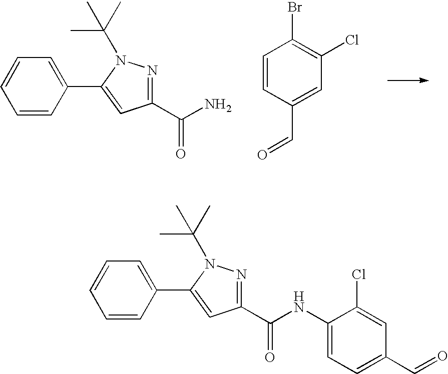

4-Bromo-3-chlorobenzaldehyde (0.595 g, 2.71 mmol), 1-tert-butyl-5-phenyl-1H-pyrazole-3-carboxamide (0.6 g, 2.466 mmol), cesium carbonate (0.964 g, 2.96 mmol), xantphos (0.086 g, 0.148 mmol), and tris(dibenzylideneacetone)dipalladium(0) (0.045 g, 0.049 mmol) were combined neat under nitrogen and then dilute with dioxane (5.14 mL). The mixture was degassed with a stream of nitrogen for about 5 minutes then heated to 100° C. overnight. The reaction was cooled and THF (15 mL) was added, the mixture was filtered through Celite, the Celite washed with THF (2×15 mL). The filtrate was concentrated to afford 0.9 g of a red oil, which was triturated with ACN (5 mL) and ether and then filtered. The solid was washed by ACN (2×5 mL) and dried to afford 1-tert-butyl-N-(2-chloro-4-formylphenyl)-5-phenyl-1H-pyrazole-3-carboxamide (0.5 g, 1.309 mmol, 53.1% yield) as a pale yellow solid: LCMS (table 1, Method a) Rₜ=3.95 min.; MS m/z: 382.16 (M+H)⁺. ¹H NMR (400 MHz, DMSO) δ ppm 9.93 (s, 1H), 9.84 (s, 1H), 8.59 (d, J=8.45 Hz, 1H), 8.11 (s, 1H), 7.96 (d, J=8.44 Hz, 1H), 7.56-7.42 (m, 5H), 6.76 (s, 1H), 1.52-1.44 (m, 9H).

### Step F. Preparation of 1-(4-(1-tert-butyl-5-phenyl-1H-pyrazole-3-carboxamido)-3-chlorobenzyl)azetidine-3-carboxylic acid

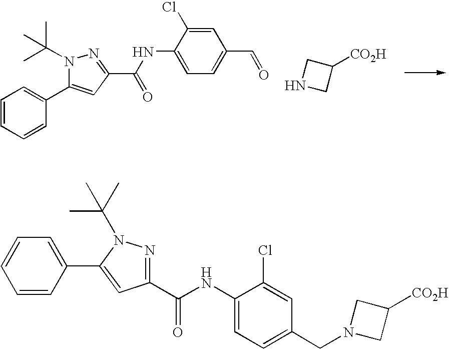

A 40 mL reaction vial equipped with rubber septum and nitrogen inlet needle was charged with 1-tert-butyl-N-(2-chloro-4-formylphenyl)-5-phenyl-1H-pyrazole-3-carboxamide (0.5 g, 1.309 mmol) and azetidine-3-carboxylic acid (0.159 g, 1.571 mmol) in methanol (12 mL) to give a white suspension. The reaction mixture was heated at 40° C. Acetic acid (0.375 mL, 6.55 mmol) was added dropwise and the reaction stirred for 6 h. Sodium cyanoborohydride (0.082 g, 1.309 mmol) was added in one portion. The reaction mixture was stirred over the weekend at 40° C. to give a colorless suspension. The precipitate was collected by vacuum filtration, washed with cold methanol (2×3 mL) and water (5×10 mL), and then dried in a vacuum oven (55° C.) overnight to afford 1-(4-(1-tert-butyl-5-phenyl-1H-pyrazole-3-carboxamido)-3-chlorobenzyl)azetidine-3-carboxylic acid (0.444 g, 0.951 mmol, 72.6% yield) as a white solid: LCMS (table 1, Method a) Rₜ=2.97 min.; MS m/z: 467.29 (M+H)⁺, ¹H NMR (400 MHz, DMSO) ppm 9.55 (s, 1H), 8.12 (d, J=8.30 Hz, 1H), 7.56-7.41 (m, 6H), 7.28 (dd, J=8.39, 1.27 Hz, 1H), 6.69 (s, 1H), 3.54 (s, 2H), 3.40 (s, J=3.96 Hz, 3H), 3.21 (s, J=2.99 Hz, 4H), 1.46 (s, 9H).

What is claimed is:

1. A compound of Formula (I) 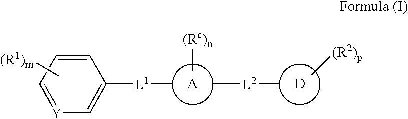
and pharmaceutically acceptable salts, isomers, prodrugs and biologically active metabolites thereof wherein
Y is N is or CH;
A is selected from the group consisting of optionally substituted heteroaryl, optionally 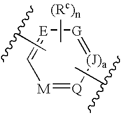
substituted heterocyclyl and
wherein
a is 0 or 1 and E, G, J, Q and M are each independently selected from the group consisting of CRₐ, O, N and S provided that at least one of E, G, J, Q and M is CRₐ;
no more than one of E, G, J, Q and M is O; and
no more than one of E, G, J, Q and M is S;
L¹ and L² are each independently selected from the group consisting of a bond, —C(O)NH—, —NHC(O)—, —SO₂NH—, —NHSO₂—, —CH₂N(H)—, —N(H)CH₂—, —CH₂S— and —SCH₂—, provided that either L¹ or L² is a bond but L¹ and L² are not bonds at the same time;
D is selected from the group consisting of aryl, heteroaryl, heterocyclyl and (C₃-C₉)cycloalkyl;
R¹ and R² are each independently selected from the group consisting of halogen, CF₃, CN, OH, OCF₃, optionally substituted (C₁-C₆)alkyl, —C(O)—O—(C₁-C₆)alkyl, NR^{a}R^{b}, —(CH₂)ₓ-optionally substituted aryl, —(CH₂)ₓ-optionally substituted (C₃-C₆)cyclyl, —(CH₂)ₓ-optionally substituted heteroaryl, —(CH₂)ₓ-optionally substituted heterocyclyl, —NR^{a}-optionally substituted (C₃-C₆)cycloalkyl, —O-optionally substituted (C₁-C₆)alkyl, —O-optionally substituted (C₃-C₆)cycloalkyl, —O-heterocyclyl —O-aryl, —O-heteroaryl, —NR^{a}-optionally substituted heteraryl, —NR^{a}-optionally substituted aryl, SO₂NR^{a}R^{b} and CH₂NR^{a}NR provided that R¹ and R² are not both —(CH₂)ₓ-optionally substituted heterocyclyl or —(CH₂)ₓ-optionally substituted heteroaryl at the same time;
R^{a} and R^{b} are each independently selected from H and optionally substituted (C₁-C₆)alkyl;
R^{c} is independently selected from the group consisting of CF₃, CCl₃, optionally substituted (C₁-C₆)alkyl, —C(O)-optionally substituted (C₁-C₆)alkyl, —C(O)—O-optionally substituted (C₁-C₆)alkyl and oxo;
m is 0, 1 or 2;
n is 0, 1 or 2;
p is 0, 1 or 2; and
x is 0, 1 or 2;
provided that the compound is not

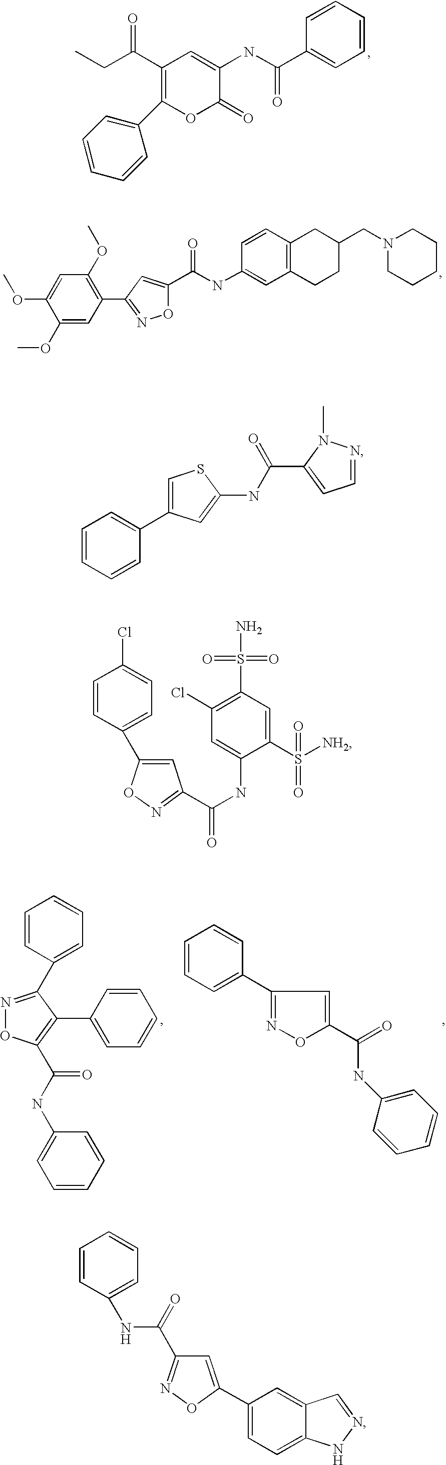

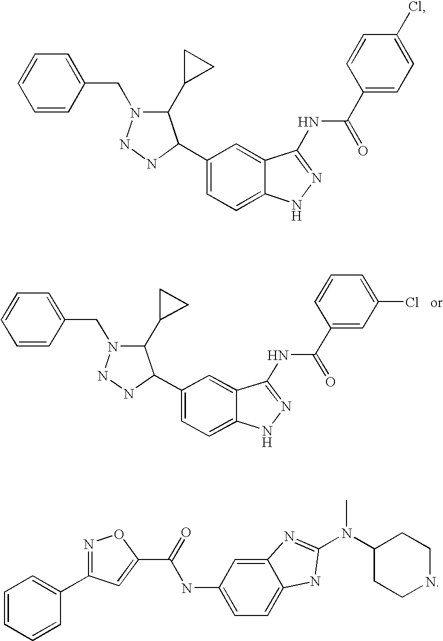

2. The compound according to claim 1, wherein A is

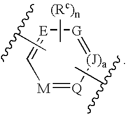

3. The compound according to claim 2, wherein A is selected from the optionally substituted group consisting of furanyl, imidazolyl, isoxazolyl, oxadiazolyl, oxazolyl, pyranyl, pyrazolyl, pyrrolyl, thiazolyl, thienyl and 1H-[1,2,4]triazolyl.

4. The compound according to claim 3, wherein D is selected from the group consisting of benzofuranyl, indanyl, indazolyl, indolyl, 2,3-dihydro-1H-indolyl, oxadiazolyl, phenyl, pyrazolyl, pyridinyl, tetrahydroisoquinolinyl, tetrahydroquinolinyl, thienyl and

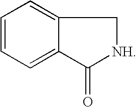

5. The compound according to claim 4, wherein R¹ and R² are independently selected from the group consisting of Br, C₁, CF₃, CN, OH, OCF₃, CH₃, —CH(CH₃)₂, OCH₃, OCH(CH₃)₂, —C(O)OCH₂CH₃, -optionally substituted (C₁-C₆)alkyl, —NR^{a}-optionally substituted (C₁-C₆)alkyl, N(CH₃)₂, —(CH₂)ₓ-optionally substituted aryl, —(CH₂)ₓ-optionally substituted (C₃-C₆)cycloalkyl, —(CH₂)ₓ-optionally substituted heteroaryl, morpholinyl, —(CH₂)ₓ-optionally substituted azetidinyl, —(CH₂)ₓ-optionally substituted pyrrolidinyl, —(CH₂)ₓ-optionally substituted piperidinyl, —NH-optionally substituted (C₃-C₆)alkyl, NH-optionally substituted (C₃-C₆)cycloalkyl, —O-optionally substituted (C₃-C₆)cycloalkyl and —O-tetrahydrofuranyl.

6. The compound according to claim 5, wherein A is selected from the optionally substituted group consisting of isoxazolyl, oxadiazolyl, oxazolyl, pyranyl, pyrazolyl, thienyl and 1H-[1,2,4]triazolyl.

7. The compound according to claim 6, wherein Y is CH.

8. The compound according to claim 7, wherein L¹ and L² are selected from the group consisting of a bond, —C(O)NH—, —NHC(O)—, SO₂NH— and —NHSO₂—.

9. The compound according to claim 8, wherein D is selected from the group consisting of indanyl, indazolyl, phenyl, pyrazolyl, tetrahydroisoquinolinyl, tetrahydroquinolinyl and

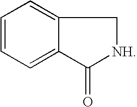

10. The compound according to claim 9, wherein R¹ and R² are each independently selected from the group consisting of C₁, CF₃, CH₃, OCF₃, OCH₃, OCH(CH₃)₂, —C(O)OCH₂CH₃, N(CH₃)₂, morpholinyl, —(CH₂)-optionally substituted azetidinyl, —(CH₂)-optionally substituted pyrrolidinyl, —(CH₂)-optionally substituted piperidinyl, NH-optionally substituted (C₃-C₆)cycloalkyl and —O-tetrahydrofuranyl.

11. The compound according to claim 10, wherein D is selected from the group consisting of indanyl, phenyl, pyrazolyl, tetrahydroisoquinolinyl and tetrahydroquinolinyl.

12. The compound according to claim 11, wherein A is selected from the optionally substituted group consisting of isoxazolyl, oxadiazolyl, oxazolyl, pyranyl, pyrazolyl, pyrrolyl, thiazolyl, thienyl and 1H-[1,2,4]triazolyl.

13. The compound according to claim 12, wherein L¹ and L² are selected from the group consisting of a bond, —C(O)NH— and —NHC(O)—.

14. The compound according to claim 13, wherein R¹ and R² are each independently selected from the group consisting of Cl, CF₃, CH₃, OCF₃, OCH₃, OCH(CH₃)₂, —C(O)OCH₂CH₃, N(CH₃)₂, —(CH₂)-optionally substituted azetidinyl, —(CH₂)-optionally substituted pyrrolidinyl, —(CH₂)-optionally substituted piperidinyl, and NH-optionally substituted (C₃-C₆)cycloalkyl.

15. The compound according to claim 14, wherein A is selected from the optionally substituted group consisting of isoxazolyl, oxadiazolyl, pyranyl, pyrazolyl, thienyl and 1H-[1,2,4]triazolyl.

16. The compound according to claim 15, wherein D is selected from the group consisting of indanyl, phenyl and pyrazolyl.

17. The compound according to claim 16, wherein each R^{c} is independently selected from the group consisting of CF₃, CCl₃, t-butyl, —C(O)—OCH₂CH₃, —C(O)—OCH₂CH₂CH₃, and oxo.

18. The compound according to claim 17 and pharmaceutically acceptable salts thereof wherein A is selected from the optionally substituted group consisting of isoxazolyl, pyrazolyl, thienyl and 1H-[1,2,4]triazolyl.

19. The compound according to claim 18, wherein each R^{c} is independently selected from the group consisting of CF₃, CCl₃, —C(O)—OCH₂CH₃, —C(O)—OCH₂CH₂CH₃ and oxo.

20. A method of treating a condition in a patient comprising administering a therapeutically effective amount of a compound of claim 1 or a physiologically acceptable salt thereof to said patient, wherein said condition is selected from the group consisting of rheumatoid arthritis, osteoarthritis, asthma, chronic obstructive pulmonary disease, sepsis, psoriasis, psoriatic arthritis, inflammatory bowel disease, Crohn's disease, lupus, multiple sclerosis, juvenile chronic arthritis, Lyme arthritis, reactive arthritis, septic arthritis, spondyloarthropathy, systemic lupus erythematosus, an ocular condition, a cancer, a solid tumor, fibrosarcoma, osteoma, melanoma, retinoblastoma, a rhabdomyosarcoma, glioblastoma, neuroblastoma, teratocarcinoma, an cancers such as lung, breast, stomach, bladder, colon, pancreas, ovarian, prostate and rectal cancer and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, alpha-1 antitrypsin deficiency, amyotrophic lateral sclerosis, anemia, angina pectoris, anterior horn cell degeneration, anti cd3 therapy, antiphospholipid syndrome, anti-receptor hypersensitivity reactions, hypersensitivity reactions, hyperkinetic movement disorders, hypersensitivity pneumonitis, hypertension, hypokinetic movement disorders, aortic and peripheral aneurysms, hypothalamic-pituitary-adrenal axis evaluation, aortic dissection, arterial hypertension, arteriosclerosis, arteriovenous fistula, ataxia, spinocerebellar degenerations, streptococcal myositis, structural lesions of the cerebellum, Subacute sclerosing panencephalitis, Syncope, syphilis of the cardiovascular system, systemic anaphylaxis, systemic inflammatory response syndrome, systemic onset juvenile rheumatoid arthritis, T-cell or FAB ALL, Telangiectasia, thromboangitis obliterans, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, atrial fibrillation (sustained or paroxysmal), atrial flutter, atrioventricular block, B cell lymphoma, bone graft rejection, bone marrow transplant (BMT) rejection, small bowel transplant rejection, spinal ataxia, bundle branch block, Burkitt's lymphoma, burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia, chronic alcoholism, chronic inflammatory pathologies, chronic lymphocytic leukemia, chronic salicylate intoxication, colorectal carcinoma, congestive heart failure, conjunctivitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetic ateriosclerotic disease, Diffuses Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, Epstein Barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, gram negative sepsis, gram positive sepsis, granulomas due to intracellular organisms, hairy cell leukemia, Hallerrorden-Spatz disease, hay fever, heart transplant rejection, hemachromatosis, hemodialysis, hemolytic uremic syndrome/thrombolytic thrombocytopenic purpura, hemorrhage, idiopathic pulmonary fibrosis, antibody mediated cytotoxicity, Asthenia, infantile spinal muscular atrophy, inflammation of the aorta, influenza A, ionizing radiation exposure, iridocyclitis/uveitis/optic neuritis, juvenile rheumatoid arthritis, juvenile spinal muscular atrophy, kidney transplant rejection, legionella, leishmaniasis, lipedema, liver transplant rejection, lymphederma, malaria, malignant Lymphoma, malignant histiocytosis, malignant melanoma, meningococcemia, metabolic/idiopathic, migraine headache, mitochondrial multi-system disorder, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, Kaposi's sarcoma, Hodgkin's disease, lymphoma, myeloma, leukaemia, malignant ascites, hematopoietic cancers, Crow-Fukase (POEMS) syndrome (polyneuropathy, organomegaly, endocrinopathy, monoclonal gammopathy, and skin changes syndrome), a diabetic condition such as insulin-dependent diabetes mellitus glaucoma, diabetic retinopathy or microangiopathy, sickle cell anaemia, chronic inflammation, synovitis, glomerulonephritis, graft rejection, Lyme disease, von Hippel Lindau disease, pemphigoid, Paget's disease, fibrosis, sarcoidosis, cirrhosis, thyroiditis, hyperviscosity syndrome, Osler-Weber-Rendu disease, chronic occlusive pulmonary disease, asthma or edema following burns, trauma, radiation, stroke, hypoxia, ischemia, ovarian hyperstimulation syndrome, post perfusion syndrome, post pump syndrome, post-MI cardiotomy syndrome, preeclampsia, menometrorrhagia, endometriosis, pulmonary hypertension, infantile hemangioma, or infection by Herpes simplex, Herpes Zoster, human immunodeficiency virus, parapoxvirus, protozoa or toxoplasmosis, Progressive supranucleo Palsy, primary pulmonary hypertension, radiation therapy, Raynaud's phenomenon and disease, Refsum's disease, regular narrow QRS tachycardia, renovascular hypertension, restrictive cardiomyopathy, sarcoma, senile chorea, Senile Dementia of Lewy body type, shock, skin allograft, skin changes syndrome, ocular or macular edema, ocular neovascular disease, scleritis, radial keratotomy, uveitis, vitritis, myopia, optic pits, chronic retinal detachment, post-laser treatment complications, conjunctivitis, Stargardt's disease, Eales disease, retinopathy, macular degeneration, restenosis, ischemia/reperfusion injury, ischemic stroke, vascular occlusion, carotid obstructive disease, ulcerative colitis, inflammatory bowel disease, diabetes, diabetes mellitus, insulin dependent diabetes mellitus, allergic diseases, dermatitis scleroderma, graft versus host disease, organ transplant rejection (including but not limited to bone marrow and solid organ rejection), acute or chronic immune disease associated with organ transplantation, sarcoidosis, disseminated intravascular coagulation, Kawasaki's disease, nephrotic syndrome, chronic fatigue syndrome, Wegener's granulomatosis, Henoch-Schoenlein purpurea, microscopic vasculitis of the kidneys, chronic active hepatitis, septic shock, toxic shock syndrome, sepsis syndrome, cachexia, infectious diseases, parasitic diseases, acquired immunodeficiency syndrome, acute transverse myelitis, Huntington's chorea, stroke, primary biliary cirrhosis, hemolytic anemia, malignancies, Addison's disease, idiopathic Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, peripheral vascular disorders, peritonitis, pernicious anemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis A, Hepatitis B, Hepatitis C, His bundle arrythmias, HIV infection/HIV neuropathy, common varied immunodeficiency (common variable hypogammaglobulinaemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, pneumocystis carinii pneumonia, pneumonia, connective tissue disease associated interstitial lung disease, mixed connective tissue disease, associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, acute and chronic pain (different forms of pain), Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, toxicity, transplants, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci infection, mental disorders (e.g., depression and schizophrenia), Th2 Type and Th1 Type mediated diseases, and diseases involving inappropriate vascularization, e.g., diabetic retinopathy, retinopathy of prematurity, choroidal neovascularization due to age-related macular degeneration, and infantile hemangiomas in human beings. In addition, such compounds may be useful in the treatment of disorders such as ascites, effusions, and exudates, including, e.g., macular edema, cerebral edema, acute lung injury, adult respiratory distress syndrome, proliferative disorders such as restenosis, fibrotic disorders such as hepatic cirrhosis and atherosclerosis, mesangial cell proliferative disorders such as diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, preeclampsia, menometrorrhagia, cat scratch fever, rubeosis, neovascular glaucoma and retinopathies such as those associated with diabetic retinopathy, retinopathy of prematurity, age-related macular degeneration, acute idiopathic polyneuritis, acuter or chronic immune disease associated with organ transplantation, acute inflammatory demyelinating polyradiculoneuropathy, acute ischemia, adult Still's disease, allergy, anaphylaxis, anti-phospholipid antibody syndrome, aplastic anemia, atopic eczema, atopic dermatitis, autoimmune dermatitis, autoimmune diabetes, autoimmune disorder associated with streptococcus infection, autoimmune enteropathy, autoimmune hepatitis, autoimmune hearing loss, autoimmune lymphoproliferative syndrome, autoimmune myocarditis, autoimmune neutropenia, autoimmune premature ovarian failure, autoimmune thrombocytopenia, autoimmune uveitis, Behcet's disease, blepharitis, bronchiectasis, bullous pemphigoid, catastrophic antiphospholipid syndrome, celiac disease, cervical spondylosis, chronic ischemia, cicatricial pemphigoid, clinical isolated syndrome with risk for multiple sclerosis, childhood onset psychiatric disorder, dacrocystitis, dermatomyositis, disc herniation, disc prolapse, drug induced immune hemolytic anemia, endophthalmitis, episcleritis, erythema multiforme, erythema multiforme major, gestational pemphigoid, Guillain-Barre syndrome, heart failure, Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF/UIP, iritis, keratitis, keratojuntivitis sicca, Kussmaul disease or Kussmaul-Meier disease, Landry's paralysis, Langerhan's cell hisiocytosis, livedo reticularis, microscopic polyangiitis, morbus bechterev, motor neuron disorders, mucous membrane pemphigoid, primary progressive multiple sclerosis, secondary progressive multiple sclerosis, relapsing remitting multiple sclerosis, multiple organ failure, myelodysplastic syndrome, nerve root disorder, neuropathy, Non-A Non-B hepatitis, osteolysis, ovarian cancer, pauciarticular JRA, peripheral artery occlusive disease (PAOD), periphral vascular disease (PVD), peripheral artery disease, phlebitis, polychondritis, polymyalgia rheumatica, poliosis, polyarticular JRA, polyendocrine deficiency syndrome, polymyositis, post-pump syndrome, primary parkinsonism, prostatitis, psoratic arthropathy, pure red cell aplasia, primary adrenal insufficiency, Reiter's disease, recurrent neuromyelitis optica, rheumatic heart disease, SAPHO (synovitis, acne, pustulosis, hyperostosis, and osteitis), scleroderma, secondary amyloidosis, shock lung, sciatica, secondary adrenal insufficiency, septic arthritis, seronegative arthopathy, silicone associated connective tissue disease, Sneddon-Wilkinson Dermatosis, spondilitis ankylosans, Stevens-Johnson Syndrome, systemic inflammatory response syndrome, temporal arteritis, toxoplasmic retinitis, toxic epidermal necrolysis, TRAPS (Tumor Necrosis factor receptor), type 1 allergic reaction, type II diabetes, urticaria, usual interstitial pneumonia, vernal conjunctivitis, viral retinitis, Vogt-Koyanagi-Harada syndrome (VKH syndrome) and wet macular degeneration.
